(19) United States
(12) Patent Application Publication
(10) Pub. No.: US 2022/0151977 A1
(43) Pub. Date: May 19, 2022

(54) Generation Of Water-Soluble Cannabinoids Utilizing Protein Cannabinoid-Carriers

(71) Applicant: Trait Biosciences, Inc., Los Alamos, NM (US)

(72) Inventors: Jennifer Stamps, Los Alamos, NM (US); Elton Carvalho Goncalves, Los Alamos, NM (US); Richard T. Sayre, Los Alamos, NM (US); Tawanda Zidenga, White Rock, NM (US); Erick Scott LeBrun, White Rock, NM (US); Timothy Travers, Los Alamos, NM (US)

(21) Appl. No.: 17/428,581

(22) PCT Filed: Feb. 4, 2020
(86) PCT No.: PCT/US20/16672

§ 371 Date: Aug. 4, 2021

Related U.S. Application Data
(60) Provisional application No. 62/800,708, filed on Feb. 4, 2019.
(60) Provisional application No. 62/810,435, filed on Feb. 26, 2019.

(51) Int. Cl. A61K 31/352; A61K 47/64; A61K 31/05

(52) U.S. Cl. CPC A61K 31/352; A61K 31/05; A61K 47/64

(57) ABSTRACT

The inventive technology includes novel systems, methods, and compositions for the generation of water-soluble short-chain fatty acid phenolic compounds, preferably cannabinoids, terpenes, and other volatile compounds produced in Cannabis. In particular, the inventive technology includes novel systems, methods, and compositions to solubilize short-chain fatty acid phenolic coin-pounds, such as cannabinoids, via binding to a water soluble and readily digested carrier protein such as: lipocalins, lipocalin-like, odorant-binding proteins, and odorant-binding-like proteins.

This International PCT Application claims the benefit of and priority to U.S. Provisional Application No. 62/800,708, filed Feb. 4, 2019, and U.S. Provisional Application No. 62/810,435, filed Feb. 26, 2019. The entire specification and figures of the above-referenced applications are hereby incorporated, in their entirety by reference.

### TECHNICAL FIELD

The inventive technology includes novel systems, methods, and compositions for the generation of water-soluble short-chain fatty acid phenolic compounds, preferably cannabinoids, terpenes, and other volatile compounds produced in Cannabis. In particular, the inventive technology includes novel systems, methods, and compositions to solubilize short-chain fatty acid phenolic compounds, such as cannabinoids, via binding to a water soluble and readily digested carrier protein such as: lipocalins, lipocalin-like, odorant-binding proteins, and odorant-binding-like proteins.

### BACKGROUND OF THE INVENTION

Cannabinoids are a class of specialized compounds synthesized by Cannabis. They are formed by condensation of terpene and phenol precursors. They include these more abundant forms: Δ⁹-tetrahydrocannabinol (THC), cannabidiol (CBD), cannabichromene (CBC), and cannabigerol (CBG). Another cannabinoid, cannabinol (CBN), is formed from THC as a degradation product and can be detected in some plant strains. Typically, THC, CBD, CBC, and CBG occur together in different ratios in the various plant strains. These cannabinoids are generally lipophilic, nitrogen-free, mostly phenolic compounds and are derived biogenetically from a monoterpene and phenol, the acid cannabinoids from a monoterpene and phenol carboxylic acid, and have a C21 base. Cannabinoids also find their corresponding carboxylic acids in plant products. In general, the carboxylic acids have the function of a biosynthetic precursor. For example, the tetrahydrocannabinols Δ⁹- and Δ⁸-THC arise in vivo from the THC carboxylic acids by decarboxylation and likewise, CBD from the associated cannabidiolic acid.

Importantly, cannabinoids are hydrophobic small molecules and, as a result, are highly insoluble. Due to this insolubility, cannabinoids such as THC and CBD may need to be efficiently solubilized to facilitate transport, storage, and adsorption through certain tissues and organs. As described in, U.S. Pat. No. 8,410,064 by Pandya et al., cannabinoids may be subject to cytochrome P450 oxidation and subsequent UDP-glucuronosyltransferase (UGT)-dependent glucuronidation in the body after consumption. The resulting glucuronide of the oxidized cannabinoids is the main metabolite found in urine, and thus, this solubilization process plays a critical role in the metabolic clearance of cannabinoids. In another embodiment outlined in PCT/US18/24409 and PCT/US18/41710 (both of which are incorporated herein in their entirety by reference), by Sayre et al., cannabinoids may be glycosylated in vivo to form water-soluble glycoside compounds.

As outlined below, cannabinoids may be solubilized by binding to certain carrier proteins. For example, cannabinoids, and other short-chain fatty acid phenolic compounds, may be transported in biological fluids (such as blood) and tissues (including the intracellular milieu) by these so-called carrier proteins. Generally, the binding to these carrier proteins molecules effectively increases the water-solubility of fatty acids and other lipophilic molecules, thereby facilitating their transport through aqueous environments as well as their transfer across cellular membranes. Human and homologous non-human carrier proteins may offer an opportunity for use in the solubilization of cannabinoids among other compounds. One area where water-soluble cannabinoids has seen renewed interest is in the fields of cannabinoid-infused consumer products. However, the ability to effectively solubilize cannabinoids has limited their applicability. To overcome these limitations, many manufacturers of cannabinoid-infused products have adopted the use of traditional pharmaceutical delivery methods of using nanoemulsions of cannabinoids. This nanoemulsion process essentially coats the cannabinoid in a hydrophilic compound, such as oil or other similar compositions. However, the use of nanoemulsions is limited both technically, and from a safety perspective.

First, a large number of surfactants and cosurfactants are required for nanoemulsion stabilization. Moreover, the stability of nanoemulsions is inherently unstable, and may be disturbed by slight fluctuations in temperature and pH, and is further subject to the “oswald ripening effect” or ORE. ORE describes the process whereby molecules on the surface of particles are more energetically unstable than those within. Therefore, the unstable surface molecules often go into solution shrinking the particle over time and increasing the number of free molecules in solution. When the solution is supersaturated with the molecules of the shrinking particles, those free molecules will redeposit on the larger particles. Thus, small particles decrease in size until they disappear and large particles grow even larger. This shrinking and growing of particles will result in a larger mean diameter of a particle size distribution (PSD). Over time, this causes emulsion instability and eventually phase separation.

Second, nanoemulsions may not be safe for human consumption. For example, nanoemulsions were first developed as a method to deliver small quantities of pharmaceutical compounds having poor solubility. However, the ability to “hide” a compound, such as a cannabinoid, in a nanoemulsion may allow the cannabinoid to be delivered to parts of the body where it was previously prevented from entering, as well as accumulating in tissues and organs where cannabinoids and nanoparticles would not typically be found. Additionally, such nanoemulsions, as well as other water-compatible strategies, do not address one of the major-shortcomings of cannabinoid-infused commercial consumables, namely the strong unpleasant smell and taste. Moreover, such water-compatible strategies deliver inconsistent and delayed cannabinoid uptake in the body which may result in consumers ingesting a higher dose of cannabinoid-infused product than is recommended, as well as delayed, inconsistent, and unpredictable medical and/or psychotropic experiences.

As a result, there is a need for more effective strategies to both solubilize cannabinoids, and other associated compounds, such as terpenes and the like, in a way that is both cost-effective, as well as safe to consumers. Notably, organisms have long been utilizing protein associations to make hydrophobic molecules water soluble for biological processes. As outlined below, cannabinoids may be solubilized by binding to certain carrier proteins. Generally, the binding to these carrier protein molecules effectively increases the water-solubility of fatty acids and other lipophilic molecules, thereby facilitating their transport through aqueous environments as well as their transfer across cellular membranes. Human and homologous non-human carrier proteins may offer an opportunity for use in the solubilization of cannabinoids among other compounds.

Most, although not all, Odorant binding proteins (OBPs) belong to a class of proteins known as lipocalins, which allow the transport of hydrophobic molecules to, from, and within cells. Lipocalins are an ancient and functionally diverse family of mostly extracellular proteins. Lipocalins can be found in gram negative bacteria, vertebrate cells, and invertebrate cells, and in plants. Lipocalins have been associated with many biological processes, among them immune response, olfaction, biological prostaglandin synthesis, retinoid binding, and cancer cell interactions.

As noted in Table 4 below, Lipocalins may generally include a highly symmetrical all β-structure dominated by a single eight-stranded antiparallel β-sheet closed back on itself to form a continuously hydrogen-bonded β-barrel. This β-barrel encloses a ligand-binding site composed of both an internal cavity and an external loop scaffold. The structural diversity of cavity and scaffold gave rise to a variety of different binding specificities, each capable of accommodating ligands of different size, shape, and chemical character. Lipocalins generally bind small hydrophobic ligands such as retinoids, fatty acids, steroids, odorants, and pheromones, and interact with cell surface receptors. Notably, Lipocalins can be found in both animal as well as plant species. This combination of factors makes these Lipocalins and lipocalin-like proteins ideal for binding hydrophobic molecules including cannabinoids, terpenes, and volatiles which offer many benefits including improved water-solubility as well as potential stability enhancement. One manifestation of these proteins, Odorant Binding Proteins (OBPs), are used by organisms to bind and solubilize pheromones, terpenoids, other odor volatiles, and other hydrophobic molecules including phenolic compounds possessing non-polar short chain fatty acids. OBPs are also known to be highly stable proteins, tolerant of heat, organic solvents, and toxins. Notably, OBPs play crucial role in olfaction. The very first step in olfaction is to deliver odor molecules from the environment to the olfactory receptors. Humans and animals have special proteins called odorant-binding proteins (OBPs). These proteins bind to odor molecules as they arrive in the mucosa of the olfactory epithelium, solubilize them into the aqueous environment, and transport them to olfactory receptors, which are located on the dendrites of olfactory sensory neurons in the olfactory epithelium within the noses of humans and animals. Vertebrate OBPs are members of large lipocalins family and share the eight stranded beta barrel. Insects have two types OBPs: general odorant-binding proteins (GOBPs) and the pheromonebinding proteins (PBPs). They are completely different from their vertebrate counterpart both in sequence and three-dimensional folding. Insect OBPs contain an alpha helical barrel and six highly conserved cysteines. Another class of putative OBPs, named chemosensory proteins (CSPs) has been reported in different orders of insects, including Lepidoptera. In spite of the sequence and structural difference, their general chemical properties indicate similar functions in olfactory transduction. They also function to remove and breakdown odorants so the receptor can continue to bind incoming odor molecules. OBPs are relatively promiscuous. They can be studied in E. coli and are easy to manipulate. This combination of factors makes OBPs ideal for binding hydrophobic molecules including cannabinoids, terpenes, and other volatiles thereby offering many benefits including improved water-solubility as well as potential stability enhancement.

As will be discussed in more detail below, the current inventive technology overcomes the limitations of traditional cannabinoid emulsion systems while meeting the objectives of a truly effective and scalable cannabinoid production, solubilization, and isolation system.

### SUMMARY OF THE INVENTION

Generally, the inventive technology relates to systems, methods and compositions to solubilize short-chain fatty acid phenolic compounds, such as cannabinoids, terpenes and other volatile compounds found in cannabinoid-producing plants such as Cannabis. In one embodiment, a cannabinoid-carrier protein may include OBPs. In one aspect, human and homologous non-human OBPs may act as carrier proteins for use in the solubilization of cannabinoids. In addition to this, chimeric proteins and engineered OBPs with planned mutations may offer increased efficacy for this solubilization. In one embodiment, a cannabinoid-carrier protein may include members of the lipocalins family of proteins, and preferably lipocalin proteins from plants or animals. In one aspect, human and homologous non-human OBPs may act as carrier proteins for use in the solubilization of cannabinoids. In addition to this, chimeric proteins and engineered Lipocalins with planned mutations may offer increased efficacy for this solubilization.

One aspect of the present invention may include the increase of water-solubility of target hydrophobic molecules including cannabinoids, terpenes, and other volatiles, preferably from Cannabis. In this embodiment, the inventive technology includes a suite of novel synthetic/bio-synthetic odorant binding homolog proteins for the binding of cannabinoids which may increase the water-solubility of the hydrophobic cannabinoids ultimately resulting in safer and more palatable solutions for medicine and recreation. In this embodiment, the inventive technology may further include a suite of LC-carriers, as well as novel synthetic/bio-synthetic LC-carrier homolog proteins for the binding of cannabinoids which may increase the water-solubility of the hydrophobic cannabinoids ultimately resulting in safer and more palatable solutions for medicine and recreation.

Another aspect of the present invention may include the use of naturally occurring OBPs and LC proteins to increase water-solubility of target hydrophobic molecules including cannabinoids, terpenes, and volatiles. In this embodiment, the inventive technology includes a suite of naturally occurring organismal odorant binding for the binding of target hydrophobic molecules which may increase the water-solubility ultimately resulting in safer, more consistent, and more palatable solutions for medical, industrial, and recreational applications. In this embodiment, the inventive technology further includes a suite of naturally occurring organismal LC carriers for the binding of target hydrophobic molecules which may increase the water-solubility ultimately resulting in safer, more consistent, and more palatable solutions for medical, industrial, and recreational applications.

Another aim of the present invention may include the transport, storage, and isolation of target hydrophobic molecules including cannabinoids, terpenes, and volatiles. In this embodiment, the inventive technology includes a suite of novel synthetic/bio-synthetic and naturally occurring organismal proteins to bind target hydrophobic molecules for the purpose of isolating the molecules, transporting the molecules, or storing the target molecules. In this embodiment, the inventive technology further includes a suite of novel synthetic/bio-synthetic and naturally occurring L/OBP-carrier proteins to bind target hydrophobic molecules for the purpose of isolating the molecules, transporting the molecules, or storing the target molecules.

Another aim of the present invention may include the creation of chimeric proteins derived from proteins listed in the aforementioned aims. In this embodiment, the inventive technology includes the creation of new and novel chimera or modified proteins based on amino acid sequences, and preferably in the L/OBP family of proteins to improve target hydrophobic molecule interactions. In this embodiment, the inventive technology further includes the creation of new and novel chimera or modified proteins based on amino acid sequences identified in the lipocalins, and preferably LC-carrier and OBP-carrier proteins to improve target hydrophobic molecule interactions.

As used herein, proteins from the Lipocalin family, and proteins from the class of Lipocalins identified as OBPs, that have binding affinity directed to one or more cannabinoids such as CBD and THC, may generally be referred to individually and/or collectively as “Lipocalin and/or Odorant Binding Protein-carrier(s)” or “L/OBP-carrier(s).” In one embodiment, “Lipocalin and/or Odorant Binding Protein-carrier(s)” or “L/OBP-carrier(s) may include the amino acid sequences according to: SEQ ID NOs. 1-46, and SEQ ID NOs. 113-148. The terms “Lipocalin and/or Odorant Binding Protein-carrier(s)” or “L/OBP-carrier(s)” may also include all homologs, or orthologs having affinity directed to one or more cannabinoids.

As used herein, proteins from the Lipocalin family that have binding affinity directed to one or more cannabinoids such as CBD and THC, may generally be referred to individually and/or collectively as “Lipocalin Cannabinoid-carrier(s)” or “LC-carrier(s).” In one embodiment, “Lipocalin Cannabinoid-carrier(s)” or “LC-carrier(s) may include the amino acid sequences according to: SEQ ID NOs. 1-29. The terms “Lipocalin Cannabinoid-carrier(s)” or “LC-carrier(s)” may further include all homologs, or orthologs having affinity directed to one or more cannabinoids.

As used herein, from the class of Lipocalins identified as OBPs that have binding affinity directed to one or more cannabinoids such as CBD and THC, may generally be referred to individually and/or collectively as “Odorant Binding Protein-carriers(s)” or “OBP-carrier(s).” In one embodiment, “Odorant Binding Protein-carriers(s)” or “OBP-carrier(s)” may include the amino acid sequences according to: SEQ ID NOs. 113-148. The terms Odorant Binding Protein-carriers(s)” or “OBP-carrier(s)” may further include all homologs, or orthologs having affinity directed to one or more cannabinoids.

As used herein, proteins from the Lipocalin family, and proteins from the class of Lipocalins identified as OBPs, that have binding affinity directed to one or more cannabinoids such as CBD and THC, and that may be genetically modified, for example through the addition of a secretion signal, or one or more amino acid residue mutations, or a truncated version of a wild type Lipocalin or OBP may generally be referred to individually and/or collectively as an “engineered Lipocalin and/or engineered Odorant Binding Protein-carrier(s)” or “engineered L/OBP-carrier(s).” In one embodiment, “engineered Lipocalin and/or Odorant Binding Protein-carrier(s)” or “engineered L/OBP-carrier(s) may include the amino acid sequences according to: SEQ ID NOs. 30-46, or SEQ ID NOs. 1-46, and 113-148 coupled with one or more secretion signals selected from SEQ ID NO. 47, and SEQ ID NOs. 106-112.

As used herein, proteins from the Lipocalin family that have binding affinity directed to one or more cannabinoids such as CBD and THC, and that may be genetically modified, for example through the addition of a secretion signal, or one or more amino acid residue mutations, or a truncated version of a wild type Lipocalin protein may generally be referred to individually and/or collectively as “engineered Lipocalin Cannabinoid-carrier(s)” or “LC-carrier(s).” In one embodiment, “engineered Lipocalin Cannabinoid-carrier(s)” or “LC-carrier(s)” may include the amino acid sequences according to: SEQ ID NOs. 30-46, or SEQ ID NOs. 1-46 coupled with one or more secretion signals selected from SEQ ID NO. 47, and SEQ ID NOs. 106-112.

As used herein, from the class of Lipocalins identified as OBPs that have binding affinity directed to one or more cannabinoids such as CBD and THC, and that may be genetically modified, for example through the addition of a secretion signal, or one or more amino acid residue mutations, or a truncated version of a wild type OBP may generally be referred to individually and/or collectively as an “engineered Odorant Binding Protein-carriers(s)” or “engineered OBP-carrier(s).” In one embodiment, engineered Odorant Binding Protein-carriers(s)” or “engineered OBP-carrier(s)” may include the amino acid sequences according to: SEQ ID NOs. 113-148 coupled with one or more secretion signals selected from SEQ ID NO. 47, and SEQ ID NOs. 106-112. Notably, the term L/OBP-carrier protein may also generally encompass engineered L/OBP-carrier proteins.

Another aspect of the current invention may include novel methods and compositions for increasing the water solubility of one or more cannabinoid compounds via binding to a select Lipocalin proteins and/or OBPs. In this embodiment, L/OBP-carriers may be utilized to solubilize, transport, and store cannabinoid compounds in in vitro, ex vivo, and in vivo systems. In specific preferred aspects, non-human homologs of L/OBP-carriers, such as plant L/OBP-carriers, or engineered L/OBP-carrier may be utilized to solubilize, transport, and store, for example, THC, CBD, and other cannabinoids, terpenoids, and volatile compounds produced in Cannabis and other cannabinoid producing plants, or even synthetically generated cannabinoids.

Another aspect of the current invention includes novel methods and compositions for increasing the water solubility of one or more cannabinoid compounds via binding to a select chimeric or genetically modified, sometimes referred to as an engineered, L/OBP-carrier. In this aspect, a novel chimeric L/OBP-carrier construct may be rationally designed from homologs of plant or animal L/OBP-carriers to allow for enhanced binding of cannabinoid molecules to a single protein chain. In one specific aspect, a novel chimeric L/OBP-carrier construct may be rationally designed from one or more homologs of a Lipocalin or OBP to allow for enhanced binding of THC, CBD, or other cannabinoid molecules to a single protein chain. In another aspect, one or more L/OBP-carriers, and preferably an LC-carrier may be genetically modified to produce a truncated portion of a wild-type LC-carrier protein that may retain the LC-carrier protein's binding affinity, and ability to solubilize one or more target cannabinoids.

Another aspect of the current invention may include systems, methods, and compositions for the solubilization of cannabinoids, terpenoids and other short-chain fatty acid phenolic compounds in cell cultures that express one or more L/OBP-carrier, or engineered L/OBP-carrier proteins. Exemplary cell cultures may include bacterial, yeast, plant, algae and fungi cell cultures. In another aspect, L/OBP-carrier, or engineered L/OBP-carrier proteins, may be coupled with secretion signals to allow such proteins to be more easily exported from the cell culture into the surrounding supernatant or media. In this aspect of the invention, a L/OBP-carrier protein, the terms generally encompassing L/OBP-carrier proteins, or engineered L/OBP-carrier proteins that bind to one or more target compounds, and preferably cannabinoids, may be exported out of a cell through the action of the secretion signal that may direct posttranslational protein translocation into the endoplasmic reticulum (ER), or in alternative embodiments, a secretion signal that may direct cotranslational translocation across the ER membrane where it may assume its three-dimensional form and bind one or more cannabinoid or other compounds as described herein. In one preferred embodiment, a L/OBP-carrier protein may be generated in a cell culture, preferably a bacterial, yeast, plant or fungi cell culture, and then be exported out of the cell through natural cellular action, or through the action of the secretion signal where it may assume its three dimensional form and bind one or more cannabinoid or other compounds that may be present, preferably by addition of said compound, such as: a quantity of an isolated cannabinoid; a quantity of a plurality of cannabinoids; or Cannabis extract, to the culture's supernatant.

In another aspect of the invention, an L/OBP-carrier protein may be exported out of a cell through the action of the secretion signal after it has assumed a transitory and or final three dimensional form and may further be bound to one or more cannabinoid or other compounds as described herein. In one preferred embodiment, a L/OBP-carrier protein may be generated in a cell culture, preferably a bacterial, yeast, plant or fungi cell culture, and more preferably a plant suspension culture of a cannabinoid-producing plant such as Cannabis, where it may assume a transitory or final three dimensional form and bind one or more cannabinoids or other compounds that may be present or produced in the cell.

Another aspect of the current invention may include systems, methods and compositions for the solubilization of cannabinoids, terpenoids and other short-chain fatty acid phenolic compounds in whole plants and plant cell cultures. In certain embodiments, such plants or cell cultures may be genetically modified to direct cannabinoid synthesis to the cytosol, as opposed to a trichome structure. One or more L/OBP-carrier proteins may be coupled with a secretion signal, preferable in a plant cell culture, to allow such proteins to be exported from the cell into the surrounding media. Expression of exportable and non-exportable L/OBP-carrier proteins may be co-expressed with one or more catalase and/or one or more myb transcription factors which may enhance cannabinoid production in a Cannabis plant or cell culture.

Another aspect of the current invention may include systems, methods and compositions for the coupled glycosylation and solubilization of cannabinoids, terpenoids and other short-chain fatty acid phenolic compounds in whole cannabinoid-producing plants and cell cultures, preferably Cannabis. In this embodiment, such Cannabis plants or cell cultures may be genetically modified to direct cannabinoid synthesis to the cytosol, as opposed to a trichome structure. Such Cannabis plant or cell culture may be further genetically modified to express one or more heterologous glycosyltransferases having glycosylation activity towards at least one cannabinoid (for example SEQ ID NOs. 73-88, and SEQ ID NOs. 102-103), In additional embodiments, a plant or cell may be further genetically modified to express one or more heterologous glycosyltransferases, wherein in said polynucleotides encoding such glycosyltransferases may be codon-optimized for expression in an exogenous system, such as in yeast (for example SEQ ID NOs. 90-101). In additional embodiments, a heterologous or exogenous, the terms being generally interchangeable, cytochrome P450 and/or a P450 oxidoreductase may be expressed. In this configuration a heterologous cytochrome P450 (for example SEQ ID NOs. 63-64, and SEQ ID NOs. 67-68) may hydroxylate a cannabinoid to form a hydroxylated cannabinoid and/or oxidizes a hydroxylated cannabinoid to form a cannabinoid carboxylic acid. Further, in this embodiment, a heterologous P450 oxidoreductase (for example SEQ ID NOs. 65-66, and SEQ ID NOs. 69-70) may facilitate electron transfer from a nicotinamide adenine dinucleotide phosphate (NADPH) to said cytochrome P450.

As noted above, a heterologous glycosyltransferase may glycosylate a cannabinoid compound and thereby produce a water-soluble cannabinoid glycoside. This glycosylated cannabinoid may bind to a heterologous L/OBP-carrier also expressed in the Cannabis plant or cell that may be coupled with a secretion signal, to allow the carrier proteins to be exported from the cell into the surrounding media. Expression of exportable and non-exportable L/OBP-carriers may be co-expressed with one or more catalase and/or one or more myb transcription factors. The glycosylated cannabinoids bound to the L/OBP-carrier, being further coupled with a tag in some embodiments, may be isolated, while in still further embodiments, the L/OBP-carrier protein may be disrupted by a protease, or other protein disrupting detergent and the like, such that the glycosylated cannabinoid may be released from the L/OBP-carrier and may be further isolated or reconstituted to their original forms through the action of a glycosidase that may remove the sugar moiety.

Another aspect of the current invention may include systems, methods, and compositions for the coupled glycosylation and solubilization of cannabinoids, terpenoids and other short-chain fatty acid phenolic compounds in non-cannabinoid-producing plants and cell cultures, preferably a tobacco cell culture. In this embodiment, a tobacco cell culture may endogenously express one or more glycosyltransferases having glycosylation activity towards at least one cannabinoid. The tobacco cell culture may optionally be genetically modified to express a heterologous cytochrome P450, and a P450 oxidoreductase. In this configuration a heterologous cytochrome P450 may hydroxylate a cannabinoid added to a tobacco cell culture for example, to form a hydroxylated cannabinoid and/or oxidizes a hydroxylated cannabinoid to form a cannabinoid carboxylic acid. Further, in this embodiment, a heterologous P450 oxidoreductase may facilitate electron transfer from a nicotinamide adenine dinucleotide phosphate (NADPH) to said cytochrome P450. As noted above, the endogenously expressed heterologous glycosyltransferases (fore example, NtGT1, 2, 3, 4 or 5 as identified below) may glycosylate one or more cannabinoids introduced to the tobacco cell culture converting it into a water-soluble cannabinoid-glycoside. This glycosylated cannabinoid may bind to a heterologous L/OBP-carrier co-expressed or added to the tobacco cell culture. In this aspect, an expression of an exportable L/OBP-carrier may be co-expressed with one or more catalase and/or one or more myb transcription factors. The glycosylated cannabinoids bound to the L/OBP-carrier, being further coupled with a tag in some embodiments, may be isolated, while in still further embodiments, the carrier protein may be disrupted by a protease or other protein disrupting detergent and the like such that the glycosylated cannabinoids may be released from the carrier protein and may be further isolated or reconstituted to their original forms through the action of a glycosidase.

Another aspect of the current invention may include systems, methods and compositions for the coupled glycosylation and solubilization of cannabinoids, terpenoids and other short-chain fatty acid phenolic compounds in a cell cultures, preferably a yeast cell culture. In these embodiments, yeast cultures may be genetically modified to biosynthesize one or more cannabinoids. The yeast cell culture may be further genetically modified to express one or more heterologous glycosyltransferases having glycosylation activity towards at least one cannabinoid, as well as in some embodiments, a heterologous cytochrome P450 and/or a P450 oxidoreductase.

As noted above, heterologous glycosyltransferases may glycosylate the cannabinoid making it water-soluble. This glycosylated cannabinoid may bind to a heterologous L/OBP-carrier protein also expressed in the yeast culture which may further be coupled with a secretion signal, to allow the carrier proteins to be exported from the yeast cell into the surrounding media. Expression of exportable and non-exportable L/OBP-carrier may be co-expressed with a catalase. The glycosylated cannabinoids bound to the L/OBP-carrier being further coupled with a tag in some embodiments, may be isolated, while in still further embodiments, the carrier protein may be disrupted by a protease or other protein disrupting detergent and the like such that the glycosylated cannabinoids may be released from the carrier protein and may be further isolated or reconstituted to their original forms through the action of a glycosidase.

Another aspect of the current invention may include systems, methods and compositions for the coupled glycosylation and solubilization of cannabinoids, terpenoids and other short-chain fatty acid phenolic compounds in a cell cultures, preferably yeast, bacteria, fungi or algal cell culture. In these embodiments, a yeast cultures may be genetically modified to express one or more heterologous glycosyltransferases having glycosylation activity towards at least one cannabinoid, as well as in some embodiments, a heterologous cytochrome P450 and/or a P450 oxidoreductase. As noted above, in one preferred embodiment, a quantity of cannabinoids may be added to the cell culture, and preferably a yeast cell culture, where heterologous glycosyltransferases may glycosylate the cannabinoid making it water-soluble. This glycosylated cannabinoid may bind to a heterologous L/OBP-carrier co-expressed in the yeast culture which may further be coupled with a secretion signal, to allow the carrier proteins to be exported from the yeast cell into the surrounding media. The glycosylated cannabinoids bound to the L/OBP-carrier, being further coupled with a tag in some embodiments, may be isolated, while in still further embodiments, the carrier protein may be disrupted by a protease or other protein disrupting detergent and the like such that the glycosylated cannabinoids may be released from the carrier protein and may be further isolated or reconstituted to their original forms through the action of a glycosidase.

Another aspect of the current invention may include one or more heterologous glycosyltransferases coupled with the expression of an L/OBP-carrier optionally having secretion signal, and in some embodiments a tag, which may be expressed in a plant, yeast or bacterial cell culture. Another aspect of the current invention may include one or more heterologous glycosyltransferases coupled with the addition of an L/OBP-carrier to a plant, yeast, or bacterial cell culture.

Another aspect of the current invention may include one or more endogenously expressed glycosyltransferases coupled with the expression of an L/OBP-carrier, and preferable an engineered L/OBP-carrier having secretion signal, and in some embodiments a tag, that may be expressed in a plant, yeast or bacterial cell culture. Another aspect of the current invention may include one or more endogenously expressed glycosyltransferases coupled with the addition of an L/OBP-carrier to a plant cell culture.

Another aspect of the current invention may include the increase of CBD and/or THC water solubility for transport via binding to an L/OBP-carrier. In this embodiment, plant or other non-human homologs of L/OBP-carriers may be utilized to solubilize, transport, and/or store CBD and closely-related cannabinoids. Another aspect of the current invention may include the increase of CBD water solubility for transport via binding to an L/OBP-carrier. In one preferred aspect, a novel engineered LC-carrier construct may be rationally designed from one or more LC-carriers to generate improved truncated proteins that may bind to, and solubilize a CBD molecule to a single protein chain. Such truncated or engineered LC-carriers may exhibit enhanced cannabinoid docking, as well as more favorable stoichiometry such that less protein may be used to solubilize/deliver a quantifiable amount of a target cannabinoid which may enhance the carrier proteins ability to be used in formulations for various commercial products and the like.

Another aspect of the inventive technology may include polynucleotides encoding one or more L/OBP-carrier proteins being heterologously expressed in a genetically modified microorganism, such as a yeast, bacteria, fungi, algae or. In one preferred aspect, of the inventive technology may include genetically modified bacteria that express at least one polynucleotide encoding one or more heterologous L/OBP-carriers-carrier, and preferably one or more engineered L/OBP-carrier proteins. Another aspect of the inventive technology may include novel engineered L/OBP-carrier-carrier amino acid and their corresponding nucleotide sequences.

Another aspect of the inventive technology provides for a method of enhancing the solubility and stability of cannabinoids, terpenoids and/or other short-chain fatty acid phenolic compounds utilizing L/OBP-carrier proteins. In a preferred embodiment, a nucleotide sequence encoding a L/OBP-carrier protein may be genetically engineered to express a rationally designed L/OBP-carrier protein having cannabinoid affinity or binding sites having enhanced affinity for cannabinoids such that the engineered L/OBP-carrier protein may bind cannabinoids with a higher affinity thereby increasing the solubility and stability of the cannabinoid in a solution or other form.

Another aspect of the invention includes compositions of novel engineered L/OBP-carrier polynucleotides and proteins and their method or manufacture. Another aspect of the invention includes compositions of novel engineered L/OBP-carrier polynucleotides and proteins and their method or manufacture. Another aspect of the invention involves the identification of L/OBP-carrier proteins that may have endogenous cannabinoid or other affinity sites. Another aspect of the invention involves the rational design of engineered L/OBP-carrier proteins, and preferably truncated LC-carrier proteins that have affinity directed toward one or more cannabinoids, and that may further be genetically engineered for expression in an in vivo system, such as bacteria with the addition of a start sequence encoding a methionine amino acid residue. In one preferred aspect, an engineered LC-carrier may include a truncated LC-carrier having a β-barrel ligand-binding site composed of both an internal cavity and an external loop scaffold that binds to one or more cannabinoids.

Another aspect of the invention includes compositions of novel consumer products that incorporate one or more solubilized cannabinoids bound to L/OBP-carrier proteins and/or engineered L/OBP-carrier proteins.

Additional embodiment may further include one or more of the following embodiments:
1. A method of solubilizing a cannabinoid comprising the steps of:
generating a Olfactory-Binding Protein (OBP)-carrier protein having affinity towards at least one cannabinoid; and
introducing said OBP-carrier protein to said at least one cannabinoid, wherein said OBP-carrier protein binds said at least one cannabinoid to form a water-soluble protein-cannabinoid composition.
   2. The method of embodiment 1, wherein the OBP-carrier protein comprises an OBP-carrier protein having an amino acid sequence selected from the group of consisting of: SEQ ID NOs. 113-148, or a homolog having affinity towards at least one cannabinoid thereof.
   3. The method of embodiment 2, wherein said step of generating an OBP-carrier protein comprises the step of generating an OBP-carrier protein in a protein production system selected from the group consisting of:
a bacterial cell culture;
a yeast cell culture;
a plant cell culture;
a fungi cell culture;
an algae cell culture;
a bioreactor production system; and
a plant.
   4. The method of embodiment 3, wherein the OBP-carrier protein is coupled with a secretion signal.
   5. The method of embodiment 4, wherein said secretion signal comprises a secretion signal selected from the group consisting of: SEQ ID NO. 47, and SEQ ID NOs. 106-112.
   6. The method of embodiments 3 and 5, wherein the OBP-carrier protein is introduced to said at least one cannabinoid in said protein production system.
   7. The method of embodiment 1, wherein the at least one cannabinoid comprises a cannabinoid selected from the group consisting of: cannabidiol (CBD), cannabidiolic acid (CBDA), Δ⁹-tetrahydrocannabinol (THC), tetrahydrocannabinolic acid (THCA), and (cannabigerolic acid) CBGA).
   8. The method of embodiment 1, wherein said OBP-carrier protein having affinity towards at least one cannabinoid comprises an OBP-carrier protein having a β-barrel enclosed cannabinoid-binding site having an internal cavity, and an external loop scaffold structure.
   9. The method of embodiments 1 and 8, wherein said OBP-carrier protein is in solution.
   10. The method of embodiment 1 and 8, wherein the OBP-carrier protein undergoes lyophilisation.
   11. An isolated polynucleotide that encodes one or more amino acid sequences selected from the group of consisting of: SEQ ID NOs. 113-148, or a homolog having affinity towards at least one cannabinoid thereof.
   12. The polynucleotide of embodiment 11, wherein said polynucleotide is operably linked to a promotor forming an expression vector.
   13. The polynucleotide of embodiment 11, wherein said polynucleotide is codon optimized for expression in a microorganism, or plant cell, and is further operably linked to a promotor forming an expression vector.
   14. A genetically modified organism expressing at least one of the expression vectors of embodiments 12 and 13.
   15. A solubilized cannabinoid composition comprising:
an carrier protein having a β-barrel enclosed cannabinoid-binding site having an internal cavity, and an external loop scaffold structure bound to at least one cannabinoid to form a water-soluble protein-cannabinoid composition.
   16. The composition of claim 15, wherein the carrier protein comprises an carrier protein having an amino acid sequence selected from the group of consisting of: SEQ ID NOs. 1-46, and 113-148, or a homolog having affinity towards at least one cannabinoid thereof.
   17. The composition of embodiments 15 and 16, wherein said water-soluble protein-cannabinoid composition is introduced to a consumer product meant for human-consumption, or a pharmaceutical composition for administration of a therapeutically effective dose to a subject in need thereof; or a prodrug for administration of a therapeutically effective dose to a subject in need thereof.
   18. The composition of embodiment 15, wherein the carrier protein is coupled with a secretion signal.
   19. The composition of embodiment 18, wherein said secretion signal comprises a secretion signal selected from the group consisting of: SEQ ID NO. 47, and SEQ ID NOs. 106-112.
   20. The composition of claim embodiment 15 and 16, wherein the at least one cannabinoid comprises a cannabinoid selected from the group consisting of: cannabidiol (CBD), cannabidiolic acid (CBDA), Δ⁹-tetrahydrocannabinol (THC), tetrahydrocannabinolic acid (THCA), and (cannabigerolic acid) CBGA).
   21. The composition of embodiment 15, wherein said carrier protein having affinity towards at least one cannabinoid comprises an OBP-carrier protein having a β-barrel enclosed cannabinoid-binding site having an internal cavity, and an external loop scaffold structure.
   22. The composition of embodiment 15, wherein said carrier protein having affinity towards at least one cannabinoid comprises an Lipocalin Cannabinoid (LC)-carrier protein having a β-barrel enclosed cannabinoid-binding site having an internal cavity, and an external loop scaffold structure.
   23. The genetically modified organism of embodiments 13 and 14, wherein said genetically modified organism is selected from the group consisting of:
a genetically modified bacterial cell
a genetically modified yeast cell,
a genetically modified plant cell,
a genetically modified fungi cell,
a genetically modified algae cell, and
a genetically modified plant.
   24. A method of solubilizing a cannabinoid comprising the steps of:
establishing a cell culture of genetically modified yeast, plant, or bacteria cells that express a nucleotide sequence encoding a heterologous Olfactory Binding Protein (OBP)-carrier protein operably linked to a promotor wherein said heterologous OBP-carrier protein exhibits affinity towards one or more cannabinoids;
introducing one or more cannabinoids to the genetically modified yeast, plant, or bacteria cell culture; and
wherein said OBP-carrier protein binds said one or more cannabinoids to form a water-soluble protein-cannabinoid composition.
   25. The method of embodiment 24, wherein the step of introducing comprises the step of introducing one or more cannabinoids to a genetically modified yeast, plant, or bacteria cell culture in a fermenter or suspension cell culture.
   26. The method of embodiment 24, wherein the step of introducing comprises the step of biosynthesizing one or more cannabinoids in a genetically modified yeast, plant, or bacteria cell culture wherein said heterologous OBP-carrier protein binds said one or more biosynthesized cannabinoids to form a water-soluble protein-cannabinoid composition.
   27. The method of embodiment 24, wherein said heterologous OBP-carrier protein comprises a heterologous OBP-carrier protein having an amino acid sequence selected from the group of consisting of: SEQ ID NOs. 113-148, or a homolog having affinity towards at least one cannabinoid thereof.
   28. The method of embodiments 24 and 27, wherein said heterologous OBP-carrier protein is coupled with a tag.
   29. The method of embodiments 24 and 27, wherein said heterologous OBP-carrier protein is coupled with a secretion signal.
   30. The method of embodiment 29, wherein said secretion signal comprises a secretion signal selected from the group consisting of: SEQ ID NO. 47, and SEQ ID NOs. 106-112.
   31. The method of embodiment 24, wherein the at least one cannabinoid comprises a cannabinoid selected from the group consisting of: cannabidiol (CBD), cannabidiolic acid (CBDA), Δ⁹-tetrahydrocannabinol (THC), tetrahydrocannabinolic acid (THCA), and (cannabigerolic acid) CBGA).
   32. The method of embodiment 24, and further comprising the of step of genetically modifying the OBP-carrier protein form an engineered OBP-carrier protein having enhanced affinity for at least one cannabinoid, such genetic modification comprising one or more of the following:
replacing one or more amino acid residues of the OBP-carrier protein cannabinoid binding pocket with side chains pointing towards orientated toward the binding cavity;
replacing one or more amino acid residues of the OBP-carrier protein cannabinoid binding pocket having a hydrophilic side chain with amino acid residues having a hydrophobic side chain; and
replacing one or more small hydrophobic amino acid residues of the OBP-carrier protein cannabinoid binding pocket with larger hydrophobic amino acid residues.
   33. The OBP-carrier protein of embodiments 1, 13, 24 and 32, wherein the OBP-carrier protein is further genetically modified to decrease potential antigenicity.
   34. The OBP-carrier protein of embodiments 1, 13, 24 and 32, wherein the OBP-carrier protein is further genetically modified to decrease aggregation propensity.
   35. The water-soluble protein-cannabinoid composition of any of the embodiments above wherein said water-soluble protein-cannabinoid composition is introduced to a consumer product meant for human-consumption, or a pharmaceutical composition for administration of a therapeutically effective dose to a subject in need thereof; or a prodrug for administration of a therapeutically effective dose to a subject in need thereof.
   36. A genetically modified Cannabis plant expressing a nucleotide sequence operably linked to a promoter encoding at least one Olfactory Binding Protein (OBP)-carrier protein.
   37. The Cannabis plant of embodiment 36 and wherein said FABP-carrier protein comprises a FABP-carrier protein selected from the group consisting of: an amino acid sequence according to SEQ ID NOs. 113-148.
   38. The Cannabis plant of embodiments 36 and 37, and further comprising the step of expressing a nucleotide sequence operably linked to a promoter encoding one or more cannabinoid synthases having its trichome targeting sequence disrupted or removed.
   39. The Cannabis plant of embodiment 38, wherein one or more cannabinoid synthase genes has been disrupted or knocked out.
   40. The Cannabis plant of embodiment 39, wherein said one or more cannabinoid synthases having its trichome targeting sequence disrupted or removed is selected from the group consisting of the nucleotide sequence identified as: SEQ ID NOs. 55-57.
   41. The Cannabis plant of embodiment 36, and further comprising the step of expressing at least one myb transcription factor.
   42. The Cannabis plant of embodiment 40, wherein said at least one myb transcription factor is selected from the group consisting of: SEQ ID NOs. 58-62.
   43. The Cannabis plant of embodiment 36, and further comprising the step of expressing at least one catalase.
   44. The Cannabis plant of embodiment 43, wherein said at least one catalase is selected from the group consisting of: SEQ ID NOs. 48-52.
   45. The Cannabis plant of embodiment 36, and further comprising the step of expressing at least one heterologous glycosyltransferase.
   46. The Cannabis plant of embodiment 45, wherein said at least one at least one heterologous glycosyltransferase is selected from the group consisting of: SEQ ID NOs. 73-88, and SEQ ID NOs. 102-103.
   47. A method of solubilizing a cannabinoid comprising the steps of:
generating a Lipocalin Carrier (LP)-carrier protein having affinity towards at least one cannabinoid; and
introducing said LC-carrier protein to said at least one cannabinoid, wherein said LC-carrier protein binds said at least one cannabinoid to form a water-soluble protein-cannabinoid composition.
   48. The method of embodiment 47, wherein the LC-carrier protein comprises an LC-carrier protein having an amino acid sequence selected from the group of consisting of: SEQ ID NOs. 1-29, and 30-46 or a homolog having affinity towards at least one cannabinoid thereof.
   49. The method of embodiment 48, wherein said step of generating an LC-carrier protein comprises the step of generating an LC-carrier protein in a protein production system selected from the group consisting of:
a bacterial cell culture;
a yeast cell culture;
a plant cell culture;
a fungi cell culture;
an algae cell culture;
a bioreactor production system; and
a plant.
   50. The method of embodiment 49, wherein the LC-carrier protein is coupled with a secretion signal.
   51. The method of embodiment 50, wherein said secretion signal comprises a secretion signal selected from the group consisting of: SEQ ID NO. 47, and SEQ ID NOs. 106-112.
   52. The method of embodiments 49 and 51, wherein the LC-carrier protein is introduced to said at least one cannabinoid in said protein production system.
   53. The method of embodiment 47, wherein the at least one cannabinoid comprises a cannabinoid selected from the group consisting of: cannabidiol (CBD), cannabidiolic acid (CBDA), Δ⁹-tetrahydrocannabinol (THC), tetrahydrocannabinolic acid (THCA), and (cannabigerolic acid) CBGA).
   54. The method of embodiment 47, wherein said LC-carrier protein having affinity towards at least one cannabinoid comprises an LC-carrier protein having a β-barrel enclosed cannabinoid-binding site having an internal cavity, and an external loop scaffold structure.
   55. The method of embodiments 47 and 54, wherein the LC-carrier comprises an engineered LC-carrier protein further comprising a truncated LC-carrier protein forming a β-barrel enclosed cannabinoid-binding site having an internal cavity, and an external loop scaffold structure.
   56. The method of embodiment 55, wherein said engineered LC-carrier protein comprises an engineered LC-carrier protein having an amino acid sequence selected from the group of consisting of: SEQ ID NOs. 30-46.
   57. An isolated polynucleotide that encodes one or more amino acid sequences selected from the group of consisting of: SEQ ID NOs. 1-29, and 30-46, or a homolog having affinity towards at least one cannabinoid thereof.
   58. The polynucleotide of embodiment 57, wherein said polynucleotide is operably linked to a promotor forming an expression vector.
   59. The polynucleotide of embodiment 57, wherein said polynucleotide is codon optimized for expression in a microorganism, or plant cell, and is further operably linked to a promotor forming an expression vector.
   60. A genetically modified organism expressing at least one of the expression vectors of embodiments 58 and 59.
   61. The genetically modified organism of embodiments 60, wherein said genetically modified organism is selected from the group consisting of:
a genetically modified bacterial cell
a genetically modified yeast cell,
a genetically modified plant cell,
a genetically modified fungi cell,
a genetically modified algae cell, and
a genetically modified plant.
   62. A method of solubilizing a cannabinoid comprising the steps of:
establishing a cell culture of genetically modified yeast, plant, or bacteria cells that express a nucleotide sequence encoding a heterologous Lipocalin Carrier (LC)-carrier protein operably linked to a promotor wherein said heterologous LC-carrier protein exhibits affinity towards one or more cannabinoids;
introducing one or more cannabinoids to the genetically modified yeast, plant, or bacteria cell culture; and
wherein said LC-carrier protein binds said one or more cannabinoids to form a water-soluble protein-cannabinoid composition.
   63. The method of embodiment 62, wherein the step of introducing comprises the step of introducing one or more cannabinoids to a genetically modified yeast, plant, or bacteria cell culture in a fermenter or suspension cell culture.
   64. The method of embodiment 62, wherein the step of introducing comprises the step of biosynthesizing one or more cannabinoids in a genetically modified yeast, plant, or bacteria cell culture wherein said heterologous LC-carrier protein binds said one or more biosynthesized cannabinoids to form a water-soluble protein-cannabinoid composition.
   65. The method of embodiment 62, wherein said heterologous LC-carrier protein comprises a heterologous LC-carrier protein having an amino acid sequence selected from the group of consisting of: SEQ ID NOs. 1-29, and 30-46, or a homolog having affinity towards at least one cannabinoid thereof.
   66. The method of embodiments 62 and 65, wherein said heterologous LC-carrier protein is coupled with a tag.
   67. The method of embodiments 62 and 65, wherein said heterologous LC-carrier protein is coupled with a secretion signal.
   68. The method of embodiment 67, wherein said secretion signal comprises a secretion signal selected from the group consisting of: SEQ ID NO. 47, and SEQ ID NOs. 106-112.
   69. The method of embodiment 62, wherein the at least one cannabinoid comprises a cannabinoid selected from the group consisting of: cannabidiol (CBD), cannabidiolic acid (CBDA), Δ⁹-tetrahydrocannabinol (THC), tetrahydrocannabinolic acid (THCA), and (cannabigerolic acid) CBGA).
   70. The method of embodiment 62, and further comprising the of step of genetically modifying the LC-carrier protein form an engineered LC-carrier protein having enhanced affinity for at least one cannabinoid, such genetic modification comprising one or more of the following:
replacing one or more amino acid residues of the LC-carrier protein cannabinoid binding pocket with side chains pointing towards orientated toward the binding cavity;
replacing one or more amino acid residues of the LC-carrier protein cannabinoid binding pocket having a hydrophilic side chain with amino acid residues having a hydrophobic side chain; and
replacing one or more small hydrophobic amino acid residues of the LC-carrier protein cannabinoid binding pocket with larger hydrophobic amino acid residues.
   71. The LC-carrier protein of embodiments 62 and 70, wherein the LC-carrier protein is further genetically modified to decrease aggregation propensity or potential antigenicity.
   72. The LC-carrier protein of embodiments 1, 13, 24 and 32, wherein said LC-carrier protein a plant LC-carrier.
   73. The method of embodiments 62 and 65, wherein said LC-carrier protein having affinity towards at least one cannabinoid comprises an LC-carrier protein having a β-barrel enclosed cannabinoid-binding site having an internal cavity, and an external loop scaffold structure.
   74. The method of embodiments 62 and 73, wherein the LC-carrier comprises an engineered LC-carrier protein further comprising a truncated LC-carrier protein forming a β-barrel enclosed cannabinoid-binding site having an internal cavity, and an external loop scaffold structure.
   75. The method of embodiment 74, wherein said engineered LC-carrier protein comprises an engineered LC-carrier protein having an amino acid sequence selected from the group of consisting of: SEQ ID NOs. 30-46.
   76. The water-soluble protein-cannabinoid composition of any of the embodiments above wherein said water-soluble protein-cannabinoid composition is introduced to a consumer product meant for human-consumption, or a pharmaceutical composition for administration of a therapeutically effective dose to a subject in need thereof; or a prodrug for administration of a therapeutically effective dose to a subject in need thereof.
   77. A genetically modified Cannabis plant expressing a nucleotide sequence operably linked to a promoter encoding at least one Lipocalin Carrier (LC)-carrier protein.
   78. The Cannabis plant of embodiment 36 and wherein said FABP-carrier protein comprises a FABP-carrier protein selected from the group consisting of: an amino acid sequence according to SEQ ID NOs. 1-29, and 30-46.
   79. The Cannabis plant of embodiments 77 and 78, and further comprising the step of expressing a nucleotide sequence operably linked to a promoter encoding one or more cannabinoid synthases having its trichome targeting sequence disrupted or removed.
   80. The Cannabis plant of embodiment 79, wherein one or more cannabinoid synthase genes has been disrupted or knocked out.
   81. The Cannabis plant of embodiment 80, wherein said one or more cannabinoid synthases having its trichome targeting sequence disrupted or removed is selected from the group consisting of the nucleotide sequence identified as: SEQ ID NOs. 55-57.
   82. The Cannabis plant of embodiment 77, and further comprising the step of expressing at least one myb transcription factor.
   83. The Cannabis plant of embodiment 82, wherein said at least one myb transcription factor is selected from the group consisting of: SEQ ID NOs. 58-62.
   84. The Cannabis plant of embodiment 77, and further comprising the step of expressing at least one catalase.
   85. The Cannabis plant of embodiment 84, wherein said at least one catalase is selected from the group consisting of: SEQ ID NOs. 48-52.
   86. The Cannabis plant of embodiment 77, and further comprising the step of expressing at least one heterologous glycosyltransferase.
   87. The Cannabis plant of embodiment 86, wherein said at least one at least one heterologous glycosyltransferase is selected from the group consisting of: SEQ ID NOs. 73-88, and SEQ ID NOs. 102-103.

Additional aspects of the invention may be evident from the specification and figures below.

### BRIEF DESCRIPTION OF THE FIGURES

FIG. 1. Representative model homology of 10 cannabinoid lipocalin proteins in an overlapping configuration. (A) Top image demonstrates a generally conserved β-barrel cannabinoid binding pocket. (B) Bottom is a side view of representative lipocalin templates. Purple regions represent conserved domain, gray regions represent side chains.

FIG. 2. (A)(B) Representative Cannabinoid (CBD) docked in conserved β-barrel binding pocket of exemplary plant cannabinoid carrier protein.

FIG. 3. β-barrel binding pockets of 10 template lipocalins on left and simulated 36 OBP proteins on right in an overlapping configuration demonstrating a generally conserved β-barrel binding pocket.

FIG. 4. β-sheet structures of 10 template lipocalins on left and simulated 36 OBP proteins on right in an overlapping configuration demonstrating a generally conserved β-barrel binding pocket.

FIG. 5. Exemplary cannabinoid (THC) simulated docked structure of odorant binding protein XP_00687726.1 identified as amino acid sequence SEQ ID NO. 120, further having a generally conserved β-barrel binding pocket and β-sheet structure.

FIG. 6. Vector map of modified pET24a (+).

FIG. 7. Small scale protein expression of (A) full length green algae lipocalin. Lane 1: lysate. Lane 2: supernatant after cell lysis. Lane 3: Pellet after cell lysis. Expected band size is 39.8 kDa. (B) His-tag lipocalin poppyseed and oilseed. Expected band sizes are around 23.4 kDa and 20.3 kDa respectively. The lipocalin expression was confirmed with SDS-PAGE according to molecular weight. Lysate shows the total protein expression, supernatant and pellet shows soluble and insoluble protein respectively. All lipocalin were expressed as insoluble protein.

FIG. 8. ANS displacement for analysis of lipocalin binding to THC and CBD. (A) full length lipocalin from algae (B) truncated lipocalin from algae (C) lipocalin from oilseed D) lipocalin from poppy seed (E) odorant binding protein 1 (OBP1) from naked mole rat (F) odorant binding protein 2 (OBP2) mouse. (G) Average relative change in fluorescence as a measure of binding of cannabinoid to protein. All the four proteins bind to both THC and CBD. Notably, truncated algae lipocalin binds to THC better than full length. OBP2 demonstrated the highest binding to CBD and THC. The change of emission spectra upon ligand binding correlates with change to aromatic residues exposure due to interaction with the ligand.

### MODE FOR CARRYING OUT THE INVENTION

In certain embodiments, the invention may include the use of L/OBP-carrier proteins to solubilize cannabinoids, terpenes/terpenoids, and other short-chain fatty acid phenolic compounds. In another embodiment, the present invention may include the usage of novel and organismal proteins for the isolation, transportation, or storage of target hydrophobic molecules including cannabinoids, terpenes, and volatiles. In a preferred embodiment, one or more L/OBP-carrier proteins according SEQ ID NO. 1-46, and SEQ ID NO. 1-46, as well as the homologs and orthologs of said sequences, may be combined with target hydrophobic molecules, such as a cannabinoid, to aid in solubilization, extraction, isolation, or storage.

In one embodiment, the invention may include systems, methods and compositions to solubilize cannabinoids, terpenes/terpenoids, and other short-chain fatty acid phenolic compounds utilizing L/OBP-carrier proteins as generally described herein. In this embodiment, the use of L/OBP-carrier protein compositions to solubilize cannabinoids may facilitate the solubilization, extraction, isolation, or storage in in vitro, ex vivo, and in vivo systems, as well as their use in consumer products where enhanced solubility may improve the product's characteristics or price as well as their use in commercial products where enhanced solubility may improve the product's characteristics or price.

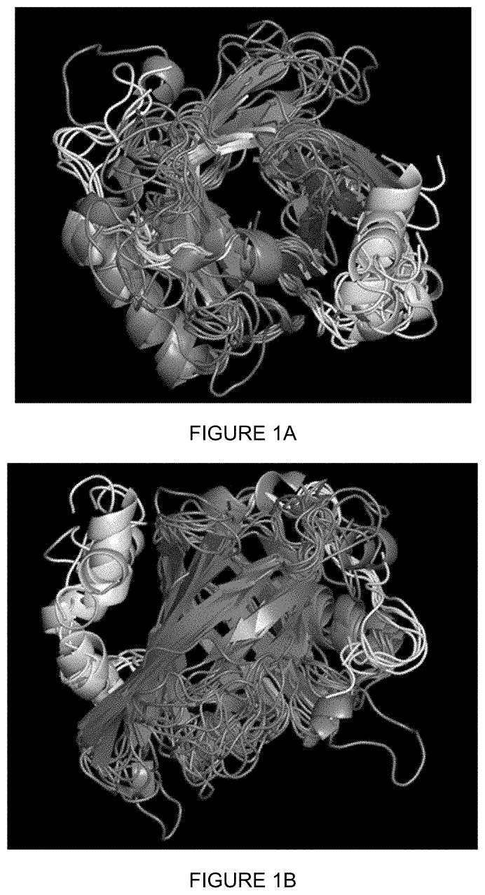

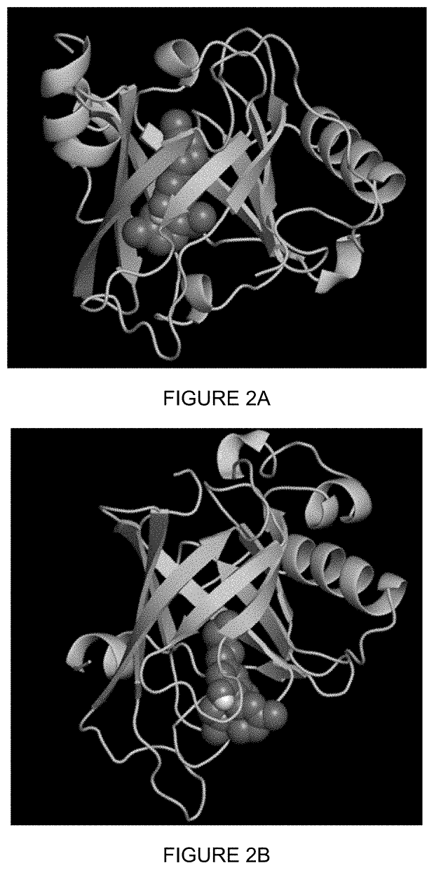

As noted below, in one embodiment, the present invention includes the generation and use of one or more L/OBP-carrier proteins to bind to, and solubilize target hydrophobic molecules, and preferably cannabinoids. In a preferred embodiment, L/OBP-carrier proteins as outlined in Tables 1-2, or the exemplary amino acid sequences identified as SEQ ID NOs. 1-46, and 113-148, may be combined with one or more cannabinoids or other target hydrophobic molecules resulting in an increase to the water-solubility of the complex. Notably, in one particular embodiment, as demonstrated in FIGS. 1-2, LC-carrier proteins having an affinity for one or more cannabinoids may be generated from the plant lipocalins family with simulated structural backbones with close homology to identified plant lipocalin structures identified in Table 4. As shown in FIG. 1 below, across this genus of plant-derived LC-carrier proteins having affinity for one or more cannabinoid or other similar compounds may include common structural features.

As shown in FIG. 1, which demonstrates 10 exemplary plant LC-carrier protein structures that maintain a conserved β-barrel binding pocket as further shown in FIG. 2. The three-dimensional structure of the LC-carrier proteins that have affinity for one or more cannabinoid or other similar compounds also preserve the β-barrel binding pocket as shown in FIG. 1 when overlaid one on-top of another also. In one preferred embodiment, a cannabinoid, such as THC, CBD, or other similar cannabinoid compound may be introduced to a full-length or truncated LC-carrier protein having a β-barrel binding pocket as shown in FIG. 2. In one embodiment, an exemplary LC-carrier protein may bind one or more cannabinoids, such as CBD as demonstrated in Table 2, and FIG. 2, respectively.

As used herein, the terms LC-carrier or LC-carrier protein specifically encompasses plant lipocalins, and plant-lipocalin-like proteins, for example, as generally identified below in SEQ ID NO. 2-46, as well as artificial amino acid sequence identified as SEQ ID NO. 1, which describes an artificial novel unique consensus sequence based on a family of homologous plant sequences that is unique from any characterized plant sequence having affinity for one or more cannabinoids. As used herein, the terms LC-carrier or LC-carrier proteins also specifically encompasses binding domains or fragments or partial sequences of identified LC-carrier proteins, such as those identified in SEQ ID NOs. 1-29, that may exhibit affinity towards one or more cannabinoids. In some embodiments, a partial sequence may include those sequences identified as SEQ ID NO. 30-46, as well as any protein that may incorporate one or more of these fragments, for example as a chimera fusion protein, or a dimer, trimer etc. . . . or other multiprotein complex configuration of the same. Additionally, LC-carrier proteins may be generically used to explicitly describe proteins, regardless of family or classification, that exhibits a β-barrel binding pocket, a β-sheet structure, as well as several alpha-helices and side-chain formations that form an affinity region for a cannabinoid, terpene or other short-chain fatty acid phenolic compounds. Finally, the term “LC-carrier or LC-carrier proteins” explicitly encompasses LC-carrier like proteins, LC-carrier homologs, LC-carrier orthologs, lipocalins-like, and conserved, or semi-conserved binding affinity regions, sequences or motifs having affinity for a cannabinoid, terpene or other short-chain fatty acid phenolic compounds.

In another embodiment, the present invention may include the usage of modified OBP-carrier proteins, proteins designed from novel and organismal proteins for increasing the water-solubility of target hydrophobic molecules including cannabinoids, terpenes, and volatiles and the isolation, transportation, or storage of said molecules. In a preferred embodiment, OBP-carrier proteins as identified in outlined in Table 1 and SEQ ID NOs. 113-148, and may be combined with target hydrophobic molecules to aid in solubilization, extraction, isolation, or storage, as well as their use in commercial products where enhanced solubility may improve the product's characteristics or price.

As noted above, in one embodiment, the present invention includes the generation and use of OBP-carrier proteins to target hydrophobic molecules including cannabinoids, terpenes, and other volatiles. In a preferred embodiment, OBP-carrier proteins as outlined in Table 1, or the exemplary amino acid sequences identified as SEQ ID NOs. 113-148, may be combined with cannabinoids or other target hydrophobic molecules resulting in an increase to the water-solubility of the complex. Notably, as demonstrated in Table, 1 OBP-carrier proteins having an affinity for cannabinoid may be from the lipocalins family with simulated structural backbones with close homology to identified lipocalin template structures identified in Table 1. As shown in FIG. 1 above, across this genus of lipocalin proteins having affinity for one or more cannabinoid or other similar compounds may include common structural features.

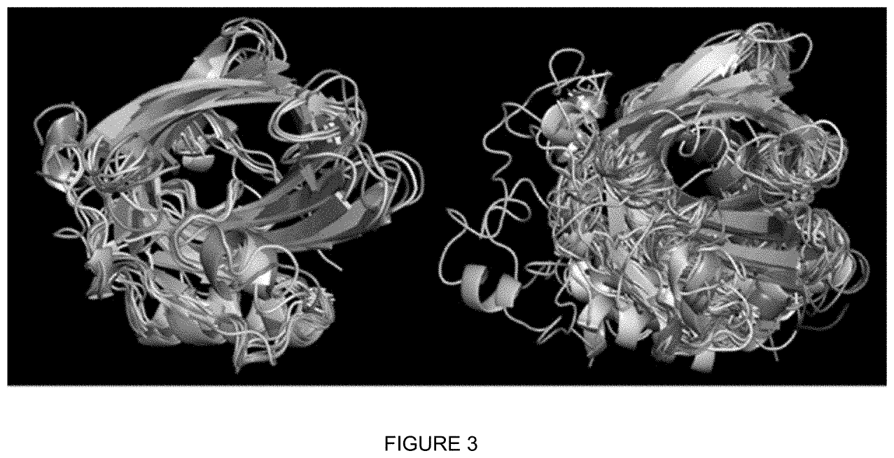

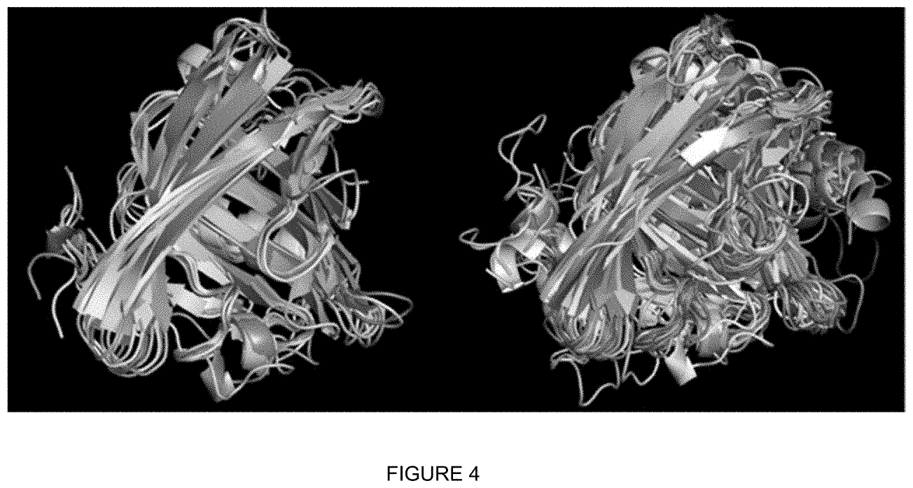

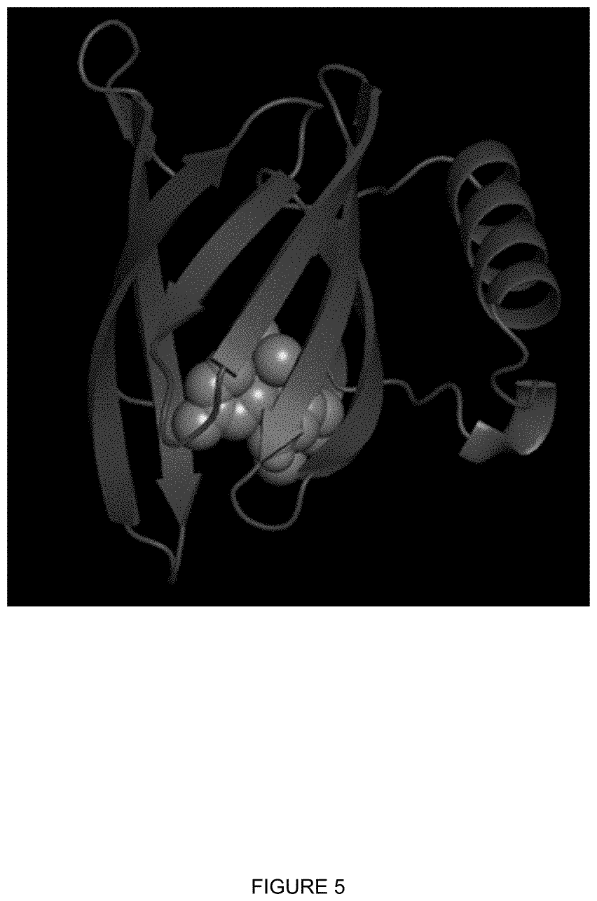

As shown in FIG. 3, which demonstrate 10 template or known lipocalins protein structures maintain a β-barrel binding pocket and β-sheet structure as shown in FIG. 4. The three-dimensional structure of the 26 predicted lipocalins protein that have affinity for one or more cannabinoid or other similar compounds also preserve the β-barrel binding pocket as shown in FIG. 1 and the β-sheet structure when overlaid one on-top of another also. In one preferred embodiment, a cannabinoid, such as THC, CBD, or other cannabinoid compound may bind to a protein having a β-barrel binding pocket and β-sheet structure as shown in FIG. 4. In one embodiment, an exemplary OBP-carrier protein may bind one or more cannabinoids, such as THC as demonstrated in Table 1 and FIG. 5.

As used herein, “OBP-carrier” or “OBP-carrier proteins” explicitly includes OBP and non-plant lipocalins that have affinity for a cannabinoid, terpene or other short-chain fatty acid phenolic compounds. Additionally, “OBP-carrier” or “OBP-carrier proteins” may be generically used to explicitly describe proteins, regardless of family or classification, that exhibits a β-barrel binding pocket and β-sheet structure that forms an affinity region for a cannabinoid, terpene or other short-chain fatty acid phenolic compounds. Finally, the term “OBP-carrier” or “OBP-carrier proteins” explicitly encompasses OBP-carrier-like proteins, OBP-carrier homologs, OBP-carrier orthologs, non-plant lipocalins-like, homologs of non-plant lipocalins, and orthologs of non-plant lipocalins having affinity for a cannabinoid, terpene or other short-chain fatty acid phenolic compounds.

In another embodiment, the current invention may include the rational design of novel L/OBP-carrier protein constructs to increase cannabinoid water solubility via binding. In a preferred embodiment, an L/OBP-carrier proteins, for example as identified in SEQ ID NO. 1-29, and 113-148, or a homolog thereof, may be used to solubilize cannabinoids and other compounds in both in vitro and in vivo systems. Additional embodiments may include the generation of genetically modified L/OBP-carrier protein that may be used to solubilize cannabinoids. In this embodiment, site-direct mutations may be engineered into an L/OBP-carrier protein, or in some instances a wild-type L/OBP-carrier protein may be truncated to retain only amino acid sequences needed to bind one or more target cannabinoids. In another embodiment, such site-directed mutations may be rationally designed such that one or more mutations may be made near a cannabinoid, or other binding site. Such rationally designed mutations may modulate the compounds binding affinity with the L/OBP-carrier protein. In this preferred embodiment, rationally designed mutations may increase its strength of binding with a cannabinoid, terpene, or other short-chain fatty acid phenolic compound. In some further embodiments, rationally designed mutations may enhance binding affinity for the L/OBP-carrier protein that is compound specific. In this embodiment, mutations at and/or near the cannabinoid affinity site may be rationally designed to increase its strength of binding with, for example, THC, CBD or other cannabinoids as identified herein.

In another embodiment of the current invention, a wild type L/OBP-carrier protein may be established and then rationally designed through site-directed mutation(s) that may decrease the aggregation propensity and potential antigenicity for the L/OBP-carrier protein.

In another embodiment, the current invention may include the rational design of mutations at and/or near the cannabinoid binding site of an L/OBP-carrier protein to enhance its binding affinity for THC, CBD or other related cannabinoids. In one preferred embodiment, these mutations may be designed into one or more of the amino acid sequences identified as SEQ ID NO. 1-46, and 113-148, or a sequence incorporating the fragment thereof, for example as identified as SEQ ID NO. 30-46, using a combination of in vitro, in vivo studies as well as bioinformatics approaches such as computational docking, binding affinity estimation, and molecular dynamics simulations. Such bioinformatics applications may be further employed to identify additional potential L/OBP-carrier proteins, as well as direct specific point-mutations to modulate or enhance cannabinoid binding affinity. The above L/OBP-carrier proteins are provided as exemplary embodiments only and are not considered limited of the variety of L/OBP-carrier proteins that may be encompassed by this disclosure. Nor are they limiting as to the number of punitive cannabinoid, or other short-fatty-acid phenolic compound affinity sites that may be engineered in an L/OBP-carrier protein. Consideration of which may include the desired type of short-fatty-acid phenolic compound to be bound by the L/OBP-carrier protein, as well as steric considerations resulting from the addition of such modified affinity motifs presented in the three-dimensional folded protein. Naturally, certain modifications may be made to an L/OBP-carrier protein that may alter the affinity strength of one or more existing cannabinoid affinity sites. For example, in one exemplary embodiment, an L/OBP-carrier protein may have a micromolar affinity for a cannabinoid, while an engineered L/OBP-carrier protein, whether modified through one or more point mutations, or through truncation, may be engineered to have a nanomolar or greater affinity for cannabinoids. As one of ordinary skill in the art would recognize, a ligand, such as a cannabinoid, or other short-chain fatty acid phenolic compound, with nanomolar (nM) dissociation constant may bind more tightly to a particular protein than a ligand with micromolar (μM) dissociation constant. As a result, in certain embodiments of the inventive technology, engineered L/OBP-carrier proteins may be generated that have a customized dissociation constant. This customized dissociation constant may be engineered according to the specifications of a particular application. For example, in one application an engineered L/OBP-carrier protein may be engineered to have one or more cannabinoid affinity sites having nanomolar (nM) or greater dissociation constant. Such engineered L/OBP-carrier proteins may be useful for long-term storage of cannabinoids in solution, or for applications including various commercial and other consumer products where the engineered L/OBP-carrier protein may be exposed to artificial, or natural environmental conditions, as well as other chemical processes that might degrade the protein structure and prematurely release the cannabinoid. Alternatively, in one application an engineered L/OBP-carrier protein may be engineered to have one or more cannabinoid affinity sites having micromolar (μM) dissociation constant. Such engineered L/OBP-carrier protein may allow for one or more cannabinoid compounds to be more easily released from the L/OBP-carrier. In one preferred embodiment, an engineered L/OBP-carrier protein may include one or more a cannabinoid affinity sites having a macro- or micromolar (μM) dissociation that may allow for greater release, as compared for example to nanomolar (nM) dissociation, and bioavailability of the cannabinoid upon consumption. Naturally, the number and scope of engineered L/OBP-carrier protein are provided as exemplary embodiments only and are not considered limiting of the variety of L/OBP-carrier proteins that may form an L/OBP-scaffold. As noted above, for amino acid sequences for engineered LC-carrier protein such as those identified in SEQ ID NO. 1 and 30-46 in particular.

As noted above, cannabinoid producing strains of Cannabis, as well as other plants may be utilized with the inventive technology. In certain preferred embodiments, Cannabis plant material may be harvested and undergo cannabinoid extraction through one or more of the methods generally known in the art. These extracted cannabinoids, terpenoids and other short chain fatty acid phenolic compounds, may be introduced to a quantity of L/OBP-carrier proteins, and preferably engineered L/OBP-carrier proteins to be solubilized as described herein.

In one embodiment, yeast cells may be transformed with artificially created expression vectors encoding one or more L/OBP-carrier proteins, preferably one or more engineered L/OBP-carrier proteins. In this preferred embodiment, the nucleotide sequences encoding the L/OBP-carrier or engineered L/OBP-carrier protein(s) may be codon optimized for exogenous expression. Additional embodiments may include operably linked genetic control elements such as promotors and/or enhancers as well as post-transcriptional regulatory elements that may also be expressed in transgenic yeast such that the presence, quantity and activity of any L/OBP-carrier or engineered L/OBP-carrier proteins present in the yeast culture may be modified and/or calibrated. In a preferred embodiment, the yeast strain may be further modified to generate high-levels of L/OBP-carrier protein. In another preferred embodiment, the yeast strain may include genetically modified yeast cells selected from the group consisting of: genetically modified Pichia pastoris cells, genetically modified Saccharomyces cerevisiae cells, and/or genetically modified Kluyveromyces marxianus cells

In one embodiment, bacterial cells may be transformed with artificially created expression vectors encoding one or more L/OBP-carrier proteins, preferably an engineered L/OBP-carrier protein. In this preferred embodiment, the nucleotide sequences encoding the L/OBP-carrier proteins may be codon optimized for exogenous expression. Additional embodiments may include genetic control elements such as operably linked promotors and/or enhancers as well as post-transcriptional regulatory elements that may also be expressed in transgenic bacteria such that the presence, quantity and activity of any L/OBP-carrier or engineered L/OBP-carrier protein(s) present in the bacteria culture may be modified and/or calibrated. In a preferred embodiment, the bacterial strain may include a high expression strain of bacteria, such as E. coli strain BL21(DE3) for optimal protein expression.

As noted above, in one embodiment the inventive technology may include individual expression or synthesis of one or more L/OBP-carrier or engineered L/OBP-carrier proteins each having a selected molecular tag. In a preferred embodiment, an L/OBP-carrier protein, for example engineered from the amino acid sequences SEQ ID NO. 1-46, and 113-148, or a homolog thereof, may each be configured to contain a poly-His or His-6 tag, which may be used later for protein purification. In this embodiment, the expressed L/OBP-carrier protein may be detected and purified because the string of histidine residues binds to several types of immobilized metal ions, including nickel, cobalt and copper, under appropriate buffer conditions.

In one embodiment of the inventive technology, a cell culture, such as a plant, yeast or bacterial culture, may be genetically modified to express a tagged heterologous L/OBP-carrier and/or engineered L/OBP-carrier protein may be allowed to grow to a desired level of cell or optical density, or in other instances until a desired level of L/OBP-carrier and/or engineered L/OBP-carrier proteins have accumulated in the cultured cells and/or media, for example through the addition of a secretion signal that directs the L/OBP-carrier and/or engineered L/OBP-carrier protein to be exported from the cell. In one embodiment, a secretion signal that may direct posttranslational protein translocation into the endoplasmic reticulum (ER), or in alternative embodiments, a secretion signal that may direct cotranslational translocation across the ER membrane. In an additional embodiment, all, or a portion of the cells containing the accumulated L/OBP- and/or engineered L/OBP-carrier proteins may then be harvested from the culture and/or media, which in a preferred embodiment may be an industrial-scale fermenter or other apparatus suitable for the large-scale culturing of or other microorganisms. The harvested cells may be lysed such that the accumulated L/OBP-carrier and/or engineered L/OBP-carrier proteins may be released to the surrounding lysate. Additional steps may include treating this lysate. Examples of such treatment may include filtering, centrifugation or screening to remove extraneous cellular material as well as chemical treatments to improve later L/OBP-carrier and/or engineered L/OBP-carrier protein yields.

The L/OBP-carrier and/or engineered L/OBP-carrier protein may be further isolated and purified. In one preferred embodiment, the cell lysate may be processed utilizing affinity chromatography or other purification methods. In this preferred embodiment, an affinity column having a ligand configured to bind with one or more of the tags coupled with the L/OBP-carrier and/or engineered L/OBP-carrier protein, for example, a poly-His or His-6 tag, among others, may be immobilized or coupled to a solid support. The lysate may then be passed over the column such that the tagged L/OBP-carrier and/or engineered L/OBP-carrier protein, having specific binding affinity to the ligand become bound and immobilized. In some embodiments, non-binding and non-specific binding proteins that may have been present in the lysate may be removed. Finally, the L/OBP-carrier and/or engineered L/OBP-carrier protein may be eluted or displaced from the affinity column by, for example, a corresponding protein, tag or other compound that may displace or disrupt the tag-ligand bond. The eluted L/OBP-carrier and/or engineered L/OBP-carrier proteins may be collected and further purified or processed. Notably, in other embodiments, L/OBP-carrier proteins may be commercially obtained and used consistent with the embodiments described herein.

All L/OBP-carrier amino sequences described herein include homologs of said sequences which may have between 75-99.9% homology. Where a sequence encoding an L/OBP-carrier having a conserved, or semi-conserved binding affinity site for a cannabinoid or other compound described herein, such as the artificial sequence identified in SEQ ID NO. 1, or L/OBP-carrier fragments identified in SEQ ID NOs. 30-46, may be incorporated into a variety of proteins, and thus increase the range of effective homologies that may be encompassed within the inventive technology.

Another embodiment of the inventive technology includes the generation of novel genetically modified cannabinoid-carrier proteins that may have enhanced affinity for cannabinoid compounds. In one preferred embodiment, the inventive technology includes the generation of novel genetically modified cannabinoid-carrier LC-carrier protein engineered from, for example SEQ ID NO. 1, and 30-46, or a homolog thereof that may have affinity for cannabinoids. In this embodiment, such engineered LC-carrier proteins may include a wild type or pre-generated L/OBP-carrier, such as identified in for example SEQ ID NO. 1-46, or a homolog thereof, which may be genetically modified to produce an engineered LC-carrier. Such novel truncated or engineered LC-carriers may exhibit enhanced cannabinoid docking, as well as more favorable stoichiometry such that less protein may be used to solubilize/deliver a quantifiable amount of a target cannabinoid which may enhance the carrier proteins ability to be used in formulations for various commercial products and the like.

Another embodiment of the inventive technology provides for systems and methods of high-capacity cannabinoid solubilization. In this preferred embodiment, a polynucleotide configured to express one or more L/OBP-carrier proteins, for example SEQ ID NO. 1-46, and 113-148, or a homolog thereof, may be coupled with a tag for purification or isolation purposes and further operably linked to a promoter forming an expression vector. This expression vector may be used to transform a microorganism which may express one or more tagged L/OBP-carrier proteins, and/or tagged engineered L/OBP-carrier proteins which may be further isolated, preferably through affinity purification. The isolated tagged L/OBP-carrier proteins, and/or tagged engineered L/OBP-carrier proteins, may be placed into a bio-reactor or other suitable in vitro, ex vivo, or in vivo, environment where they may be introduced to one or more cannabinoids, terpenoids, and/or other short-chain fatty-acid phenolic compounds. The tagged L/OBP-carrier proteins, and/or tagged engineered L/OBP-carrier proteins, may solubilize the cannabinoids, terpenoids, and/or other short-chain fatty-acid phenolic compounds through affinity binding to one or more affinity site. The solubilized cannabinoids may be isolated and used for commercial, pharmaceutical and other applications as generally described herein.

Another embodiment of the invention provides for methods of masking the typical unpleasant smell and taste of cannabinoid-infused commercial products and beverages. For example, in this embodiment an L/OBP-carrier, and preferably an engineered L/OBP-carrier protein, may bind to one or more cannabinoids and allow it to be solubilized in a liquid solution. In this solubilized state, the carrier protein allows for the masking of the cannabinoid's natural smell and taste. Moreover, in additional embodiments, an L/OBP-carrier and/or engineered L/OBP-carrier protein may bind to, and solubilize one or more terpenes or flavonoids, the compounds in Cannabis primarily responsible for its distinctive smell. In this manner, the invention may generate cannabinoid-infused commercial products, such as consumables and beverages that eliminate, mask or ameliorate the undesired smell and taste of the cannabinoid and terpene compounds.

Another embodiment of the invention provides for methods of generating solubilized cannabinoids, terpenes and other short-chain fatty-acid phenolic compounds that may have a more rapid metabolic uptake or bioavailability upon ingestion. In this embodiment, a L/OBP-carrier and/or engineered L/OBP-carrier protein may bind to one or more cannabinoids and allow it to be solubilized such that upon ingestion it may be more readily taken up by the body, for example, through the association with the aforementioned carrier protein. This embodiment may allow for not only a more rapid uptake of the target compound, but allow for consistent consumer experiences, as well as facilitate a safe and effective consumer-controlled dosing of cannabinoids and other compounds. Such carrier proteins may further protect the cannabinoid, or other compounds from being degraded by chemical processes in the body, such as would be present in the stomach or intestines enhancing bioavailability. This embodiment may further allow for lower amounts of cannabinoid and terpene compounds to be used in infused consumables and beverages as a result of this improved bioavailability. For example, absent this enhance bioavailability of the solubilized cannabinoids and terpenes, a large portion of the compounds may not be efficiently taken up by the body and may be eventually eliminated through natural chemical degradation or other strategies to metabolically clear the compounds from the body.

Another embodiment of the invention provides for methods of generating precise doses and/or formulations and/or ratios of cannabinoids, terpenoids, and/or other short-chain fatty-acid phenolic compounds. In a preferred embodiment, a polynucleotide may be generated that is configured to express one or more L/OBP-carrier and/or engineered L/OBP-carrier proteins configured to have binding affinity motifs that selectively bind an individual or class of cannabinoid, terpenoids, and/or other short-chain fatty-acid phenolic compounds. Again, this selective L/OBP-carrier protein may be coupled with a tag for purification or isolation purposes and may be operably linked to a promoter forming an expression vector. This expression vector may be used to transform a microorganism, such as bacteria, yeast, or algae, which may express the tagged selective L/OBP-carrier protein which may be further isolated, preferably through affinity purification. The isolated selective L/OBP-carrier protein may be placed into a bio-reactor, cell culture or other suitable environment where they may be introduced to one or more cannabinoid, terpenoids, and/or other short-chain fatty-acid phenolic compounds. The L/OBP-carrier protein may selectively solubilize a quantity of cannabinoid, terpenoids, and/or other short-chain fatty-acid phenolic compounds, consistent with its endogenous and/or engineered affinity characteristics. The solubilized cannabinoid, terpenoids, and/or other short-chain fatty-acid phenolic compounds may be used for commercial, pharmaceutical, and other applications as generally described herein.

Another aspect of the invention provides for methods of generating precise mixed doses, ratios, and/or formulations of cannabinoids, terpenoids, and/or other short-chain fatty acid phenolic compounds. In a preferred embodiment, a first polynucleotide may be generated that is configured to express a L/OBP-carrier protein, and preferably an engineered L/OBP-carrier protein configured to have a selective binding affinity motif(s) that selectively bind an individual or class of cannabinoid, terpenoid, and/or other short-chain fatty-acid phenolic compounds. An additional polynucleotide may be generated that is configured to express an L/OBP-carrier protein, and preferably an engineered L/OBP-carrier protein configured to have a cannabinoid binding affinity motif(s) that selectively binds a different individual or class of cannabinoid, terpenoid, and/or other short-chain fatty-acid phenolic compounds. Both selective L/OBP-carrier proteins may be coupled with a tag for purification or isolation purposes and may be incorporated into one or more expression vectors being operably linked to a promotor. Such expression vector(s) may be used to transform a microorganism, such as bacteria, yeast, or algae, which may express the tagged selective engineered L/OBP-carrier proteins which may be further isolated, preferably through affinity purification. The isolated selective L/OBP-carrier proteins may be placed into a bio-reactor, cell culture, or other suitable environment where they may be introduced to one or more cannabinoids, terpenoids, and/or other short-chain fatty-acid phenolic compounds. The first L/OBP-carrier protein may selectively solubilize a quantity of individual or class of cannabinoid, terpenoid, and/or other short-chain fatty-acid phenolic compound consistent with the number and type of its endogenous and/or engineered affinity sites. The additional L/OBP-carrier protein may selectively solubilize a quantity of a separate individual or class of cannabinoid, terpenoid, and/or other short-chain fatty-acid phenolic compound consistent with the number and type of its endogenous and/or engineered affinity sites. The solubilized cannabinoid, terpenoids, and/or other short-chain fatty-acid phenolic compounds may be used for commercial, pharmaceutical, and other applications as generally described herein.

Another aspect of the invention may include in vitro systems and methods to solubilize cannabinoids, terpenoids, and/or other short-chain fatty-acid phenolic compounds. In a preferred embodiment, L/OBP-carrier proteins, for example SEQ ID NO. 1-46, or homologs thereof, and/or engineered LC-carrier proteins, for example engineered from SEQ ID NO. 1, and 20-46, or homologs thereof, may be artificially synthesized in vitro and then placed into a bio-reactor, cell culture, or other suitable environment where they may be introduced to one or more cannabinoids, terpenoids, and/or other short-chain fatty-acid phenolic compounds. The L/OBP-carrier proteins and/or engineered L/OBP-carrier proteins may solubilize the cannabinoids, terpenoids, and/or other short-chain fatty acid phenolic compounds as generally described herein. The solubilized compounds, such as cannabinoids, may be used for commercial, pharmaceutical and other applications as generally described herein.

Another embodiment of the inventive technology provides for direct systems and methods of high-capacity cannabinoid solubilization. In this preferred embodiment, a polynucleotide configured to express one or more L/OBP-carrier, and/or engineered L/OBP-carrier proteins, for example SEQ ID NOs. 1-46, or a protein that incorporates a portion or fragment of SEQ ID NOs. 1-46, such as SEQ ID NOs. 30-46, or a homolog thereof, and may further be coupled with a tag for purification or isolation purposes. This polynucleotide may be operably linked to a promoter forming an expression vector. This expression vector may be used to transform a microorganism, such as yeast or bacteria, which may be grown in an industrial scale fermenter or other like apparatus known in the art for high-level protein production. While in culture, the genetically modified microorganism may express one or more tagged L/OBP-carrier proteins, and/or tagged engineered L/OBP-carrier protein. Glycosylated or un-glycosylated short-chain fatty-acid phenolic compounds, such as cannabinoids, terpenes, and other volatiles may be extracted from cannabinoid-producing plants or artificially biosynthesized and added to the cell culture and be solubilized by the L/OBP-carrier proteins as generally described herein.

In one embodiment, the L/OBP-carrier proteins and/or engineered L/OBP-carrier proteins produced in a cell culture may be coupled with a secretion signal to enable exportation to the culture's media or supernatant. In this aspect of the invention, an L/OBP-carrier protein and/or engineered L/OBP-carrier protein may be exported out of a cell through the action of the secretion signal that may direct post-translational protein translocation into the endoplasmic reticulum (ER), or in alternative embodiments, a secretion signal that may direct cotranslational translocation across the ER membrane where it may assume its three-dimensional form and bind one or more cannabinoid or other compounds as described herein. In one preferred embodiment, an L/OBP-carrier protein and/or engineered L/OBP-carrier may be generated in a cell culture, preferably a bacterial, yeast, plant, algal, or fungi cell culture, and then be exported out of the sell through the action of the secretion signal where, in some embodiments, it may assume it's three dimensional form and bind one or more cannabinoid or other compounds that may be present, preferably by addition of said compound to the culture's supernatant.

In another aspect of the invention, an L/OBP-carrier protein and/or engineered L/OBP-carrier may be exported out of a cell through the action of the secretion signal after it has assumed a transitory and or final three dimensional form and may further be bound to one or more cannabinoid or other compounds as described herein. In one preferred embodiment, an L/OBP-carrier protein and/or engineered L/OBP-carrier may be generated in a cell culture, preferably a bacterial, yeast, plant, algal, or fungi cell culture, and more preferably a plant suspension culture of a cannabinoid-producing plant such as Cannabis, where it may assume a transitory or final three dimensional form and bind one or more cannabinoid or other compounds that may be present or produced in the cell.

Another embodiment of the inventive technology provides for direct systems and methods of high-capacity cannabinoid solubilization. In this preferred embodiment, a polynucleotide configured to express one or more L/OBP-carrier or engineered L/OBP-carrier proteins, or protein incorporating an L/OBP cannabinoid binding domain, may be coupled with a tag for purification or isolation purposes. Such polynucleotide may be operably linked to a promoter forming an expression vector. This expression vector may be used to transform a bacterium which may be grown in an industrial scale fermenter or other like apparatus known in the art for high-level protein production. While in culture, the genetically modified bacteria may express one or more tagged L/OBP-carrier proteins and/or tagged engineered L/OBP-carrier proteins that may also be coupled with a secretion signal. Short-chain fatty-acid phenolic compounds, such as cannabinoids, terpenes, and other volatiles, may be extracted from cannabinoid-producing plants or artificially biosynthesized and added to the cell culture, preferably in a fermenter or other appropriate device. The L/OBP-carrier proteins and/or engineered L/OBP-carrier proteins produced in culture may be introduced to one or more cannabinoids, terpenoids, and/or other short-chain fatty-acid phenolic compounds in the culture. The L/OBP-carrier proteins and/or engineered L/OBP-carrier proteins may bind to and solubilize one or more cannabinoids, terpenoids, and/or other short-chain fatty-acid phenolic compounds. The tagged L/OBP-carrier proteins and/or engineered L/OBP-carrier proteins, and their bound compounds, may be isolated utilizing affinity chromatography or other purification methods. The solubilized cannabinoids may be used for commercial, pharmaceutical, and other applications as generally described herein.

Another embodiment of the inventive technology provides for direct systems and methods of high-capacity cannabinoid solubilization. In this preferred embodiment, a polynucleotide configured to express one or more L/OBP-carrier and/or engineered L/OBP-carrier proteins or protein incorporating a L/OBP cannabinoid binding domain, may be coupled with a tag for purification or isolation purposes and may further be coupled with a secretion tag. Such polynucleotide may be operably linked to a promoter forming an expression vector. This expression vector may be used to transform a yeast cell which may be grown in industrial scale fermenter or other like apparatus known in the art for high-level protein production. While in culture, the genetically modified yeast may express one or more tagged L/OBP-carrier proteins and/or tagged engineered L/OBP-carrier proteins. Short-chain fatty-acid phenolic compounds, such as cannabinoids, terpenes, and other volatiles, may be extracted from cannabinoid-producing plants or artificially biosynthesized and added to the cell culture. The isolated L/OBP-carrier proteins, and/or engineered L/OBP-carrier proteins produced in culture may be introduced to one or more cannabinoids, terpenoids, and/or other short-chain fatty-acid phenolic compounds in the culture. The L/OBP-carrier proteins and/or engineered L/OBP-carrier proteins may bind to and solubilize one or more cannabinoids, terpenoids, and/or other short-chain fatty-acid phenolic compounds. The tagged L/OBP-carrier proteins and/or engineered L/OBP-carrier proteins, and their bound compounds, may be isolated utilizing affinity chromatography or other purification methods. The solubilized cannabinoids may be used for commercial, pharmaceutical, and other applications as generally described herein.

Another embodiment of the inventive technology provides for systems and methods of high-capacity cannabinoid solubilization coupled with cannabinoid biosynthesis in microorganisms genetically engineered to produce cannabinoids. Implementing cannabinoid biosynthesis strategies proposed by: Carvalho A, et al.; US Pat. App. No. US20180371507, by Paulos et al.; and WO2017139496, by Hussain et al.; (all of which are incorporated herein by reference) for the generation of cannabinoids in microorganisms such as yeast, fungi, algae, and bacteria, in one embodiment the inventive technology may include systems and methods for solubilization of cannabinoids produced in non-cannabinoid producing microorganisms or artificial chemically-synthesized cannabinoids.

In one embodiment, one or more metabolic pathways for cannabinoid biosynthesis may be reconstructed in z microorganism, such as bacteria, fungi, or yeast. Such pathways may be reconstructed through the expression of a plurality of heterologous genes necessary for the biosynthesis of precursor and cannabinoid compounds. In one preferred embodiment, a microorganism, such as bacteria, yeast, or fungi, may be genetically engineered to produce one or more cannabinoids, terpenes, or other short-chain fatty acid phenolic compounds. The microorganism may be further genetically modified to express a polynucleotide encoding one or more L/OBP-carriers or a homolog thereof, such as those identified in SEQ ID NOs. 1-46, and 113-148, or homologs thereof. In one preferred embodiment, an engineered L/OBP-carrier protein may bind to and solubilize one or more exogenously biosynthesized cannabinoids. This engineered L/OBP-carrier protein may be tagged to facilitate isolation and purification as generally described herein and may further be coupled with a secretion signal.

In another aspect of the invention, an L/OBP-carrier protein and/or engineered L/OBP-carrier may be exported out of a cell through the action of the secretion signal where it may bind to one or more cannabinoid or other compounds located externally to a cell. In one preferred embodiment, an L/OBP-carrier protein and/or engineered L/OBP-carrier may be generated in a cell culture, preferably a bacterial, yeast, plant, algae, or fungi cell culture, and more preferably a plant suspension culture of a cannabinoid-producing plant such as Cannabis, where it may be exported out of the cell and bind one or more cannabinoid or other compounds that may be present in the external cellular environment.

In another aspect of the invention, an L/OBP-carrier protein and/or engineered L/OBP-carrier having a secretion signal may be expressed in a genetically modified yeast culture and exported out of a cell through the action of the secretion signal. In one preferred embodiment, a heterologous polynucleotide may express one or more exportable L/OBP-carrier proteins and/or exportable engineered L/OBP-carrier proteins having a secretion signal. In one embodiment, a secretion signal may direct post-translational protein translocation into the endoplasmic reticulum (ER). In additional embodiments, a secretion signal may direct cotranslational translocation of the carrier protein across the ER membrane.

Notably, protein translocation is the process by which peptides are transported across a membrane bilayer. Translocation of proteins across the membrane of the membrane of the ER is known to occur in one of two ways: cotranslationally, in which translocation is concurrent with peptide synthesis by the ribosome, or posttranslationally, in which the protein is first synthesized in the cytosol and later is transported into the ER.

In eukaryotic organisms such as yeast, proteins that are targeted for translocation across the ER membrane have a distinctive amino-terminal signal sequence, such as the amino acid sequence identified in SEQ ID NO. 106, which is recognized by the signal recognition particle (SRP). The SRP in eukaryotes is a large ribonucleoprotein which, when bound to the ribosome and the signal sequence of the nascent peptide, is able to arrest protein translation by blocking tRNA entry. The ribosome is targeted to the ER membrane through a series of interactions, starting with the binding of the SRP by the SRP receptor. The signal sequence of the nascent peptide chain is then transferred to the protein channel, Sec61. The binding of SRP to its receptor causes the SRP to dissociate from the ribosome, and the SRP and SRP receptor also dissociate from each other following GTP hydrolysis. As the SRP and SRP receptor dissociate from the ribosome, the ribosome is able to bind directly Sec61.

The Sec61 translocation channel (known as SecY in prokaryotes) is a highly conserved heterotrimeric complex composed of α-, β- and γ-subunits. The pore of the channel, formed by the α-subunit, is blocked by a short helical segment which may become unstructured during the beginning of protein translocation, allowing the peptide to pass through the channel. The signal sequence of the nascent peptide intercalates into the walls of the channel, through a side opening known as the lateral gate. During translocation, the signal sequence is cleaved by a signal peptide peptidase, freeing the amino terminus of the growing peptide.

During cotranslational translocation in eukaryotes, the ribosome provides the motive power that pushes the growing peptide into the ER lumen. During posttranslational translocation, additional proteins are necessary to ensure that the peptide moves uni-directionally into the ER membrane. In eukaryotes, posttranslational translocation requires the Sec62/Sec63 complex and the chaperone protein BiP. BiP is a member of the Hsp70 family of ATPases, a group which is characterized as having an N-terminal nucleotide-binding domain (NBD), and a C-terminal substrate-binding domain (SBD) which binds to peptides. The nucleotide binding state of the NBD determines whether the SBD can bind to a substrate peptide, in this case an L/OBP-carrier or engineered L/OBP-carrier protein. While the NBD is bound to ATP, the SBD is in an open state, allowing for peptide release, while in the ADP state, the SBD is closed and peptide-bound. The primary role of the membrane protein complex Sec62/Sec63 is to activate the ATPase activity of BiP via a J-domain located on the lumen-facing portion of Sec63. The SBD of BiP binds non-specifically to the peptide as it enters the ER lumen, and keeps the peptide from sliding backwards in a ratchet-type mechanism.

Again, in one preferred embodiment, a L/OBP-carrier and/or engineered L/OBP-carrier protein may be modified to include at least one secretion signal that may facilitate vesicle transport of the protein out of the cell, preferably a yeast cell. In one embodiment, an L/OBP-carrier and/or engineered L/OBP-carrier protein may be modified to include a secretion signal which directs posttranslational protein translocation into the ER. In one preferred embodiment, a secretion signal which directs posttranslational protein translocation into the ER may be identified in amino acid SEQ ID NO. 47 (see below) which encodes an N-terminal secretion signal from α-factor mating pheromone in S. cerevisiae. The secretion signal is made up of a 19 amino acid ‘presequence’ which directs posttranslational protein translocation into the ER, and a 66-amino acid ‘pro region’ mediating receptor-dependent packaging into ER-derived COPAY transport vesicles.

SEQ ID NO. 47:
MRFPSIFTAVLFAASSALAAPVNTTTEDETAQIPAEAVIGYSDLEGD
FDVAVLPFSNSTNNGLLFINTTIASIAAKEEGVSLEKR

In another embodiment, an L/OBP-carrier and/or engineered L/OBP-carrier protein may be modified to include a secretion signal which directs cotranslational translocation across the ER membrane. In one preferred embodiment, an enhanced secretion signal which directs cotranslational translocation across the ER membrane may be identified in amino acid sequence of SEQ ID NO. 106, where the 19 amino acid ‘presequence’ is replaced with the enhanced ‘presequence’ (blue) with the Ost1 (OST=oligosaccharyltransferase) signal sequence identified by amino acid SEQ ID NO. 107:

MRQVWFSWIVGLFLCFFNVSSA

In this preferred embodiment, an enhanced secretion signal may be identified according to SEQ ID NO. 106:

MRQVWFSWIVGLFLCFFNVSSAAPVNTTTEDETAQIPAEAVIGYSDL
EGDFDVAVLPFSNSTNNGLLFINTTIASIAAKEEGVSLEKR

Again, in a preferred embodiment, one or more of the L/OBP-carrier and/or engineered L/OBP-carrier proteins identified herein may be modified and expressed, preferably in a yeast cell, to include a secretion signal which directs post-translational protein translocation into the ER, such signal preferably being SEQ ID NO. 47. Such exportable engineered L/OBP-carrier proteins, such as exemplary amino acid sequence identified as SEQ ID NO. 1-46, may bind to, and solubilize one or more cannabinoids located in the cell, or more preferably they may solubilize one or more cannabinoids outside in the cell, such as cannabinoids added to a cell culture supernatant. The exportable L/OBP-carrier and/or engineered L/OBP-carrier proteins, having solubilized one or more target cannabinoids or other compounds identified herein may be further isolated.

In another embodiment, an engineered L/OBP-carrier protein, such as those identified in SEQ ID NO. 1-46, and 113-148, may be modified and expressed, preferably in a yeast cell, to include an enhanced secretion signal which directs cotranslational translocation across the ER membrane, such signal preferably being. SEQ ID NO. 106 which include the Ost1 signal sequence identified as amino acid sequence SEQ ID NO. 76 coupled with the 66-amino acid ‘pro region’ of the N-terminal secretion signal from α-factor mating pheromone in S. cerevisiae. Such enhanced exportable L/OBP-carrier and/or engineered L/OBP-carrier proteins may bind to, and solubilize one or more cannabinoids located in the cell, or more preferably one or more cannabinoids located outside in the cell, such as cannabinoids added to a cell culture supernatant. The exportable L/OBP-carrier and/or engineered L/OBP-carrier proteins, having solubilized one or more target cannabinoids or other compound identified herein, may be further isolated.

Specific embodiments may include a polynucleotide that expresses a sequence as SEQ ID NOs. 1-46, 113-148 or a homolog thereof coupled with at least one secretion signal identified as the amino acid sequence identified in SEQ ID NO 47 or 106.

Additional embodiments also feature a method for producing L/OBP-carrier and/or engineered L/OBP-carrier polypeptides. The method includes culturing a recombinant bacteria cells in a culture medium under conditions that allow the L/OBP-carrier and/or engineered L/OBP-carrier polypeptides to be secreted into the culture medium, the recombinant bacterium cell comprising at least one exogenous nucleic acid, the exogenous nucleic acid comprising first and second nucleic acid sequences, wherein the first nucleic acid sequence encodes a signal peptide and the second nucleic acid sequence encodes an L/OBP-carrier and/or engineered L/OBP-carrier polypeptides, wherein the first and second nucleic acid sequences are operably linked to produce a fusion polypeptide comprising the signal peptide and the L/OBP-carrier and/or engineered L/OBP-carrier polypeptides, and wherein upon secretion of the fusion or chimera polypeptide from the cell into the culture medium, the signal peptide may be removed from the cannabinoid-containing polypeptide. The method further can include isolating the L/OBP-carrier and/or engineered L/OBP-carrier polypeptides from the culture medium.

In another aspect of the invention, an L/OBP-carrier protein and/or engineered L/OBP-carrier may be exported out of a bacterial cell through the action of a secretion signal where the it L/OBP-carrier protein and/or engineered L/OBP-carrier may be secreted in an unfolded conformation and bind to one or more cannabinoid or other compounds located externally to a cell. In one preferred embodiment, an L/OBP-carrier protein and/or engineered L/OBP-carrier may be generated in a cell culture, preferably a bacterial cell culture, where it may be exported out of the cell and bind one or more cannabinoid or other compounds that may be present in the external cellular environment. In this embodiment, an L/OBP-carrier protein and/or engineered L/OBP-carrier may be coupled with a secretion signal that may direct the carrier protein to be secreted from a bacterium through a SEC-mediated secretion pathway.

Notably, in bacteria, translated peptides may be actively translocated post-translationally through a SecY channel by a protein called SecA. SecA is composed of a nucleotide-binding domain, a polypeptide crosslinking domain, and helical wing and scaffold domains. During translocation, a region of the helical scaffold domain forms a two-finger helix which inserts into the cytoplasmic side of the SecY channel, thereby pushing the translocating carrier peptide through. A tyrosine found on the tip of the two-finger helix plays a critical role in translocation, and is thought to make direct contact with the translocating peptide. The polypeptide crosslinking domain (PPXD) forms a clamp which may open as the translocating peptide is being pushed into the SecY channel by the two-finger helix, and close as the two-finger helix resets to its “up” position. The conformational changes of SecA are powered by its nuclease activity, with one ATP being hydrolyzed during each cycle. This SEC system secretes proteins having a consensus signal peptide that is similar to, but distinct from, that of the Tat system as described below. The Sec signal sequence lacks an N-terminal consecutive-arginine sequence and has a relatively hydrophobic central region and a relatively short signal sequence compared with that of Tat. Exemplary Sec signal sequences may be identified as SEQ ID NO. 108.

Again, in one preferred embodiment, an L/OBP-carrier and/or engineered L/OBP-carrier protein may be modified to include at least one Sec-mediated secretion signal that may facilitate translocation of transport of the unfolded carrier protein out of a bacterial cell via a Sec-secretion pathway. In one embodiment, an L/OBP-carrier and/or engineered L/OBP-carrier protein may be modified to include a secretion signal which directs post-translational protein translocation. In one preferred embodiment, a secretion signal which directs posttranslational protein translocation may be identified in amino acid SEQ ID NO. 108 which encodes an exemplary Sec-signal sequence from E. coli L-asparaginase II.

Again, in a preferred embodiment, one or more of the L/OBP-carrier and/or engineered L/OBP-carrier proteins may be selected from SEQ ID NOs. 1-46, and 113-148, and may be modified and expressed, preferably in a bacterial cell, to include a secretion signal which directs posttranslational protein translocation of the unfolded protein, such signal preferably being SEQ ID NO. 109, or homologous or similar Sec-secretion signal sequence, which may encode an exemplary Sec-secretion signal sequence. Such exportable engineered L/OBP-carrier proteins may be translocated from a bacterial cell to the external environment where they may come into contact with, bind to, and solubilize one or more cannabinoids located outside in the cell, such as cannabinoids added to a cell culture supernatant. The exportable L/OBP-carrier and/or engineered L/OBP-carrier proteins, having solubilized one or more target cannabinoids or other compounds identified herein may be further isolated.

In another aspect of the invention, an L/OBP-carrier protein and/or engineered L/OBP-carrier may be exported out of a bacterial cell through the action of a secretion signal where the L/OBP-carrier protein and/or engineered L/OBP-carrier may assume its folded three-dimensional configuration prior to secretion. In this embodiment, an L/OBP-carrier protein and/or engineered L/OBP-carrier may bind to one or more cannabinoid or other compounds located internally or externally to the cell. In one preferred embodiment, an L/OBP-carrier protein and/or engineered L/OBP-carrier may be generated in a cell culture, preferably a bacterial cell culture, where it may be exported out of the cell and into the external cellular environment. In this embodiment, an L/OBP-carrier protein and/or engineered L/OBP-carrier may be coupled with a secretion signal that may direct the carrier protein to be secreted from a bacterium through a TAT-mediated secretion pathway.

Unlike the Sec system, the Tat system is involved in the transport of pre-folded protein substrates. Proteins are targeted to the Tat pathway by possession of N-terminal tripartite signal peptides. The signal peptides include a conserved twin-arginine motif in the N-region of Tat signal peptide. The motif has been defined as R-R-x-Φ-Φ, where Φ represents a hydrophobic amino acid. In E. coli the Tat pathway comprises the three-membrane protein TatA, TatB and TatC. A fourth protein TatE forms a minor component of the Tat machinery and has a similar function to TatA. Because of the ability to secrete pre-folded protein substrates, the Tat pathway may be especially suited for secreting a high level of heterologous L/OBP-carrier and/or engineered L/OBP-carrier proteins. Estimates of Tat substrates in organisms other than Bacillus subtilits and E. coli have been based predominantly in in silico analysis of genome sequences using programs trained to recognize specific features of tat targeting sequences. An exemplary Tat signal sequences may be identified as SEQ ID NO. 109.

Again, in one preferred embodiment, an L/OBP-carrier and/or engineered L/OBP-carrier protein may be modified to include at least one Tat-mediated secretion signal that may facilitate translocation of transport of the folded carrier protein out of a bacterial cell. In one embodiment, an L/OBP-carrier and/or engineered L/OBP-carrier protein may be modified to include a secretion signal which directs posttranslational protein translocation via a Tet-secretion pathway.

In one preferred embodiment, a secretion signal which directs posttranslational protein translocation may be identified in amino acid SEQ ID NO. 109 or homologous or similar Tat-secretion signal sequence which encodes an exemplary tat signal peptide for E. coli strain k12 periplasmic nitrate reductase.

Again, in a preferred embodiment, one or more of the L/OBP-carrier and/or engineered L/OBP-carrier proteins may be selected from SEQ ID NOs. 1-46, and 113-148, and may be modified and expressed, preferably in a bacterial cell, to include a secretion signal which directs posttranslational protein translocation of the folded protein via a Tet-secretion pathway, such signal preferably being SEQ ID NO. 109 or homologous or similar Tat-secretion signal sequence. Such exportable engineered L/OBP-carrier proteins may be translocated from a bacterial cell already having one or more bound cannabinoids, or other compounds. In alternative embodiments, an exportable engineered L/OBP-carrier protein may be translocated from a bacterial cell where it may come into contact with, bind to, and solubilize one or more cannabinoids located outside in the cell, such as cannabinoids added to a cell culture supernatant. The exportable L/OBP-carrier and/or engineered L/OBP-carrier proteins, having solubilized one or more target cannabinoids or other compounds identified herein may be further isolated.

In another embodiment, the invention includes a recombinant plant or plant cell producing an L/OBP-carrier and/or engineered L/OBP-carrier proteins. The plant or plant cell can include at least one exogenous nucleic acid encoding an L/OBP-carrier and/or engineered L/OBP-carrier proteins, wherein the plant or plant cell is from a species of Cannabis. The plant or plant cell can include at least one exogenous nucleic acid encoding an L/OBP-carrier and/or engineered L/OBP-carrier proteins, wherein the plant or plant cell is from a species of Nicotiana. The plant or plant cell can include at least one exogenous nucleic acid encoding an L/OBP-carrier and/or engineered L/OBP-carrier proteins, wherein the plant or plant cell is from a species other than Nicotiana. The exogenous nucleic acid further can include a regulatory control element such as a promoter (e.g., a tissue-specific promoter such as leaves, roots, stems, or seeds).

A polypeptide can be expressed in monocot plants and/or dicot plants. Techniques for introducing nucleic acids into plants are known in the art, and include, without limitation, Agrobacterium-mediated transformation, viral vector-mediated transformation, electroporation, and particle gun transformation (also referred to as biolistic transformation). See, for example, U.S. Pat. Nos. 5,538,880; 5,204,253; 6,329,571; and U.S. Pat. No. 6,013,863; Richards et al., Plant Cell. Rep. 20:48-20 54 (2001); Somleva et al., Crop Sci. 42:2080-2087 (2002); Sinagawa-Garcia et al., Plant Mol Biol (2009) 70:487-498; and Lutz et al., Plant Physiol., 2007, Vol. 145, pp. 1201-1210. In some instances, intergenic transformation of plastids can be used as a method of introducing a polynucleotide into a plant cell. In some instances, the method of introduction of a polynucleotide into a plant comprises chloroplast transformation. In some instances, the leaves and/or stems can be the target tissue of the introduced polynucleotide. If a cell or cultured tissue is used as the recipient tissue for transformation, plants can be regenerated from transformed cultures if desired, by techniques known to those skilled in the art.

Other suitable methods for introduce polynucleotides include electroporation of protoplasts, polyethylene glycol-mediated delivery of naked DNA into plant protoplasts, direct gene transformation through imbibition (e.g., introducing a polynucleotide to a dehydrated plant), transformation into protoplasts (which can comprise transferring a polynucleotide through osmotic or electric shocks), chemical transformation (which can comprise the use of a polybrene-spermidine composition), microinjection, pollen-tube pathway transformation (which can comprise delivery of a polynucleotide to the plant ovule), transformation via liposomes, shoot apex method of transformation (which can comprise introduction of a polynucleotide into the shoot and regeneration of the shoot), sonication-assisted Agrobacterium transformation (SAAT) method of transformation, infiltration (which can comprise a floral dip, or injection by syringe into a particular part of the plant (e.g., leaf)), silicon-carbide mediated transformation (SCMT) (which can comprise the addition of silicon carbide fibers to plant tissue and the polynucleotide of interest), electroporation, and electrophoresis. Such expression may be from transient or stable transformations.

Additional embodiments also feature a method for producing an L/OBP-carrier and/or engineered L/OBP-carrier polypeptides in plants and preferably a plant cell in culture. The method includes culturing a recombinant plant cell in a culture medium under conditions that allow the L/OBP-carrier and/or engineered L/OBP-carrier polypeptides to be secreted into the culture medium, the recombinant bacterium cell comprising at least one exogenous nucleic acid, the exogenous nucleic acid comprising first and second nucleic acid sequences, wherein the first nucleic acid sequence encodes a signal peptide and the second nucleic acid sequence encodes an L/OBP-carrier and/or engineered L/OBP-carrier polypeptides, wherein the first and second nucleic acid sequences are operably linked to produce a fusion polypeptide comprising the signal peptide and the L/OBP-carrier and/or engineered L/OBP-carrier polypeptides, and wherein upon secretion of the fusion or chimera polypeptide from the plant cell into the culture medium, the signal peptide may be removed from the L/OBP-carrier and/or engineered L/OBP-carrier polypeptide. The method further can include isolating the L/OBP-carrier and/or engineered L/OBP-carrier polypeptides from the culture medium.

In another aspect of the invention, an L/OBP-carrier protein and/or engineered L/OBP-carrier may be exported out of a plant cell through the action of a secretion signal where the L/OBP-carrier protein and/or engineered L/OBP-carrier may be secreted via a plant protein secretion pathway. In a preferred embodiment, L/OBP-carrier protein and/or engineered L/OBP-carrier may be coupled with an N-terminal signal peptide which may direct their translocation to the extracellular region via the Endoplasmic Reticulum-Golgi apparatus and the subsequent endomembrane system. In one preferred embodiment, an L/OBP-carrier protein and/or engineered L/OBP-carrier may be generated in a plant, and preferably a plant cell culture, where it may be exported out of the cell and bind one or more cannabinoid or other compounds that may be present in the external cellular environment. In this embodiment, an L/OBP-carrier protein and/or engineered L/OBP-carrier may be coupled with a secretion signal that may direct the carrier protein to be secreted from a plant cell via the Endoplasmic Reticulum-Golgi apparatus and the subsequent endomembrane system.

Again, in one preferred embodiment, an L/OBP-carrier and/or engineered L/OBP-carrier protein may be modified to include at least one plant secretion signal that may facilitate translocation of transport of the protein out of a plant cell. In one embodiment, an L/OBP-carrier and/or engineered L/OBP-carrier protein may be modified to include a secretion signal which directs translocation out of a cell. In one preferred embodiment, a secretion signal which directs protein translocation from a plant cell may be identified in amino acid SEQ ID NO. 110, which encodes an exemplary secretion signal from an extracellular Arabidopsis protease Ara12 (At5g67360). Additional examples include the amino acid SEQ ID NO. 111, which encodes an exemplary secretion signal from a barley (Hordeum vulgare) alpha amylase. Still further examples include the amino acid SEQ ID NO. 112, which encodes an exemplary secretion signal from a rice a-Amylase.

Again, in a preferred embodiment, one or more of the L/OBP-carrier and/or engineered L/OBP-carrier proteins may be selected from SEQ ID NOs. 1-46, and 113-148, or one or more homologs, and may be modified and expressed, preferably in a plant cell, to include a secretion signal which directs protein translocation out of the plant cell, such signal preferably being SEQ ID NO. 110, 111, and 112. Such exportable engineered L/OBP-carrier proteins may be translocated from a plant cell already having one or more bound cannabinoids, or other compounds. In alternative embodiments, an exportable engineered L/OBP-carrier protein may be translocated from a plant cell where it may come into contact with, bind to, and solubilize one or more cannabinoids located outside in the cell, such as cannabinoids added to a cell culture supernatant. The exportable L/OBP-carrier and/or engineered L/OBP-carrier proteins, having solubilized one or more target cannabinoids or other compounds identified herein may be further isolated.

In another embodiment, one or more of the L/OBP-carrier and/or engineered L/OBP-carrier proteins may be secreted from a plant cell in culture using the Hydroxyproline-Glycosylation (Hyp-Glyco) technology. In this embodiment, one or more of the L/OBP-carrier and/or engineered L/OBP-carrier proteins may be selected from SEQ ID NOs. 1-46, and 113-148, or a homolog thereof, and may be modified and expressed, preferably in a plant cell and further fused with Hyp-rich repetitive peptide (HypRP) tag that directs extensive Hyp-O-glycosylation in plant cells resulting in arabinogalactan polysaccharides populating this repetitive peptide fusion facilitating the secretion of the expressed protein from cultured plant cells. In certain embodiments, a catalase enzyme may be co-expressed with cannabinoid biosynthesis genes and L/OBP-carrier proteins, as well as L/OBP-transporters or other genes that may reduce cannabinoid biosynthesis toxicity and/or facilitate transport of the solubilized cannabinoids through or out of the cell. In one embodiment a heterologous catalase is selected from the group consisting of: the amino acid sequence SEQ ID NO. 48, the amino acid sequence SEQ ID NO. 49, the amino acid sequence SEQ ID NO. 50, the amino acid sequence SEQ ID NO. 51, the amino acid sequence SEQ ID NO. 52 and a sequence having at least 80% homology to amino acid sequence SEQ ID NO. 48, SEQ ID NO. 49, SEQ ID NO. 50, SEQ ID NO. 51 and SEQ ID NO. 52.

Another embodiment of the inventive technology provides for systems and methods of high-capacity cannabinoid solubilization coupled with cannabinoid biosynthesis in cannabinoid producing plants or plants engineered to produce cannabinoids. In this preferred embodiment, cannabinoid biosynthesis may be redirected from the plant's trichome to be localized in the plant cell's cytosol. In certain embodiments, a cytosolic cannabinoid production system may be established as directed in PCT/US18/24409 and PCT/US18/41710, both by Sayre et al. (These applications are both incorporated by reference with respect to their disclosure related to cytosolic cannabinoid production and/or modification in whole, and plant cell systems).

In one embodiment, a cytosolic cannabinoid production and solubilization system may include the in vivo creation of one or more recombinant proteins that may allow cannabinoid biosynthesis to be localized to the cytosol where one or more heterologous L/OBP-carrier proteins may also be expressed and present in the cytosol. This inventive feature allows not only higher levels of cannabinoid production and accumulation, but efficient production of cannabinoids in suspension cell cultures. Even more importantly, this inventive feature allows cannabinoid production and accumulation without a trichome structure in whole plants, allowing cells that would not traditionally produce cannabinoids, such as cells in Cannabis leaves and stalks, to become cannabinoid-producing cells

More specifically, in this preferred embodiment, one or more cannabinoid synthases may be modified to remove all or part of an N-terminal extracellular trichome targeting. An exemplary N-terminal trichome targeting sequence for THCA synthase is identified as SEQ ID NO. 53, while an N-terminal trichome targeting sequence for CBDA synthase is identified as SEQ ID NO. 54. Co-expression with this cytosolic-targeted synthase with a heterologous L/OBP-carrier protein, and preferably an engineered L/OBP-carrier protein, may allow the localization of cannabinoid synthesis, accumulation and solubilization to the cytosol. The cannabinoid carrier proteins may be later isolated with their bound cannabinoid molecules through a water-based extraction process due to their solubility, as opposed to traditional chemical or super-critical CO₂ extractions methods.

As noted below, in certain embodiments cannabinoid biosynthesis may be coupled with cannabinoid glycosylation in a cell cytosol. For example, in one preferred embodiment a cytosol-targeted glycosyltransferase (for example SEQ ID NOs. 73-74) may be expressed in a cell, preferably a cannabinoid producing cell, and even more preferably a Cannabis cell. Such cytosolic targeted enzymes may be co-expressed with heterologous catalase and cannabinoid transporters or other genes that may reduce cannabinoid biosynthesis toxicity and/or facilitate transport through or out of the cell.

In one embodiment a heterologous catalase is selected from the group consisting of: the amino acid sequence SEQ ID NO. 48, the amino acid sequence SEQ ID NO. 49, the amino acid sequence SEQ ID NO. 50, the amino acid sequence SEQ ID NO. 51, the amino acid sequence SEQ ID NO. 52 and a sequence having at least 80% homology to amino acid sequence SEQ ID NO. 48, SEQ ID NO. 49, SEQ ID NO. 50, SEQ ID NO. 51 and SEQ ID NO. 52.

Such cytosolic targeted enzymes may also be co-expressed with one or more myb transcriptions factors that may enhance metabolite flux through the cannabinoid biosynthetic pathway which may increase cannabinoid production. In one embodiment a myb transcription factor may be endogenous to Cannabis, or an ortholog thereof. Examples of endogenous or endogenous like, myb transcription factor may include SEQ ID NO. 58 and 59, or orthologs thereof. In one embodiment a myb transcription factor may be heterologous to Cannabis. A heterologous myb transcription factor may be selected from the group consisting of a nucleotide sequence that expresses: amino acid sequence SEQ ID NO. 60, amino acid sequence SEQ ID NO. 61, amino acid sequence SEQ ID NO. 62.

In an alternative embodiment, isolated heterologous L/OBP-carrier proteins, and preferably engineered L/OBP-carrier proteins, may be added to a cell culture of a cannabinoid-producing plant, preferably a Cannabis suspension cell culture, having a cytosolic cannabinoid production system. In this preferred embodiment, one or more cannabinoid may be produced in the cytosol and transported into the surrounding culture media through passive or active transport mechanisms. Once the cannabinoids have been transported to the surrounding culture media, a quantity of L/OBP-carrier proteins, and preferably engineered L/OBP carrier proteins, may be added to the media and bind to and solubilize one or more cannabinoids. This media may then be removed and replenished, such that the solubilized cannabinoids bound to L/OBP-carrier proteins may be further isolated from the media as generally described herein. In one embodiment, the L/OBP-carrier proteins may be later isolated with their bound cannabinoid molecules through a water-based extraction process due to their solubility, as opposed to traditional chemical or super-critical CO₂ extractions methods. In this way, a cell culture of a cannabinoid producing plant may form a continuous production platform for solubilized cannabinoids. Another embodiment of the invention may include the generation of an expression vector comprising this polynucleotide, namely a cannabinoid synthase lacking an N-terminal extracellular trichome targeting sequence and a heterologous L/OBP-carrier gene, operably linked to a promoter. This expression vector may be used to create a genetically altered plant or parts thereof and its progeny comprising this polynucleotide operably linked to a promoter, wherein said plant or parts thereof and its progeny produce said proteins. For example, seeds and pollen contain this expression vector, a genetically altered plant cell comprising this expression vector such that said plant cell produces said chimeric protein. Another embodiment comprises a tissue culture comprising a plurality of the genetically altered plant cells having this expression vector.

One preferred embodiment of the invention may include a genetically altered cannabinoid-producing plant or cell expressing a cytosolic-targeted cannabinoid synthase protein having a cannabinoid synthase N-terminal extracellular targeting sequence (See e.g., SEQ IDs. 53-54) inactivated or removed. In one embodiment, a cytosolic targeted THCA synthase (ctTHCAs) may be identified as SEQ ID NO. 55, while in another embodiment, cytosolic targeted CBDA synthase (cytCBDAs) is identified as SEQ ID NOs. 56-57, respectively. Such cytosolic-targeted cannabinoid synthase proteins may be operably linked to a promoter. Another embodiment provides a method for constructing a genetically altered plant or part thereof having solubilization of cannabinoids in the plant's cytosol compared to a non-genetically altered plant or part thereof, the method comprising the steps of: introducing a polynucleotide encoding a cannabinoid synthase into a plant or part thereof to provide a genetically altered plant or part thereof, wherein the cannabinoid synthase N-terminal extracellular targeting sequence has been disrupted or removed and further expressing a polynucleotide encoding a cannabinoid-carrier L/OBPs, such as those identified in SEQ ID NO. 1-46, and 113-148, or more preferably an engineered LC-carrier protein, such as those engineered from SEQ ID NOs. 30-46, or a homolog thereof.

Notably, in a preferred embodiment, one or more endogenous cannabinoid synthase genes may be disrupted and/or knocked out and replaced with cytosolic-targeted cannabinoid synthase proteins as described herein. The disrupted endogenous cannabinoid synthase gene(s) may be the same or different than the expressed cytosolic-targeted cannabinoid synthase protein. Methods of disrupting or knocking-out a gene are known in the art and could be accomplished by one of ordinary skill without undue experimentation, for example through CRISPR, Talen, and zinc-finger exonuclease systems, as well as heterologous recombination techniques.

In another embodiment, one or more endogenous cannabinoid synthase genes may be disrupted and/or knocked out in a Cannabis plant or suspension cell culture wherein one or more cannabinoid synthase genes has been disrupted and/or knocked out is selected from the group consisting of: a CBG synthase gene; a THCA synthase, a CBDA synthase, and a CBCA synthase. In this embodiment, the Cannabis plant or suspension cell culture may express a polynucleotide encoding one or more cannabinoid synthases having its trichome targeting sequence disrupted and/or removed which may be selected from the group consisting of: a CBG synthase gene having its trichome targeting sequence disrupted and/or removed; a THCA synthase having its trichome targeting sequence disrupted and/or removed; a CBDA synthase having its trichome targeting sequence disrupted and/or removed; and a CBCA synthase having its trichome targeting sequence disrupted and/or removed.

The current invention may further include systems, methods and compositions for the solubilization of cannabinoids, terpenoids and other short-chain fatty acid phenolic compounds in cell cultures. Exemplary cell cultures may include bacterial, yeast, plant, algae and fungi cell cultures. L/OBP-carrier, and preferable engineered L/OBP-carrier proteins, may be coupled with secretion signals to allow such proteins to be exported from the cell culture into the surrounding media. In this embodiment, an L/OBP-carrier or engineered L/OBP-carrier protein may be engineered to include a secretion signal that may allow it to be exported from a cell. In one preferred embodiment, one or more of sequences identified as SEQ ID NOs. 1-46, and 113-148 may be coupled with a secretion signal. In one preferred embodiment, one or more of sequences identified as SEQ ID NOs. 1-46, and 113-148 may be coupled with the N-terminal secretion signal identified in SEQ ID NO. 47 or SEQ ID NO. 106. One exemplary exportable L/OBP-carrier protein may include SEQ ID NO. 1-46, and 113-148 or an engineered LC-carrier protein engineered from SEQ ID NO. 30-46 or may be coupled with the secretion signal identified as amino acid sequence SEQ ID NO. 47 or 106 to form an enhanced exportable an engineered L/OBP-carrier protein. Naturally, such examples are meant to be illustrative of the type and number of exportable L/OBP-carrier and engineered L/OBP-carrier proteins within the scope of the current invention.

Another aspect of the current invention may include systems, methods and compositions for the solubilization of cannabinoids, terpenoids and other short-chain fatty acid phenolic compounds in whole plants and plant cell cultures. In certain embodiments, such plants or cell cultures may be genetically modified to direct cannabinoid synthesis to the cytosol, as opposed to a trichome structure. Further, L/OBP-carrier, and preferable engineered L/OBP-carrier proteins may be coupled with a secretion signal, for example as identified in SEQ ID NO. 47, to allow such proteins to be exported from the cell into the surrounding media. Expression of exportable and non-exportable L/OBP-carriers and preferable engineered L/OBP-carrier proteins may be co-expressed with one or more catalase and/or myb transcription factors

Another embodiment of the inventive technology may include the generation of a powder containing solubilized cannabinoids. In one preferred embodiment, cannabinoids, terpenes, and other short-chain fatty acid phenolic compounds may be solubilized by association with L/OBP-carrier proteins. L/OBP-carrier proteins, having solubilized a quantity of cannabinoids, may undergo lyophilisation, to form an L/OBP-carrier protein powder containing the solubilized cannabinoids. In a preferred embodiment, an engineered L/OBP-carrier protein may solubilize a quantity of cannabinoids through one of the methods generally described herein and then may further undergo lyophilisation, to form an L/OBP-carrier and/or engineered L/OBP-carrier powder containing the solubilized cannabinoids. This powder may have enhanced properties, such as enhanced cannabinoid affinity to provide greater retention and shelf-life to the cannabinoids in the powdered composition. Additionally, this cannabinoid infused powder may be reintroduced to a liquid such that the cannabinoids are re-dissolved in the liquid. This powder may be used, for example, by consumers that wish to add a quantity of one or more cannabinoids to a beverage or other consumable product. It may also be used for pharmaceutical preparations and for proper cannabinoid dosing. This type of soluble cannabinoid-infused powder may be used as a food additive, or even coupled with flavoring agents to be used as a beverage additive. The presence of the L/OBP-carrier proteins, as well as the enhanced cannabinoid affinity and binding capacity, may allow less powder to be used to achieve an equivalent dose, whether in a pharmaceutical or consumer beverage/consumable product.

Other embodiments may allow for the creation of high-concentration solutions of solubilized cannabinoids bound to L/OBP-carrier proteins. Such solutions may allow a user to generate liquid-based food and beverage additives of varying concentrations. Such solutions may further allow a user to generate liquid-based food and beverage additives of varying types of cannabinoids or combinations of cannabinoids and/or terpenes and the like. Due to the enhanced characteristics of certain engineered L/OBP-carriers, in particular the ability to bind individual cannabinoid molecules utilizing on a truncated part of a protein chain, such solutions may achieve higher than normal concentrations of solubilized cannabinoids while limited quantities of protein content. Also, due to the enhanced affinity characteristics of certain engineered L/OBP-carriers compared to other solubilization solutions like nanoemulsions, liquid solutions having solubilized cannabinoids may achieve a longer-shelf life.

In another embodiment, the inventive technology may include novel systems, methods and compositions to decrease potential antigenicity for the L/OBP-carrier proteins. In one preferred embodiment, the recognition sequences of one or more L/OBP-carriers or preferably engineered L/OBP-carrier proteins that correspond to the formation of one or more post-translational glycosylation sites or motifs may be disrupted. In this embodiment, site-directed mutagenesis of recognition sequences that allow for post-translational glycosylation for the sequences identified as SEQ ID NO. 1-46, and 113-148 or a homolog thereof may be accomplished. The removal of such glycosylation sites in an L/OBP-carrier, or preferably an engineered L/OBP-carrier protein, may result in decreased antigenicity.

In one preferred embodiment, the invention may include a pharmaceutical composition as active ingredient an effective amount or dose of one or more L/OBP-carrier and/or engineered L/OBP-carrier proteins coupled with one or more cannabinoids, terpenes or other short-chain fatty acid phenolic compounds. In some instances, the active ingredient may be provided together with pharmaceutically tolerable adjuvants and/or excipients in the pharmaceutical composition. Such pharmaceutical composition may optionally be in combination with one or more further active ingredients. In one embodiment, one of the aforementioned L/OBP-carrier and/or engineered L/OBP-carrier proteins coupled with one or more cannabinoids, terpenes or other short-chain fatty acid phenolic compounds may act as a prodrug. The term “prodrug” refers to a precursor of a biologically active pharmaceutical agent (drug). Prodrugs must undergo a chemical or a metabolic conversion to become a biologically active pharmaceutical agent. A prodrug can be converted ex vivo to the biologically active pharmaceutical agent by chemical transformative processes. In vivo, a prodrug is converted to the biologically active pharmaceutical agent by the action of a metabolic process, an enzymatic process, or a degradative process that removes the prodrug moiety to form the biologically active pharmaceutical agent. In one embodiment, a mean L/OBP-carrier protein pro-drug and preferably engineered L/OBP-carrier protein pro-drug according to the invention proteins release the bound cannabinoid or other compound to form the therapeutically effective dose according to the invention.

The terms “effective amount” or “effective dose” or “dose” are interchangeably used herein and denote an amount of the pharmaceutical compound having a prophylactically or therapeutically relevant effect on a disease or pathological conditions, i.e. which causes in a tissue, system, animal or human a biological or medical response which is sought or desired, for example, by a researcher or physician. Pharmaceutical formulations can be administered in the form of dosage units which comprise a predetermined amount of active ingredient per dosage unit. The concentration of the prophylactically or therapeutically active ingredient in the formulation may vary from about 0.1 to 100 wt %. Preferably, the compound of formula (I) or the pharmaceutically acceptable salts thereof are administered in doses of approximately 0.5 to 1000 mg, more preferably between 1 and 700 mg, and most preferably 5 and 100 mg per dose unit. Generally, such a dose range is appropriate for total daily incorporation. In other terms, the daily dose is preferably between approximately 0.02 and 100 mg/kg of body weight. The specific dose for each patient depends, however, on a wide variety of factors as already described in the present specification (e.g. depending on the condition treated, the method of administration and the age, weight and condition of the patient). Preferred dosage unit formulations are those which comprise a daily dose or part-dose, as indicated above, or a corresponding fraction thereof of an active ingredient. Furthermore, pharmaceutical formulations of this type can be prepared using a process which is generally known in the pharmaceutical art.

As noted above, the present invention allows the scaled production of water-soluble or solubilized cannabinoids (the terms being generally used to denote a cannabinoid or other compound, such as a terpene or short-chain fatty acid phenolic compound that is water-soluble or may be dissolved in water). Because of this solubility, the invention allows for the addition of such solubilized cannabinoid to a variety of compositions without requiring oils and/or emulsions that are generally required to maintain the generally hydrophobic cannabinoid compounds in suspension. As a result, the present invention may allow for the production of a variety of compositions for the food and beverage industry, as well as pharmaceutical applications that do not required oils or emulsion suspensions and the like.

In one embodiment, the invention may include aqueous compositions containing one or more solubilized cannabinoids that may be introduced to a food or beverage. In a preferred embodiment, the invention may include an aqueous solution containing one or more solubilized cannabinoids. In this embodiment, one or more cannabinoids, terpenes, or other short-chain fatty acid phenolic compounds may be solubilized through binding to an L/OBP-carrier protein, and preferably an engineered L/OBP-carrier protein. Here, the solubilized cannabinoids may be generated in vivo as generally described herein, or in vitro. In additional embodiments, the solubilized cannabinoid may be an isolated non-psychoactive, such as CBD and the like. Such selection of one or more cannabinoids may be due to specific affinity specificities in an L/OBP-carrier or engineered L/OBP-carrier protein for one cannabinoid over another. Moreover, in this embodiment, the aqueous solution may contain one or more of the following: saline, purified water, propylene glycol, deionized water, and/or an alcohol such as ethanol, as well as a pH buffer that may allow the aqueous solution to be maintained at a pH below 7.4. Additional embodiments may include the addition of an acid or base, such as formic acid, or ammonium hydroxide.

In another embodiment, the invention may include a consumable food additive having at least one solubilized cannabinoid. In this embodiment, one or more cannabinoids, terpenes or other short-chain fatty acid phenolic compounds may be solubilized through binding to an L/OBP-carrier protein, and preferably an engineered L/OBP-carrier protein. Here, the solubilized cannabinoids may be generated in vivo as generally described herein, or in vitro. This consumable food additive may further include one or more food additive polysaccharides, such as dextrin and/or maltodextrin, as well as an emulsifier. Example emulsifiers may include, but not be limited to: gum arabic, modified starch, pectin, xanthan gum, gum ghatti, gum tragacanth, fenugreek gum, mesquite gum, mono-glycerides and di-glycerides of long chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono- and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, or combinations thereof.

The consumable food additive of the invention may be a homogenous composition and may further comprise a flavoring agent. Exemplary flavoring agents may include: sucrose (sugar), glucose, fructose, sorbitol, mannitol, corn syrup, high fructose corn syrup, saccharin, aspartame, sucralose, acesulfame potassium (acesulfame-K), and neotame. The consumable food additive of the invention may also contain one or more coloring agents. Exemplary coloring agents may include: FD&C Blue Nos. 1 and 2, FD&C Green No. 3, FD&C Red Nos. 3 and 40, FD&C Yellow Nos. 5 and 6, Orange B, Citrus Red No. 2, annatto extract, beta-carotene, grape skin extract, cochineal extract or carmine, paprika oleoresin, caramel color, fruit and vegetable juices, saffron, Monosodium glutamate (MSG), hydrolyzed soy protein, autolyzed yeast extract, disodium guanylate or inosinate. In one embodiment, this powdered lyophilized L/OBP-carrier protein, having solubilized a quantity of cannabinoids, may be a food additive. In certain preferred embodiments, one or more flavoring agents may be added to a quantity of powdered or lyophilized L/OBP-carrier proteins having solubilized a quantity of cannabinoids.

The consumable food additive of the invention may also contain one or more surfactants, such as glycerol monostearate and polysorbate 80. The consumable food additive of the invention may also contain one or more preservatives. Exemplary preservatives may include ascorbic acid, citric acid, sodium benzoate, calcium propionate, sodium erythorbate, sodium nitrite, calcium sorbate, potassium sorbate, BHA, BHT, EDTA, or tocopherols. The consumable food additive of the invention may also contain one or more nutrient supplements, such as: thiamine hydrochloride, riboflavin, niacin, niacinamide, folate or folic acid, beta carotene, potassium iodide, iron or ferrous sulfate, alpha tocopherols, ascorbic acid, Vitamin D, amino acids, multi-vitamin, fish oil, co-enzyme Q-10, and calcium.

In one embodiment, the invention may include a consumable fluid containing at least one solubilized cannabinoid, terpenoid, or other short chain fatty acid phenolic compound. In one preferred embodiment, this consumable fluid may be added to a drink or beverage to infuse it with the solubilized cannabinoid generated through binding to an L/OBP-carrier protein, preferable an engineered L/OBP-carrier protein, in an in vivo system as generally herein described, or through an in vitro process. The consumable fluid may include a food additive polysaccharide such as maltodextrin and/or dextrin, which may further be in an aqueous form and/or solution. For example, in one embodiment, an aqueous maltodextrin solution may include a quantity of sorbic acid and an acidifying agent to provide a food grade aqueous solution of maltodextrin having a pH of 2-4 and a sorbic acid content of 0.02-0.1% by weight.

In certain embodiments, the consumable fluid may include water, as well as an alcoholic beverage; a non-alcoholic beverage, a noncarbonated beverage, a carbonated beverage, a cola, a root beer, a fruit-flavored beverage, a citrus-flavored beverage, a fruit juice, a fruit-containing beverage, a vegetable juice, a vegetable containing beverage, a tea, a coffee, a dairy beverage, a protein containing beverage, a shake, a sports drink, an energy drink, and a flavored water. The consumable fluid may further include at least one additional ingredient, including but not limited to: xanthan gum, cellulose gum, whey protein hydrolysate, ascorbic acid, citric acid, malic acid, sodium benzoate, sodium citrate, sugar, phosphoric acid, and water. In certain embodiments, the consumable fluid of the invention may be generated by addition of a quantity of solubilized cannabinoid in powder of liquid form as generally described herein to an existing consumable fluid, such as a branded beverage or drink.

In one embodiment, the invention may include a consumable gel having at least one solubilized cannabinoid and gelatin in an aqueous solution. In a preferred embodiment, the consumable gel may include a one or more cannabinoids, terpenes or other short-chain fatty acid phenolic compounds solubilized through binding to an L/OBP-carrier protein, and preferably an engineered L/OBP-carrier protein. Here, the solubilized cannabinoids may be generated in vivo as generally described herein, or in vitro.

Additional embodiments may include a liquid composition having at least one cannabinoid solubilized by an L/OBP-carrier protein, and preferably an engineered L/OBP-carrier protein, in a first quantity of water; and at least one of: xanthan gum, cellulose gum, whey protein hydrolysate, ascorbic acid, citric acid, malic acid, sodium benzoate, sodium citrate, sugar, phosphoric acid, and/or a sugar alcohol. In one preferred embodiment, the composition may further include a quantity of ethanol. Here, the amount of solubilized cannabinoids may include: less than 10 mass % water; more than 95 mass % water; about 0.1 mg to about 1000 mg of the solubilized cannabinoid; about 0.1 mg to about 500 mg of the solubilized cannabinoid; about 0.1 mg to about 200 mg of the solubilized cannabinoid; about 0.1 mg to about 100 mg of the solubilized cannabinoid; about 0.1 mg to about 100 mg of the solubilized cannabinoid; about 0.1 mg to about 10 mg of the solubilized cannabinoid; about 0.5 mg to about 5 mg of the solubilized cannabinoid; about 1 mg/kg to 5 mg/kg (body weight) in a human of the solubilized cannabinoid.

In alternative embodiments, the composition may include at least one cannabinoid solubilized by an L/OBP-carrier protein, and preferably an engineered L/OBP-carrier protein, in the range of 50 mg/L to 300 mg/L; at least one solubilized cannabinoid in the range of 50 mg/L to 100 mg/L; at least one solubilized cannabinoid in the range of 50 mg/L to 500 mg/L; at least one solubilized cannabinoid over 500 mg/L; at least one solubilized cannabinoid under 50 mg/L. Additional embodiments may include one or more of the following additional components: a flavoring agent; a coloring agent; and/or caffeine.

In one embodiment, the invention may include a liquid composition having at least one cannabinoid solubilized by an L/OBP-carrier protein, and preferably an engineered L/OBP-carrier protein, being solubilized in said first quantity of water and a first quantity of ethanol in a liquid state. In a preferred embodiment, a first quantity of ethanol in a liquid state may be between 1% to 20% weight by volume of the liquid composition. In this embodiment, a solubilized cannabinoid may include a cannabinoid solubilized by an L/OBP-carrier protein, a terpenoid/terpene solubilized by an L/OBP-carrier protein, or a mixture of both. Such solubilized cannabinoids may be generated in an in vivo and/or in vitro system as herein identified. In a preferred embodiment, the ethanol or ethyl alcohol component may be up to about ninety-nine point nine-five percent (99.95%) by weight and the solubilized cannabinoid about zero point zero five percent (0.05%) by weight.

Examples of the preferred embodiment may include liquid ethyl alcohol compositions having at least one cannabinoid solubilized by an L/OBP-carrier protein, and preferably an engineered L/OBP-carrier protein, wherein said ethyl alcohol has a proof greater than 100, and/or less than 100. Additional examples of a liquid composition containing ethyl alcohol and at least one cannabinoid solubilized by an L/OBP-carrier protein, and preferably an engineered L/OBP-carrier protein, may include, beer, wine and/or distilled spirits.

Additional embodiments of the invention may include a chewing gum composition having a first quantity of at least one cannabinoid solubilized by an L/OBP-carrier protein, and preferably an engineered L/OBP-carrier protein. In a preferred embodiment, a chewing gum composition may further include a gum base comprising a buffering agent selected from the group consisting of acetates, glycinates, phosphates, carbonates, glycerophosphates, citrates, borates, and mixtures thereof. Additional components may include at least one sweetening agent and at least one flavoring agent. As noted above, in a preferred embodiment, at least one cannabinoid solubilized by an L/OBP-carrier protein, and preferably an engineered L/OBP-carrier protein, may be generated in vivo, or in vivo respectively.

In one embodiment, the chewing gum composition described above may include:
0.01 to 1% by weight of at least one solubilized cannabinoid;
25 to 85% by weight of a gum base;
10 to 35% by weight of at least one sweetening agent; and
1 to 10% by weight of a flavoring agent.

Here, such flavoring agents may include: menthol flavor, eucalyptus, cinnamon, mint flavor and/or L-menthol. Sweetening agents may include one or more of the following: xylitol, sorbitol, isomalt, aspartame, sucralose, acesulfame potassium, and saccharin. Additional preferred embodiment may include a chewing gum having a pharmaceutically acceptable excipient selected from the group consisting of: fillers, disintegrants, binders, lubricants, and antioxidants. The chewing gum composition may further be non-disintegrating and also include one or more coloring and/or flavoring agents.

The invention may further include a composition for a cannabinoid infused solution comprising essentially of: water and/or purified water, at least one cannabinoid solubilized by an L/OBP-carrier protein and preferably an engineered L/OBP-carrier protein, and at least one flavoring agent. A solubilized cannabinoid infused solution of the invention may further include a sweetener selected from the group consisting of: glucose, sucrose, invert sugar, corn syrup, stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same. Additional components of the solubilized cannabinoid infused solution may include, but not be limited to: sodium chloride, sodium chloride solution, glycerin, a coloring agent, and a demulcent. As to this last potential component, in certain embodiments, a demulcent may include: pectin, glycerin, honey, methylcellulose, and/or propylene glycol. As noted above, in a preferred embodiment, a solubilized cannabinoid may include at least one solubilized cannabinoid wherein such solubilized cannabinoids may be generated in vivo and/or in vitro respectively.

The invention may further include a composition for a solubilized cannabinoid infused anesthetic solution having water, or purified water, at least one solubilized cannabinoid, and at least one oral anesthetic. In a preferred embodiment, an anesthetic may include benzocaine, and/or phenol in a quantity of between 0.1% to 15% volume by weight.

Additional embodiments may include a solubilized cannabinoid infused anesthetic solution having a sweetener which may be selected from the group consisting of: glucose, sucrose, invert sugar, corn syrup, stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same. Additional components of a solubilized cannabinoid infused solution may include, but not be limited to: sodium chloride, sodium chloride solution, glycerin, a coloring agent, and a demulcent. In a preferred embodiment, a demulcent may be selected from the group consisting of: pectin, glycerin, honey, methylcellulose, and propylene glycol. As noted above, in a preferred embodiment, a solubilized cannabinoid may include at least one cannabinoid solubilized by an L/OBP-carrier protein, and preferably an engineered L/OBP-carrier protein, or a mixture of the two. In this embodiment, such solubilized cannabinoids may have been generated in vivo and/or in vitro respectively.

The invention may further include a composition for a hard lozenge for rapid delivery of solubilized cannabinoids through the oral mucosa. In this embodiment, such a hard lozenge composition may include: a crystalized sugar base, and at least one solubilized cannabinoid, wherein the hard lozenge has moisture content between 0.1 to 2%. In this embodiment, the solubilized cannabinoid may be added to the sugar base when it is in a liquefied form and prior to the evaporation of the majority of water content. Such a hard lozenge may further be referred to as a candy.

In a preferred embodiment, a crystalized sugar base may be formed from one or more of the following: sucrose, invert sugar, corn syrup, and isomalt or a combination of the same. Additional components may include at least one acidulant. Examples of acidulants may include, but not be limited to: citric acid, tartaric acid, fumaric acid, and malic acid. Additional components may include at least one pH adjustor. Examples of pH adjustors may include, but not be limited to: calcium carbonate, sodium bicarbonate, and magnesium trisilicate.

In another preferred embodiment, the composition may include at least one anesthetic. Example of such anesthetics may include benzocaine, and phenol. In this embodiment, first quantity of anesthetic may be between 1 mg to 15 mg per lozenge. Additional embodiments may include a quantity of menthol. In this embodiment, such a quantity of menthol may be between 1 mg to 20 mg. The hard lozenge composition may also include a demulcent, for example: pectin, glycerin, honey, methylcellulose, propylene glycol, and glycerin. In this embodiment, a demulcent may be in a quantity between 1 mg to 10 mg. As noted above, in a preferred embodiment, a solubilized cannabinoid may include at least one cannabinoid solubilized by an L/OBP-carrier protein, and preferably an engineered L/OBP-carrier protein, or a mixture of the two. In this embodiment, such solubilized cannabinoid may have been generated in vivo and/or in vitro respectively.

The invention may include a chewable lozenge for rapid delivery of solubilized cannabinoids through the oral mucosa. In a preferred embodiment, the compositions may include: a glycerinated gelatin base, at least one sweetener, and at least one solubilized cannabinoid dissolved in a first quantity of water. In this embodiment, a sweetener may include a sweetener selected from the group consisting of: glucose, sucrose, invert sugar, corn syrup, stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same.

Additional components may include at least one acidulant. Examples of acidulants may include, but not be limited to: citric acid, tartaric acid, fumaric acid, and malic acid. Additional components may include at least one pH adjustor. Examples of pH adjustors may include, but not be limited to: calcium carbonate, sodium bicarbonate, and magnesium trisilicate.

In another preferred embodiment, the composition may include at least one anesthetic. Example of such anesthetics may include benzocaine and phenol. In this embodiment, first quantity of anesthetic may be between 1 mg to 15 mg per lozenge. Additional embodiments may include a quantity of menthol. In this embodiment, such a quantity of menthol may be between 1 mg to 20 mg. The chewable lozenge composition may also include a demulcent, for example: pectin, glycerin, honey, methylcellulose, propylene glycol, and glycerin. In this embodiment, a demulcent may be in a quantity between 1 mg to 10 mg. As noted above, in a preferred embodiment, a solubilized cannabinoid may include at least one cannabinoid solubilized by an L/OBP-carrier protein, and preferably an engineered L/OBP-carrier protein, or a mixture of the two. In this embodiment, such solubilized cannabinoid may be generated in vivo or in vitro respectively.

The invention may include a soft lozenge for rapid delivery of solubilized cannabinoids through the oral mucosa. In a preferred embodiment, the compositions may include: a polyethylene glycol base, at least one sweetener, and at least one solubilized cannabinoid dissolved in a first quantity of water. In this embodiment, a sweetener may include sweetener selected from the group consisting of: glucose, sucrose, invert sugar, corn syrup, stevia extract powder, stevioside, steviol, aspartame, saccharin, saccharin salts, sucralose, potassium acetosulfam, sorbitol, xylitol, mannitol, erythritol, lactitol, alitame, miraculin, monellin, and thaumatin or a combination of the same. Additional components may include at least one acidulant. Examples of acidulants may include, but not be limited to: citric acid, tartaric acid, fumaric acid, and malic acid. Additional components may include at least one pH adjustor. Examples of pH adjustors may include, but not be limited to: calcium carbonate, sodium bicarbonate, and magnesium trisilicate.

In another preferred embodiment, the composition may include at least one anesthetic. Example of such anesthetics may include benzocaine and phenol. In this embodiment, first quantity of anesthetic may be between 1 mg to 15 mg per lozenge. Additional embodiments may include a quantity of menthol. In this embodiment, such a quantity of menthol may be between 1 mg to 20 mg. The soft lozenge composition may also include a demulcent, for example: pectin, glycerin, honey, methylcellulose, propylene glycol, and glycerin. In this embodiment, a demulcent may be in a quantity between 1 mg to 10 mg. As noted above, in a preferred embodiment, a solubilized cannabinoid may include at least one cannabinoid solubilized by an L/OBP-carrier protein, and preferably an engineered L/OBP-carrier protein, or a mixture of the two. In this embodiment, such solubilized cannabinoid may be generated in vivo or in vitro respectively.

In another embodiment, the invention may include a tablet or capsule consisting essentially of a solubilized cannabinoid and a pharmaceutically acceptable excipient. Examples may include solid, semi-solid, and aqueous excipients such as: maltodextrin, whey protein isolate, xanthan gum, guar gum, diglycerides, monoglycerides, carboxymethyl cellulose, glycerin, gelatin, polyethylene glycol and water-based excipients. In this embodiment, the cannabinoid solubilized by an L/OBP-carrier protein, and preferably an engineered L/OBP-carrier protein, may have an improved shelf-life, composition stability, and bioavailability upon injection.

In a preferred embodiment, a solubilized cannabinoid may include at least one cannabinoid solubilized by an L/OBP-carrier protein, and preferably an engineered L/OBP-carrier protein, or a mixture of the two. In this embodiment, such solubilized cannabinoids may be generated in vivo or in vitro respectively. Examples of such in vivo systems being generally described herein, including in plant, as well as cell culture systems including cannabis cell culture, tobacco cell culture, bacterial cell cultures, fungal cell cultures, and yeast cell culture systems. In one embodiment, a tablet or capsule may include an amount of solubilized cannabinoid of 5 milligrams or less. Alternative embodiments may include an amount of solubilized cannabinoid between 5 milligrams and 200 milligrams. Still other embodiments may include a tablet or capsule having an amount of solubilized cannabinoid that is more than 200 milligrams. Still other embodiments may include a tablet or capsule having an amount of solubilized cannabinoid that is more than 500 milligrams.

The invention may further include a method of manufacturing and packaging a solubilized cannabinoid dosage, consisting of the following steps: 1) preparing a fill solution with a desired concentration of a solubilized cannabinoids in a liquid carrier wherein said cannabinoid is dissolved in said liquid carrier; 2) encapsulating said fill solution in capsules; 3) packaging said capsules in a closed packaging system; and 4) removing atmospheric air from the capsules. In one embodiment, the step of removing atmospheric air consists of purging the packaging system with an inert gas, such as, for example, nitrogen gas, such that said packaging system provides a room temperature stable product. In one preferred embodiment, the packaging system may include a plaster package, which may be constructed of material that minimizes exposure to moisture and air.

In one embodiment, a preferred liquid carrier may include a water-based carrier, such as for example an aqueous sodium chloride solution. In a preferred embodiment, a solubilized cannabinoid may include at least one cannabinoid solubilized by an L/OBP-carrier protein, and preferably an engineered L/OBP-carrier protein, or a mixture of the two. In this embodiment, such solubilized cannabinoids may be generated in vivo or in vitro respectively. In one embodiment, a desired solubilized cannabinoid concentration may be about 1-10% w/w, while in other embodiments it may be about 1.5-6.5% w/w. Alternative embodiments may include an amount of solubilized cannabinoid between 5 milligrams and 200 milligrams. Still, other embodiments may include a tablet or capsule having amount of solubilized cannabinoid that is more than 200 milligrams. Other embodiments may include a tablet or capsule having an amount of solubilized cannabinoid that is more than 500 milligrams.

The invention may include an oral pharmaceutical solution, such as a sub-lingual spray having solubilized cannabinoids and a liquid carrier. One embodiment may include a solubilized cannabinoid, 30-33% w/w water, about 50% w/w alcohol, 0.01% w/w butylated hydroxylanisole (BHA) or 0.1% w/w ethylenediaminetetraacetic acid (EDTA) and 5-21% w/w co-solvent, having a combined total of 100%, wherein said co-solvent is selected from the group consisting of propylene glycol, polyethylene glycol, and combinations thereof, and wherein said solubilized cannabinoid is at least one cannabinoid solubilized by an L/OBP-carrier protein, and preferably an engineered L/OBP-carrier protein, or a mixture of the two. In an alternative embodiment, such a oral pharmaceutical solution may consist essentially of 0.1 to 5% w/w of said solubilized cannabinoid, about 50% w/w alcohol, 5.5% w/w propylene glycol, 12% w/w polyethylene glycol and 30-33% w/w water. In a preferred composition, the alcohol component may be ethanol.

The invention may include an oral pharmaceutical solution, such as a sublingual spray, consisting essentially of about 0.1% to 1% w/w solubilized cannabinoids, about 50% w/w alcohol, 5.5% w/w propylene glycol, 12% w/w polyethylene glycol, 30-33% w/w water, 0.01% w/w butylated hydroxyanisole, having a combined total of 100%, and wherein said solubilized cannabinoid is at least one cannabinoid solubilized by an L/OBP-carrier protein, and preferably an engineered L/OBP-carrier protein, or a mixture of the two that may be further generated in vitro and/or in vivo respectively. In an alternative embodiment, such a oral pharmaceutical solution may consist essentially of 0.54% w/w solubilized cannabinoid, 31.9% w/w water, 12% w/w polyethylene glycol 400, 5.5% w/w propylene glycol, 0.01% w/w butylated hydroxyanisole, 0.05% w/w sucralose, and 50% w/w alcohol, wherein the a the alcohol components may be ethanol.

The invention may include a solution for nasal and/or sublingual administration of a solubilized cannabinoid including: 1) an excipient of propylene glycol, ethanol anhydrous, or a mixture of both; and 2) a solubilized cannabinoid which may include at least one cannabinoid solubilized by an L/OBP-carrier protein, and preferably an engineered L/OBP-carrier protein, or a mixture of the two that may be further generated in vitro and/or in vivo respectively. In a preferred embodiment, the composition may further include a topical decongestant, which may include phenylephrine hydrochloride, Oxymetazoline hydrochloride, and Xylometazoline in certain preferred embodiments. The composition may further include an antihistamine, and/or a steroid. Preferably, the steroid component is a corticosteroid selected from the group consisting of: neclomethasone dipropionate, budesonide, ciclesonide, flunisolide, fluticasone furoate, fluticasone propionate, mometasone, and triamcinolone acetonide. In alternative embodiments, the solution for nasal and/or sublingual administration of a solubilized cannabinoid may further comprise at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, and propylene glycol.

The invention may further include an aqueous solution for nasal and/or sublingual administration of a solubilized cannabinoid comprising: a water and/or saline solution; and a solubilized cannabinoid which may include at least one cannabinoid solubilized by an L/OBP-carrier protein, and preferably an engineered L/OBP-carrier protein, or a mixture of the two that may be further generated in vitro and/or in vivo respectively. In a preferred embodiment, the composition may further include a topical decongestant, which may include phenylephrine hydrochloride, Oxymetazoline hydrochloride, and Xylometazoline in certain preferred embodiments. The composition may further include an antihistamine and/or a steroid. Preferably, the steroid component is a corticosteroid selected from the group consisting of: neclomethasone dipropionate, budesonide, ciclesonide, flunisolide, fluticasone furoate, fluticasone propionate, mometasone, and triamcinolone acetonide. In alternative embodiments, the aqueous solution may further comprise at least one of the following: benzalkonium chloride solution, benzyl alcohol, boric acid, purified water, sodium borate, polysorbate 80, phenylethyl alcohol, microcrystalline cellulose, carboxymethylcellulose sodium, dextrose, dipasic, sodium phosphate, edetate disodium, monobasic sodium phosphate, or propylene glycol.

The invention may include a topical formulation for the transdermal delivery of solubilized cannabinoids. In a preferred embodiment, a topical formulation for the transdermal delivery of solubilized cannabinoids which may include at least one cannabinoid solubilized by an L/OBP-carrier protein, and preferably an engineered L/OBP-carrier protein, or a mixture of the two, and a pharmaceutically acceptable excipient. The solubilized cannabinoids may be generated in vitro and/or in vivo respectively. Preferably a pharmaceutically acceptable excipient may include one or more: gels, ointments, cataplasms, poultices, pastes, creams, lotions, plasters and jellies or even polyethylene glycol. Additional embodiments may further include one or more of the following components: a quantity of capsaicin; a quantity of benzocaine; a quantity of lidocaine; a quantity of camphor; a quantity of benzoin resin; a quantity of methylsalicilate; a quantity of triethanolamine salicylate; a quantity of hydrocortisone; or a quantity of salicylic acid.

The invention may include a gel for transdermal administration of a solubilized cannabinoid which may include at least one cannabinoid solubilized by an L/OBP-carrier protein, and preferably an engineered L/OBP-carrier protein or a mixture of the two and which may be generated in vitro and/or in vivo. In this embodiment, the mixture preferably contains from 15% to about 90% ethanol, about 10% to about 60% buffered aqueous solution or water, about 0.1 to about 25% propylene glycol, from about 0.1 to about 20% of a gelling agent, from about 0.1 to about 20% of a base, from about 0.1 to about 20% of an absorption enhancer and from about 1% to about 25% polyethylene glycol, and a solubilized cannabinoid as generally described herein.

In another embodiment, the invention may further include a transdermal composition having a pharmaceutically effective amount of a solubilized cannabinoid for delivery of the cannabinoid to the bloodstream of a user. This transdermal composition may include a pharmaceutically acceptable excipient and at least one solubilized cannabinoid, which may include at least one cannabinoid solubilized by an L/OBP-carrier protein, and preferably an engineered L/OBP-carrier protein, or a mixture of the two and which may be generated in vitro and/or in vivo, wherein the solubilized cannabinoid is capable of diffusing from the composition into the bloodstream of the user. In a preferred embodiment, a pharmaceutically acceptable excipient to create a transdermal dosage form selected from the group consisting of: gels, ointments, cataplasms, poultices, pastes, creams, lotions, plasters and jellies. The transdermal composition may further include one or more surfactants. In one preferred embodiment, the surfactant may include a surfactant-lecithin organogel, which may further be present in an amount of between about 95% and about 98% w/w. In an alternative embodiment, a surfactant-lecithin organogel comprises lecithin and PPG-2 myristyl ether propionate and/or high molecular weight polyacrylic acid polymers. The transdermal composition may further include a quantity of isopropyl myristate.

The invention may further include transdermal composition having one or more permeation enhancers to facilitate transfer of the solubilized cannabinoid across a dermal layer. In a preferred embodiment, a permeation enhancer may include one or more of the following: propylene glycol monolaurate, diethylene glycol monoethyl ether, an oleoyl macrogolglyceride, a caprylocaproyl macrogolglyceride, and an oleyl alcohol.

The invention may also include a liquid cannabinoid liniment composition consisting of water, isopropyl alcohol solution, and a solubilized cannabinoid, which may include at least one cannabinoid solubilized by an L/OBP-carrier protein, and preferably an engineered L/OBP-carrier protein or a mixture of the two and which may be generated in vitro and/or in vivo. This liquid cannabinoid liniment composition may further include approximately 97.5% to about 99.5% by weight of 70% isopropyl alcohol solution and from about 0.5% to about 2.5% by weight of a solubilized cannabinoid mixture.

Based on the improved solubility and other physical properties, as well as cost advantages, improved cannabinoid affinity and capacity, extended shelf-life, and scalability of the invention's in vivo or in vitro solubilized cannabinoid production platform, the invention may include one or more commercial infusions. For example, commercially available products, such a lip balm, soap, shampoos, lotions, creams, and cosmetics may be infused with one or more solubilized cannabinoids.

The invention may further include a novel composition that may be used to supplement a cigarette or other tobacco-based product. In this embodiment, the composition may include at least one solubilized cannabinoid in a powder as already described, or dissolved in an aqueous solution. This aqueous solution may be introduced to a tobacco product, such as a cigarette and/or a tobacco leaf such that the aqueous solution may evaporate generating a cigarette and/or a tobacco leaf that contains the aforementioned solubilized cannabinoid(s), which may further have been generated in vivo as generally described herein.

In one embodiment, the invention may include one or more methods of treating a medical condition in a mammal. In this embodiment, the novel method may include of administering a therapeutically effective amount of a solubilized cannabinoid, such as an in vivo or in vitro cannabinoid solubilized by an L/OBP-carrier protein, and preferably an engineered L/OBP-carrier protein, or a mixture of the two, wherein the medical condition is selected from the group consisting of: obesity, post-traumatic stress syndrome, anorexia, nausea, emesis, pain, wasting syndrome, HIV-wasting, chemotherapy induced nausea and vomiting, alcohol use disorders, anti-tumor, amyotrophic lateral sclerosis, glioblastoma multiforme, glioma, increased intraocular pressure, glaucoma, cannabis use disorders, Tourette's syndrome, dystonia, multiple sclerosis, inflammatory bowel disorders, arthritis, dermatitis, Rheumatoid arthritis, systemic lupus erythematosus, anti-inflammatory, anti-convulsant, anti-psychotic, anti-oxidant, neuroprotective, anti-cancer, immunomodulatory effects, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, synovitis, and juvenile rheumatoid arthritis. In a preferred embodiment, the pharmaceutical composition may be administered by a route selected from the group consisting of: transdermal, topical, oral, buccal, sublingual, intra-venous, intra-muscular, vaginal, rectal, ocular, nasal and follicular. The amount of solubilized cannabinoids may be a therapeutically effective amount, which may be determined by the patient's age, weight, medical condition cannabinoid-delivered, route of delivery, and the like. In one embodiment, a therapeutically effective amount may be 50 mg or less of a solubilized cannabinoid. In another embodiment, a therapeutically effective amount may be 50 mg or more of a solubilized cannabinoid.

It should be noted that for any of the above composition, unless otherwise stated, an effective amount of solubilized cannabinoids may include amounts between: 0.01 mg to 0.1 mg; 0.01 mg to 0.5 mg; 0.01 mg to 1 mg; 0.01 mg to 5 mg; 0.01 mg to 10 mg; 0.01 mg to 25 mg; 0.01 mg to 50 mg; 0.01 mg to 75 mg; 0.01 mg to 100 mg; 0.01 mg to 125 mg; 0.01 mg to 150 mg; 0.01 mg to 175 mg; 0.01 mg to 200 mg; 0.01 mg to 225 mg; 0.01 mg to 250 mg; 0.01 mg to 275 mg; 0.01 mg to 300 mg; 0.01 mg to 225 mg; 0.01 mg to 350 mg; 0.01 mg to 375 mg; 0.01 mg to 400 mg; 0.01 mg to 425 mg; 0.01 mg to 450 mg; 0.01 mg to 475 mg; 0.01 mg to 500 mg; 0.01 mg to 525 mg; 0.01 mg to 550 mg; 0.01 mg to 575 mg; 0.01 mg to 600 mg; 0.01 mg to 625 mg; 0.01 mg to 650 mg; 0.01 mg to 675 mg; 0.01 mg to 700 mg; 0.01 mg to 725 mg; 0.01 mg to 750 mg; 0.01 mg to 775 mg; 0.01 mg to 800 mg; 0.01 mg to 825 mg; 0.01 mg to 950 mg; 0.01 mg to 875 mg; 0.01 mg to 900 mg; 0.01 mg to 925 mg; 0.01 mg to 950 mg; 0.01 mg to 975 mg; 0.01 mg to 1000 mg; 0.01 mg to 2000 mg; 0.01 mg to 3000 mg; 0.01 mg to 4000 mg; 01 mg to 5000 mg; 0.01 mg to 0.1 mg/kg; 0.01 mg to 0.5 mg/kg; 01 mg to 1 mg/kg; 0.01 mg to 5 mg/kg; 0.01 mg to 10 mg/kg; 0.01 mg to 25 mg/kg; 0.01 mg to 50 mg/kg; 0.01 mg to 75 mg/kg; and 0.01 mg to 100 mg/kg.

The solubilized cannabinoids compounds of the present invention are useful for a variety of therapeutic applications. For example, the compounds are useful for treating or alleviating symptoms of diseases and disorders involving CB1, CB2, GPR119, 5HT_{1A}, μ and δ-OPR receptors, and TRP channels, including appetite loss, nausea and vomiting, pain, multiple sclerosis and epilepsy. For example, they may be used to treat pain (i.e. as analgesics) in a variety of applications including but not limited to pain management. In additional embodiments, such solubilized cannabinoids may be used as an appetite suppressant. Additional embodiments may include administering the solubilized cannabinoids compounds.

By “treating,” the present inventors mean that the compound is administered in order to alleviate symptoms of the disease or disorder being treated. Those of skill in the art will recognize that the symptoms of the disease or disorder that is treated may be completely eliminated or may simply be lessened. Further, the compounds may be administered in combination with other drugs or treatment modalities, such as with chemotherapy or other cancer-fighting drugs.

Implementation may generally involve identifying patients suffering from the indicated disorders and administering the compounds of the present invention in an acceptable form by an appropriate route. The exact dosage to be administered may vary depending on the age, gender, weight, and overall health status of the individual patient, as well as the precise etiology of the disease. However, in general, for administration in mammals (e.g. humans), dosages in the range of from about 0.01 to about 300 mg of compound per kg of body weight per 24 hr., and more preferably about 0.01 to about 100 mg of compound per kg of body weight per 24 hr., may be effective.

Administration may be oral or parenteral, including intravenously, intramuscularly, subcutaneously, intradermal injection, intraperitoneal injection, etc., or by other routes (e.g. transdermal, sublingual, oral, rectal and buccal delivery, inhalation of an aerosol, etc.). In a preferred embodiment of the invention, the solubilized cannabinoid are provided orally or intravenously.

The compounds may be administered in the pure form or in a pharmaceutically acceptable formulation including suitable elixirs, binders, and the like (generally referred to as a “secondary carrier”) or as pharmaceutically acceptable salts (e.g. alkali metal salts such as sodium, potassium, calcium or lithium salts, ammonium, etc.) or other complexes. It should be understood that the pharmaceutically acceptable formulations include liquid and solid materials conventionally utilized to prepare both injectable dosage forms and solid dosage forms such as tablets and capsules and aerosolized dosage forms. In addition, the compounds may be formulated with aqueous or oil based vehicles. Water may be used as the carrier for the preparation of compositions (e.g. injectable compositions), which may also include conventional buffers and agents to render the composition isotonic. Other potential additives and other materials (preferably those which are generally regarded as safe [GRAS]) include: colorants; flavorings; surfactants (TWEEN, oleic acid, etc.); solvents, stabilizers, elixirs, and binders or encapsulants (lactose, liposomes, etc). Solid diluents and excipients include lactose, starch, conventional disintergrating agents, coatings and the like. Preservatives such as methyl paraben or benzalkium chloride may also be used. Depending on the formulation, it is expected that the active composition will consist of about 1% to about 99% of the composition and the secondary carrier will constitute about 1% to about 99% of the composition. The pharmaceutical compositions of the present invention may include any suitable pharmaceutically acceptable additives or adjuncts to the extent that they do not hinder or interfere with the therapeutic effect of the active compound.

The administration of the compounds of the present invention may be intermittent, bolus dose, or at a gradual or continuous, constant, or controlled rate to a patient. In addition, the time of day and the number of times per day that the pharmaceutical formulation is administered may vary and are best determined by a skilled practitioner such as a physician. Further, the effective dose can vary depending upon factors such as the mode of delivery, gender, age, and other conditions of the patient, as well as the extent or progression of the disease. The compounds may be provided alone, in a mixture containing two or more of the compounds, or in combination with other medications or treatment modalities.

As used herein, a “cannabinoid” is a chemical compound (such as cannabinol, THC or cannabidiol) that is found in the plant species Cannabis among others like: Echinacea; Acmella oleracea; Helichrysum umbraculigerum; Radula marginata (Liverwort) and Theobroma cacao, and metabolites and synthetic analogues thereof that may or may not have psychoactive properties. Cannabinoids therefore include (without limitation) compounds (such as THC) that have high affinity for the cannabinoid receptor (for example Ki<250 nM), and compounds that do not have significant affinity for the cannabinoid receptor (such as cannabidiol, CBD). Cannabinoids also include compounds that have a characteristic dibenzopyran ring structure (of the type seen in THC) and cannabinoids which do not possess a pyran ring (such as cannabidiol). Hence a partial list of cannabinoids includes THC, CBD, dimethyl heptylpentyl cannabidiol (DMHP-CBD), 6,12-dihydro-6-hydroxy-cannabidiol (described in U.S. Pat. No. 5,227,537, incorporated by reference); (3S,4R)-7-hydroxy-Δ6-tetrahydrocannabinol homologs and derivatives described in U.S. Pat. No. 4,876,276, incorporated by reference; (+)-4-[4-DMH-2,6-diacetoxy-phenyl]-2-carboxy-6,6-dimethylbicyclo[3.1.1]hept-2-en, and other 4-phenylpinene derivatives disclosed in U.S. Pat. No. 5,434,295, which is incorporated by reference; and cannabidiol (−)(CBD) analogs such as (−)CBD-monomethylether, (−)CBD dimethyl ether; (−)CBD diacetate; (−)3′-acetyl-CBD monoacetate; and ±AF11, all of which are disclosed in Consroe et al., J. Clin. Pharmacol. 21:428S-436S, 1981, which is also incorporated by reference. Many other cannabinoids are similarly disclosed in Agurell et al., Pharmacol. Rev. 38:31-43, 1986, which is also incorporated by reference.

As claimed herein, the term “cannabinoid” may also be generically applied to describe all cannabinoids, short-chain fatty acid phenolic compounds, endocannabinoids, phytocannabinoids, as well as terpenes that have affinity for one or more L/OBP-carrier proteins and/or engineered L/OBP-carrier proteins, or their homologs as generally described herein. Moreover, as used herein, the term “solubilized cannabinoid” describes a “cannabinoid,” that binds to or interacts with one or more L/OBP-carrier proteins and/or engineered L/OBP-carrier proteins, or their homologs as generally described herein. Examples of cannabinoids are tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C4, cannabidivarinic acid, cannabidiorcol, delta-9-tetrahydrocannabinolic acid A, delta-9-tetrahydrocannabinolic acid B, delta-9-tetrahydrocannabinolic acid-C4, delta-9-tetrahydrocannabivarinic acid, delta-9-tetrahydrocannabivarin, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, delta-9-cis-tetrahydrocannabinol, 3,4,5,6-tetrahydro-7-hydroxy-alpha-alpha-2-trimethyl-9-n-propyl-2,6-methano-2H-1-benzoxocin-5-methanol-cannabiripsol, trihydroxy-delta-9-tetrahydrocannabinol, and cannabinol. Examples of cannabinoids within the context of this disclosure include tetrahydrocannabinol and cannabidiol.

The term “endocannabinoid” refers to compounds including arachidonoyl ethanolamide (anandamide, AEA), 2-arachidonoyl ethanolamide (2-AG), 1-arachidonoyl ethanolamide (1-AG), and docosahexaenoyl ethanolamide (DHEA, synaptamide), oleoyl ethanolamide (OEA), eicsapentaenoyl ethanolamide, prostaglandin ethanolamide, docosahexaenoyl ethanolamide, linolenoyl ethanolamide, 5(Z),8(Z),11(Z)-eicosatrienoic acid ethanolamide (mead acid ethanolamide), heptadecanoul ethanolamide, stearoyl ethanolamide, docosaenoyl ethanolamide, nervonoyl ethanolamide, tricosanoyl ethanolamide, lignoceroyl ethanolamide, myristoyl ethanolamide, pentadecanoyl ethanolamide, palmitoleoyl ethanolamide, docosahexaenoic acid (DHA). Particularly preferred endocannabinoids are AEA, 2-AG, 1-AG, and DHEA.

Terpenoids a.k.a. isoprenoids, are a large and diverse class of naturally occurring organic chemicals similar to terpenes, derived from five-carbon isoprene units assembled and modified in a number of varying configurations. Most are multi-cyclic structures that differ from one another not only in functional groups but also in their basic carbon skeletons. Terpenoids are essential for plant metabolism, influencing general development, herbivory defense, pollination and stress response. These compounds have been extensively used as flavoring and scenting agents in cosmetics, detergents, food and pharmaceutical products. They also display multiple biological activities in humans, such as anti-inflammatory, anti-microbial, antifungal and antiviral. Cannabis terpenoid profiles define the aroma of each plant and share the same precursor (geranyl pyrophosphate) and the same synthesis location (glandular trichomes) as phytocannabinoids. The terpenoids most commonly found in Cannabis extracts include: limonine, myrcene, alpha-pinene, linalool, beta-caryophyllene, caryophyllene oxide, nerolidol, and phytol. Terpenoids are mainly synthesized in two metabolic pathways: mevalonic acid pathway (a.k.a. HMG-CoA reductase pathway, which takes place in the cytosol) and MEP/DOXP pathway (a.k.a. The 2-C-methyl-D-erythritol 4-phosphate/1-deoxy-D-xylulose 5-phosphate pathway, non-mevalonate pathway, or mevalonic acid-independent pathway, which takes place in plastids). Geranyl pyrophosphate (GPP), which is used by Cannabis plants to produce cannabinoids, is formed by condensation of dimethylallyl pyrophosphate (DMAPP) and isopentenyl pyrophosphate (IPP) via the catalysis of GPP synthase. Alternatively, DMAPP and IPP are ligated by FPP synthase to produce farnesyl pyrophosphate (FPP), which can be used to produce sesquiterpenoids. Geranyl pyrophospliate (GPP) can also be converted into monoterpenoids by limonene synthase. Some examples of terpenes, and their classification, are as follows. Hemiterpenes: Examples of hemiterpenes, which do not necessarily have an odor, are 2-methyl-1,3-butadiene, hemialboside, and hymenoside. [0086] Monoterpenes: pinene, a-pinene, β-pinene, cis-pinane, trans-pinane, cis-pinanol, trans-pinanol (Erman and Kane (2008) Chem. Biodivers. 5:910-919), limonene; linalool; myrcene; eucalyptol; a-phellandrene; β-phellandrene; a-ocimene; β-ocimene, cis-ocimene, ocimene, Δ-3-carene; fenchol; sabinene, borneol, isoborneol, camphene, camphor, phellandrene, a-phellandrene, a-terpinene, geraniol, linalool, nerol, menthol, myrcene, terpinolene, a-terpinolene, β-terpinolene, γ-terpinolene, A-terpinolene, a-terpineol, and trans-2-pinanol. Sesquiterpenes: caryophyllene, caryophyllene oxide, humulene, a-humulene, a-bisabolene; β-bisabolene; santalol; selinene; nerolidol, bisabolol; a-cedrene, β-cedrene, β-eudesmol, eudesm-7(11)-en-4-ol, selina-3,7(11)-diene, guaiol, valencene, a-guaiene, β-guaiene, Δ-guaiene, guaiene, farnesene, a-farnesene, β-farnesene, elemene, a-elemene, β-elemene, γ-elemene, Δ-elemene, germacrene, germacrene A, germacrene B, germacrene C, germacrene D, and germacrene E. Diterpenes: oridonin, phytol, and isophytol. Triterpenes: ursolic acid, oleanolic acid. Terpenoids, also known as isoprenoids, are a large and diverse class of naturally occurring organic chemicals similar to terpenes, derived from five-carbon isoprene units assembled and modified in a number of ways. Most are multicyclic structures that differ from one another not only in functional groups but also in their basic carbon skeletons. Plant terpenoids are used extensively for their aromatic qualities.

A protein has “homology” or is “homologous” to a second protein if the amino acid sequence encoded by a gene has a similar amino acid sequence to that of the second gene. Alternatively, a protein has homology to a second protein if the two proteins have “similar” amino acid sequences. (Thus, the term “homologous proteins” is defined to mean that the two proteins have similar amino acid sequences). More specifically, in certain embodiments, the term “homologous” with regard to a contiguous nucleic acid sequence, refers to contiguous nucleotide sequences that hybridize under appropriate conditions to the reference nucleic acid sequence. For example, homologous sequences may have from about 75%-100, or more generally 80% to 100% sequence identity, such as about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; and about 100%. The property of substantial homology is closely related to specific hybridization. For example, a nucleic acid molecule is specifically hybridizable when there is a sufficient degree of complementarity to avoid non-specific binding of the nucleic acid to non-target sequences under conditions where specific binding is desired, for example, under stringent hybridization conditions, and would fall within the range of a homolog. In another embodiment, expression optimization, for example for a mammalian lipocalin or odorant binding protein, to be expressed in yeast may be considered homologous and having a variable sequence identity due to the variable codon positions. Additional embodiments may also include homology to include redundant nucleotide codons.

The term “homolog”, used with respect to an original enzyme or gene of a first family or species, refers to distinct enzymes or genes of a second family or species which are determined by functional, structural or genomic analyses to be an enzyme or gene of the second family or species which corresponds to the original enzyme or gene of the first family or species. Most often, homologs will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes.

The term “operably linked,” when used in reference to a regulatory sequence and a coding sequence, means that the regulatory sequence affects the expression of the linked coding sequence. “Regulatory sequences,” or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell. An “inducible” promoter may be a promoter which may be under environmental control. Tissue-specific, tissue-preferred, cell type specific, and inducible promoters constitute the class of “non-constitutive” promoters. A “constitutive” promoter is a promoter which may be active under most environmental conditions or in most cell or tissue types.

As used herein, the term “transformation” or “genetically modified” refers to the transfer of one or more nucleic acid molecule(s) into a cell. A plant is “transformed” or “genetically modified” by a nucleic acid molecule transduced into the plant when the nucleic acid molecule becomes stably replicated by the plant. As used herein, the term “transformation” or “genetically modified” encompasses all techniques by which a nucleic acid molecule can be introduced into, such as a plant.

The term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; or can be regulatory in nature, etc. There are various types of vectors including virus, plasmid, bacteriophages, cosmids, and bacteria.

As is known in the art, different organisms preferentially utilize different codons for generating polypeptides. Such “codon usage” preferences may be used in the design of nucleic acid molecules encoding the proteins and chimeras of the invention in order to optimize expression in a particular host cell system.

An “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of a cassette assists in the assembly of the expression vectors. An expression vector is a replicon, such as plasmid, phage, virus, chimeric virus, or cosmid, and which contains the desired polynucleotide sequence operably linked to the expression control sequence(s).

A polynucleotide sequence is operably linked to an expression control sequence(s) (e.g., a promoter and, optionally, an enhancer) when the expression control sequence controls and regulates the transcription and/or translation of that polynucleotide sequence.

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides. The Table below, contains information about which nucleic acid codons encode which amino acids.

### Amino Acid Nucleic Acid Codons

| Amino Acid | Nucleic Acid Codons |
|---|---|
| Ala/A | GCT, GCC, GCA, GCG |
| Arg/R | CGT, CGC, CGA, CGG, AGA, AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |

Moreover, because the proteins are described herein, one can chemically synthesize a polynucleotide which encodes these polypeptides/chimeric proteins. Oligonucleotides and polynucleotides that are not commercially available can be chemically synthesized e.g., according to the solid phase phosphoramidite triester method first described by Beaucage and Caruthers, Tetrahedron Letts. 22:1859-1862 (1981), or using an automated synthesizer, as described in Van Devanter et al., Nucleic Acids Res. 12:6159-6168 (1984). Other methods for synthesizing oligonucleotides and polynucleotides are known in the art. Purification of oligonucleotides is by either native acrylamide gel electrophoresis or by anion-exchange HPLC as described in Pearson & Reanier, J. Chrom. 255:137-149 (1983).

The term “plant” or “plant system” includes whole plants, plant organs, progeny of whole plants or plant organs, embryos, somatic embryos, embryo-like structures, protocorms, protocorm-like bodies (PLBs), and culture and/or suspensions of plant cells. Plant organs comprise, e.g., shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like). The invention may also include Cannabaceae and other Cannabis strains, such as C. sativa generally.

The term “expression,” as used herein, or “expression of a coding sequence” (for example, a gene or a transgene) refer to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “nucleic acid” or “nucleic acid molecules” include single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), mRNA (messenger RNA), miRNA (micro-RNA), hpRNA (hairpin RNA), tRNA (transfer RNA), whether charged or discharged with a corresponding acetylated amino acid), and cRNA (complementary RNA). The term “deoxyribonucleic acid” (DNA) is inclusive of cDNA, genomic DNA, and DNA-RNA hybrids.

The terms “nucleic acid segment” and “nucleotide sequence segment,” or more generally “segment,” will be understood by those in the art as a functional term that includes both genomic sequences, ribosomal RNA sequences, transfer RNA sequences, messenger RNA sequences, operon sequences, and smaller engineered nucleotide sequences that encoded or may be adapted to encode, peptides, polypeptides, or proteins.

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide. It should be noted that any reference to a SEQ ID, or sequence specifically encompasses that sequence, as well as all corresponding sequences that correspond to that first sequence. For example, for any amino acid sequence identified, the specific specifically includes all compatible nucleotide (DNA and RNA) sequences that give rise to that amino acid sequence or protein, and vice versa.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hair-pinned, circular, and padlocked conformations.

As used herein with respect to DNA, the term “coding sequence,” “structural nucleotide sequence,” or “structural nucleic acid molecule” refers to a nucleotide sequence that is ultimately translated into a polypeptide, via transcription and mRNA, when placed under the control of appropriate regulatory sequences. With respect to RNA, the term “coding sequence” refers to a nucleotide sequence that is translated into a peptide, polypeptide, or protein. The boundaries of a coding sequence are determined by a translation start codon at the 5′-terminus and a translation stop codon at the 3′-terminus. Coding sequences include, but are not limited to: genomic DNA; cDNA; EST; and recombinant nucleotide sequences. Notably, all amino acid sequence identified herein also explicitly include the corresponding nucleotide coding sequence.

The term “sequence identity” or “identity,” as used herein in the context of two nucleic acid or polypeptide sequences, refers to the residues in the two sequences that are the same when aligned for maximum correspondence over a specified comparison window.

The term “recombinant” when used with reference, e.g., to a cell, or nucleic acid, protein, or vector, indicates that the cell, organism, nucleic acid, protein, or vector has been modified by the introduction of a heterologous nucleic acid or protein, or the alteration of a native nucleic acid or protein, or that the cell is derived from a cell so modified. Thus, for example, recombinant cells may express genes that are not found within the native (nonrecombinant or wild-type) form of the cell or express native genes that are otherwise abnormally expressed—over-expressed, under expressed, or not expressed at all.

The terms “approximately” and “about” refer to a quantity, level, value, or amount that varies by as much as 30%, or in another embodiment by as much as 20%, and in a third embodiment by as much as 10% to a reference quantity, level, value or amount. As used herein, the singular form “a,” “an,” and “the” include plural references unless the context clearly dictates otherwise.

As used herein, “heterologous” or “exogenous” in reference to a nucleic acid is a nucleic acid that originates from a foreign species, or is synthetically designed, or, if from the same species, is substantially modified from its native form in composition and/or genomic locus by deliberate human intervention. A heterologous protein may originate from a foreign species or, if from the same species, is substantially modified from its original form by deliberate human intervention. By “host cell” is meant a cell which contains an introduced nucleic acid construct and supports the replication and/or expression of the construct.

### EXAMPLES

### Example 1: Identification of Targets Proteins

The present inventors identified 1427 plant based lipocalin proteins from public databases. These protein targets were clustered into 75 homology families (90% homology) and extracted centroids and consensus sequences. The present inventors then identified unique consensus sequences from centroid sequences and pooled for 87 representative proteins. Here, 17 of these proteins resulted in high confidence binding to one or more target cannabinoid(s). Manual trimming of lipocalin domains in remaining proteins resulted in the identification of another 12 PLs with high confidence binding to one or more target cannabinoid(s). One of these proteins, it turns out, possesses two lipocalin domains. As shown in Table 2 below, the 29 modeled structures were then docked with an exemplary cannabinoid, CBD, of which 7 models showed CBD binding properly within the beta-barrel binding pocket. The remaining reflected surface binding properties. Binding affinities ranged from 0.6 nM to 5.7 μM.

Similarly, the present inventors scanned and identified top OBP-carrier targets as outlined in Table 1 that may be combined with cannabinoids or other target hydrophobic molecules resulting in an increase to the water-solubility of the complex. Notably, as demonstrated in Table, 1 OBPs having an affinity for cannabinoid may be from the lipocalins family with simulated structural backbones with close homology to identified lipocalin template structures identified. As noted in FIG. 3, across this genus of lipocalin proteins having affinity for one or more cannabinoid or other similar compounds may include common structural features. Again, shown in FIG. 3, which demonstrated 10 template or known lipocalins protein structures maintain a β-barrel binding pocket and β-sheet structure as shown in FIG. 4. The three-dimensional structure of the 26 predicted lipocalins protein that have affinity for one or more cannabinoid or other similar compounds also preserve the β-barrel binding pocket as shown in FIG. 3 and the β-sheet structure when overlaid one on-top of another also. In one preferred embodiment, a cannabinoid, such as THC, or other similar compound may to a lipocalins protein having a β-barrel binding pocket and β-sheet structure as shown in FIG. 4. In one embodiment, an exemplary OBP may bind one or more cannabinoids, such as THC as demonstrated in Table 1 and FIG. 5.

### Example 2. OBP and Lipocalin Binding to Cannabinoids by ANS Displacement

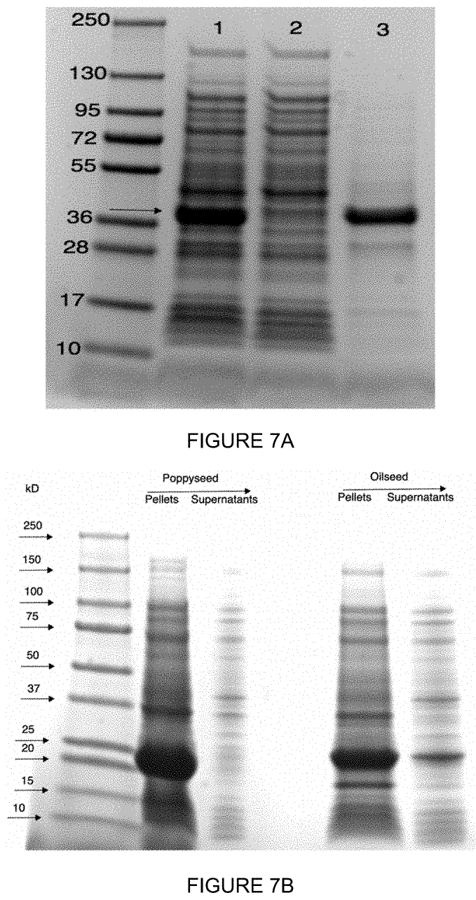

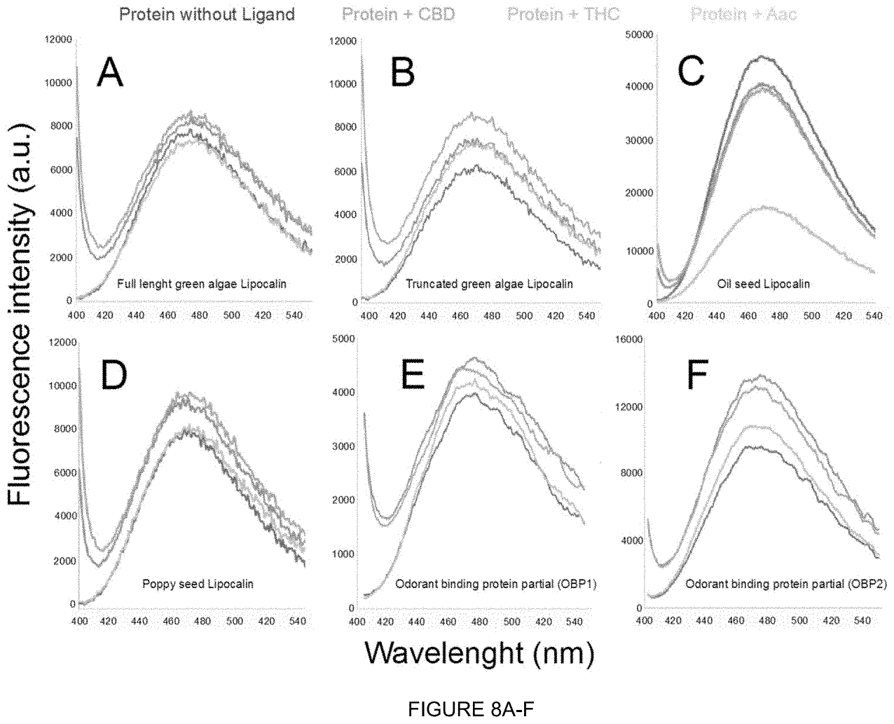

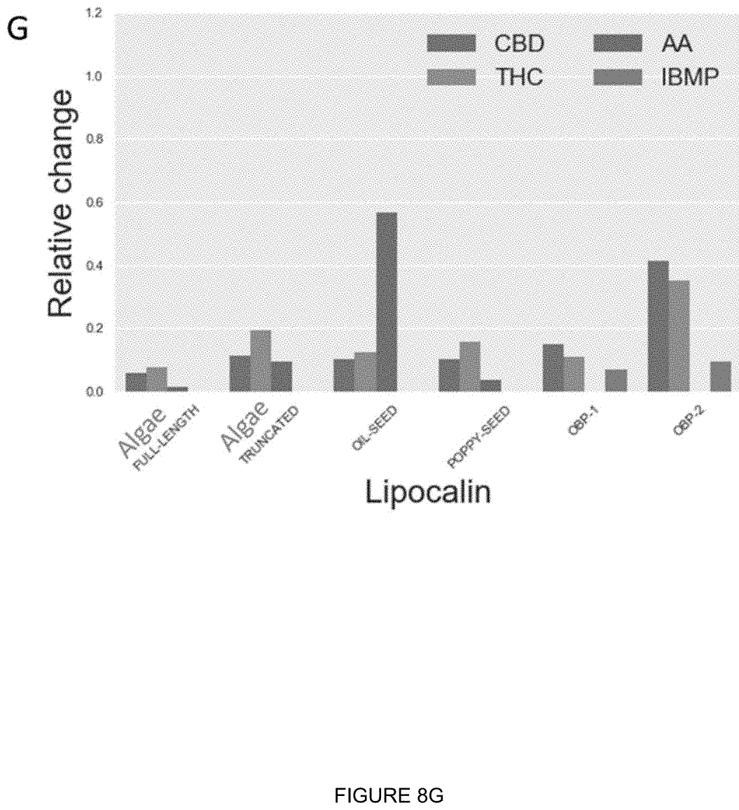

Exemplary OBPs and Lipocalins with high predicted binding affinity to cannabinoids were selected for overexpression, purification and binding assays. Lipocalin (LC-carrier) expression was confirmed with SDS-PAGE according to molecular weight (FIG. 7). Binding of the lipocalins (SEQ ID Nos. 1, 10, 30, and 33) to exemplary cannabinoids CBD and THC was determined by ANS displacement. All the four proteins were shown to bind to both THC and CBD (FIG. 8). Overall, OBP2 (OBP-carrier SEQ ID NO. 121) exhibited the highest binding affinity to CBD and THC. The present inventors further tested both a full length and a truncated (to optimize binding) lipocalin from the algae Micractinium conductrix. As generally shown in FIG. 8C, the truncated algae lipocalin having only those residues that are annotated or predicted to be directly part of the lipocalin beta-barrel fold binds to THC better than full length. (Examples annotated below in Table 3)

### Example 2. Materials and Methods

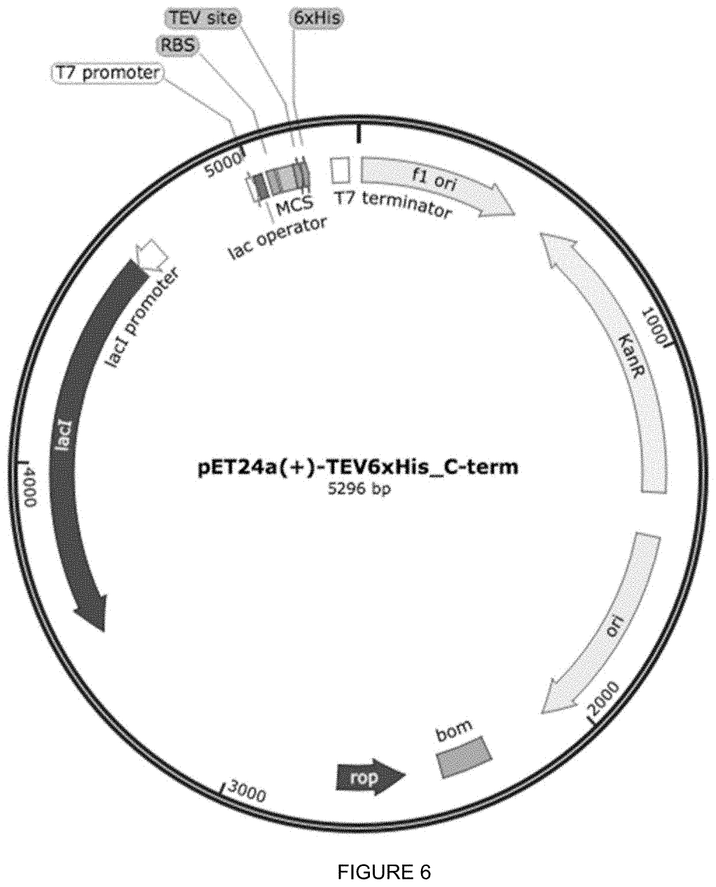

Cloning, transformation and protein expression in E. coli: Lipocalins and odorant binding proteins (OBPs) were cloned in a bacteria expression system using a modified pET 24a(+) vector (from GenScript, FIG. 6) and transformed in BL21 (DE3) competent cells. This vector is under the control of the strong T7 promoter, and has 6×His tag at the C-terminal of the protein sequence for purification. One colony was inoculated in 10 ml of LB and grown overnight for small scale protein expression. Next day, the culture was diluted 1:100 in LB medium and grown until OD reached 0.5. Protein expression was induced with 400 μM of isopropyl-β-d-thio-galactoside (IPTG) for 3 hours at 30 C and with shaking at 250 rpm. After 3 hours of growth, the cells were harvested and washed with 50 mM Tris-HCl and cell pellets were stored at −80° C. for further protein purification.

Protein purification: Cell pellets of 500 ml cell culture were thawed and resuspended in 15 ml of cell lysis containing 50 mM of Tris-HCl and protease inhibitors. Cells were lysed using Ultrasonic—Homogenizer, Biologics Inc Model 3000. After sonication lysed cells were spun down at 14,000 rpm for 10 min. Pellets were dissolved in the detergent-based buffer SoluLyse with multiple washing steps to extract protein from inclusion bodies according to SoluLyse manufacturers (Genlantis, San Diego, Calif.). Proteins from inclusion bodies were unfolded in 9M Urea and 5 mM DTT and refolded by dilution with 50 mM Tris-HCl and 150 mM NaCl pH 8 (Cabantous et al 2005). The refolded protein sample was spun down at 14,000 rpm for 10 min, the supernatant of refolded protein was applied to TALON resin and incubated for 1 hour at 4 degrees. His-tag protein was eluted with 200 mM Imidazole.

Ligand binding assays-ANS binding studies: Binding assays of cannabinoids to proteins were assessed by 8-anilino-1-naphthalenesulfonic acid (ANS, Thermofisher scientific, Waltham, Mass.) displacement. ANS is a fluorescent probe commonly used to measure conformational changes due to ligand binding. ANS binds mostly to hydrophobic sites in the protein (Yu and Strobel, 1996; Huang et al., 2016). 2 μM of protein was labelled with 20 μM of ANS. 100 μM stocks of exemplary cannabinoids cannabidiol (CBD), delta 9 tetrahydrocannabinol (THC) and Arachidonic acid were prepared in 10% of MeOH. Final concentration of each ligand was 33 μM. Arachidonic acid was used as a positive control for lipocalins and 2-isobutyl-3-methoxypyrazine (IBMP) for OBP respectively. Protein-ANS complex were excited at 390 nm and emission scan were recorded from 400 to 550 nm. All the experiments were done at 20° C. on a FluoroMax Spectrofluorometer.

### TABLES

TABLE 1
OBP lipocalins and simulated structure binding affinity to CBD and THC.

| | | THC | CBD |
|---|---|---|---|
| | | binding | binding |
| SEQ ID | | affinity | affinity |
| NO. | Protein ID | (kcal/mol) | (kcal/mol) |
| 148 | >EHA98383.1 Odorant-binding protein, partial [Heterocephalus glaber] | −5.51202 | −9.05076 |
| 121 | >XP_021009736.1 odorant-binding protein 1a-like [Mus caroli] | −5.27397 | −8.00003 |
| 146 | >XP_015353183.1 PREDICTED: odorant-binding protein 2b [Marmota | −8.11365 | −7.82024 |
| | marmota marmota] | | |
| 119 | >XP_008510274.1 PREDICTED: odorant-binding protein 2b-like [Equus | −7.496 | −7.69297 |
| | przewalskii] | | |
| 118 | >XP_012860280.1 PREDICTED: odorant-binding protein 2b-like [Echinops | −5.28992 | −7.38496 |
| | telfairi] | | |
| 122 | >XP_010604424.1 PREDICTED: odorant-binding protein [Fukomys | −8.09741 | −7.29234 |
| | damarensis] | | |
| 145 | >XP_021496743.1 odorant-binding protein 2a-like [Meriones unguiculatus] | −7.47672 | −7.28502 |
| 134 | >XP_004467463.1 odorant-binding protein 2b-like, partial [Dasypus | −7.72069 | −7.10146 |
| | novemcinctus] | | |
| 116 | >XP_027289850.1 odorant-binding protein 1b-like [Cricetulus griseusl | −4.52561 | −6.96519 |
| 141 | >XP_017899208.1 PREDICTED: odorant-binding protein-like [Capra hircus] | −6.40871 | −6.4312 |
| 120 | >XP_006877726.1 PREDICTED: odorant-binding protein-like [Chrysochloris | −7.11659 | −6.40555 |
| | asiatica] | | |
| 132 | >AAI22740.1 Odorant-binding protein-like [Bos taurus] | −7.06834 | −6.174 |
| 117 | >XP_006997496.1 PREDICTED: odorant-binding protein-like [Peromyscus | −6.36833 | −6.07852 |
| | maniculatus bairdii] | | |
| 136 | >XP_005372051.1 odorant-binding protein 1b-like [Microtus ochrogaster] | −5.59057 | −5.79454 |
| 142 | >XP_005346795.1 odorant-binding protein 2a-like [Microtus ochrogaster] | −7.01444 | −5.76349 |
| 129 | >XP_006835766.1 PREDICTED: odorant-binding protein-like [Chrysochloris | −5.13815 | −5.73119 |
| | asiatica] | | |
| 137 | >XP_021044251.1 odorant-binding protein 1a-like [Mus pahari] | −6.12296 | −5.72859 |
| 127 | >XP_006981169.1 PREDICTED: odorant-binding protein 2b-like [Peromyscus | −6.01789 | −5.32485 |
| | maniculatus bairdii] | | |
| 139 | >XP_004593691.1 PREDICTED: odorant-binding protein 2a [Ochotona | −6.68611 | −5.18765 |
| | princeps] | | |
| 135 | >XP_021010322.1 odorant-binding protein 1a-like [Mus caroli] | −6.23697 | −5.15617 |
| 133 | >XP 021045351.1 odorant-binding protein 1a-like, partial [Mus pahari] | −5.95383 | −5.14368 |
| 115 | >AIA65159.1 odorant binding protein 6 [Mus musculus musculus] | −5.31138 | −4.98043 |
| 119 | >XP_025132251.1 odorant-binding protein-like [Bubalus bubalis] | −5.53553 | −4.96312 |
| 125 | >XP_026333965.1 odorant-binding protein-like [Ursus arctos horribilis] | −4.34215 | −4.8448 |
| 138 | >KFO22773.1 Odorant-binding protein, partial [Fukomys damarensis] | −5.36065 | −4.61026 |
| 128 | >XP_014651019.1 PREDICTED: odorant-binding protein-like [Ceratotherium | −5.33005 | −4.51758 |
| | simum simum] | | |
| 114 | >NP_775171.1 odorant-binding protein 2a precursor [Rattus norvegicus] | −5.78556 | −4.51292 |
| 140 | >XP_003515366.1 odorant-binding protein 1a-like, partial [Cricetulus griseus] | −4.87291 | −4.31407 |
| 130 | >XP_005228600.1 odorant-binding protein-like [Bos taurus] | −5.46965 | −4.16188 |
| 113 | >NP_001119793.1 odorant binding protein 1b-like precursor [Mus musculus] | −6.64778 | −4.1559 |
| 35 | >XP_021117221.1 odorant-binding protein 2a-like [Heterocephalus glaber] | −5.55058 | −4.09064 |
| 126 | >XP_022374058.1 odorant-binding protein-like [Enhydra lutris kenyoni] | −4.65612 | −4.07627 |
| 143 | >XP_025118236.1 odorant-binding protein 2b-like [Bubalus bubalis] | −4.68564 | −3.40049 |
| 124 | >XP_025132613.1 odorant-binding protein-like [Bubalus bubalis] | −4.37815 | −3.37441 |
| 123 | >XP_026251381.1 odorant-binding protein 2b [Urocitellus parryii] | −4.6128 | −3.2619 |
| 144 | >XP_021496742.1 odorant-binding protein 2a-like [Meriones unguiculatus] | −5.99046 | −2.93976 |

TABLE 2
Plant lipocalins and simulated structure binding affinity to CBD and THC.

| | | THC | CBD |
|---|---|---|---|
| | | binding | binding |
| SEQ ID | | affinity | affinity |
| NO | Protein ID | (kcal/mol) | (kcal/mol) |
| 30 | >PSC68250.1 lipocalin-like domain [Micractinium conductrix] ** | −11.89843 | −12.57893 |
| 31 | >GAY52233.1 hypothetical protein CUMW_140330 [Citrus unshiu] | −5.80451 | −11.55021 |
| 25 | >NP 001276072.1 uncharacterized protein LOC102629088 [Citrus sinensis] | −8.01907 | −9.9839 |
| 1 | >Cluster63. ** | −8.8672 | −9.47932 |
| 4 | >AED96994.1 temperature-induced lipocalin [Arabidopsis thaliana] | −8.64671 | −8.86141 |
| 32 | >XP_003083465.1 Calycin-like [Ostreococcus tauri] | −6.94246 | −8.73101 |
| 33 | >OVA10565.1 Lipocalin/cytosolic fatty-acid binding domain [Macleaya | −7.66175 | −8.61909 |
| | cordata] | | |
| 23 | >PON79417.1 Lipocalin, bacterial [Parasponia andersonii] | −9.47908 | −8.58605 |
| 34 | >RLM75271.1 chloroplast lipocalin [Panicum miliaceum]. | −9.20508 | −8.51746 |
| 22 | >BAS79732.1 0s02g0612900 [Oryza sativa Japonica Group] | −6.47718 | −8.18968 |
| 35 | >NP_001306974.1 virus resistant/susceptible lipocalin [Solanum lycopersicum] | −6.27961 | −7.93453 |
| 19 | >PNX83699.1 temperature induced lipocalin [Trifolium pratense] | −6.09607 | −7.67605 |
| 40 | >BAS91118.1 Os04g0626400 [Oryza sativa Japonica Group] | −6.62506 | −7.25462 |
| 38 | >XP_010674669.1 PREDICTED: chloroplastic lipocalin [Beta vulgaris subsp. | −7.24293 | −7.24308 |
| | vulgaris]. ** | | |
| 24 | >GAV79982.1 Lipocalin 2 domain-containing protein [Cephalotus follicularis] | −5.91621 | −7.23258 |
| 36 | >KVH88723.1 Calycin [Cynara cardunculus var. scolymus] | −6.83237 | −7.20913 |
| 39 | >XP_024388985.1 apolipoprotein D-like [Physcomitrella patens] | −8.51821 | −6.88018 |
| 21 | >CDY32728.1 BnaA02g07900D [Brassica napus] | −8.78175 | −6.70346 |
| 5 | >BAT05618.1 Os08g0440100 [Oryza sativa Japonica Group] | −6.59436 | −6.64461 |
| 3 | >ACG48164.1 TIL-2 - Zea mays Temperature-induced lipocalin-2 [Zea mays] | −5.19434 | −6.53798 |
| 41 | >XP_007508739.1 predicted protein [Bathycoccus prasinos] | −6.08615 | −6.16951 |
| 37 | >KVH88723.1 Calycin [Cynara cardunculus var. scolymus] | −7.69504 | −6.08507 |
| 20 | >PNX64844.1 outer membrane lipoprotein blc-like [Trifolium pratense] | −7.75003 | −6.07673 |
| 17 | >KHG29526.1 lipocalin [Gossypium arboreum] | −8.68485 | −6.00903 |
| 42 | >OTF96447.1 putative chloroplastic lipocalin [Helianthus annuus] | −5.78231 | −5.83667 |
| 43 | >AEE78341.1 chloroplastic lipocalin [Arabidopsis thaliana] | −7.20569 | −4.97852 |
| 44 | >ACG35741.1 CHL - Zea mays Chloroplastic lipocalin [Zea mays] | −5.41836 | −4.89755 |
| 45 | >CDY32726.1 BnaA02g07880D [Brassica napus] | −6.42392 | −4.87333 |
| 46 | >CDY21802.1 BnaA06g20710D [Brassica napus] | −4.75948 | −4.35157 |
| 7 | >CDY62697.1 BnaA10g29280D [Brassica napus] | −3.39223 | −3.85676 |

TABLE 3
OBP and Lipocalin binding to cannabinoids

| Protein | | Purification |
|---|---|---|
| WT | Organism | Status |
| Full length | Green algae (Micractinium | Binds to CBD |
| Lipocalin like-domain | conductrix) | and THC |
| (SEQ ID NO. 10) | | |
| Modified lipocalin | Green algae (Micractinium | Binds to CBD |
| Lipocalin like domain | conductrix) | and THC |
| (SEQ ID NO. 30) | | |
| Lipocalin/cytosolic fatty- | Five seed poppy (Macleaya | Binds to CBD |
| acid binding domain | cordata) | and THC |
| (SEQ ID NO. 33) | | |
| Modified | Oilseed rape | Binds to CBD |
| Lipocalin: Custom 63 | (Brassica napus) | and THC |
| (SEQ ID NO. 1) | | |
| Odorant-binding protein, | Heterocephalus glaber | Binds to THC |
| partial (OBP1) | (naked mole- rat) | and CBD |
| (SEQ ID NO. 148) | | |
| Odorant binding protein | Mouse Mus caroli (Ryukyu | Binds to THC |
| 1a-like (OBP2) | mouse) | and CBD |
| (SEQ ID NO. 121) | | |

TABLE 4
Structural features of exemplary plant lipocalins and lipocalin-like proteins

| | | | | | | | | Conserved | |
|---|---|---|---|---|---|---|---|---|---|
| | Precursor/Mature | | Cleavage | | | | Conserved | N- | |
| | Molecular Mass | Subcellular | Site | SCR1 | SCR2 | SCR3 | Cys | glycosyl. | Other |
| Protein | (kDa) | Localisation | Position^{*} | GxWY | TDY | R | Residues | Sites | Domains |
| AtTIL-1 | 21 / 20 | membrane | C-terminal | yes | D only | yes | 0 | 1 | no |
| OsTIL-1 | 22 / 20 | membrane | C-terminal | yes | D only | yes | 0 | 1 | no |
| TaTIL-1 | 22 / 20 | membrane | C-terminal | yes | D only | yes | 0 | 1 | no |
| OsTIL-2 | 21 / 19 | ND | C-terminal | yes | D only | yes | 0 | 1 | no |
| AtCHL | 39 / 26 | chloroplast | N-terminal | yes | yes | yes | 8 | 0 | no |
| OsCHL | 37 / 26 | chloroplast | N-terminal | yes | yes | yes | 8 | 0 | no |
| AtVDE | 52 / 40 | chloroplast | N-terminal | yes | no | yes | 14 | 1 | yes^{**} |
| OsVDE | 50 / 40 | chloroplast | N-terminal | yes | no | yes | 14 | 1 | yes^{**} |
| TaVDE | 52 / 40 | chloroplast | N-terminal | yes | no | yes | 14 | 0 | yes^{**} |
| AtZEP | 74 / 68 | chloroplast | N-terminal | yes | no | no | 6 | 1 | yes^{***} |
| OsZEP | 68 / 63 | chloroplast | N-terminal | yes | no | no | 5 | 1 | yes^{***} |

At, Arabidopsis thaliana;
Ta, Triticum aestivum (wheat);
Os, Oryza sativa (rice);
Cys, Cysteine;
ND, not determined.
*C-terminal, GPI anchor site; N-terminal, signalpeptide.
**N-terminal cyteine-rich region and C-terminal glutamic acid-rich region.
***N-terminal ADP-binding site and C-terminal FAD-binding site.

SEQUENCE LISTINGS

SEQ ID NO. 1
Amino Acid
Cluster63 Unique
Artificial
MTSTEKKDMKAVKGLDLERYMGRWYEIASFPSRFQPKDGVDTRATYTLNPDGTVHVLNETWNGGKRGFIQ
GSAYKADPKSDEAKLKVKFFVPPFLPVIPVTGDYWVLYIDPEYQHAVIGQPSRSYLWILSRTAHMEEETY
KQLVEKAVEEGYDVSKLHKTPQSDTPPESNTAPDDTKGVWWLKSIFGK
SEQ ID NO. 2
Amino Acid
AEE78341.1 chloroplastic lipocalin
Arabidopsis thaliana
MILLSSSISLSRPVSSQSFSPPAATSTRRSHSSVTVKCCCSSRRLLKNPELKCSLENLFEIQALRKCFVS
GFAAILLLSQAGQGIALDLSSGYQNICQLGSAAAVGENKLTLPSDGDSESMMMMMMRGMTAKNFDPVRYS
GRWFEVASLKRGFAGQGQEDCHCTQGVYTFDMKESAIRVDTFCVHGSPDGYITGIRGKVQCVGAEDLEKS
ETDLEKQEMIKEKCFLRFPTIPFIPKLPYDVIATDYDNYALVSGAKDKGFVQVYSRTPNPGPEFIAKYKN
YLAQFGYDPEKIKDTPQDCEVTDAELAAMMSMPGMEQTLINQFPDLGLRKSVQFDPFTSVFETLKKLVPL
YFK
SEQ ID NO. 3
Amino Acid
ACG48164.1 TIL-2-Zea mays Temperature-induced lipocalin-2
Zea mays
MAMQVVRNLDLERYAGRWYEIACFPSRFQPKTGTNTRATYTLNPDGTVKVVNETWADGRRGHIEGTAWRA
DPASDEAKLKVRFYVPPFLPLIPVTGDYWVLHIDADYQYALVGQPSRNYLWILCRQPHMDESVYKELVER
AKEEGYDVSKLRKTAHPDPPPESEQSPRDGGMWWVKSIFGK
SEQ ID NO. 4
Amino Acid
AED96994.1 temperature-induced lipocalin
Arabidopsis thaliana
MTEKKEMEVVKGLNVERYMGRWYEIASFPSRFQPKNGVDTRATYTLNPDGTIHVLNETWSNGKRGFIEGS
AYKADPKSDEAKLKVKFYVPPFLPIIPVTGDYWVLYIDPDYQHALIGQPSRSYLWILSRTAQMEEETYKQ
LVEKAVEEGYDISKLHKTPQSDTPPESNTAPEDSKGVWWFKSLFGK
SEQ ID NO. 5
Amino Acid
BAT05618.1 Os08g0440100
Oryza sativa Japonica Group
MKVVRNLDLERYMGRWYEIACFPSRFQPRDGTNTRATYTLAGDGAVKVLNETWTDGRRGHIEGTAYRADP
VSDEAKLKVKFYVPPFLPIFPVVGDYWVLHVDDAYSYALVGQPSLNYLWILCRQPHMDEEVYGQLVERAK
EEGYDVSKLKKTAHPDPPPETEQSAGDRGVWWIKSLFGR
SEQ ID NO. 6
Amino Acid
BAS91118.1 Os04g0626400
Oryza sativa Japonica Group
MVLALLLGSSSSSLAAPHPACSSRRKCRPAGRNNFRCSLHDKVPLNAHGVLSTKLLSCLAASLVFISPPC
QAIPAETFVQPKLCQVAVVAAIDKAAVPLKFDSPSDDGGTGLMMKGMTAKNFDPIRYSGRWFEVASLKRG
FAGQGQEDCHCTQGVYSFDEKSRSIQVDTFCVHGGPDGYITGIRGRVQCLSEEDMASAETDLERQEMIKG
KCFLRFPTLPFIPKEPYDVLATDYDNYAVVSGAKDTSFIQIYSRTPNPGPEFIEKYKSYAANFGYDPSKI
KDTPQDCEVMSTDQLGLMMSMPGMTEALTNQFPDLKLSAPVAFNPFTSVFDTLKKLVELYFK
SEQ ID NO. 7
Amino Acid
CDY62697.1 BnaA10g29280D
Brassica napus
MTSTEKKDMNAVKGLDLERYMGRWYEIASFPSRFQPKDGVDTRATYTLNPDGTVHVLNETWNGGKRGFIQ
GSAYKADPKSDEAKLKVKFFVPPFLPVIPVTGDYWVLYIDPQYQHAVIGQPSRSYLWILSRTAHMEEETY
KQLVEKAVEEGYDVSKLHKTPQSDTPPESNTAPDDTKGVWWLKSIFGK
SEQ ID NO. 8
Amino Acid
XP_024388985.1 apolipoprotein D-like
Physcomitrella patens
MASVGASSVWHCILLLAMVVLTGEGARAKRILHTEAPSPSQGVCSNPPTVSNVSLEAYSGVWYEIGSTAL
VKARIERDLICATARYSVIPDGDLAGSIRVRNEGYNIRTGEFAHAIGTATVVSPGRLEVKFFPGAPGGDY
RIIYLSGKAEDKYNVAIVYSCDESVPGGSQSLFILSREPELDDEDDDDDDYDDDDETLSRLLNFVRDLGI
VFEPNNEFILTPQDPITCGRNGYDD
SEQ ID NO. 9
Amino Acid
CDY32726.1 BnaA02g07880D
Brassica napus
MMYVKVLMMVIAIWFVPMTYSNGAEAPAGDVAEAPGADAFNNDWYDARSTFYGDIHGGDTLKKKEEEKMT
TQNKEMEVVKDLDLERYMGRWYEIASFPSIFQPKNGIDTRATYTLNPDGTVDVLNETWNSGKRVFIQGSA
YKTDPKSDEAKFKVKFYVPPFLPIIPVTGDYWVLYIDPEYQHAVIGQPSRSYLWILSRTAHVEEETYKQL
LEKAVEEGYDVSKLHKTPQSDTPPESNAAPNDTKDQMLK
SEQ ID NO. 10
Amino Acid
PSC68250.1 lipocalin-like domain
Micractinium conductrix
MHVSTRQPCGAAPTAWPAQRPRSSPRRLACSAVLRDDARGVLQQAGLKLAAAAAAVLLAAPLHAGAASMP
ANAPLPALPPAPFDIEQSKQSKLLFDPMAYSGRWYEVASLKRGFAGEGQQDCHCTQGIYTPKEGGPEGAI
KLEVDTFCVHGGPGGRLSGIQGSVSCADPLLLSYLPEFQTEMEMVEGFVAKCALRFDSLAFLPPEPYVVL
RTDYTSYALVRGAKDRSFVQIYSRTPNPGAKFIAEQKAVLGQLGYPANDIVDTPQDCPEMAPQAMMAAMN
RGMSSTPTMPASTPPALAMAGYDLGPAAVVLGEEAPAPVKGIAFDRLRNPLESLKNVFSLFN
SEQ ID NO. 11
Amino Acid
GAY52233.1 hypothetical protein CUMW_140330
Citrus unshiu
MVNVIHQTSPALLQCCPSPPFANSIYRGNPRKKVYKCSFDNPISNKMVIGHVTRHLLSGLAASIIFLSQT
NQVVAADLPHFHNICQLASATDSMPTLPIELGSDERSGMLMMMRGMTAKDFDPVRYSGRWFEVASLKRGF
AGQGQEDCHCTQGVYTFDKEKPAIQVDTFCVHGGPDGYITGIRGNVQCLPEEELEKNVTDLEKQEMIKGK
CYLRFPTLPFIPKEPYDVIATDYDNFALVSGAKDKSFIQIYSRTPTPGPEFIEKYKSYLANFGYDPNKIK
DTPQDCEVISNSQLAAMMSMSGMQQALTNQFPDLELKSPLALNPFTSVLDTLKKLLELYFKK
SEQ ID NO. 12
Amino Acid
ACG35741.1 CHL-Zea mays Chloroplastic lipocalin
Zea mays
MVLLLLGCSPASSRPDCSPASRRRCSTAGQKMVRCSLNEETQLNKHGLVSKQLISCLAASLVFVSPPSQA
IPAETFARPGLCQIATVAAIDSASVPLKFDNPSDDVSTGMMMRGMTAKNFDPVRYSGRWFEVASLKRGFA
GQGQEDCHCTQGVYSFDEKARSIQVDTFCVHGGPDGYITGIRGRVQCLSEEDIASAETDLERQEMVRGKC
FLRFPTLPFIPKEPYDVLATDYDNYAIVSGAKDTSFIQIYSRTPNPGPEFIDKYKSYVANFGYDPSKIKD
TPQDCEYMSSDQIALMMSMPGMNEALTNQFPDLKLKAPVALNPFTSVFDTLKKLLELYFK
SEQ ID NO. 13
Amino Acid
OVA10565.1 Lipocalin/cytosolic fatty-acid binding domain
Macleaya cordata
MVLIQASPLSSPPLLRVIPANRTLACSLQQPASGTKVIAKHVLSGVAVSLIFLSQTNQVFAAEPSHYSNL
CQLAAVTDKGVTLPLEEGSDGRKGQLMMMRGMSAKNFDPIRYSGRWFEVASLKRGFAGSGQEDCHCTQGV
YTFDSEAPAIQVDTFCVHGGPDGYITGIRGKVQCLSEEDLEKNETDLEKRVMIREKCYLRFPTLPFIPKE
PYDVIATDYDNFALVSGAKDTSFIQIYSRTPNPGPEFIEKYKSYLGNYGYDPSMIKDTPQDCEVMSNSQL
AAMMSMSGMQQALTNQFPSLELKAPVEFNPFTSVFGTLKKLVELYFK
SEQ ID NO. 14
Amino Acid
OTF96447.1 putative chloroplastic lipocalin
Helianthus annuus
MAYPQSAIATGKSLLLLAPSHSPPISRTNISFKCYSTQSPLSISTKDAAAAAKHVLAAGLAACFMLLSPS
NQVLAIELSHNSLCQIASASNNVPTLEASNLMMMRGMTARNFDPVRYSGRWYEVASLKGGFAGQGQGDCH
CTQGVYTIDMKTPAIQVDTFCVHGGPDGYITGIRGNVQCLSEEETEKTETDLERKEMIKEKCYLRFPTLP
FIPKEPYDVLDTDYDNFALVSGAKDKSFIQIYSRTPNPGTEFIEKYKLVLADFGYDASKIKDTPQDCEVS
DSRLAAMMSMNGMQQALTNQFPDLELKSAVEFNPFTSVFDTFKKLVQLYFK
SEQ ID NO. 15
Amino Acid
XP_010674669.1 PREDICTED: chloroplastic lipocalin
Beta vulgaris subsp. vulgaris
MQVIKMSLPSPVLHRSSFSSSRGKPVNLVVRCSIDRPASENAIPKHIISGLVASCIFFSQANLVYGTDLP
RHNSICQLADVSSNKVPFPLDENASDANDKVIMMMMRGMSAKNFDPVRYAGRWFEVASLKRGFAGQGQED
CHCTQGVYTFDMETPAIQVDTFCVHGGPDGYITGIRGKVQCLSEEDKELKETDLERQEMIKEKCYLRFPT
LPFIPKEPYDVIATDYDHFALVSGAKDKSFIQIYSRTPNPGPEFIEKYKNYLADFGYDPNKTKDTPQDCQ
VMSNTQLASMMSQNGMQQVLNNQFPDLGLKASVEFNPFTSVLETLKKLVELYFK
SEQ ID NO. 16
Amino Acid
XP_007508739.1 predicted protein
Bathycoccus prasinos
MLQTRCCLRRKNDFASSSLLVALLAIAACASSFVTPALAGGLGRERRCPPVPTVSDVSIEAYASKPWYVQ
AQLPNRYQPVENLFCVRAVYTVTSPTTLDVFNFARKGSVEGEPSNEDMVLNAFIPDVDVKSKLKVGPKFV
PRALYGDYWIVAYEEEEGWAIISGGQPTIFVSDGLCTTESGNQGLWLFTREKEVSEELVETMKKKANALG
IDTSMLVTVQQTGCEYP
SEQ ID NO. 17
Amino Acid
KHG29526.1 lipocalin
Gossypium arboreum
MEVVKNLDIQRYMGKWYEIASFPSFFQPKKGENTSAFYTLKEDGTVHVLNETFVNGKKDSIEGTAYKADP
KSDEAKLKVKFYVPPFLPIIPVTGDYWVLYIDEDYQYVLVGGPTKKYLWILCRQKHMDEEIYNMLEQKAK
DLGYDVSKLHKTPQSDSTPEGEHVPQEKGFWWIKSLFGK
SEQ ID NO. 18
Amino Acid
XP 003083465.1 Calycin-like
Ostreococcus tauri
MTRRLRGHHAQRAVARLGAVALALALTRSHAFVLGVEASEECARVEPVDPFDLDAYVEAEWYVAAQKPTS
YQPTRDLFCVRANYTVVDERTISIWNTANRDGVDGSPRNADGRFKLRGLIEDPNMPSKIAVGMRFLPRFL
YGPYWVVATDVSPGDAEFDERGYSWAIISGGQPTISRGNGLCEPSGGLWLFVRDPEVSEEVVSKMKEKCE
SLGIDPDVLIPVTQEGCSFPTLP
SEQ ID NO. 19
Amino Acid
PNX83699.1 temperature induced lipocalin
Trifolium pratense
MGNNKEIEVVKGVDLERYMGRWYEIASFPSFFQPNNGENTRATYTLNSDGTVHVLNETWNKGKKNSIEGS
AYKANPNSDEAKLKVKFYVPPFLPIIPVTGDYWILYLDEDYQYALIGGPTTKYLWILSRKTHLDDEIYNQ
LIEKAKEEGYDVTKLHKTPQTDPPPPEQEGPQPKGIWSLFGK
SEQ ID NO. 20
Amino Acid
PNX64844.1 outer membrane lipoprotein blc-like
Trifolium pratense
MANKEMEVAKGVDLKRYMGRWYEIACFPSRFQPSDGCNTRATYTLKDDGTVNVLNETWSGGKRSYIEGTA
YKADPNSDEAKLKVKFYVPPFLPIIPVTGDYWVLHLDDDYSYALIGQPSRNYLWSPLTIAQLGELSWERH
HIWSLGWNPGDSTYSP
SEQ ID NO. 21
Amino Acid
CDY32728.1 BnaA02g07900D
Brassica napus
MTTQKKEMEVVKDLDLERYMGRWYEIASFPSIFQPKNGVDTRATYTLNPDGTVHVLNETWNGGKRAFIQG
SAYKTDPKSDEAKFKVKFYVPPFLPIIPVTGDYWVLYIDPEYQHAVIGQPSRSYLWILSRTAHVEEETYK
QLLQKAVEEGYDGDTPPESNAAPDDTKGVWWFKSMFGK
SEQ ID NO. 22
Amino Acid
BAS79732.1 Os02g0612900
Oryza sativa Japonica Group
MAAAAVEKKSGSEMTVVRGLDVARYMGRWYEIASLPNFFQPRDGRDTRATYALRPDGATVDVLNETWTSS
GKRDYIKGTAYKADPASDEAKLKVKFYLPPFLPVIPVVGDYWVLYVDDDYQYALVGEPRRKDLWILCRQT
SMDDEVYGRLLEKAKEEGYDVEKLRKTPQDDPPPESDAAPTDTKGTWWFKSLFGK
SEQ ID NO. 23
Amino Acid
PON79417.1 Lipocalin, bacterial
Parasponia andersonii
MAKKEMEVVKGLDLKRYMGKWYEIASFPSFFQPRNGVNTRATYTLNGDGTVKVLNETWSD
DKRDYIEGTAYKADPNSDEAKLKVKFYVPPFLPIIPVVGDYWVLYIDDDYQVALIGQPSRKYLWILARQT
HIDEEIYNQLVQRAKDEGYDVSKLNKTPQSDPPPEGDGPNDTKGIWWIKSLFGK
SEQ ID NO. 24
Amino Acid
GAV79982.1 Lipocalin_2 domain-containing protein
Cephalotus follicularis
MPKTVMKVVKDLDIPRYMGRWYEIASFPSRFQPKNGEDTRATYTLKEDGTINVLNETWTDGKRGYIEGTA
YKADATSNEAKLKVKFYVPPFLPIIPVVGDYWVLFIDDDYQYALIGQPSRKYLWILSRKTHLDDEIYNEL
VEKAKGEGYDVSKLHKTIQHDPPPEGEDGPKDTKGIWWIKSILGK
SEQ ID NO. 25
Amino Acid
NP_001276072.1 uncharacterized protein LOC102629088
Citrus sinensis
MASKKEMEVVRGLDIKRYMGRWYEIASFPSRNQPKNGADTRATYTLNEDGTVHVRNETWSDGKRGSIEGT
AYKADPKSDEAKLKVKFYVPPFFPIIPVVGNYWVLYIDDNYQYALIGEPTRKYLWILCREPHMDEAIYNQ
LVEKATSEGYDVSKLHRTPQSDNPPEAEESPQDTKGIWWIKSIFGK
SEQ ID NO. 26
Amino Acid
RLM75271.1 chloroplast lipocalin
Panicum miliaceum
MVLVALGCSPASSLPARSLTSRRKCSTTRQRIVRCSLNEETPLNKHGVVSKQIISCVAASLVFISPPSQA
IPAETSAQLGLCQIATVAAINSASVPLKFDSPSDEGSAGMMMMKGMTAKNFDPVRYSGRWFEVASLKRGF
AGQGQEDCHCTQGVCSFDEKSRSIQVDTFCVHGGPDGYITGIRGREPYDVLATDYDNYAIVSGAKDTSFI
QIYSRTPNPGPEFIKKYKSYVANFGYDPSKIKDTPQDCEYMSSDQLALMISMPGMNEALTNQFPDLKLKA
PIALNPFTSQQNSSEPVTDGAQPLLQDLSGKATAGPPTTSEERAAYAMASRSATKRGWSFVGGG
SEQ ID NO. 27
Amino Acid
KVH88723 .1 Calycin
Cynara cardunculus var. scolymus
MANKEMEVVKGVDLQRYMGRWYEIASFPSRFQPKDGINTRATYKLNEDGTINVLNETWSGGKRGYIEGTA
YKADPKSDEAKLKVKFYVPPFLPIIPVTGDYWVLYLDDDYRYALIGQPSRRYLWILSRQNHLDEEIYNQL
LEKAKEEGYDVSKLKKTTQTDPAPETDDAPADSKGDKAKAQEEQWQNTLEHKHILETCGLIKMEVAKGVD
LERYMGRWYEIASIPSRDQPKNGTNTRATYTLNSDGTVHVLNETWSDGKRGFIEGTAYKADPKSDEAKLK
VKFYVPPFLPIIPVTGDYWVLYLDDDYQYALIGQPSRNSLWILSRQNHLDEEIYEQLVQKAKEVGYDVSK
LKKTTHADTPPETEDAPADNKGIWWLKSIFGK
SEQ ID NO. 28
Amino Acid
NP_001306974.1 virus resistant/susceptible lipocalin
Solanum lycopersicum
MAALSASAHVRIRTFFHSSFTNNKISNFSQQFKLENYTTITTITTSKRSISIPALAPKTTENSASQLQST
SDSVKDSENINLKGWAEFAKNVSGEWDGFGADFSKQGEP1ELPESVVPGAYREWEVKVFDWQTQCPTLAR
DDDAFSFMYKFIRLLPTVGCEADAATRYSIDERNISDANVAAFAYQSTGCYVAAWSNNHDGNYNTAPYLS
WELEHCLIDPGDKESRVRIVQVVRLQDSKLVLQNIKVFCEHWYGPFRNGDQLGGCAIQDSAFASTKALDP
AEVIGVWEGKHAISSYNNAPEKVIQELVDGSTRKTVRDELDLVVLPRQLWCCLKGIAGGETCCEVGWLFD
QGRAITSKCIFSDNGKLKEIAIACESAAPAQ
SEQ ID NO. 29
Amino Acid
CDY21802.1 BnaA06g20710D
Brassica napus
MVSNIITSLSMTLVLPQSFTRPANTRCSVVRRINSRSHYSDRIICSLENPTESKEALRKHFVSGFAAILL
LSQAGQGVALDLSSRYHNICQLGSASVEGNKPTLPLDDDPEAMMMMMMRGMTAKNFDPVRYSGRWFEVAS
LKRGFAGQGQEDCHCTQGVYTFDMKEPAIRVDTFCVHGSPDGYITGIRGKVQCVGAQDLEKTETDLEKQE
MIKEKCYLRFPTIPFIPKLPYDVIATDYDNYALVSGAKDRSFVQVYSRTPNPGPEFIAKYKDYLAQFGYD
PEKIKDTPQDCEVMSDGQLAAMMSMPGMEKTLTNQFPDLELRKSVQFDPFTSVFETLKKLVPLYFK
SEQ ID NO. 30
Amino Acid
PSC68250.1 lipocalin-like domain (partial)
Micractinium conductrix
MAYSGRWYEVASLKRGFAGEGQQDCHCTQGIYTPKEGGPEGAIKLEVDTFCVHGGPGGRLSGIQGSVSCA
DPLLLSYLPEFQTEMEMVEGFVAKCALRFDSLAFLPPEPYVVLRTDYTSYALVRGAKDRSFVQIYSRTPN
PGAKFIAEQKAVLGQLGYPANDIVDTPQDCPEMAPQ
SEQ ID NO. 31
Amino Acid
GAY52233.1 hypothetical protein CUMW_140330 (partial)
Citrus unshiu
MVRYSGRWFEVASLKRGFAGQGQEDCHCTQGVYTFDKEKPAIQVDTFCVHGGPDGYITGIRGNVQCLPEE
ELEKNVIDLEKQEMIKGKCYLRFPTLPFIPKEPYDVIATDYDNFALVSGAKDKSFIQIYSRTPTPGPEFI
EKYKSYLANFGYDPNKIKDTPQ
SEQ ID NO. 32
Amino Acid
XP_003083465.1 Calycin-like (partial)
Ostreococcus tauri
MLDAYVEAEWYVAAQKPTSYQPTRDLFCVRANYTVVDERTISIWNTANRDGVDGSPRNADGRFKLRGLIE
DPNMPSKIAVGMRFLPRFLYGPYWVVATDVSPGDAEFDERGYSWAIISGGQPTISRGNGLCEPSGGLWLF
VRDPEVSEEVVSKMKEKCESLGIDPDVLIPVTQEGCSFPTLP
SEQ ID NO. 33
Amino Acid
OVA10565.1 Lipocalin/cytosolic fatty-acid binding domain (partial)
Macleaya cordata
MIRYSGRWFEVASLKRGFAGSGQEDCHCTQGVYTFDSEAPAIQVDTFCVHGGPDGYITGIRGKVQCLSEE
DLEKNETDLEKRVMIREKCYLRFPTLPFIPKEPYDVIATDYDNFALVSGAKDTSFIQIYSRTPNPGPEFI
EKYKSYLGNYGYDPSMIKDTPQ
SEQ ID NO. 34
Amino Acid
RLM75271.1 chloroplast lipocalin (partial)
Panicum mihaceum
MVRYSGRWFEVASLKRGFAGQGQEDCHCTQGVCSFDEKSRSIQVDTFCVHGGPDGYITGIRGREPYDVLA
TDYDNYAIVSGAKDTSFIQIYSRTPNPGPEFIKKYKSYVANFGYDPSKIKDTPQ
SEQ ID NO. 35
Amino Acid
NP_001306974.1 virus resistant/susceptible lipocalin (partial)
Solanum lycopersicum
MFAKNVSGEWDGFGADFSKQGEPIELPESVVPGAYREWEVKVFDWQTQCPTLARDDDAFSFMYKFIRLLP
TVGCEADAATRYSIDERNISDANVAAFAYQSTGCYVAAWSNNHDGNYNTAPYLSWELEHCLIDPGDKESR
VRIVQVVRLQDSKLVLQNIKVFCEHTNYGPF
SEQ ID NO. 36
Amino Acid
KVH88723.1 Calycin (partial; first lipocalin domain for this protein)
Cynara cardunculus var. scolymus
MVDLQRYMGRWYEIASFPSRFQPKDGINTRATYKLNEDGTINVLNETWSGGKRGYIEGTAYKADPKSDEA
KLKVKFYVPPFLPIIPVTGDYWVLYLDDDYRYALIGQPSRRYLWILSRQNHLDEEIYNQLLEKAKEEGYD
VSKLKKTTQTDPAP
SEQ ID NO 37
Amino Acid
KVH88723.1 Calycin (partial; second lipocalin domain for this protein)
Cynara cardunculus var. scolymus
MVDLERYMGRWYEIASIPSRDQPKNGINTRATYTLNSDGTVHVLNETWSDGKRGFIEGTAYKADPKSDEA
KLKVKFYVPPFLPIIPVTGDYWVLYLDDDYQYALIGQPSRNSLWILSRQNHLDEEIYEQLVQKAKEVGYD
VSKLKKTTHADTPP
SEQ ID NO. 38
Amino Acid
XP_010674669.1 PREDICTED: chloroplastic lipocalin (partial)
Beta vulgaris subsp. vulgaris
MVRYAGRWFEVASLKRGFAGQGQEDCHCTQGVYTFDMETPAIQVDTFCVHGGPDGYITGIRGKVQCLSEE
DKELKETDLERQEMIKEKCYLRFPTLPFIPKEPYDVIATDYDHFALVSGAKDKSFIQIYSRTPNPGPEFI
EKYKNYLADFGYDPNKTKDTPQ
SEQ ID NO. 39
Amino Acid
XP_024388985.1 apolipoprotein D-like (partial)
Physcomitrella patens
MVSLEAYSGVWYEIGSTALVKARIERDLICATARYSVIPDGDLAGSIRVRNEGYNIRTGEFAHAIGTATV
VSPGRLEVKFFPGAPGGDYRIIYLSGKAEDKYNVAIVYSCDESVPGGSQSLFILSREPELDDEDDDDDDY
DDDDETLSRLLNFVRDLGIVFEPNNEFILTPQDPITCGRNGYDD
SEQ ID NO. 40
Amino Acid
BAS91118.1 Os04g0626400 (partial)
Oryza sativa Japonica Group
MIRYSGRWFEVASLKRGFAGQGQEDCHCTQGVYSFDEKSRSIQVDTFCVHGGPDGYITGIRGRVQCLSEE
DMASAETDLERQEMIKGKCFLRFPTLPFIPKEPYDVLATDYDNYAVVSGAKDTSFIQIYSRTPNPGPEFI
EKYKSYAANFGYDPSKIKDTPQ
SEQ ID NO. 41
Amino Acid
XP_007508739.1 predicted protein (partial)
Bathycoccus prasinos
MIEAYASKPTNYVQAQLPNRYQPVENLFCVRAVYTVTSPTTLDVFNFARKGSVEGEPSNEDMVLNAFIPDV
DVKSKLKVGPKFVPRALYGDYWIVAYEEEEGTNAIISGGQPTIFVSDGLCTTESGNQGLWLFTREKEVSEE
LVETMKKKANALGIDTSMLVTVQQTGCEYP
SEQ ID NO. 42
Amino Acid
OTF96447.1 putative chloroplastic lipocalin (partial)
Hehanthus annuus
MVRYSGRWYEVASLKGGFAGQGQGDCHCTQGVYTIDMKTPAIQVDTFCVHGGPDGYITGIRGNVQCLSEE
ETEKTETDLERKEMIKEKCYLRFPTLPFIPKEPYDVLDTDYDNFALVSGAKDKSFIQIYSRTPNPGTEFI
EKYKLVLADFGYDASKIKDTPQ
SEQ ID NO. 43
Amino Acid
AEE78341.1 chloroplastic lipocalin (partial)
Arabidopsis thaliana
MVRYSGRWFEVASLKRGFAGQGQEDCHCTQGVYTFDMKESAIRVDTFCVHGSPDGYITGIRGKVQCVGAE
DLEKSETDLEKQEMIKEKCFLRFPTIPFIPKLPYDVIATDYDNYALVSGAKDKGFVQVYSRTPNPGPEFI
AKYKNYLAQFGYDPEKIKDTPQ
SEQ ID NO. 44
Amino Acid
ACG35741.1 CHL-Zea mays Chloroplastic lipocalin (partial)
Zea mays
MVRYSGRWFEVASLKRGFAGQGQEDCHCTQGVYSFDEKARSIQVDTFCVHGGPDGYITGIRGRVQCLSEE
DIASAETDLERQEMVRGKCFLRFPTLPFIPKEPYDVLATDYDNYAIVSGAKDTSFIQIYSRTPNPGPEFI
DKYKSYVANFGYDPSKIKDTPQ
SEQ ID NO. 45
Amino Acid
CDY32726.1 BnaA02g07880D (partial)
Brassica napus
MLDLERYMGRWYEIASFPSIFQPKNGIDTRATYTLNPDGTVDVLNETWNSGKRVFIQGSAYKTDPKSDEA
KFKVKFYVPPFLPIIPVTGDYWVLYIDPEYQHAVIGQPSRSYLWILSRTAHVEEETYKQLLEKAVEEGYD
VSKLHKTPQSDTPP
SEQ ID NO. 46
Amino Acid
CDY21802.1 BnaA06g20710D (partial)
Brassica napus
MVRYSGRWFEVASLKRGFAGQGQEDCHCTQGVYTFDMKEPAIRVDTFCVHGSPDGYITGIRGKVQCVGAQ
DLEKTETDLEKQEMIKEKCYLRFPTIPFIPKLPYDVIATDYDNYALVSGAKDRSFVQVYSRTPNPGPEFI
AKYKDYLAQFGYDPEKIKDTPQ
SEQ ID NO. 47
N-terminal secretion signal
S. cerevisiae
MRFPSIFTAVLFAASSALAAPVNITTEDETAQIPAEAVIGYSDLEGDFDVAVLPFSNSTNNGLLFINTTI
ASIAAKEEGVSLEKR
SEQ ID NO. 48
Amino Acid
Catalase
Arabidopsis thaliana
MDPYKYRPASSYNSPFFTTNSGAPVWNNNSSMTVGPRGLILLEDYHLVEKLANFDRERIPERVVHARGAS
AKGFFEVTHDISNLICADFLRAPGVQTPVIVRFSTVIHARGSPETLRDPRGFAVKFYTREGNFDLVGNNF
PVFFIRDGMKFPDIVHALKPNPKSHIQENWRILDFFSHHPESLNMFTFLFDDIGIPQDYRHMDGSGVNTY
MLINKAGKAHYVKFHWKPTCGVKSLLEEDAIRLGGTNHSHATQDLYDSIAAGNYPEWKLFIQIIDPADED
KFDFDPLDVIKTWPEDILPLQPVGRMVLNKNIDNFFAENEQLAFCPAIIVPGIHYSDDKLLQTRVFSYAD
TQRHRLGPNYLQLPVNAPKCAHHNNHHEGFMNFMHRDEEVNYFPSRYDQVRHAEKYPTPPAVCSGKRERC
IIEKENNFKEPGERYRTFTPERQERFIQRWIDALSDPRITHEIRSIWISYWSQADKSLGQKLASRLNVRP
SI
SEQ ID NO. 49
Amino Acid
Catalase HPII (KatE)
Escherichia coli
MSQHNEKNPHQHQSPLHDSSEAKPGMDSLAPEDGSHRPAAEPTPPGAQPTAPGSLKAPDTRNEKLNSLED
VRKGSENYALTTNQGVRIADDQNSLRAGSRGPTLLEDFILREKITHFDHERIPERIVHARGSAAHGYFQP
YKSLSDITKADFLSDPNKITPVFVRFSTVQGGAGSADTVRDIRGFATKFYTEEGIFDLVGNNTPIFFIQD
AHKFPDFVHAVKPEPHWAIPQGQSAHDTFWDYVSLQPETLHNVMWAMSDRGIPRSYRTMEGFGIHTFRLI
NAEGKATFVRFHWKPLAGKASLVWDEAQKLTGRDPDFHRRELWEAIEAGDFPEYELGFQLIPEEDEFKFD
FDLLDPTKLIPEELVPVQRVGKMVLNRNPDNFFAENEQAAFHPGHIVPGLDFTNDPLLQGRLFSYTDTQI
SRLGGPNFHEIPINRPTCPYHNFQRDGMHRMGIDTNPANYEPNSINDNWPRETPPGPKRGGFESYQERVE
GNKVRERSPSFGEYYSHPRLFWLSQTPFEQRHIVDGFSFELSKVVRPYIRERVVDQLAHIDLTLAQAVAK
NLGIELTDDQLNITPPPDVNGLKKDPSLSLYAIPDGDVKGRVVAILLNDEVRSADLLAILKALKAKGVHA
KLLYSRMGEVTADDGTVLPIAATFAGAPSLTVDAVIVPCGNIADIADNGDANYYLMEAYKHLKPIALAGD
ARKFKATIKIADQGEEGIVEADSADGSFMDELLTLMAAHRVWSRIPKIDKIPA
SEQ ID NO. 50
Amino Acid
Catalase 1
Arabidopsis thaliana
MDPYRVRPSSAHDSPFFTTNSGAPVWNNNSSLTVGTRGPILLEDYHLLEKLANFDRERIPERVVHARGAS
AKGFFEVTHDITQLTSADFLRGPGVQTPVIVRFSTVIHERGSPETLRDPRGFAVKFYTREGNFDLVGNNF
PVFFVRDGMKFPDMVHALKPNPKSHIQENWRILDFFSHHPESLHMFSFLFDDLGIPQDYRHMEGAGVNTY
MLINKAGKAHYVKFHWKPTCGIKCLSDEEAIRVGGANHSHATKDLYDSIAAGNYPQWNLFVQVMDPAHED
KFDFDPLDVTKIWPEDILPLQPVGRLVLNKNIDNFFNENEQIAFCPALVVPGIHYSDDKLLQTRIFSYAD
SQRHRLGPNYLQLPVNAPKCAHHNNHHDGFMNFMHRDEEVNYFPSRLDPVRHAEKYPTTPIVCSGNREKC
FIGKENNFKQPGERYRSWDSDRQERFVKRFVEALSEPRVTHEIRSIWISYWSQADKSLGQKLATRLNVRP
NF
SEQ ID NO. 51
Amino Acid
Catalase 2
Arabidopsis thaliana
MDPYKYRPASSYNSPFFTTNSGAPVWNNNSSMTVGPRGPILLEDYHLVEKLANFDRERIPERVVHARGAS
AKGFFEVTHDISNLICADFLRAPGVQTPVIVRFSTVIHERGSPETLRDPRGFAVKFYTREGNFDLVGNNF
PVFFIRDGMKFPDMVHALKPNPKSHIQENWRILDFFSHHPESLNMFTFLFDDIGIPQDYRHMDGSGVNTY
MLINKAGKAHYVKFHWKPTCGVKSLLEEDAIRVGGTNHSHATQDLYDSIAAGNYPEWKLFIQIIDPADED
KFDFDPLDVIKTWPEDILPLQPVGRMVLNKNIDNFFAENEQLAFCPAIIVPGIHYSDDKLLQTRVFSYAD
TQRHRLGPNYLQLPVNAPKCAHHNNHHEGFMNFMHRDEEVNYFPSRYDQVRHAEKYPTPPAVCSGKRERC
IIEKENNFKEPGERYRTFTPERQERFIQRWIDALSDPRITHEIRSIWISYWSQADKSLGQKLASRLNVRP
SI
SEQ ID NO. 52
Amino Acid
Catalase 3
Arabidopsis thaliana
MDPYKYRPSSAYNAPFYTTNGGAPVSNNISSLTIGERGPVLLEDYHLIEKVANFTRERIPERVVHARGIS
AKGFFEVTHDISNLTCADFLRAPGVQTPVIVRFSTVVHERASPETMRDIRGFAVKFYTREGNFDLVGNNT
PVFFIRDGIQFPDVVHALKPNPKTNIQEYWRILDYMSHLPESLLTWCWMFDDVGIPQDYRHMEGFGVHTY
TLIAKSGKVLFVKFHWKPTCGIKNLTDEEAKVVGGANHSHATKDLHDAIASGNYPEWKLFIQTMDPADED
KFDFDPLDVTKIWPEDILPLQPVGRLVLNRTIDNFFNETEQLAFNPGLVVPGIYYSDDKLLQCRIFAYGD
TQRHRLGPNYLQLPVNAPKCAHHNNHHEGFMNFMHRDEEINYYPSKFDPVRCAEKVPTPTNSYTGIRTKC
VIKKENNFKQAGDRYRSWAPDRQDRFVKRWVEILSEPRLTHEIRGIWISYWSQADRSLGQKLASRLNVRP
SI
SEQ ID NO. 53
Amino Acid
THCA Synthase Trichome targeting domain
Cannabis
MNCSAFSFWFVCKIIFFFLSFHIQISIA
SEQ ID NO. 54
Amino Acid
CBDA Synthase Trichome targeting domain
Cannabis
MKCSTFSFWFVCKIIFFFFSFNIQTSIA
SEQ ID NO. 55
Amino Acid
Cytosolic targeted THCA Synthase (ctTHCAs)
Cannabis
NPRENFLKCFSKHIPNNVANPKLVYTQHDQLYMSILNSTIQNLRFISDTTPKPLVIVTPSNNSHIQATIL
CSKKVGLQIRTRSGGHDAEGMSYISQVPFVVVDLRNMHSIKIDVHSQTAWVEAGATLGEVYYWINEKNEN
LSFPGGYCPTVGVGGHFSGGGYGALMRNYGLAADNIIDAHLVNVDGKVLDRKSMGEDLFWAIRGGGGENF
GIIAAWKIKLVDVPSKSTIFSVKKNMEIHGLVKLFNKWQNIAYKYDKDLVLMTHFITKNITDNHGKNKTT
VHGYFSSIFHGGVDSLVDLMNKSFPELGIKKTDCKEFSWIDTTIFYSGVVNFNTANFKKEILLDRSAGKK
TAFSIKLDYVKKPIPETAMVKILEKLYEEDVGAGMYVLYPYGGIMEEISESAIPFPHRAGIMYELWYTAS
WEKQEDNEKHINWVRSVYNFTTPYVSQNPRLAYLNYRDLDLGKTNHASPNNYTQARIWGEKYFGKNFNRL
VKVKTKVDPNNFFRNEQSIPPLPPHHH
SEQ ID NO. 56
DNA
Cytostolic CBDA synthase (cytCBDAs)
Cannabis sativa
ATGAATCCTCGAGAAAACTTCCTTAAATGCTTCTCGCAATATATTCCCAATAATGCAACAAATCTAAAAC
TCGTATACACTCAAAACAACCCATTGTATATGTCTGTCCTAAATTCGACAATACACAATCTTAGATTCAC
CTCTGACACAACCCCAAAACCACTTGTTATCGTCACTCCTTCACATGTCTCTCATATCCAAGGCACTATT
CTATGCTCCAAGAAAGTTGGCTTGCAGATTCGAACTCGAAGTGGTGGTCATGATTCTGAGGGCATGTCCT
ACATATCTCAAGTCCCATTTGTTATAGTAGACTTGAGAAACATGCGTTCAATCAAAATAGATGTTCATAG
CCAAACTGCATGGGTTGAAGCCGGAGCTACCCTTGGAGAAGTTTATTATTGGGTTAATGAGAAAAATGAG
AATCTTAGTTTGGCGGCTGGGTATTGCCCTACTGTTTGCGCAGGTGGACACTTTGGTGGAGGAGGCTATG
GACCATTGATGAGAAACTATGGCCTCGCGGCTGATAATATCATTGATGCACACTTAGTCAACGTTCATGG
AAAAGTGCTAGATCGAAAATCTATGGGGGAAGATCTCTTTTGGGCTTTACGTGGTGGTGGAGCAGAAAGC
TTCGGAATCATTGTAGCATGGAAAATTAGACTGGTTGCTGTCCCAAAGTCTACTATGTTTAGTGTTAAAA
AGATCATGGAGATACATGAGCTTGTCAAGTTAGTTAACAAATGGCAAAATATTGCTTACAAGTATGACAA
AGATTTATTACTCATGACTCACTTCATAACTAGGAACATTACAGATAATCAAGGGAAGAATAAGACAGCA
ATACACACTTACTTCTCTTCAGTTTTCCTTGGTGGAGTGGATAGTCTAGTCGACTTGATGAACAAGAGTT
TTCCTGAGTTGGGTATTAAAAAAACGGATTGCAGACAATTGAGCTGGATTGATACTATCATCTTCTATAG
TGGTGTTGTAAATTACGACACTGATAATTTTAACAAGGAAATTTTGCTTGATAGATCCGCTGGGCAGAAC
GGTGCTTTCAAGATTAAGTTAGACTACGTTAAGAAACCAATTCCAGAATCTGTATTTGTCCAAATTTTGG
AAAAATTATATGAAGAAGATATAGGAGCTGGGATGTATGCGTTGTACCCTTACGGTGGTATAATGGATGA
GATTTCAGAATCAGCAATTCCATTCCCTCATCGAGCTGGAATCTTGTATGAGTTATGGTACATATGTAGT
TGGGAGAAGCAAGAAGATAACGAAAAGCATCTAAACTGGATTAGAAATATTTATAACTTCATGACTCCTT
ATGTGTCCAAAAATCCAAGATTGGCATATCTCAATTATAGAGACCTTGATATAGGAATAAATGATCCCAA
GAATCCAAATAATTACACACAAGCACGTATTTGGGGTGAGAAGTATTTTGGTAAAAATTTTGACAGGCTA
GTAAAAGTGAAAACCCTGGTTGATCCCAATAACTTTTTTAGAAACGAACAAAGCATCCCACCTCTACCAC
GGCATCGTCATTAA
SEQ ID NO. 57
Amino Acid
Cytostolic CBDA synthase (cytCBDAs)
Cannabis sativa
MNPRENFLKCFSQYIPNNATNLKLVYTQNNPLYMSVLNSTIHNLRFTSDTTPKPLVIVTPSHVSHIQGTILCSKKVG
LQIRTRSGGHDSEGMSYISQVPFVIVDLRNMRSIKIDVHSQTAWVEAGATLGEVYYWVNEKNENLSLAAGYCPTVCA
GGHFGGGGYGPLMRNYGLAADNIIDAHLVNVHGKVLDRKSMGEDLFWALRGGGAESFGIIVAWKIRLVAVPKSTMFS
VKKIMEIHELVKLVNKWQNIAYKYDKDLLLMTHFITRNITDNQGKNKTAIHTYESSVFLGGVDSLVDLMNKSFPELG
IKKTDCRQLSWIDTIIFYSGVVNYDTDNENKEILLDRSAGQNGAFKIKLDYVKKPIPESVFVQILEKLYEEDIGAGM
YALYPYGGIMDEISESAIPFPHRAGILYELWYICSWEKQEDNEKHLNWIRNIYNFMTPYVSKNPRLAYLNYRDLDIG
INDPKNPNNYTQARIWGEKYFGKNFDRLVKVKTLVDPNNFFRNEQSIPPLPRHRH
SEQ ID NO. 58
DNA
MYB12-like
Cannabis
ATGAAGAAGAACAAATCAACTAGTAATAATAAGAACAACAACAGTAATAATATCATCAAAAACGACATCGTATCATC
ATCATCATCAACAACAACAACATCATCAACAACTACAGCAACATCATCATTTCATAATGAGAAAGTTACTGTCAGTA
CTGATCATATTATTAATCTTGATGATAAGCAGAAACGACAATTATGTCGTTGTCGTTTAGAAAAAGAAGAAGAAGAA
GAAGGAAGTGGTGGTTGTGGTGAGACAGTAGTAATGATGCTAGGGTCAGTATCTCCTGCTGCTGCTACTGCTGCTGC
AGCTGGGGGCTCATCAAGTTGTGATGAAGACATGTTGGGTGGTCATGATCAACTGTTGTTGTTGTGTTGTTCTGAGA
AAAAAACGACAGAAATTTCATCAGTGGTGAACTTTAATAATAATAATAATAATAATAAGGAAAATGGTGACGAAGTT
TCAGGACCGTACGATTATCATCATCATAAAGAAGAGGAAGAAGAAGAAGAAGAAGATGAAGCATCTGCATCAGTAGC
AGCTGTTGATGAAGGGATGTTGTTGTGCTTTGATGACATAATAGATAGCCACTTGCTAAATCCAAATGAGGTTTTGA
CTTTAAGAGAAGATAGCCATAATGAAGGTGGGGCAGCTGATCAGATTGACAAGACTACTTGTAATAATACTACTATT
ACTACTAATGATGATTATAACAATAACTTGATGATGTTGAGCTGCAATAATAACGGAGATTATGTTATTAGTGATGA
TCATGATGATCAGTACTGGATAGACGACGTCGTTGGAGTTGACTTTTGGAGTTGGGAGAGTTCGACTACTACTGTTA
TTACCCAAGAACAAGAACAAGAACAAGATCAAGTTCAAGAACAGAAGAATATGTGGGATAATGAGAAAGAGAAACTG
TTGTCTTTGCTATGGGATAATAGTGATAACAGCAGCAGTTGGGAGTTACAAGATAAAAGCAATAATAATAATAATAA
TAATGTTCCTAACAAATGTCAAGAGATTACCTCTGATAAAGAAAATGCTATGGTTGCATGGCTTCTCTCCTGA
SEQ ID NO. 59
Amino Acid
MYB 12
Cannabis
MKKNKSTSNNKNNNSNNIIKNDIVSSSSSTITTSSTTTATSSFHNEKVTVSTDHIINLDDKQKRQLCRCR
LEKEEEEEGSGGCGETVVMMLGSVSPAAATAAAAGGSSSCDEDMLGGHDQLLLLCCSEKKTTEISSVVNF
NNNNNNNKENGDEVSGPYDYHHHKEEEEEEEEDEASASVAAVDEGMLLCFDDIIDSHLLNPNEVLTLRED
SHNEGGAADQIDKTTCNNTTITTNDDYNNNLMMLSCNNNGDYVISDDHDDQYWIDDVVGVDFWSWESSTT
TVITQEQEQEQDQVQEQKNMWDNEKEKLLSLLWDNSDNSSSWELQDKSNNNNNNNVPNKCQEITSDKENA
MVAWLLS
SEQ ID NO. 60
Amino Acid
MYB8-orthologue for CAN738
Humulus lupulus
MGRAPCCEKVGLKKGRWTSEEDEILTKYIQSNGEGCWRSLPKNAGLLRCGKSCRLRWINYLRADLKRGNI
SSEEEDIIIKLHSTLGNRWSLIASHLPGRTDNEIKNYWNSHLSRKIHTFRRCNNTITHHHHLPNLVTVIK
VNLPIPKRKGGRTSRLAMKKNKSSTSNQNSSVIKNDVGSSSSTITTSVHQRTTITTPTMDDQQKRQLSRC
RLEEKEDQDGASTGTVVMMLGQAAAVGSSCDEDMLGHDQLSFLCCSEEKTTENSMTNLKENGDHEVSGPY
DYDHRYEKETSVDEGMLLCFNDIIDSNLLNPNEVLTLSEESLNLGGALMDTTTSTTTNNNNYSLSYNNNG
DCVISDDHDQYWLDDVVGVDFWSWESSTTVTQEQEQEQEQEQEQEQEQEQEQEHHHQQDQKKNTWDNEKE
KMLALLWDSDNSNWELQDNNNYHKCQEITSDKENAMVAWLLS
SEQ ID NO. 61
Amino Acid
atMYB12-orthologue for CAN739
Arabidopsis thaliana
MGRAPCCEKVGIKRGRWTAEEDQILSNYIQSNGEGSWRSLPKNAGLKRCGKSCRLRWINYLRSDLKRGNI
TPEEEELVVKLHSTLGNRWSLIAGHLPGRTDNEIKNYWNSHLSRKLHNFIRKPSISQDVSAVIMTNASSA
PPPPQAKRRLGRTSRSAMKPKIHRTKTRKTKKTSAPPEPNADVAGADKEALMVESSGAEAELGRPCDYYG
DDCNKNLMSINGDNGVLTFDDDIIDLLLDESDPGHLYTNTTCGGDGELHNIRDSEGARGFSDTWNQGNLD
CLLQSCPSVESFLNYDHQVNDASTDEFIDWDCVWQEGSDNNLWHEKENPDSMVSWLLDGDDEATIGNSNC
ENFGEPLDHDDESALVAWLLS
SEQ ID NO. 62
Amino Acid
MYB112-orthologue for CAN833
Arabidopsis thaliana
MNISRTEFANCKTLINHKEEVEEVEKKMEIEIRRGPWTVEEDMKLVSYISLHGEGRWNSLSRSAGLNRTG
KSCRLRWLNYLRPDIRRGDISLQEQFIILELHSRWGNRWSKIAQHLPGRTDNEIKNYWRTRVQKHAKLLK
CDVNSKQFKDTIKHLWMPRLIERIAATQSVQFTSNHYSPENSSVATATSSTSSSEAVRSSFYGGDQVEFG
TLDHMTNGGYWFNGGDTFETLCSFDELNKWLIQ
SEQ ID NO. 63
DNA
Cytochrome P450 (CYP3A4)
Mus musculus
ATGAACTTGTTTTCTGCTTTGTCTTTGGATACTTTGGTTTTGTTGGCTATTATTTTGGTTTTGTTGTACA
GATACGGTACTAGAACTCATGGTTTGTTTAAGAAGCAAGGTATTCCAGGTCCAAAGCCATTGCCATTTTT
GGGTACTGTTTTGAACTACTACACTGGTATTTGGAAGTTTGATATGGAATGTTACGAAAAGTACGGTAAG
ACTTGGGGTTTGTTTGATGGTCAAACTCCATTGTTGGTTATTACTGATCCAGAAACTATTAAGAACGTTT
TGGTTAAGGATTGTTTGTCTGTTTTTACTAACAGAAGAGAATTTGGTCCAGTTGGTATTATGTCTAAGGC
TATTTCTATTTCTAAGGATGAAGAATGGAAGAGATACAGAGCTTTGTTGTCTCCAACTTTTACTTCTGGT
AGATTGAAGGAAATGTTTCCAGTTATTGAACAATACGGTGATATTTTGGTTAAGTACTTGAGACAAGAAG
CTGAAAAGGGTATGCCAGTTGCTATGAAGGATGTTTTGGGTGCTTACTCTATGGATGTTATTACTTCTAC
TTCTTTTGGTGTTAACGTTGATTCTTTGAACAACCCAGAAGATCCATTTGTTGAAGAAGCTAAGAAGTTT
TTGAGAGTTGATTTTTTTGATCCATTGTTGTTTTCTGTTGTTTTGTTTCCATTGTTGACTCCAGTTTACG
AAATGTTGAACATTTGTATGTTTCCAAACGATTCTATTGAATTTTTTAAGAAGTTTGTTGATAGAATGCA
AGAATCTAGATTGGATTCTAACCAAAAGCATAGAGTTGATTTTTTGCAATTGATGATGAACTCTCATAAC
AACTCTAAGGATAAGGATTCTCATAAGGCTTTTTCTAACATGGAAATTACTGTTCAATCTATTATTTTTA
TTTCTGCTGGTTACGAAACTACTTCTTCTACTTTGTCTTTTACTTTGTACTGTTTGGCTACTCATCCAGA
TATTCAAAAGAAGTTGCAAGCTGAAATTGATAAGGCTTTGCCAAACAAGGCTACTCCAACTTGTGATACT
GTTATGGAAATGGAATACTTGGATATGGTTTTGAACGAAACTTTGAGATTGTACCCAATTGTTACTAGAT
TGGAAAGAGTTTGTAAGAAGGATGTTGAATTGAACGGTGTTTACATTCCAAAGGGTTCTATGGTTATGAT
TCCATCTTACGCTTTGCATCATGATCCACAACATTGGCCAGATCCAGAAGAATTTCAACCAGAAAGATTT
TCTAAGGAAAACAAGGGTTCTATTGATCCATACGTTTACTTGCCATTTGGTATTGGTCCAAGAAACTGTA
TTGGTATGAGATTTGCTTTGATGAACATGAAGTTGGCTGTTACTAAGGTTTTGCAAAACTTTTCTTTTCA
ACCATGTCAAGAAACTCAAATTCCATTGAAGTTGTCTAGACAAGGTATTTTGCAACCAGAAAAGCCAATT
GTTTTGAAGGTTGTTCCAAGAGATGCTGTTATTACTGGTGCTTAA
SEQ ID NO. 64
Amino Acid
Cytochrome P450 (CYP3A4)
Mus musculus
MNLFSALSLDTLVLLAIILVLLYRYGTRTHGLFKKQGIPGPKPLPFLGTVLNYYTGIWKFDMECYEKYGK
TWGLFDGQTPLLVITDPETIKNVLVKDCLSVFTNRREFGPVGIMSKAISISKDEEWKRYRALLSPTFTSG
RLKEMFPVIEQYGDILVKYLRQEAEKGMPVAMKDVLGAYSMDVITSTSFGVNVDSLNNPEDPFVEEAKKF
LRVDFFDPLLFSVVLFPLLTPVYEMLNICMFPNDSIEFFKKFVDRMQESRLDSNQKHRVDFLQLMMNSHN
NSKDKDSHKAFSNMEITVQSIIFISAGYETTSSTLSFTLYCLATHPDIQKKLQAEIDKALPNKATPTCDT
VMEMEYLDMVLNETLRLYPIVTRLERVCKKDVELNGVYIPKGSMVMIPSYALHHDPQHWPDPEEFQPERF
SKENKGSIDPYVYLPFGIGPRNCIGMRFALMNMKLAVTKVLQNFSFQPCQETQIPLKLSRQGILQPEKPI
VLKVVPRDAVITGA
SEQ ID NO. 65
DNA
P450 oxidoreductase gene (CYP oxidoreductase)
Mus musculus
ATGGGTGATTCTCATGAAGATACTTCTGCTACTGTTCCAGAAGCTGTTGCTGAAGAAGTTTCTTTGTTTT
CTACTACTGATATTGTTTTGTTTTCTTTGATTGTTGGTGTTTTGACTTACTGGTTTATTTTTAAGAAGAA
GAAGGAAGAAATTCCAGAATTTTCTAAGATTCAAACTACTGCTCCACCAGTTAAGGAATCTTCTTTTGTT
GAAAAGATGAAGAAGACTGGTAGAAACATTATTGTTTTTTACGGTTCTCAAACTGGTACTGCTGAAGAAT
TTGCTAACAGATTGTCTAAGGATGCTCATAGATACGGTATGAGAGGTATGTCTGCTGATCCAGAAGAATA
CGATTTGGCTGATTTGTCTTCTTTGCCAGAAATTGATAAGTCTTTGGTTGTTTTTTGTATGGCTACTTAC
GGTGAAGGTGATCCAACTGATAACGCTCAAGATTTTTACGATTGGTTGCAAGAAACTGATGTTGATTTGA
CTGGTGTTAAGTTTGCTGTTTTTGGTTTGGGTAACAAGACTTACGAACATTTTAACGCTATGGGTAAGTA
CGTTGATCAAAGATTGGAACAATTGGGTGCTCAAAGAATTTTTGAATTGGGTTTGGGTGATGATGATGGT
AACTTGGAAGAAGATTTTATTACTTGGAGAGAACAATTTTGGCCAGCTGTTTGTGAATTTTTTGGTGTTG
AAGCTACTGGTGAAGAATCTTCTATTAGACAATACGAATTGGTTGTTCATGAAGATATGGATACTGCTAA
GGTTTACACTGGTGAAATGGGTAGATTGAAGTCTTACGAAAACCAAAAGCCACCATTTGATGCTAAGAAC
CCATTTTTGGCTGCTGTTACTACTAACAGAAAGTTGAACCAAGGTACTGAAAGACATTTGATGCATTTGG
AATTGGATATTTCTGATTCTAAGATTAGATACGAATCTGGTGATCATGTTGCTGTTTACCCAGCTAACGA
TTCTACTTTGGTTAACCAAATTGGTGAAATTTTGGGTGCTGATTTGGATGTTATTATGTCTTTGAACAAC
TTGGATGAAGAATCTAACAAGAAGCATCCATTTCCATGTCCAACTACTTACAGAACTGCTTTGACTTACT
ACTTGGATATTACTAACCCACCAAGAACTAACGTTTTGTACGAATTGGCTCAATACGCTTCTGAACCATC
TGAACAAGAACATTTGCATAAGATGGCTTCTTCTTCTGGTGAAGGTAAGGAATTGTACTTGTCTTGGGTT
GTTGAAGCTAGAAGACATATTTTGGCTATTTTGCAAGATTACCCATCTTTGAGACCACCAATTGATCATT
TGTGTGAATTGTTGCCAAGATTGCAAGCTAGATACTACTCTATTGCTTCTTCTTCTAAGGTTCATCCAAA
CTCTGTTCATATTTGTGCTGTTGCTGTTGAATACGAAGCTAAGTCTGGTAGAGTTAACAAGGGTGTTGCT
ACTTCTTGGTTGAGAACTAAGGAACCAGCTGGTGAAAACGGTAGAAGAGCTTTGGTTCCAATGTTTGTTA
GAAAGTCTCAATTTAGATTGCCATTTAAGCCAACTACTCCAGTTATTATGGTTGGTCCAGGTACTGGTGT
TGCTCCATTTATGGGTTTTATTCAAGAAAGAGCTTGGTTGAGAGAACAAGGTAAGGAAGTTGGTGAAACT
TTGTTGTACTACGGTTGTAGAAGATCTGATGAAGATTACTTGTACAGAGAAGAATTGGCTAGATTTCATA
AGGATGGTGCTTTGACTCAATTGAACGTTGCTTTTTCTAGAGAACAAGCTCATAAGGTTTACGTTCAACA
TTTGTTGAAGAGAGATAAGGAACATTTGTGGAAGTTGATTCATGAAGGTGGTGCTCATATTTACGTTTGT
GGTGATGCTAGAAACATGGCTAAGGATGTTCAAAACACTTTTTACGATATTGTTGCTGAATTTGGTCCAA
TGGAACATACTCAAGCTGTTGATTACGTTAAGAAGTTGATGACTAAGGGTAGATACTCTTTGGATGTTTG
GTCTTAA
SEQ ID NO. 66
Amino Acid
P450 oxidoreductase (CYP oxidoreductase)
Mus musculus
MGDSHEDTSATVPEAVAEEVSLFSTTDIVLFSLIVGVLTYWFIFKKKKEEIPEFSKIQTTAPPVKESSFV
EKMKKTGRNIIVFYGSQTGTAEEFANRLSKDAHRYGMRGMSADPEEYDLADLSSLPEIDKSLVVFCMATY
GEGDPTDNAQDFYDWLQETDVDLTGVKFAVFGLGNKTYEHFNAMGKYVDQRLEQLGAQRIFELGLGDDDG
NLEEDFITWREQFWPAVCEFFGVEATGEESSIRQYELVVHEDMDTAKVYTGEMGRLKSYENQKPPFDAKN
PFLAAVTTNRKLNQGTERHLMHLELDISDSKIRYESGDHVAVYPANDSTLVNQIGEILGADLDVIMSLNN
LDEESNKKHPFPCPTTYRTALTYYLDITNPPRTNVLYELAQYASEPSEQEHLHKMASSSGEGKELYLSWV
VEARRHILAILQDYPSLRPPIDHLCELLPRLQARYYSIASSSKVHPNSVHICAVAVEYEAKSGRVNKGVA
TSWLRTKEPAGENGRRALVPMFVRKSQFRLPFKPTTPVIMVGPGTGVAPFMGFIQERAWLREQGKEVGET
LLYYGCRRSDEDYLYREELARFHKDGALTQLNVAFSREQAHKVYVQHLLKRDKEHLWKLIHEGGAHIYVC
GDARNMAKDVQNTFYDIVAEFGPMEHTQAVDYVKKLMTKGRYSLDVWS
SEQ ID NO. 67
DNA
Cytochrome P450 (CYP3A4)
Human
ATGGCTTTGATTCCTGATTTGGCTATGGAAACTAGATTGTTGTTGGCTGTTTCATTGGTTTTGTTGTATT
TGTATGGAACTCATTCACATGGATTGTTTAAAAAATTGGGAATTCCTGGACCTACTCCTTTGCCTTTTTT
GGGAAATATTTTGTCATATCATAAAGGATTTTGCATGTTTGATATGGAATGCCATAAAAAATATGGAAAA
GTTTGGGGATTTTATGATGGACAACAACCTGTTTTGGCTATTACTGATCCTGATATGATTAAAACTGTTT
TGGTTAAAGAATGCTATTCAGTTTTTACTAATAGAAGACCTTTTGGACCTGTTGGATTTATGAAATCAGC
TATTTCAATTGCTGAAGATGAAGAATGGAAAAGATTGAGATCATTGTTGTCACCTACTTTTACTTCAGGA
AAATTGAAAGAAATGGTTCCTATTATTGCTCAATATGGAGATGTTTTGGTTAGAAATTTGAGAAGAGAAG
CTGAAACTGGAAAACCTGTTACTTTGAAAGATGTTTTTGGAGCTTATTCAATGGATGTTATTACTTCAAC
TTCATTTGGAGTTAATATTGATTCATTGAATAATCCTCAAGATCCTTTTGTTGAAAATACTAAAAAATTG
TTGAGATTTGATTTTTTGGATCCTTTTTTTTTGTCAATTACTGTTTTTCCTTTTTTGATTCCTATTTTGG
AAGTTTTGAATATTTGCGTTTTTCCTAGAGAAGTTACTAATTTTTTGAGAAAATCAGTTAAAAGAATGAA
AGAATCAAGATTGGAAGATACTCAAAAACATAGAGTTGATTTTTTGCAATTGATGATTGATTCACAAAAT
TCAAAAGAAACTGAATCACATAAAGCTTTGTCAGATTTGGAATTGGTTGCTCAATCAATTATTTTTATTT
TTGCTGGATGCGAAACTACTTCATCAGTTTTGTCATTTATTATGTATGAATTGGCTACTCATCCTGATGT
TCAACAAAAATTGCAAGAAGAAATTGATGCTGTTTTGCCTAATAAAGCTCCTCCTACTTATGATACTGTT
TTGCAAATGGAATATTTGGATATGGTTGTTAATGAAACTTTGAGATTGTTTCCTATTGCTATGAGATTGG
AAAGAGTTTGCAAAAAAGATGTTGAAATTAATGGAATGTTTATTCCTAAAGGAGTTGTTGTTATGATTCC
TTCATATGCTTTGCATAGAGATCCTAAATATTGGACTGAACCTGAAAAATTTTTGCCTGAAAGATTTTCA
AAAAAAAATAAAGATAATATTGATCCTTATATTTATACTCCTTTTGGATCAGGACCTAGAAATTGCATTG
GAATGAGATTTGCTTTGATGAATATGAAATTGGCTTTGATTAGAGTTTTGCAAAATTTTTCATTTAAACC
TTGCAAAGAAACTCAAATTCCTTTGAAATTGTCATTGGGAGGATTGTTGCAACCTGAAAAACCTGTTGTT
TTGAAAGTTGAATCAAGAGATGGAACTGTTTCAGGAGCT
SEQ ID NO. 68
Amino Acid
Cytochrome P450 (CYP3A4)
Human
MALIPDLAMETRLLLAVSLVLLYLYGTHSHGLFKKLGIPGPTPLPFLGNILSYHKGFCMFDMECHKKYGK
VWGFYDGQQPVLAITDPDMIKTVLVKECYSVFTNRRPFGPVGFMKSAISIAEDEEWKRLRSLLSPTFTSG
KLKEMVPIIAQYGDVLVRNLRREAETGKPVTLKDVFGAYSMDVITSTSFGVNIDSLNNPQDPFVENTKKL
LRFDFLDPFFLSITVFPFLIPILEVLNICVFPREVTNFLRKSVKRMKESRLEDTQKHRVDFLQLMIDSQN
SKETESHKALSDLELVAQSIIFIFAGCETTSSVLSFIMYELATHPDVQQKLQEEIDAVLPNKAPPTYDTV
LQMEYLDMVVNETLRLFPIAMRLERVCKKDVEINGMFIPKGVVVMIPSYALHRDPKYWTEPEKFLPERFS
KKNKDNIDPYIYTPFGSGPRNCIGMRFALMNMKLALIRVLQNFSFKPCKETQIPLKLSLGGLLQPEKPVV
LKVESRDGTVSGA
SEQ ID NO. 69
DNA
P450 oxidoreductase gene (oxred)
Human
ATGATTAATATGGGAGATTCACATGTTGATACTTCATCAACTGTTTCAGAAGCTGTTGCTGAAGAAGTTT
CATTGTTTTCAATGACTGATATGATTTTGTTTTCATTGATTGTTGGATTGTTGACTTATTGGTTTTTGTT
TAGAAAAAAAAAAGAAGAAGTTCCTGAATTTACTAAAATTCAAACTTTGACTTCATCAGTTAGAGAATCA
TCATTIGTTGAAAAAATGAAAAAAACTGGAAGAAATATTATTGTTITITATGGATCACAAACTGGAACTG
CTGAAGAATTTGCTAATAGATTGTCAAAAGATGCTCATAGATATGGAATGAGAGGAATGTCAGCTGATCC
TGAAGAATATGATTTGGCTGATTTGTCATCATTGCCTGAAATTGATAATGCTTTGGTTGTTTTTTGCATG
GCTACTTATGGAGAAGGAGATCCTACTGATAATGCTCAAGATTTTTATGATTGGTTGCAAGAAACTGATG
TTGATTTGTCAGGAGTTAAATTTGCTGTTTTTGGATTGGGAAATAAAACTTATGAACATTTTAATGCTAT
GGGAAAATATGTTGATAAAAGATTGGAACAATTGGGAGCTCAAAGAATTTTTGAATTGGGATTGGGAGAT
GATGATGGAAATTTGGAAGAAGATTTTATTACTTGGAGAGAACAATTTTGGTTGGCTGTTTGCGAACATT
TTGGAGTTGAAGCTACTGGAGAAGAATCATCAATTAGACAATATGAATTGGTTGTTCATACTGATATTGA
TGCTGCTAAAGTTTATATGGGAGAAATGGGAAGATTGAAATCATATGAAAATCAAAAACCTCCTTTTGAT
GCTAAAAATCCTTTTTTGGCTGCTGTTACTACTAATAGAAAATTGAATCAAGGAACTGAAAGACATTTGA
TGCATTTGGAATTGGATATTTCAGATTCAAAAATTAGATATGAATCAGGAGATCATGTTGCTGTTTATCC
TGCTAATGATTCAGCTTTGGTTAATCAATTGGGAAAAATTTTGGGAGCTGATTTGGATGTTGTTATGTCA
TTGAATAATTTGGATGAAGAATCAAATAAAAAACATCCTTTTCCTTGCCCTACTTCATATAGAACTGCTT
TGACTTATTATTTGGATATTACTAATCCTCCTAGAACTAATGTTTTGTATGAATTGGCTCAATATGCTTC
AGAACCTTCAGAACAAGAATTGTTGAGAAAAATGGCTTCATCATCAGGAGAAGGAAAAGAATTGTATTTG
TCATGGGTTGTTGAAGCTAGAAGACATATTTTGGCTATTTTGCAAGATTGCCCTTCATTGAGACCTCCTA
TTGATCATTTGTGCGAATTGTTGCCTAGATTGCAAGCTAGATATTATTCAATTGCTTCATCATCAAAAGT
TCATCCTAATTCAGTTCATATTTGCGCTGTTGTTGTTGAATATGAAACTAAAGCTGGAAGAATTAATAAA
GGAGTTGCTACTAATTGGTTGAGAGCTAAAGAACCTGTTGGAGAAAATGGAGGAAGAGCTTTGGTTCCTA
TGTTTGTTAGAAAATCACAATTTAGATTGCCTTTTAAAGCTACTACTCCTGTTATTATGGTTGGACCTGG
AACTGGAGTTGCTCCTTTTATTGGATTTATTCAAGAAAGAGCTTGGTTGAGACAACAAGGAAAAGAAGTT
GGAGAAACTTTGTTGTATTATGGATGCAGAAGATCAGATGAAGATTATTTGTATAGAGAAGAATTGGCTC
AATTTCATAGAGATGGAGCTTTGACTCAATTGAATGTTGCTTTTTCAAGAGAACAATCACATAAAGTTTA
TGTTCAACATTTGTTGAAACAAGATAGAGAACATTTGTGGAAATTGATTGAAGGAGGAGCTCATATTTAT
GTTTGCGGAGATGCTAGAAATATGGCTAGAGATGTTCAAAATACTTTTTATGATATTGTTGCTGAATTGG
GAGCTATGGAACATGCTCAAGCTGTTGATTATATTAAAAAATTGATGACTAAAGGAAGATATTCATTGGA
TGTTTGGTCA
SEQ ID NO. 70
Amino Acid
P450 oxidoreductase
Human
MINMGDSHVDTSSTVSEAVAEEVSLFSMTDMILFSLIVGLLTYAFLFRKKKEEVPEFTKIQTLTSSVRES
SFVEKMKKTGRNIIVFYGSQTGTAEEFANRLSKDAHRYGMRGMSADPEEYDLADLSSLPEIDNALVVFCM
ATYGEGDPTDNAQDFYDALQETDVDLSGVKFAVFGLGNKTYEHFNAMGKYVDKRLEQLGAQRIFELGLGD
DDGNLEEDFITAREQFALAVCEHFGVEATGEESSIRQYELVVHTDIDAAKVYMGEMGRLKSYENQKPPFD
AKNPFLAAVTTNRKLNQGTERHLMHLELDISDSKIRYESGDHVAVYPANDSALVNQLGKILGADLDVVMS
LNNLDEESNKKHPFPCPTSYRTALTYYLDITNPPRTNVLYELAQYASEPSEQELLRKMASSSGEGKELYL
SAVVEARRHILAILQDCPSLRPPIDHLCELLPRLQARYYSIASSSKVHPNSVHICAVVVEYETKAGRINK
GVATNALRAKEPVGENGGRALVPMFVRKSQFRLPFKATTPVIMVGPGTGVAPFIGFIQERAALRQQGKEV
GEILLYYGCRRSDEDYLYREELAQFHRDGALTQLNVAFSREQSHKVYVQHLLKQDREHLAKLIEGGAHIY
VCGDARNMARDVQNTFYDIVAELGAMEHAQAVDYIKKLMTKGRYSLDVAS
SEQ ID NO. 71
DNA
cannabidiolic acid (CBDA) synthase
Cannabis sativa
ATGAATCCTCGAGAAAACTTCCTTAAATGCTTCTCGCAATATATTCCCAATAATGCAACAAATCTAAAAC
TCGTATACACTCAAAACAACCCATTGTATATGTCTGTCCTAAATTCGACAATACACAATCTTAGATTCAC
CTCTGACACAACCCCAAAACCACTTGTTATCGTCACTCCTTCACATGTCTCTCATATCCAAGGCACTATT
CTATGCTCCAAGAAAGTTGGCTTGCAGATTCGAACTCGAAGTGGTGGTCATGATTCTGAGGGCATGTCCT
ACATATCTCAAGTCCCATTTGTTATAGTAGACTTGAGAAACATGCGTTCAATCAAAATAGATGTTCATAG
CCAAACTGCATGGGTTGAAGCCGGAGCTACCCTTGGAGAAGTTTATTATTGGGTTAATGAGAAAAATGAG
AATCTTAGTTTGGCGGCTGGGTATTGCCCTACTGTTTGCGCAGGTGGACACTTTGGTGGAGGAGGCTATG
GACCATTGATGAGAAACTATGGCCTCGCGGCTGATAATATCATTGATGCACACTTAGTCAACGTTCATGG
AAAAGTGCTAGATCGAAAATCTATGGGGGAAGATCTCTTTTGGGCTTTACGTGGTGGTGGAGCAGAAAGC
TTCGGAATCATTGTAGCATGGAAAATTAGACTGGTTGCTGTCCCAAAGTCTACTATGTTTAGTGTTAAAA
AGATCATGGAGATACATGAGCTTGTCAAGTTAGTTAACAAATGGCAAAATATTGCTTACAAGTATGACAA
AGATTTATTACTCATGACTCACTTCATAACTAGGAACATTACAGATAATCAAGGGAAGAATAAGACAGCA
ATACACACTTACTTCTCTTCAGTTTTCCTTGGTGGAGTGGATAGTCTAGTCGACTTGATGAACAAGAGTT
TTCCTGAGTTGGGTATTAAAAAAACGGATTGCAGACAATTGAGCTGGATTGATACTATCATCTTCTATAG
TGGTGTTGTAAATTACGACACTGATAATTTTAACAAGGAAATTTTGCTTGATAGATCCGCTGGGCAGAAC
GGTGCTTTCAAGATTAAGTTAGACTACGTTAAGAAACCAATTCCAGAATCTGTATTTGTCCAAATTTTGG
AAAAATTATATGAAGAAGATATAGGAGCTGGGATGTATGCGTTGTACCCTTACGGTGGTATAATGGATGA
GATTTCAGAATCAGCAATTCCATTCCCTCATCGAGCTGGAATCTTGTATGAGTTATGGTACATATGTAGT
TGGGAGAAGCAAGAAGATAACGAAAAGCATCTAAACTGGATTAGAAATATTTATAACTTCATGACTCCTT
ATGTGTCCAAAAATTCAAGATTGGCATATCTCAATTATAGAGACCTTGATATAGGAATAAATGATCCCAA
GAATCCAAATAATTACACACAAGCACGTATTTGGGGTGAGAAGTATTTTGGTAAAAATTTTGACAGGCTA
GTAAAAGTGAAAACCCTGGTTGATCCCAATAACTTTTTTAGAAACGAACAAAGCATCCCACCTCAACCAC
GGCATCGTCATTAA
SEQ ID NO. 72
Amino Acid
Cannabidiolic acid (CBDA) synthase
Cannabis sativa
MNPRENFLKCFSQYIPNNATNLKLVYTQNNPLYMSVLNSTIHNLRFTSDTTPKPLVIVTPSHVSHIQGTI
LCSKKVGLQIRTRSGGHDSEGMSYISQVPFVIVDLRNMRSIKIDVHSQTAWVEAGATLGEVYYWVNEKNE
NLSLAAGYCPTVCAGGHFGGGGYGPLMRNYGLAADNIIDAHLVNVHGKVLDRKSMGEDLFWALRGGGAES
FGIIVAWKIRLVAVPKSTMFSVKKIMEIHELVKLVNKWQNIAYKYDKDLLLMTHFITRNITDNQGKNKTA
IHTYFSSVFLGGVDSLVDLMNKSFPELGIKKTDCRQLSWIDTIIFYSGVVNYDTDNFNKEILLDRSAGQN
GAFKIKLDYVKKPIPESVFVQILEKLYEEDIGAGMYALYPYGGIMDEISESAIPFPHRAGILYELWYICS
WEKQEDNEKHLNWIRNIYNFMTPYVSKNSRLAYLNYRDLDIGINDPKNPNNYTQARIWGEKYFGKNFDRL
VKVKTLVDPNNFFRNEQSIPPQPRHRH
SEQ ID NO. 73
DNA
UDP glycosyltransferase 76G1
Stevia rebaudiana
ATGGAAAATAAAACTGAAACTACTGTTAGAAGAAGAAGAAGAATTATTTTGTTTCCTGTTCCTTTTCAAG
GACATATTAATCCTATTTTGCAATTGGCTAATGTTTTGTATTCAAAAGGATTTTCAATTACTATTTTTCA
TACTAATTTTAATAAACCTAAAACTTCAAATTATCCTCATTTTACTTTTAGATTTATTTTGGATAATGAT
CCTCAAGATGAAAGAATTTCAAATTTGCCTACTCATGGACCTTTGGCTGGAATGAGAATTCCTATTATTA
ATGAACATGGAGCTGATGAATTGAGAAGAGAATTGGAATTGTTGATGTTGGCTTCAGAAGAAGATGAAGA
AGTTTCATGCTTGATTACTGATGCTTTGTGGTATTTTGCTCAATCAGTTGCTGATTCATTGAATTTGAGA
AGATTGGTTTTGATGACTTCATCATTGTTTAATTTTCATGCTCATGTTTCATTGCCTCAATTTGATGAAT
TGGGATATTTGGATCCTGATGATAAAACTAGATTGGAAGAACAAGCTTCAGGATTTCCTATGTTGAAAGT
TAAAGATATTAAATCAGCTTATTCAAATTGGCAAATTTTGAAAGAAATTTTGGGAAAAATGATTAAACAA
ACTAGAGCTTCATCAGGAGTTATTTGGAATTCATTTAAAGAATTGGAAGAATCAGAATTGGAAACTGTTA
TTAGAGAAATTCCTGCTCCTTCATTTTTGATTCCTTTGCCTAAACATTTGACTGCTTCATCATCATCATT
GTTGGATCATGATAGAACTGTTTTTCAATGGTTGGATCAACAACCTCCTTCATCAGTTTTGTATGTTTCA
TTTGGATCAACTTCAGAAGTTGAAAAATGAGATTTTTTGGAAATTGCTAGAGGATTGGTTGATTCAAAAC
AATCATTTTTGTGGGTTGTTAGACCTGGATTTGTTAAAGGATCAACTTGGGTTGAACCTTTGCCTGATGG
ATTTTTGGGAGAAAGAGGAAGAATTGTTAAATGGGTTCCTCAACAAGAAGTTTTGGCTCATGGAGCTATT
GGAGCTTTTTGGACTCATTCAGGATGGAATTCAACTTTGGAATCAGTTTGCGAAGGAGTTCCTATGATTT
TTTCAGATTTTGGATTGGATCAACCTTTGAATGCTAGATATATGTCAGATGTTTTGAAAGTTGGAGTTTA
TTTGGAAAATGGATGGGAAAGAGGAGAAATTGCTAATGCTATTAGAAGAGTTATGGTTGATGAAGAAGGA
GAATATATTAGACAAAATGCTAGAGTTTTGAAACAAAAAGCTGATGTTTCATTGATGAAAGGAGGATCAT
CATATGAATCATTGGAATCATTGGTTTCATATATTTCATCATTG
SEQ ID NO. 74
Amino Acid
UPD gycosyltransferase 76G1
Stevia rebaudiana
MENKTETTVRRRRRIILFPVPFQGHINPILQLANVLYSKGFSITIFHTNFNKPKTSNYPHFTFRFILDND
PQDERISNLPTHGPLAGMRIPIINEHGADELRRELELLMLASEEDEEVSCLITDALWYFAQSVADSLNLR
RLVLMTSSLFNFHAHVSLPQFDELGYLDPDDKTRLEEQASGFPMLKVKDIKSAYSNWQILKEILGKMIKQ
TRASSGVIWNSFKELEESELETVIREIPAPSFLIPLPKHLTASSSSLLDHDRIVFQWLDQQPPSSVLYVS
FGSTSEVDEKDFLEIARGLVDSKQSFLWVVRPGFVKGSTWVEPLPDGFLGERGRIVKWVPQQEVLAHGAI
GAFWTHSGWNSTLESVCEGVPMIFSDFGLDQPLNARYMSDVLKVGVYLENGWERGEIANAIRRVMVDEEG
EYIRQNARVLKQKADVSLMKGGSSYESLESLVSYISSL
SEQ ID NO. 75
Amino Acid
Glycosyltransferase (NtGT5a)
Nicotiana tabacum
MGSIGAELTKPHAVCIPYPAQGHINPMLKLAKILHHKGFHITFVNTEFNHRRLLKSRGPDSLKGLSSFRF
ETIPDGLPPCEADATQDIPSLCESTINTCLAPFRDLLAKLNDTNTSNVPPVSCIVSDGVMSFTLAAAQEL
GVPEVLFWTTSACGFLGYMHYCKVIEKGYAPLKDASDLTNGYLETTLDFIPGMKDVRLRDLPSFLRTTNP
DEFMIKFVLQETERARKASAIILNTFETLEAEVLESLRNLLPPVYPIGPLHFLVKHVDDENLKGLRSSLW
KEEPECIQWLDTKEPNSVVYVNFGSITVMTPNQLIEFAWGLANSQQTFLWIIRPDIVSGDASILPPEFVE
ETKNRGMLASWCSQEEVLSHPAIVGFLTHSGWNSTLESISSGVPMICWPFFAEQQINCWFSVIKWDVGME
IDSDVKRDEVESLVRELMVGGKGKKMKKKAMEWKELAEASAKEHSGSSYVNIEKLVNDILLSSKH
SEQ ID NO. 76
DNA
Glycosyltransferase (NtGT5a)
Nicotiana tabacum
ATGGGTTCCATTGGTGCTGAATTAACAAAGCCACATGCAGTTTGCATACCATATCCCGCCCAAGGCCATA
TTAACCCCATGTTAAAGCTAGCCAAAATCCTTCATCACAAAGGCTTTCACATCACTTTTGTCAATACTGA
ATTTAACCACCGACGTCTCCTTAAATCTCGTGGCCCTGATTCTCTCAAGGGTCTTTCTTCTTTCCGTTTT
GAGACCATTCCTGATGGACTTCCGCCATGTGAGGCAGATGCCACACAAGATATACCTTCTTTGTGTGAAT
CTACAACCAATACTTGCTTGGCTCCTTTTAGGGATCTTCTTGCGAAACTCAATGATACTAACACATCTAA
CGTGCCACCCGTTTCGTGCATCGTCTCGGATGGTGTCATGAGCTTCACCTTAGCCGCTGCACAAGAATTG
GGAGTCCCTGAAGTTCTGTTTTGGACCACTAGTGCTTGTGGTTTCTTAGGTTACATGCATTACTGCAAGG
TTATTGAAAAAGGATATGCTCCACTTAAAGATGCGAGTGACTTGACAAATGGATACCTAGAGACAACATT
GGATTTTATACCAGGCATGAAAGACGTACGTTTAAGGGATCTTCCAAGTTTCTTGAGAACTACAAATCCA
GATGAATTCATGATCAAATTTGTCCTCCAAGAAACAGAGAGAGCAAGAAAGGCTTCTGCAATTATCCTCA
ACACATTTGAAACACTAGAGGCTGAAGTTCTTGAATCGCTCCGAAATCTTCTTCCTCCAGTCTACCCCAT
AGGGCCCTTGCATTTTCTAGTGAAACATGTTGATGATGAGAATTTGAAGGGACTTAGATCCAGCCTTTGG
AAAGAGGAACCAGAGTGTATACAATGGCTTGATACCAAAGAACCAAATTCTGTTGTTTATGTTAACTTTG
GAAGCATTACTGTTATGACTCCTAATCAGCTTATTGAGTTTGCTTGGGGACTTGCAAACAGCCAGCAAAC
ATTCTTATGGATCATAAGACCTGATATTGTTTCAGGTGATGCATCGATTCTTCCACCCGAATTCGTGGAA
GAAACGAAGAACAGAGGTATGCTTGCTAGTTGGTGTTCACAAGAAGAAGTACTTAGTCACCCTGCAATAG
TAGGATTCTTGACTCACAGTGGATGGAATTCGACACTCGAAAGTATAAGCAGTGGGGTGCCTATGATTTG
CTGGCCATTTTTCGCTGAACAGCAAACAAATTGTTGGTTTTCCGTCACTAAATGGGATGTTGGAATGGAG
ATTGACAGTGATGTGAAGAGAGATGAAGTGGAAAGCCTTGTAAGGGAATTGATGGTTGGGGGAAAAGGCA
AAAAGATGAAGAAAAAGGCAATGGAATGGAAGGAATTGGCTGAAGCATCTGCTAAAGAACATTCAGGGTC
ATCTTATGTGAACATTGAAAAGTTGGTCAATGATATTCTTCTTTCATCCAAACATTAA
SEQ ID NO. 77
Amino Acid
Glycosyltransferase (NtGT5b)
Nicotiana tabacum
MGSIGAEFTKPHAVCIPYPAQGHINPMLKLAKILHHKGFHITFVNTEFNHRRLLKSRGPDSLKGLSSFRF
ETIPDGLPPCDADATQDIPSLCESTINTCLGPFRDLLAKLNDTNTSNVPPVSCIISDGVMSFTLAAAQEL
GVPEVLFWTTSACGFLGYMHYYKVIEKGYAPLKDASDLTNGYLETTLDFIPCMKDVRLRDLPSFLRTTNP
DEFMIKFVLQETERARKASAIILNTYETLEAEVLESLRNLLPPVYPIGPLHFLVKHVDDENLKGLRSSLW
KEEPECIQWLDTKEPNSVVYVNFGSITVMTPNQLIEFAWGLANSQQSFLWIIRPDIVSGDASILPPEFVE
ETKKRGMLASWCSQEEVLSHPAIGGFLTHSGWNSTLESISSGVPMICWPFFAEQQINCWFSVIKWDVGME
IDCDVKRDEVESLVRELMVGGKGKKMKKKAMEWKELAEASAKEHSGSSYVNIEKVVNDILLSSKH
SEQ ID NO. 78
DNA
Glycosyltransferase (NtGT5b)
Nicotiana tabacum
ATGGGTTCCATTGGTGCTGAATTTACAAAGCCACATGCAGTTTGCATACCATATCCCGCCCAAGGCCATA
TTAACCCCATGTTAAAGCTAGCCAAAATCCTTCATCACAAAGGCTTTCACATCACTTTTGTCAATACTGA
ATTTAACCACAGACGTCTGCTTAAATCTCGTGGCCCTGATTCTCTCAAGGGTCTTTCTTCTTTCCGTTTT
GAGACAATTCCTGATGGACTTCCGCCATGTGATGCAGATGCCACACAAGATATACCTTCTTTGTGTGAAT
CTACAACCAATACTTGCTTGGGTCCTTTTAGGGATCTTCTTGCGAAACTCAATGATACTAACACATCTAA
CGTGCCACCCGTTTCGTGCATCATCTCAGATGGTGTCATGAGCTTCACCTTAGCCGCTGCACAAGAATTG
GGAGTCCCTGAAGTTCTGTTTTGGACCACTAGTGCTTGTGGTTTCTTAGGTTACATGCATTATTACAAGG
TTATTGAAAAAGGATACGCTCCACTTAAAGATGCGAGTGACTTGACAAATGGATACCTAGAGACAACATT
GGATTTTATACCATGCATGAAAGACGTACGTTTAAGGGATCTTCCAAGTTTCTTGAGAACTACAAATCCA
GATGAATTCATGATCAAATTTGTCCTCCAAGAAACAGAGAGAGCAAGAAAGGCTTCTGCAATTATCCTCA
ACACATATGAAACACTAGAGGCTGAAGTTCTTGAATCGCTCCGAAATCTTCTTCCTCCAGTCTACCCCAT
TGGGCCCTTGCATTTTCTAGTGAAACATGTTGATGATGAGAATTTGAAGGGACTTAGATCCAGCCTTTGG
AAAGAGGAACCAGAGTGTATACAATGGCTTGATACCAAAGAACCAAATTCTGTTGTTTATGTTAACTTTG
GAAGCATTACTGTTATGACTCCTAATCAACTTATTGAATTTGCTTGGGGACTTGCAAACAGCCAACAATC
ATTCTTATGGATCATAAGACCTGATATTGTTTCAGGTGATGCATCGATTCTTCCCCCCGAATTCGTGGAA
GAAACGAAGAAGAGAGGTATGCTTGCTAGTTGGTGTTCACAAGAAGAAGTACTTAGTCACCCTGCAATAG
GAGGATTCTTGACTCACAGTGGATGGAATTCGACACTCGAAAGTATAAGCAGTGGGGTGCCTATGATTTG
CTGGCCATTTTTCGCTGAACAGCAAACAAATTGTTGGTTTTCCGTCACTAAATGGGATGTTGGAATGGAG
ATTGACTGTGATGTGAAGAGGGATGAAGTGGAAAGCCTTGTAAGGGAATTGATGGTTGGGGGAAAAGGCA
AAAAGATGAAGAAAAAGGCAATGGAATGGAAGGAATTGGCTGAAGCATCTGCTAAAGAACATTCAGGGTC
ATCTTATGTGAACATTGAGAAGGTGGTCAATGATATTCTTCTTTCGTCCAAACATTAA
SEQ ID NO. 79
Amino Acid
UDP-glycosyltransferase 73C3 (NtGT4)
Nicotiana tabacum
MATQVHKLHFILFPLMAPGHMIPMIDIAKLLANRGVITTIITTPVNANRFSSTITRAIKSGLRIQILTLK
FPSVEVGLPEGCENIDMLPSLDLASKFFAAISMLKQQVENLLEGINPSPSCVISDMGFPWTTQIAQNFNI
PRIVFHGTCCFSLLCSYKILSSNILENITSDSEYFVVPDLPDRVELTKAQVSGSTKNTTSVSSSVLKEVT
EQIRLAEESSYGVIVNSFEELEQVYEKEYRKARGKKVWCVGPVSLCNKEIEDLVTRGNKTAIDNQDCLKW
LDNFETESVVYASLGSLSRLTLLQMVELGLGLEESNRPFVWVLGGGDKLNDLEKWILENGFEQRIKERGV
LIRGWAPQVLILSHPAIGGVLTHCGWNSTLEGISAGLPMVIWPLFAEQFCNEKLVVQVLKIGVSLGVKVP
VKWGDEENVGVLVKKDDVKKALDKLMDEGEEGQVRRTKAKELGELAKKAFGEGGSSYVNLTSLIEDIIEQ
QNHKEK
SEQ ID NO. 80
DNA
UDP-glycosyltransferase 73C3 (NtGT4)
Nicotiana tabacum
ATGGCAACTCAAGTGCACAAACTTCATTTCATACTATTCCCTTTAATGGCTCCAGGCCACATGATTCCTA
TGATAGACATAGCTAAACTTCTAGCAAATCGCGGTGTCATTACCACTATCATCACCACTCCAGTAAACGC
CAATCGTTTCAGTTCAACAATTACTCGTGCCATAAAATCCGGTCTAAGAATCCAAATTCTTACACTCAAA
TTTCCAAGTGTAGAAGTAGGATTACCAGAAGGTTGCGAAAATATTGACATGCTTCCTTCTCTTGACTTGG
CTTCAAAGTTTTTTGCTGCAATTAGTATGCTGAAACAACAAGTTGAAAATCTCTTAGAAGGAATAAATCC
AAGTCCAAGTTGTGTTATTTCAGATATGGGATTTCCTTGGACTACTCAAATTGCACAAAATTTTAATATC
CCAAGAATTGTTTTTCATGGTACTTGTTGTTTCTCACTTTTATGTTCCTATAAAATACTTTCCTCCAACA
TTCTTGAAAATATAACCTCAGATTCAGAGTATTTTGTTGTTCCTGATTTACCCGATAGAGTTGAACTAAC
GAAAGCTCAGGTTTCAGGATCGACGAAAAATACTACTTCTGTTAGTTCTTCTGTATTGAAAGAAGTTACT
GAGCAAATCAGATTAGCCGAGGAATCATCATATGGTGTAATTGTTAATAGTTTTGAGGAGTTGGAGCAAG
TGTATGAGAAAGAATATAGGAAAGCTAGAGGGAAAAAAGTTTGGIGTGITGGTCCTGTTTCTTTGTGTAA
TAAGGAAATTGAAGATTTGGTTACAAGGGGTAATAAAACTGCAATTGATAATCAAGATTGCTTGAAATGG
TTAGATAATTTTGAAACAGAATCTGTGGTTTATGCAAGTCTTGGAAGTTTATCTCGTTTGACATTATTGC
AAATGGTGGAACTTGGTCTTGGTTTAGAAGAGTCAAATAGGCCTTTTGTATGGGTATTAGGAGGAGGTGA
TAAATTAAATGATTTAGAGAAATGGATTCTTGAGAATGGATTTGAGCAAAGAATTAAAGAAAGAGGAGTT
TTGATTAGAGGATGGGCTCCTCAAGTGCTTATACTTTCACACCCTGCAATTGGTGGAGTATTGACTCATT
GCGGATGGAATTCTACATTGGAAGGTATTTCAGCAGGATTACCAATGGTAACATGGCCACTATTTGCTGA
GCAATTTTGCAATGAGAAGTTAGTAGTCCAAGTGCTAAAAATTGGAGTGAGCCTAGGTGTGAAGGTGCCT
GTCAAATGGGGAGATGAGGAAAATGTTGGAGTTTTGGTAAAAAAGGATGATGTTAAGAAAGCATTAGACA
AACTAATGGATGAAGGAGAAGAAGGACAAGTAAGAAGAACAAAAGCAAAAGAGTTAGGAGAATTGGCTAA
AAAGGCATTTGGAGAAGGTGGTTCTTCTTATGTTAACTTAACATCTCTGATTGAAGACATCATTGAGCAA
CAAAATCACAAGGAAAAATAG
SEQ ID NO. 81
Amino Acid
Glycosyltransferase (NtGT1b)
Nicotiana tabacum
MKTAELVFIPAPGMGHLVPTVEVAKQLVDRHEQLSITVLIMTIPLETNIPSYTKSLSSDYSSRITLLPLS
QPETSVTMSSFNAINFFEYISSYKGRVKDAVSETSFSSSNSVKLAGFVIDMFCTAMIDVANEFGIPSYVF
YTSSAAMLGLQLHFQSLSIECSPKVHNYVEPESEVLISTYMNPVPVKCLPGIILVNDESSTMFVNHARRF
RETKGIMVNTFTELESHALKALSDDEKIPPIYPVGPILNLENGNEDHNQEYDAIMKWLDEKPNSSVVFLC
FGSKGSFEEDQVKEIANALESSGYHFLWSLRRPPPKDKLQFPSEFENPEEVLPEGFFQRTKGRGKVIGWA
PQLAILSHPSVGGFVSHCGWNSTLESVRSGVPIATWPLYAEQQSNAFQLVKDLGMAVEIKMDYREDFNTR
NPPLVKAEEIEDGIRKLMDSENKIRAKVTEMKDKSRAALLEGGSSYVALGHFVETVMKN
SEQ ID NO. 82
DNA
Glycosyltransferase (NtGT1b)
Nicotiana tabacum
ATGAAGACAGCAGAGTTAGTATTCATTCCTGCTCCTGGGATGGGTCACCTTGTACCAACTGTGGAGGTGG
CAAAGCAACTAGTCGACAGACACGAGCAGCTTTCGATCACAGTTCTAATCATGACAATTCCTTTGGAAAC
AAATATTCCATCATATACTAAATCACTGTCCTCAGACTACAGTTCTCGTATAACGCTGCTTCCACTCTCT
CAACCTGAGACCTCTGTTACTATGAGCAGTTTTAATGCCATCAATTTTTTTGAGTACATCTCCAGCTACA
AGGGTCGTGTCAAAGATGCTGTTAGTGAAACCTCCTTTAGTTCGTCAAATTCTGTGAAACTTGCAGGATT
TGTAATAGACATGTTCTGCACTGCGATGATTGATGTAGCGAACGAGTTTGGAATCCCAAGTTATGTGTTC
TACACTTCTAGTGCAGCTATGCTTGGACTACAACTGCATTTTCAAAGTCTTAGCATTGAATGCAGTCCGA
AAGTTCATAACTACGTTGAACCTGAATCAGAAGTTCTGATCTCAACTTACATGAATCCGGTTCCAGTCAA
ATGTTTGCCCGGAATTATACTAGTAAATGATGAAAGTAGCACCATGTTTGTCAATCATGCACGAAGATTC
AGGGAGACGAAAGGAATTATGGTGAACACGTTCACTGAGCTTGAATCACACGCTTTGAAAGCCCTTTCCG
ATGATGAAAAAATCCCACCAATCTACCCAGTTGGACCTATACTTAACCTTGAAAATGGGAATGAAGATCA
CAATCAAGAATATGATGCGATTATGAAGTGGCTTGACGAGAAGCCTAATTCATCAGTGGTGTTCTTATGC
TTTGGAAGCAAGGGGTCTTTCGAAGAAGATCAGGTGAAGGAAATAGCAAATGCTCTAGAGAGCAGTGGCT
ACCACTTCTTGTGGTCGCTAAGGCGACCGCCACCAAAAGACAAGCTACAATTCCCAAGCGAATTCGAGAA
TCCAGAGGAAGTCTTACCAGAGGGATTCTTTCAAAGGACTAAAGGAAGAGGAAAGGTGATAGGATGGGCA
CCCCAGTTGGCTATTTTGTCTCATCCTTCAGTAGGAGGATTCGTGTCGCATTGTGGGTGGAATTCAACTC
TGGAGAGCGTTCGAAGTGGAGTGCCGATAGCAACATGGCCATTGTATGCAGAGCAACAGAGCAATGCATT
TCAACTGGTGAAGGATTTGGGTATGGCAGTAGAGATTAAGATGGATTACAGGGAAGATTTTAATACGAGA
AATCCACCACTGGTTAAAGCTGAGGAGATAGAAGATGGAATTAGGAAGCTGATGGATTCAGAGAATAAAA
TCAGGGCTAAGGTGACGGAGATGAAGGACAAAAGTAGAGCAGCACTGCTGGAGGGCGGATCATCATATGT
AGCTCTTGGGCATTTTGTTGAGACTGTCATGAAAAACTAG
SEQ ID NO. 83
Amino Acid
Glycosyltransferase (NtGT1a)
Nicotiana tabacum
MKTTELVFIPAPGMGHLVPTVEVAKQLVDRDEQLSITVLIMTLPLETNIPSYTKSLSSDYSSRITLLQLS
QPETSVSMSSFNAINFFEYISSYKDRVKDAVNETFSSSSSVKLKGFVIDMFCTAMIDVANEFGIPSYVFY
TSNAAMLGLQLHFQSLSIEYSPKVHNYLDPESEVAISTYINPIPVKCLPGIILDNDKSGTMFVNHARRFR
ETKGIMVNTFAELESHALKALSDDEKIPPIYPVGPILNLGDGNEDHNQEYDMIMKWLDEQPHSSVVFLCF
GSKGSFEEDQVKEIANALERSGNRFLWSLRRPPPKDTLQFPSEFENPEEVLPVGFFQRTKGRGKVIGWAP
QLAILSHPAVGGFVSHCGWNSTLESVRSGVPIATWPLYAEQQSNAFQLVKDLGMAVEIKMDYREDFNKTN
PPLVKAEEIEDGIRKLMDSENKIRAKVMEMKDKSRAALLEGGSSYVALGHFVETVMKN
SEQ ID NO. 84
DNA
Glycosyltransferase (NtGT1a)
Nicotiana tabacum
ATGAAGACAACAGAGTTAGTATTCATTCCTGCTCCTGGCATGGGTCACCTTGTACCCACTGTGGAGGTGG
CAAAGCAACTAGTCGACAGAGACGAACAGCTTTCAATCACAGTTCTCATCATGACGCTTCCTTTGGAAAC
AAATATTCCATCATATACTAAATCACTGTCCTCAGACTACAGTTCTCGTATAACGCTGCTTCAACTTTCT
CAACCTGAGACCTCTGTTAGTATGAGCAGTTTTAATGCCATCAATTTTTTTGAGTACATCTCCAGCTACA
AGGATCGTGTCAAAGATGCTGTTAATGAAACCTTTAGTTCGTCAAGTTCTGTGAAACTCAAAGGATTTGT
AATAGACATGTTCTGCACTGCGATGATTGATGTGGCGAACGAGTTTGGAATCCCAAGTTATGTCTTCTAC
ACTTCTAATGCAGCTATGCTTGGACTCCAACTCCATTTTCAAAGTCTTAGTATTGAATACAGTCCGAAAG
TTCATAATTACCTAGACCCTGAATCAGAAGTAGCGATCTCAACTTACATTAATCCGATTCCAGTCAAATG
TTTGCCCGGGATTATACTAGACAATGATAAAAGTGGCACCATGTTCGTCAATCATGCACGAAGATTCAGG
GAGACGAAAGGAATTATGGTGAACACATTCGCTGAGCTTGAATCACACGCTTTGAAAGCCCTTTCCGATG
ATGAGAAAATCCCACCAATCTACCCAGTTGGGCCTATACTTAACCTTGGAGATGGGAATGAAGATCACAA
TCAAGAATATGATATGATTATGAAGTGGCTCGACGAGCAGCCTCATTCATCAGTGGTGTTCCTATGCTTT
GGAAGCAAGGGATCTTTCGAAGAAGATCAAGTGAAGGAAATAGCAAATGCTCTAGAGAGAAGTGGTAACC
GGTTCTTGTGGTCGCTAAGACGACCGCCACCAAAAGACACGCTACAATTCCCAAGCGAATTCGAGAATCC
AGAGGAAGTCTTGCCGGTGGGATTCTTTCAAAGGACTAAAGGAAGAGGAAAGGTGATAGGATGGGCACCC
CAGTTGGCTATTTTGTCTCATCCTGCAGTAGGAGGATTCGTGTCGCATTGTGGGTGGAATTCAACTTTGG
AGAGTGTTCGTAGTGGAGTACCGATAGCAACATGGCCATTGTATGCAGAGCAACAGAGCAATGCATTTCA
ACTGGTGAAGGATTTGGGGATGGCAGTGGAGATTAAGATGGATTACAGGGAAGATTTTAATAAGACAAAT
CCACCACTGGTTAAAGCTGAGGAGATAGAAGATGGAATTAGGAAGCTGATGGATTCAGAGAATAAAATCA
GGGCTAAGGTGATGGAGATGAAGGACAAAAGTAGAGCAGCGTTATTAGAAGGCGGATCATCATATGTAGC
TCTCGGGCATTTTGTTGAGACTGTCATGAAAAACTAA
SEQ ID NO. 85
Amino Acid
Glycosyltransferase (NtGT3)
Nicotiana tabacum
MKETKKIELVFIPSPGIGHLVSTVEMAKLLIAREEQLSITVLIIQWPNDKKLDSYIQSVANFSSRLKFIR
LPQDDSIMQLLKSNIFTTFIASHKPAVRDAVADILKSESNNTLAGIVIDLFCTSMIDVANEFELPTYVFY
TSGAATLGLHYHIQNLRDEFNKDITKYKDEPEEKLSIATYLNPFPAKCLPSVALDKEGGSTMFLDLAKRF
RETKGIMINTFLELESYALNSLSRDKNLPPIYPVGPVLNLNNVEGDNLGSSDQNTMKWLDDQPASSVVFL
CFGSGGSFEKHQVKEIAYALESSGCRFLWSLRRPPTEDARFPSNYENLEEILPEGFLERTKGIGKVIGWA
PQLAILSHKSTGGFVSHCGWNSTLESTYFGVPIATWPMYAEQQANAFQLVKDLRMGVEIKMDYRKDMKVM
GKEVIVKAEEIEKAIREIMDSESEIRVKVKEMKEKSRAAQMEGGSSYTSIGGFIQIIMENSQ
SEQ ID NO. 86
DNA
Glycosyltransferase (NtGT3)
Nicotiana tabacum
ATGAAAGAAACCAAGAAAATAGAGTTAGTCTTCATTCCTTCACCAGGAATTGGCCATTTAGTATCCACAG
TTGAAATGGCAAAGCTTCTTATAGCTAGAGAAGAGCAGCTATCTATCACAGTCCTCATCATCCAATGGCC
TAACGACAAGAAGCTCGATTCTTATATCCAATCAGTCGCCAATTTCAGCTCGCGTTTGAAATTCATTCGA
CTCCCTCAGGATGATTCCATTATGCAGCTACTCAAAAGCAACATTTTCACCACGTTTATTGCCAGTCATA
AGCCTGCAGTTAGAGATGCTGTTGCTGATATTCTCAAGTCAGAATCAAATAATACGCTAGCAGGTATTGT
TATCGACTTGTTCTGCACCTCAATGATAGACGTGGCCAATGAGTTCGAGCTACCAACCTATGTTTTCTAC
ACGTCTGGTGCAGCAACCCTTGGTCTTCATTATCATATACAGAATCTCAGGGATGAATTTAACAAAGATA
TTACCAAGTACAAAGACGAACCTGAAGAAAAACTCTCTATAGCAACATATCTCAATCCATTTCCAGCAAA
ATGTTTGCCGTCTGTAGCCTTAGACAAAGAAGGTGGTTCAACAATGTTTCTTGATCTCGCAAAAAGGTTT
CGAGAAACCAAAGGTATTATGATAAACACATTTCTAGAGCTCGAATCCTATGCATTAAACTCGCTCTCAC
GAGACAAGAATCTTCCACCTATATACCCTGTCGGACCAGTATTGAACCTTAACAATGTTGAAGGTGACAA
CTTAGGTTCATCTGACCAGAATACTATGAAATGGTTAGATGATCAGCCCGCTTCATCTGTAGTGTTCCTT
TGITTTGGTAGTGGTGGAAGCTTTGAAAAACATCAAGTTAAGGAAATAGCCTATGCTCTGGAGAGCAGTG
GGTGTCGGTTTTTGTGGTCGTTAAGGCGACCACCAACCGAAGATGCAAGATTTCCAAGCAACTATGAAAA
TCTTGAAGAAATTTTGCCAGAAGGATTCTTGGAAAGAACAAAAGGGATTGGAAAAGTGATAGGATGGGCA
CCTCAGTTGGCGATTTTGTCACATAAATCGACGGGGGGATTTGTGTCGCACTGTGGATGGAATTCGACTT
TGGAAAGTACATATTTTGGAGTGCCAATAGCAACCTGGCCAATGTACGCGGAGCAACAAGCGAATGCATT
TCAATTGGTTAAGGATTTGAGAATGGGAGTTGAGATTAAGATGGATTATAGGAAGGATATGAAAGTGATG
GGCAAAGAAGTTATAGTGAAAGCTGAGGAGATTGAGAAAGCAATAAGAGAAATTATGGATTCCGAGAGTG
AAATTCGGGTGAAGGTGAAAGAGATGAAGGAGAAGAGCAGAGCAGCACAAATGGAAGGTGGCTCTTCTTA
CACTTCTATTGGAGGTTTCATCCAAATTATCATGGAGAATTCTCAATAA
SEQ ID NO. 87
Amino Acid
Glycosyltransferase (NtGT2)
Nicotiana tabacum
MVQPHVLLVTFPAQGHINPCLQFAKRLIRMGIEVTFATSVFAHRRMAKTITSTLSKGLNFAAFSDGYDDG
FKADEHDSQHYMSEIKSRGSKTLKDIILKSSDEGRPVTSLVYSLLLPWAAKVAREFHIPCALLWIQPATV
LDIYYYYFNGYEDAIKGSTNDPNWCIQLPRLPLLKSQDLPSFLLSSSNEEKYSFALPTFKEQLDTLDVEE
NPKVLVNTFDALEPKELKAIEKYNLIGIGPLIPSTFLDGKDPLDSSFGGDLFQKSNDYIEWLNSKANSSV
VYISFGSLLNLSKNQKEEIAKGLIEIKKPFLWVIRDQENGKGDEKEEKLSCMMELEKQGKIVPWCSQLEV
LTHPSIGCFVSHCGWNSTLESLSSGVSVVAFPHWTDQGTNAKLIEDVWKTGVRLKKNEDGVVESEEIKRC
IEMVMDGGEKGEEMRRNAQKWKELAREAVKEGGSSEMNLKAFVQEVGKGC
SEQ ID NO. 88
DNA
Glycosyltransferase (NtGT2)
Nicotiana tabacum
ATGGTGCAACCCCATGTCCTCTTGGTGACTTTTCCAGCACAAGGCCATATTAATCCATGTCTCCAATTTG
CCAAGAGGCTAATTAGAATGGGCATTGAGGTAACTTTTGCCACGAGCGTTTTCGCCCATCGTCGTATGGC
AAAAACTACGACTTCCACTCTATCCAAGGGCTTAAATTTTGCGGCATTCTCTGATGGGTACGACGATGGT
TTCAAGGCCGATGAGCATGATTCTCAACATTACATGTCGGAGATAAAAAGTCGCGGTTCTAAAACCCTAA
AAGATATCATTTTGAAGAGCTCAGACGAGGGACGTCCTGTGACATCCCTCGTCTATTCTCTTTTGCTTCC
ATGGGCTGCAAAGGTAGCGCGTGAATTTCACATACCGTGCGCGTTACTATGGATTCAACCAGCAACTGTG
CTAGACATATATTATTATTACTTCAATGGCTATGAGGATGCCATAAAAGGTAGCACCAATGATCCAAATT
GGTGTATTCAATTGCCTAGGCTTCCACTACTAAAAAGCCAAGATCTTCCTTCTTTTTTACTTTCTTCTAG
TAATGAAGAAAAATATAGCTTTGCTCTACCAACATTTAAAGAGCAACTTGACACATTAGATGTTGAAGAA
AATCCTAAAGTACTTGTGAACACATTTGATGCATTAGAGCCAAAGGAACTCAAAGCTATTGAAAAGTACA
ATTTAATTGGGATTGGACCATTGATTCCTTCAACATTTTTGGACGGAAAAGACCCTTTGGATTCTTCCTT
TGGTGGTGATCTTTTTCAAAAGTCTAATGACTATATTGAATGGTTGAACTCAAAGGCTAACTCATCTGIG
GTTTATATCTCATTTGGGAGTCTCTTGAATTTGTCAAAAAATCAAAAGGAGGAGATTGCAAAAGGGTTGA
TAGAGATTAAAAAGCCATTCTTGTGGGTAATAAGAGATCAAGAAAATGGTAAGGGAGATGAAAAAGAAGA
GAAATTAAGTTGTATGATGGAGTTGGAAAAGCAAGGGAAAATAGTACCATGGTGTTCACAACTTGAAGTC
TTAACACATCCATCTATAGGATGTTTCGTGTCACATTGTGGATGGAATTCGACTCTGGAAAGTTTATCGT
CAGGCGTGTCAGTAGTGGCATTTCCTCATTGGACGGATCAAGGGACAAATGCTAAACTAATTGAAGATGT
TTGGAAGACAGGTGTAAGGTTGAAAAAGAATGAAGATGGTGTGGTTGAGAGTGAAGAGATAAAAAGGTGC
ATAGAAATGGTAATGGATGGTGGAGAGAAAGGAGAAGAAATGAGAAGAAATGCTCAAAAATGGAAAGAAT
TGGCAAGGGAAGCTGTAAAAGAAGGCGGATCTTCGGAAATGAATCTAAAAGCTTTTGTTCAAGAAGTTGG
CAAAGGTTGCTGA
SEQ ID NO. 89
Amino Acid
THCA Synthase
Cannabis
MNCSAFSFWFVCKIIFFFLSFHIQISIANPRENFLKCFSKHIPNNVANPKLVYTQHDQLYMSILNSTIQN
LRFISDTTPKPLVIVTPSNNSHIQATILCSKKVGLQIRTRSGGHDAEGMSYISQVPFVVVDLRNMHSIKI
DVHSQTAWVEAGATLGEVYYWINEKNENLSFPGGYCPTVGVGGHFSGGGYGALMRNYGLAADNIIDAHLV
NVDGKVLDRKSMGEDLFWAIRGGGGENFGIIAAWKIKLVDVPSKSTIFSVKKNMEIHGLVKLFNKWQNIA
YKYDKDLVLMTHFITKNITDNHGKNKTTVHGYFSSIFHGGVDSLVDLMNKSFPELGIKKTDCKEFSWIDT
IIFYSGVVNFNTANFKKEILLDRSAGKKTAFSIKLDYVKKPIPETAMVKILEKLYEEDVGAGMYVLYPYG
GIMEEISESAIPFPHRAGIMYELWYTASWEKQEDNEKHINWVRSVYNFTTPYVSQNPRLAYLNYRDLDLG
KTNHASPNNYTQARIWGEKYFGKNFNRLVKVKTKVDPNNFFRNEQSIPPLPPHHH
SEQ ID NO. 90
DNA
Glycosyltransferase (NtGT1b-codon optimized for yeast expression)
Nicotiana tabacum
ATGAAAACAACAGAACTTGTCTTCATACCCGCCCCCGGTATGGGTCACCTTGTACCCACAGTCGAAGTCG
CCAAACAACTAGTTGATAGAGACGAACAGTTGTCTATTACCGTCTTGATAATGACGTTACCCCTGGAGAC
TAATATCCCAAGTTACACCAAGAGTTTGTCCTCTGACTATTCATCCCGTATCACGTTGTTACAACTAAGT
CAACCTGAGACGAGTGTCTCAATGAGTAGTTTTAACGCCATAAACTTCTTCGAATACATTAGTTCCTATA
AGGATCGTGTTAAAGATGCCGTAAACGAGACATTCTCCTCTTCATCCTCCGTCAAACTTAAAGGATTTGT
AATCGACATGTTTTGCACGGCAATGATAGACGTGGCCAACGAGTTCGGTATTCCATCTTATGTATTCTAC
ACGTCCAACGCTGCCATGCTAGGCCTACAACTTCACTTCCAATCCTTGTCCATCGAATATTCACCTAAGG
TTCATAATTATTTAGACCCTGAATCTGAGGTAGCTATATCAACGTACATTAACCCAATACCAGTAAAATG
CTTACCCGGTATAATTCTTGACAATGATAAGAGTGGCACTATGTTCGTAAACCATGCCAGGAGATTCCGT
GAAACAAAGGGTATAATGGTAAATACTTTTGCAGAATTAGAAAGTCACGCCCTAAAGGCACTTAGTGACG
ATGAGAAAATTCCTCCAATCTATCCCGTCGGACCCATTCTAAACTTGGGTGATGGTAATGAGGATCATAA
CCAAGAGTACGACATGATAATGAAATGGCTGGATGAACAACCACACAGTTCAGTGGTTTTCCTGTGCTTC
GGTTCCAAAGGTTCATTTGAAGAAGACCAGGTTAAAGAGATAGCAAATGCTTTAGAGAGATCAGGCAATA
GGTTCCTGTGGAGTTTAAGACGTCCCCCTCCCAAGGATACTCTTCAATTCCCTTCCGAATTTGAAAACCC
CGAGGAAGTGCTACCTGTAGGATTTTTTCAAAGAACCAAAGGCAGAGGAAAAGTCATCGGATGGGCACCA
CAGCTTGCAATTCTATCTCACCCTGCCGTCGGTGGATTCGTTTCCCACTGCGGCTGGAATAGTACTTTGG
AATCAGTTAGATCAGGTGTACCCATAGCAACATGGCCTCTTTATGCAGAGCAGCAGTCCAATGCATTTCA
ATTGGTCAAGGATCTAGGTATGGCCGTCGAAATTAAAATGGATTACCGTGAGGACTTTAACAAGACTAAT
CCTCCATTGGTAAAGGCAGAGGAAATAGAAGACGGCATTAGGAAGTTGATGGACTCCGAGAATAAGATTA
GGGCAAAGGTGATGGAAATGAAAGATAAGTCCAGAGCTGCATTACTGGAAGGAGGATCCTCCTATGTTGC
ACTGGGTCACTTCGTGGAGACCGTAATGAAGAACTAA
SEQ ID NO. 91
Amino Acid
Glycosyltransferase (NtGT1b-generated from codon optimized sequence for yeast expression)
Nicotiana tabacum
MKTTELVFIPAPGMGHLVPTVEVAKQLVDRDEQLSITVLIMTLPLETNIPSYTKSLSSDYSSRITLLQLS
QPETSVSMSSFNAINFFEYISSYKDRVKDAVNETFSSSSSVKLKGFVIDMFCTAMIDVANEFGIPSYVFY
TSNAAMLGLQLHFQSLSIEYSPKVHNYLDPESEVAISTYINPIPVKCLPGIILDNDKSGTMFVNHARRFR
ETKGIMVNTFAELESHALKALSDDEKIPPIYPVGPILNLGDGNEDHNQEYDMIMKWLDEQPHSSVVFLCF
GSKGSFEEDQVKEIANALERSGNRFLWSLRRPPPKDTLQFPSEFENPEEVLPVGFFQRTKGRGKVIGWAP
QLAILSHPAVGGFVSHCGWNSTLESVRSGVPIATWPLYAEQQSNAFQLVKDLGMAVEIKMDYREDFNKTN
PPLVKAEEIEDGIRKLMDSENKIRAKVMEMKDKSRAALLEGGSSYVALGHFVETVMKN
SEQ ID NO. 92
DNA
Glycosyltransferase (NtGT2-codon optimized for yeast expression)
Nicotiana tabacum
ATGGTTCAACCACACGTCTTACTGGTTACTTTTCCAGCACAAGGCCATATCAACCCTTGCCTACAATTCG
CCAAAAGACTAATAAGGATGGGCATCGAAGTAACTTTTGCCACGAGTGTATTCGCACATAGGCGTATGGC
TAAAACTACGACATCAACTTTGTCCAAAGGACTAAACTTCGCCGCCTTCAGTGATGGCTATGACGATGGA
TTCAAAGCCGACGAACATGACAGTCAACACTACATGAGTGAAATAAAGTCCCGTGGATCTAAAACACTTA
AGGATATTATACTTAAATCCTCCGATGAGGGAAGACCCGTTACCTCTTTAGTTTATTCACTGTTACTGCC
CTGGGCTGCAAAAGTCGCCAGAGAGTTTCATATTCCTTGCGCTTTATTGTGGATCCAACCAGCTACGGTA
TTAGACATCTACTATTACTACTTCAATGGATACGAGGATGCAATAAAGGGATCAACAAACGACCCCAACT
GGTGTATTCAACTGCCTAGACTTCCTCTATTAAAAAGTCAGGACTTACCTAGTTTTTTACTGTCATCCAG
TAACGAAGAAAAATATTCATTCGCTTTACCCACCTTCAAAGAGCAGCTTGACACTTTGGATGTTGAAGAG
AACCCCAAGGTTTTGGTCAATACTTTTGACGCTTTGGAGCCAAAAGAGCTAAAGGCTATTGAAAAATATA
ACCTTATCGGCATAGGACCTTTAATCCCCTCTACTTTCTTAGATGGCAAAGACCCTCTAGATTCAAGTTT
CGGAGGTGATTTGTTTCAAAAGAGTAACGATTATATCGAGTGGCTAAATAGTAAAGCCAACTCCAGTGTG
GTCTACATTTCTTTCGGAAGTCTTCTGAATTTATCAAAAAACCAAAAGGAAGAGATCGCAAAAGGACTGA
TAGAGATAAAAAAACCTTTCTTATGGGTGATCAGAGACCAGGAAAACGGTAAAGGCGATGAGAAGGAGGA
AAAACTGTCCTGTATGATGGAGCTAGAGAAACAAGGAAAAATCGTTCCCTGGTGTTCACAGTTAGAAGTG
TTAACCCATCCATCCATAGGTTGCTTCGTATCACATTGTGGTTGGAATAGTACACTTGAAAGTCTTTCAT
CAGGCGTCTCTGTCGTCGCATTCCCCCACTGGACGGACCAGGGCACAAACGCCAAACTGATCGAAGATGT
ATGGAAGACGGGCGTCAGGCTAAAAAAAAATGAGGATGGCGTGGTAGAGAGTGAAGAGATAAAGCGTTGC
ATAGAAATGGTCATGGATGGCGGTGAAAAGGGAGAGGAAATGAGGCGTAACGCACAAAAGTGGAAGGAAC
TAGCCCGTGAAGCAGTGAAAGAAGGAGGTTCTAGTGAGATGAATTTAAAAGCTTTCGTGCAGGAAGTTGG
AAAAGGCTGCTGA
SEQ ID NO. 93
Amino Acid
Glycosyltransferase (NtGT2-generated from codon optimized sequence for yeast expression)
Nicotiana tabacum
MVQPHVLLVTFPAQGHINPCLQFAKRLIRMGIEVTFATSVFAHRRMAKTITSTLSKGLNFAAFSDGYDDG
FKADEHDSQHYMSEIKSRGSKTLKDIILKSSDEGRPVTSLVYSLLLPWAAKVAREFHIPCALLWIQPATV
LDIYYYYFNGYEDAIKGSTNDPNWCIQLPRLPLLKSQDLPSFLLSSSNEEKYSFALPTFKEQLDTLDVEE
NPKVLVNTFDALEPKELKAIEKYNLIGIGPLIPSTFLDGKDPLDSSFGGDLFQKSNDYIEWLNSKANSSV
VYISFGSLLNLSKNQKEEIAKGLIEIKKPFLWVIRDQENGKGDEKEEKLSCMMELEKQGKIVPWCSQLEV
LTHPSIGCFVSHCGWNSTLESLSSGVSVVAFPHWTDQGTNAKLIEDVWKTGVRLKKNEDGVVESEEIKRC
IEMVMDGGEKGEEMRRNAQKWKELAREAVKEGGSSEMNLKAFVQEVGKGC
SEQ ID NO. 94
DNA
Glycosyltransferase (NtGT3-codon optimized for yeast expression)
Nicotiana tabacum
ATGAAAGAGACTAAAAAAATTGAGTTAGTTTTTATCCCCAGTCCTGGTATAGGACACTTAGTCTCAACTG
TGGAGATGGCCAAACTGTTGATAGCCCGTGAAGAGCAACTTTCTATTACTGTCCTGATTATACAATGGCC
TAATGATAAAAAGCTAGACAGTTATATCCAGTCCGTCGCAAACTTTAGTTCTAGACTGAAGTTTATACGT
CTGCCCCAAGATGACTCAATCATGCAACTTTTGAAATCAAACATTTTCACGACATTCATCGCCTCTCACA
AGCCAGCTGTAAGAGACGCCGTTGCTGACATACTAAAGAGTGAAAGTAATAACACATTGGCAGGCATTGT
AATCGATCTTTTCTGCACATCCATGATCGATGTAGCCAATGAGTTTGAGCTGCCTACTTATGTGTTTTAC
ACTAGTGGCGCAGCCACGTTGGGTCTGCACTACCATATTCAAAATCTGCGTGATGAGTTTAATAAAGACA
TTACCAAATATAAGGATGAGCCAGAAGAAAAATTAAGTATAGCCACGTACCTTAACCCATTCCCTGCTAA
GTGTCTACCCTCCGTGGCATTGGATAAGGAAGGAGGATCAACGATGTTCCTAGACTTAGCTAAGAGGTTC
AGGGAGACCAAAGGCATAATGATTAACACTTTTCTTGAGCTGGAATCATACGCTCTAAACTCATTGTCTA
GAGATAAAAACTTGCCCCCTATATACCCTGTAGGCCCTGTTTTGAACTTGAACAACGTTGAGGGTGATAA
CTTGGGCTCTAGTGATCAAAATACCATGAAATGGCTGGACGACCAGCCAGCTTCTTCCGTTGTGTTCCTA
TGTTTTGGCTCAGGAGGAAGTTTCGAAAAACACCAAGTCAAAGAAATAGCTTATGCCTTAGAATCTTCCG
GATGCAGGTTCTTGTGGAGTTTGCGTAGACCCCCCACGGAAGATGCTAGGTTCCCTTCTAATTACGAAAA
CTTAGAGGAAATTTTACCAGAGGGATTTCTGGAAAGAACGAAAGGCATTGGTAAGGTCATTGGATGGGCC
CCACAGTTAGCAATCTTGTCTCACAAGTCCACAGGAGGATTCGTGTCTCATTGCGGATGGAACTCTACCC
TTGAAAGTACCTATTTCGGCGTTCCTATTGCTACTTGGCCAATGTATGCTGAACAACAGGCCAACGCTTT
TCAACTTGTTAAAGATTTGAGGATGGGTGTTGAGATCAAAATGGATTATAGGAAGGATATGAAGGTAATG
GGCAAGGAGGTTATCGTTAAGGCAGAAGAAATTGAAAAGGCCATAAGGGAAATCATGGACTCAGAATCAG
AAATCAGGGTCAAGGTCAAAGAGATGAAGGAGAAAAGTCGTGCAGCCCAAATGGAAGGAGGATCATCATA
TACCTCTATCGGCGGCTTCATTCAAATAATCATGGAGAACTCACAGTAA
SEQ ID NO. 95
Amino Acid
Glycosyltransferase (NtGT3-generated from codon optimized sequence for yeast expression)
Nicotiana tabacum
MKETKKIELVFIPSPGIGHLVSTVEMAKLLIAREEQLSITVLIIQWPNDKKLDSYIQSVANFSSRLKFIR
LPQDDSIMQLLKSNIFTTFIASHKPAVRDAVADILKSESNNTLAGIVIDLFCTSMIDVANEFELPTYVFY
TSGAATLGLHYHIQNLRDEFNKDITKYKDEPEEKLSIATYLNPFPAKCLPSVALDKEGGSTMFLDLAKRF
RETKGIMINTFLELESYALNSLSRDKNLPPIYPVGPVLNLNNVEGDNLGSSDQNTMKWLDDQPASSVVFL
CFGSGGSFEKHQVKEIAYALESSGCRFLWSLRRPPTEDARFPSNYENLEEILPEGFLERTKGIGKVIGWA
PQLAILSHKSTGGFVSHCGWNSTLESTYFGVPIATWPMYAEQQANAFQLVKDLRMGVEIKMDYRKDMKVM
GKEVIVKAEEIEKAIREIMDSESEIRVKVKEMKEKSRAAQMEGGSSYTSIGGFIQIIMENSQ
SEQ ID NO. 96
DNA
UDP-glycosyltransferase 73C3 (NtGT4-codon optimized for yeast expression)
Nicotiana tabacum
ATGGCTACTCAGGTGCATAAATTGCATTTCATTCTGTTCCCACTGATGGCTCCCGGTCACATGATCCCTA
TGATAGACATCGCAAAACTATTGGCTAACCGTGGCGTGATAACTACCATAATAACTACGCCCGTTAACGC
CAATCGTTTTTCCTCTACGATCACTAGGGCCATTAAATCAGGCCTAAGAATCCAGATTTTAACCTTAAAA
TTCCCATCAGTTGAGGTAGGCCTGCCTGAAGGATGTGAAAACATCGACATGTTGCCATCTTTGGACTTAG
CCTCTAAATTCTTTGCTGCTATTTCTATGCTTAAACAACAAGTGGAGAACTTGCTAGAGGGTATTAACCC
TAGTCCCTCATGCGTTATTTCTGACATGGGCTTCCCATGGACGACACAGATCGCTCAAAATTTCAATATT
CCTCGTATCGTATTTCATGGCACGTGTTGCTTTTCTCTTCTTTGTTCTTACAAAATCCTGTCATCCAATA
TCTTAGAGAACATTACTAGTGACTCAGAGTATTTTGTCGTGCCAGATCTGCCAGACCGTGTCGAGCTAAC
TAAGGCCCAAGTCTCTGGATCTACAAAGAATACTACATCAGTAAGTAGTTCAGTACTGAAGGAGGTTACA
GAGCAGATCAGGCTTGCAGAGGAATCATCCTACGGTGTGATAGTTAATTCCTTCGAAGAACTGGAACAGG
TGTATGAAAAAGAGTACAGAAAAGCCAGGGGCAAAAAGGTCTGGTGCGTGGGTCCTGTCTCTTTGTGCAA
CAAGGAGATTGAAGATCTTGTTACTAGAGGAAACAAAACCGCTATAGACAATCAGGATTGTCTTAAGTGG
TTAGACAACTTCGAGACTGAATCCGTCGTCTATGCAAGTTTAGGCTCACTAAGTAGGCTTACGTTACTGC
AAATGGTTGAGCTGGGATTGGGACTGGAGGAGAGTAATAGGCCATTTGTATGGGTTCTGGGAGGAGGAGA
CAAACTAAATGATCTTGAGAAATGGATATTGGAGAATGGCTTTGAACAGCGTATAAAGGAGAGAGGTGTC
CTGATACGTGGCTGGGCACCTCAAGTATTGATTTTAAGTCACCCCGCAATTGGAGGAGTTTTAACGCATT
GTGGATGGAACTCTACATTAGAGGGCATTTCAGCCGGACTACCCATGGTCACCTGGCCACTATTTGCCGA
ACAGTTCTGTAACGAAAAATTAGTAGTGCAGGTTCTTAAAATCGGTGTCTCACITGGAGTGAAGGTCCCT
GTTAAGTGGGGTGACGAAGAGAACGTAGGTGTCTTAGTGAAAAAGGATGACGTTAAAAAAGCACTGGATA
AGCTAATGGATGAGGGTGAGGAGGGCCAGGTTAGGAGGACCAAAGCCAAAGAGCTTGGTGAGTTAGCTAA
AAAAGCCTTTGGAGAGGGCGGATCATCCTACGTGAACCTAACGTCCCTAATTGAAGATATAATCGAGCAG
CAGAACCATAAGGAGAAGTAG
SEQ ID NO. 97
Amino Acid
UDP-glycosyltransferase 73C3 (NtGT4-generated from codon optimized sequence for yeast
expression)
Nicotiana tabacum
MATQVHKLHFILFPLMAPGHMIPMIDIAKLLANRGVITTIITTPVNANRFSSTITRAIKSGLRIQILTLK
FPSVEVGLPEGCENIDMLPSLDLASKFFAAISMLKQQVENLLEGINPSPSCVISDMGFPWTTQIAQNFNI
PRIVFHGTCCFSLLCSYKILSSNILENITSDSEYFVVPDLPDRVELTKAQVSGSTKNTTSVSSSVLKEVT
EQIRLAEESSYGVIVNSFEELEQVYEKEYRKARGKKVWCVGPVSLCNKEIEDLVTRGNKTAIDNQDCLKW
LDNFETESVVYASLGSLSRLTLLQMVELGLGLEESNRPFVWVLGGGDKLNDLEKWILENGFEQRIKERGV
LIRGWAPQVLILSHPAIGGVLTHCGWNSTLEGISAGLPMVTWPLFAEQFCNEKLVVQVLKIGVSLGVKVP
VKWGDEENVGVLVKKDDVKKALDKLMDEGEEGQVRRTKAKELGELAKKAFGEGGSSYVNLTSLIEDIIEQ
QNHKEK
SEQ ID NO. 98
DNA
Glycosyltransferase (NtGT5-codon optimized for yeast expression)
Nicotiana tabacum
ATGGGCTCTATCGGTGCAGAACTAACCAAGCCACACGCCGTATGCATTCCCTATCCCGCCCAGGGACACA
TAAATCCTATGCTGAAGTTAGCTAAGATACTGCATCACAAGGGCTTCCATATAACCTTCGTAAATACGGA
ATTTAATCACAGGCGTCTGCTGAAGTCCAGAGGTCCTGACTCCCTGAAAGGTCTTTCAAGTTTCAGGTTC
GAGACGATACCTGACGGACTGCCCCCATGCGAAGCTGACGCTACACAGGACATTCCTTCACTGTGTGAAT
CCACGACTAATACATGTCTAGCTCCTTTTAGAGACCTACTTGCTAAGCTAAATGATACGAATACTTCTAA
CGTCCCTCCCGTAAGTTGTATTGTCAGTGACGGAGTGATGTCATTTACCCTTGCAGCTGCACAGGAACTG
GGTGTCCCAGAGGTTTTATTTTGGACTACATCTGCTTGTGGATTCTTAGGTTACATGCACTATTGCAAAG
TCATTGAAAAAGGATATGCTCCATTAAAAGACGCATCAGACCTGACGAATGGCTATCTTGAGACAACCTT
GGACTTCATCCCCGGCATGAAGGACGTCAGGCTGAGAGACTTACCTTCCTTTCTTAGGACCACCAATCCA
GACGAATTTATGATTAAGTTTGTACTACAGGAAACTGAGCGTGCTCGTAAGGCCAGTGCCATAATACTTA
ATACCTTTGAAACCTTAGAGGCAGAGGTATTAGAATCATTAAGGAACCTTCTACCCCCCGTCTATCCAAT
CGGCCCCTTGCATTTCCTTGTCAAACACGTAGACGATGAGAACCTAAAAGGTCTACGTTCCTCACTTTGG
AAGGAGGAACCTGAATGTATTCAATGGTTAGACACCAAAGAACCTAACTCTGTCGTGTACGTGAATTTCG
GATCCATTACTGTGATGACTCCCAATCAATTAATAGAGTTCGCTTGGGGACTGGCAAACTCTCAACAGAC
CTTCCTTTGGATCATAAGGCCTGACATCGTAAGTGGTGATGCTTCCATATTACCTCCCGAGTTTGTTGAG
GAGACTAAGAACAGAGGCATGCTTGCCTCCTGGTGCTCTCAGGAGGAGGTACTATCCCATCCCGCAATAG
TGGGATTTTTGACGCACTCTGGTTGGAACTCAACTTTAGAATCAATTTCTAGTGGCGTCCCCATGATCTG
TTGGCCTTTCTTTGCTGAGCAGCAAACGAACTGCTGGTTTTCAGTGACGAAGTGGGACGTTGGAATGGAA
ATTGATTCAGATGTGAAGAGAGATGAAGTAGAGAGTTTAGTAAGAGAGTTAATGGTGGGTGGTAAAGGCA
AGAAGATGAAGAAGAAGGCAATGGAGTGGAAGGAACTGGCCGAGGCTTCAGCAAAAGAACACTCTGGCTC
CTCTTACGTCAATATCGAGAAGTTGGTTAACGATATATTACTATCTAGTAAGCACTAA
SEQ ID NO. 99
Amino Acid
Glycosyltransferase (NtGT5-generated from codon optimized sequence for yeast expression)
Nicotiana tabacum
MGSIGAELTKPHAVCIPYPAQGHINPMLKLAKILHHKGFHITFVNTEFNHRRLLKSRGPDSLKGLSSFRF
ETIPDGLPPCEADATQDIPSLCESTINTCLAPFRDLLAKLNDTNTSNVPPVSCIVSDGVMSFTLAAAQEL
GVPEVLFWTTSACGFLGYMHYCKVIEKGYAPLKDASDLTNGYLETTLDFIPGMKDVRLRDLPSFLRTTNP
DEFMIKFVLQETERARKASAIILNTFETLEAEVLESLRNLLPPVYPIGPLHFLVKHVDDENLKGLRSSLW
KEEPECIQWLDTKEPNSVVYVNFGSITVMTPNQLIEFAWGLANSQQTFLWIIRPDIVSGDASILPPEFVE
ETKNRGMLASWCSQEEVLSHPAIVGFLTHSGWNSTLESISSGVPMICWPFFAEQQINCWFSVIKWDVGME
IDSDVKRDEVESLVRELMVGGKGKKMKKKAMEWKELAEASAKEHSGSSYVNIEKLVNDILLSSKH
SEQ ID NO. 100
DNA
UDP glycosyltransferase 76G1 (UGT76G1-codon optimized for yeast expression)
Stevia rebaudiana
ATGGAGAACAAAACCGAGACAACCGTTAGGCGTAGACGTAGGATAATATTGTTTCCCGTGCCCTTTCAAG
GCCATATAAACCCAATCCTGCAGCTAGCCAACGTATTGTACTCAAAGGGCTTCAGTATAACGATCTTCCA
CACCAACTTTAATAAGCCAAAAACGTCTAATTATCCACACTTCACATTTAGATTTATACTTGATAACGAC
CCACAGGATGAAAGAATATCAAACTTGCCCACGCACGGCCCACTAGCCGGAATGAGAATACCAATAATCA
ATGAGCATGGCGCCGACGAGTTGCGTAGAGAGCTGGAATTGTTGATGCTAGCCAGTGAGGAAGACGAAGA
GGTGTCCTGCTTAATAACGGATGCACTTTGGTATTTTGCTCAATCTGTGGCCGACTCCCTTAACCTGAGG
CGTCTTGTCCTTATGACCTCCAGTCTATTCAACTTTCATGCCCATGTCTCATTGCCCCAATTTGATGAGC
TTGGCTATTTGGATCCTGATGACAAAACTAGGCTGGAGGAACAGGCTTCCGGTTTTCCCATGCTAAAGGT
TAAGGACATCAAATCCGCCTACTCAAACTGGCAGATCCTTAAGGAAATTCTTGGCAAAATGATCAAACAG
ACGAGGGCATCCAGTGGCGTCATCTGGAACTCCTTTAAGGAACTTGAAGAATCAGAACTTGAAACAGTAA
TCAGAGAAATACCTGCCCCAAGTTTCTTGATCCCTCTACCTAAGCACCTTACGGCTTCTAGTTCTTCTTT
GTTGGACCACGATCGTACTGTCTTTCAATGGTTAGATCAGCAACCCCCCTCATCAGTGCTATATGTGTCA
TTCGGTAGTACATCAGAAGTGGACGAAAAGGATTTCCTTGAGATAGCCCGTGGATTGGTGGACTCTAAAC
AGTCCTTTTTATGGGTTGTGAGACCTGGATTTGTAAAGGGATCCACGTGGGTCGAACCCTTGCCCGATGG
TTTCCTGGGTGAAAGAGGAAGGATAGTGAAGTGGGTCCCTCAGCAAGAGGTACTGGCCCATGGTGCTATA
GGTGCTTTCTGGACCCACTCCGGCTGGAATAGTACACTAGAATCCGTTTGCGAGGGTGTCCCTATGATTT
TTTCTGATTTTGGTTTAGATCAACCCCTGAATGCTAGGTACATGTCAGACGTCCTTAAAGTCGGCGTCTA
CCTAGAAAATGGCTGGGAGAGGGGTGAGATAGCAAACGCTATCAGACGTGTTATGGTAGACGAAGAGGGA
GAGTACATAAGGCAAAACGCCAGGGTCCTGAAACAAAAAGCCGATGTGTCCTTGATGAAGGGCGGCTCTT
CATACGAAAGTCTAGAAAGTCTTGTTTCTTATATTTCCTCACTATAA
SEQ ID NO. 101
Amino Acid
UDP glycosyltransferase 76G1 (UGT76G1-generated from codon optimized sequence for yeast
expression)
Stevia rebaudiana
MENKTETTVRRRRRIILFPVPFQGHINPILQLANVLYSKGFSITIFHTNFNKPKTSNYPHFTFRFILDND
PQDERISNLPTHGPLAGMRIPIINEHGADELRRELELLMLASEEDEEVSCLITDALWYFAQSVADSLNLR
RLVLMTSSLFNFHAHVSLPQFDELGYLDPDDKTRLEEQASGFPMLKVKDIKSAYSNWQILKEILGKMIKQ
IRASSGVIWNSFKELEESELETVIREIPAPSFLIPLPKHLTASSSSLLDHDRTVFQWLDQQPPSSVLYVS
FGSTSEVDEKDFLEIARGLVDSKQSFLWVVRPGFVKGSTWVEPLPDGFLGERGRIVKWVPQQEVLAHGAI
GAFWTHSGWNSTLESVCEGVPMIFSDFGLDQPLNARYMSDVLKVGVYLENGWERGEIANAIRRVMVDEEG
EYIRQNARVLKQKADVSLMKGGSSYESLESLVSYISSL
SEQ ID NO. 102
DNA
glycosyltransferase (UGT73 A10)
Lycium barbarum
ATGGGTCAATTGCATTTTTTTTTGTTTCCAATGATGGCTCAAGGTCATATGATTCCAACTTTGGATATGG
CTAAGTTGATTGCTTCTAGAGGTGTTAAGGCTACTATTATTACTACTCCATTGAACGAATCTGTTTTTTC
TAAGGCTATTCAAAGAAACAAGCAATTGGGTATTGAAATTGAAATTGAAATTAGATTGATTAAGTTTCCA
GCTTTGGAAAACGATTTGCCAGAAGATTGTGAAAGATTGGATTTGATTCCAACTGAAGCTCATTTGCCAA
ACTTTTTTAAGGCTGCTGCTATGATGCAAGAACCATTGGAACAATTGATTCAAGAATGTAGACCAGATTG
TTTGGTTTCTGATATGTTTTTGCCATGGACTACTGATACTGCTGCTAAGTTTAACATTCCAAGAATTGTT
TTTCATGGTACTAACTACTTTGCTTTGTGTGTTGGTGATTCTATGAGAAGAAACAAGCCATTTAAGAACG
TTTCTTCTGATTCTGAAACTTTTGTTGTTCCAAACTTGCCACATGAAATTAAGTTGACTAGAACTCAAGT
TTCTCCATTTGAACAATCTGATGAAGAATCTGTTATGTCTAGAGTTTTGAAGGAAGTTAGAGAATCTGAT
TTGAAGTCTTACGGTGTTATTTTTAACTCTTTTTACGAATTGGAACCAGATTACGTTGAACATTACACTA
AGGTTATGGGTAGAAAGTCTTGGGCTATTGGTCCATTGTCTTTGTGTAACAGAGATGTTGAAGATAAGGC
TGAAAGAGGTAAGAAGTCTTCTATTGATAAGCATGAATGTTTGGAATGGTTGGATTCTAAGAAGCCATCT
TCTATTGTTTACGTTTGTTTTGGTTCTGTTGCTAACTTTACTGTTACTCAAATGAGAGAATTGGCTTTGG
GTTTGGAAGCTTCTGGTTTGGATTTTATTTGGGCTGTTAGAGCTGATAACGAAGATTGGTTGCCAGAAGG
TTTTGAAGAAAGAACTAAGGAAAAGGGTTTGATTATTAGAGGTTGGGCTCCACAAGTTTTGATTTTGGAT
CATGAATCTGTTGGTGCTTTTGTTACTCATTGTGGTTGGAACTCTACTTTGGAAGGTATTTCTGCTGGTG
TTCCAATGGTTACTTGGCCAGTTTTTGCTGAACAATTTTTTAACGAAAAGTTGGTTACTCAAGTTATGAG
AACTGGTGCTGGTGTTGGTTCTGTTCAATGGAAGAGATCTGCTTCTGAAGGTGTTGAAAAGGAAGCTATT
GCTAAGGCTATTAAGAGAGTTATGGTTTCTGAAGAAGCTGAAGGTTTTAGAAACAGAGCTAGAGCTTACA
AGGAAATGGCTAGACAAGCTATTGAAGAAGGTGGTTCTTCTTACACTGGTTTGACTACTTTGTTGGAAGA
TATTTCTTCTTACGAATCTTTGTCTTCTGATTAA
SEQ ID NO. 103
Amino Acid
Glycosyltransferase (UGT73 A10)
Lycium barbarum
MGQLHFFLFPMMAQGHMIPTLDMAKLIASRGVKATIITTPLNESVFSKAIQRNKQLGIEIEIEIRLIKFP
ALENDLPEDCERLDLIPTEAHLPNFFKAAAMMQEPLEQLIQECRPDCLVSDMFLPWITDTAAKFNIPRIV
FHGTNYFALCVGDSMRRNKPFKNVSSDSETFVVPNLPHEIKLTRTQVSPFEQSDEESVMSRVLKEVRESD
LKSYGVIFNSFYELEPDYVEHYTKVMGRKSWAIGPLSLCNRDVEDKAERGKKSSIDKHECLEWLDSKKPS
SIVYVCFGSVANFTVTQMRELALGLEASGLDFIWAVRADNEDWLPEGFEERTKEKGLIIRGWAPQVLILD
HESVGAFVTHCGWNSTLEGISAGVPMVTWPVFAEQFFNEKLVTQVMRTGAGVGSVQWKRSASEGVEKEAI
AKAIKRVMVSEEAEGFRNRARAYKEMARQAIEEGGSSYTGLTILLEDISSYESLSSD
SEQ ID NO. 104
DNA
Cytosolic-targeted UDP glycosyltransferase 76G1 (cytUTG)
Stevia rebaudiana
ATGGAAAATAAAACCGAAACCACCGTCCGCCGTCGTCGCCGTATCATTCTGTTCCCGGTCCCGTTCCAGG
GCCACATCAACCCGATTCTGCAACTGGCGAACGTGCTGTATTCGAAAGGTTTCAGCATCACCATCTTCCA
TACGAACTTCAACAAGCCGAAGACCAGCAATTACCCGCACTTTACGTTCCGTTTTATTCTGGATAACGAC
CCGCAGGATGAACGCATCTCTAATCTGCCGACCCACGGCCCGCTGGCGGGTATGCGTATTCCGATTATCA
ACGAACACGGCGCAGATGAACTGCGTCGCGAACTGGAACTGCTGATGCTGGCCAGCGAAGAAGATGAAGA
AGTTTCTTGCCTGATCACCGACGCACTGTGGTATTTTGCCCAGTCTGTTGCAGATAGTCTGAACCTGCGT
CGCCTGGTCCTGATGACCAGCAGCCTGTTCAATTTTCATGCCCACGTTAGTCTGCCGCAGTTCGATGAAC
TGGGTTATCTGGACCCGGATGACAAAACCCGCCTGGAAGAACAGGCGAGCGGCTTTCCGATGCTGAAAGT
CAAGGATATTAAGTCAGCGTACTCGAACTGGCAGATTCTGAAAGAAATCCTGGGTAAAATGATTAAGCAA
ACCAAAGCAAGTTCCGGCGTCATCTGGAATAGTTTCAAAGAACTGGAAGAATCCGAACTGGAAACGGTGA
TTCGTGAAATCCCGGCTCCGAGTTTTCTGATTCCGCTGCCGAAGCATCTGACCGCGAGCAGCAGCAGCCT
GCTGGATCACGACCGCACGGTGTTTCAGTGGCTGGATCAGCAACCGCCGAGTTCCGTGCTGTATGTTAGC
TTCGGTAGTACCTCGGAAGTGGATGAAAAGGACTTTCTGGAAATCGCTCGTGGCCTGGTTGATAGCAAAC
AATCTTTCCTGTGGGTGGTTCGCCCGGGTTTTGTGAAGGGCTCTACGTGGGTTGAACCGCTGCCGGACGG
CTTCCTGGGTGAACGTGGCCGCATTGTCAAATGGGTGCCGCAGCAAGAAGTGCTGGCGCATGGCGCGATT
GGCGCGTTTTGGACCCACTCCGGTTGGAACTCAACGCTGGAATCGGTTTGTGAAGGTGTCCCGATGATTT
TCTCAGATTTTGGCCTGGACCAGCCGCTGAATGCACGTTATATGTCGGATGTTCTGAAAGTCGGTGTGTA
CCTGGAAAACGGTTGGGAACGCGGCGAAATTGCGAATGCCATCCGTCGCGTTATGGTCGATGAAGAAGGC
GAATACATTCGTCAGAATGCTCGCGTCCTGAAACAAAAGGCGGACGTGAGCCTGATGAAAGGCGGTTCAT
CGTATGAAAGTCTGGAATCCCTGGTTTCATACATCAGCTCTCTGTAA
SEQ ID NO. 105
Amino Acid
Cytosolic-targeted UDP glycosyltransferase 76G1 (cytUTG)
Stevia rebaudiana
MENKTETTVRRRRRIILFPVPFQGHINPILQLANVLYSKGFSITIFHTNFNKPKTSNYPHFTFRFILDND
PQDERISNLPTHGPLAGMRIPIINEHGADELRRELELLMLASEEDEEVSCLITDALWYFAQSVADSLNLR
RLVLMTSSLFNFHAHVSLPQFDELGYLDPDDKTRLEEQASGFPMLKVKDIKSAYSNWQILKEILGKMIKQ
TKASSGVIWNSFKELEESELETVIREIPAPSFLIPLPKHLTASSSSLLDHDRTVFQWLDQQPPSSVLYVS
FGSTSEVDEKDFLEIARGLVDSKQSFLWVVRPGFVKGSTWVEPLPDGFLGERGRIVKWVPQQEVLAHGAI
GAFWTHSGWNSTLESVCEGVPMIFSDFGLDQPLNARYMSDVLKVGVYLENGWERGEIANAIRRVMVDEEG
EYIRQNARVLKQKADVSLMKGGSSYESLESLVSYISSL
SEQ ID NO. 106
Enhanced N-terminal chimera secretion signal with Ost1 signal sequence
S. cerevisiae
MRQVWFSWIVGLFLCFFNVSSAAPVNTTTEDETAQIPAEAVIGYSDLEGDFDVAVLPFSNSTNNG
LLFINTTIASIAAKEEGVSLEKR
SEQ ID NO. 107
Enhanced Ost1 secretion signal presequence
S. cerevisiae
MRQVWFSWIVGLFLCFFNVSSA
SEQ ID NO. 108
Amino Acid
Sec signal peptide for E coli L-asparaginase II
E. Coli
MEFFKKTALAALVMGFSGAALA
SEQ ID NO. 109
Amino Acid
Tat signal peptide for E coli strain k12 periplasmic nitrate reductase
E. Coli
MKLSRRSFMKANAVAAAAAAAGLSVPGVARAVVGQQ
SEQ ID NO. 110
Amino Acid
secretion signal from an extracellular protease Ara12 (At5g67360)
Arabidopsis thalinia
MSSSFLSSTAFFLLLCLGFCHVSSS
SEQ ID NO. 111
Amino Acid
secretion signal from a alpha amylase
barley (Hordeum vulgare)
MGKKSHICCFSLLLLLFAGLASG
SEQ ID NO. 112
Amino Acid
secretion signal from a a-Amylase
rice
MKNTSSLCLLLLVVLCSLTCNSGQAAQV
SEQ ID NO. 113
Amino Acid
>NP_001119793.1 odorant binding protein Ib-like precursor
Mus musculus
MMVKFLLLALVFGLAHVHAHDHPELQGQWKTTAIMADNIDKIETSGPLELFVREITCDEGCQKMKVTFYV
KQNGQCSLTIVTGYKQEDGKTFKNQYEGENNYKLLKATSENLVFYDENVDRASRKTKLLYILGKGEALTH
EQKERLTELATQKGIPAGNL
SEQ ID NO. 114
Amino Acid
>NP_775171.1 odorant-binding protein 2a precursor
Rattus norvegicus
MKSRLLTVLLLGLMAVLKAQEAPPDDQEDFSGKWYTKATVCDRNHTDGKRPMKVFPMTVTALEGGDLEVR
ITFRGKGHCHLRRITMHKTDEPGKYTTFKGKKTFYTKEIPVKDHYIFYIKGQRHGKSYLKGKLVGRDSKD
NPEAMEEFKKFVKSKGFREE
SEQ ID NO. 115
Amino Acid
>AIA65159.1 odorant binding protein 6
Mus musculus
MAKFLLLALAFGLAHAAMEGPWKTVAIAADRVDKIERGGELRIYCRSLICEKECKEMKVTFYVLENGQCS
LTTITGYLQEDGKTCKTQYQGDNHYELVKETPENLVFYSENVDRADRKTKLIFVLGNKPLTSEENERLVK
YAVSSHIPPENIRHVLGTDT
SEQ ID NO. 116
Amino Acid
>XP_027289850.1 odorant-binding protein 1b-like
Cricetulus griseus
MEKFLLLALAVSLAHALSELEGDWWSTAIDADNVAKIANQGTLRLYFHKMTCLEGYDKLEITFYVNLSGQ
CSKTTVVVYKQEDGNYRTQYEGDTIFKPMIITKEILVFTNENVDRDSLETHLIFVAGKGDHLTHEQYGRL
EEHAKEQKIPSESIRKLLVS
SEQ ID NO. 117
Amino Acid
>XP_006997496.1 PREDICTED: odorant-binding protein-like
Peromyscus maniculatus bairdii
MVKFLLLALALGVSCAHHNNPEITPSEVDGNWRTLYIGADNVEKVLKGGPLRAYFQHMECSDECQTLTIT
FKVKVEGECQTHTVVGRKEKDGLYMTDYSGKNYFRVIEKADGIIIFHNVNVDNSGKETNVILVAAVLS
SEQ ID NO. 118
Amino Acid
>XP_012860280.1 PREDICTED: odorant-binding protein 2b-like
Echinops telfairi
MQTLVLTMLSLIGTLQAQEPLSFAMEEATITGTWYIKAMVSNKDRDVRERTLSRSPLIVTALDHGDLEIS
ITFLKNGQCREKKILMENTGEPGKFSAFGSKKQITFLELPGKDHIIVFCEGERNGKSLRKAKLLGEQL
SEQ ID NO. 119
Amino Acid
>XP_008510274.1 PREDICTED: odorant-binding protein 2b-like
Equus przewalskii
MVLSSSVSWVQDQLGHLDYGAVSRAKAAEKLKRSRMFPNVSNIFCSNEDTKYQFSLCLSADGGKRHVYIL
DLPVKDHHIFYCEGQLGGKAIRMAKLVGINPDMSLEALEEFKKFTERKGLPQDIIIMPVQTESCIPESD
SEQ ID NO. 120
Amino Acid
>XP_006877726.1 PREDICTED: odorant-binding protein-like
Chrysochloris asiatica
MQYTSNNEILSFGFYFKYDGECLPRYEYTKRQTGNYFTGIGPLNNTFKPVYVTEDVMIGLYINVSVQGVT
SYIMQLLAKENSVSQEVFDMYMDYTRQVGIPEENLIDIIKRERTGI
SEQ ID NO. 121
Amino Acid
>XP_021009736.1 odorant-binding protein la-like
Mus caroli
MVKFLLLELAFGLAHAQMYGPWKTIAIAADNVDKMEISGELRLYFHQITCEKECKKMNVTFYVDENGQCS
LITITGYLQDDGKTYRSQFQGDNHYATVRTTPENIVFYSENVDRAGRKTKLVYVVGKNGSGSLK
SEQ ID NO. 122
Amino Acid
>XP_010604424.1 PREDICTED: odorant-binding protein
Fukomys damarensis
MRILLLALAVGFACADSQINPARINGEWRSIAEAADNVEKIQEGGPLRAYLRSLNCFQGCRKLSVNFYVK
LNEDWREFSVLSEKRPSDGVYTAVYSGQNFFNISSPDDGITVFSSTNVDENGRRTRLLLLGARKDSLTQA
EESKFRQLAVENGIPEENIV
SEQ ID NO. 123
Amino Acid
>XP 026251381.1 odorant-binding protein 2b
Urocitellus parryii
MGESGRGQGDSCLDLLQITGTWYPKAFVVNMPSVPDWKGPRKVFPVTVTALEDGSWEAKTILLIKGRCLE
KKVTLQKTEEPGRYSASTDHGKKLVYIEELPESHHCIFYCESQGPGKKFRMGKLMGRSPEENLEALEEFR
KFTQRKGLLAETIFTPEQTD
SEQ ID NO. 124
Amino Acid
>XP 025132613.1 odorant-binding protein-like
Bubalus bubalis
MKVLLLSAVLGMLYAGHGEAQLLLKPFSGKWKTHYIAASNKDKITEGGPFHVYVRHVEFHANNTVDIDFY
VKSDGECVKKQVTGVKQKFFVYQVEYAGQNEGRILHLSRDAIIVSIHNVDEEGKETVFVAIISMEPAISE
MWSIDVHQDSVHCIPYRLLY
SEQ ID NO. 125
Amino Acid
>XP 026333965.1 odorant-binding protein-like
Ursus arctos horribilis
MKILLLSLVLAVVCDAQLPLIHQLTQLPGQWETMYLAASNPDKISDNGPFKGYMRRIEVDMARRQISFHF
YAKINGQCTEKSVVGGIGTNNAITVDYEGTNDFQIIDMTPNSIIGYDVNVDEEGNTTDIVLLFGRGAQAD
EKAVEKFKQFTRQRNIPEEN
SEQ ID NO. 126
Amino Acid
>XP_022374058.1 odorant-binding protein-like
Enhydra lutris kenyoni
MKVLLLSLVLVAVCDAQLSLRNALIQLPGQWKTIHLAANNAEKLSENSPFRAYVRHVDVDMTRRKIFFNF
FIKVNGECIEKSVMGTVGLYNVIHVDYEGTNNFQVVRITPNIMLAYDINVDEEGRTTDLVILAGRTHEVD
EESIEKFKELVRQRNIPEEN
SEQ ID NO. 127
Amino Acid
>XP_006981169.1 PREDICTED: odorant-binding protein 2b-like
Peromyscus maniculatus bairdii
MKNLLIFLLLGLVAVLKAQEVPSDDQEELSGTWHIKALVCDKNHTEREGPKKVFPMTVTALEGGDLEVEI
TFWKKGQCHKKKIVMHKTDEPGKYTAFKGKKVIYIQELSVKDHYIFYCEGQHHGKSRRMGKLVGRNPEEN
PEALEEFKKFAQGKGLRQEN
SEQ ID NO. 128
Amino Acid
>XP_014651019.1 PREDICTED: odorant-binding protein-like
Ceratotherium simum simum
MKILLLTLVLGLVCAAQEPQSETNFSLVSGEWKTLYVASSNIEKISENGPFRAFVRRLDFDSEGDTIAFT
FLVKVNGQCTIIHSVATKIEGNVYISDYAGINGFKILDLSENAIIGYILNVDEEGLVTKIIALLGKGNDI
NEEDIEKFKELTRQRGIPEE
SEQ ID NO. 129
Amino Acid
>XP_006835766.1 PREDICTED: odorant-binding protein-like
Chrysochloris asiatica
MKTLLVTLVLGIICAAQDSLLQDPCTQVTGPWRTTYTASDNKEAIEENHPMRVYFRYMQCMSLGLAIRVD
FYSKENDQCILQHQLGLKTSENFYTTNYAGMVDFTILYYSDRFMVMYGINTNNGKTSKVIGAITQNDDIS
DAEYQIFLSLTKAKEIPEDS
SEQ ID NO. 130
Amino Acid
>XP_005228600.1 odorant-binding protein-like
Bos Taurus
MKALLLSLVLGLLAASQGDVIDASQFTGRWLTHFIAAENIDKITEGAPFHIFMRYIEFDEENGTIHFHFY
IKKNGECIEKYVSGLKEENFYAVDYSGHNEFQVISGDKNTLITHNLNVDEDGRETEMVGLFGLSDVVDPN
RIEEFKNVVREKGIPEENIR
SEQ ID NO. 131
Amino Acid
>XP_025132251.1 odorant-binding protein-like
Bubalus bubalis
MKVLLLSAVLGLLYAGHGEAQLLLKPFSGKWKTHYIAASNKDKITEGGPFHVYVRHVEFHANNTVDINFY
VKSDGECVKKQVTGVKQKFFVYQVEYAGQNEVRILHLSPDTIIVSIHNVDEEGKETVFVAIIGKRDRISN
LDNYNKFKKETEDRGIPEENI
SEQ ID NO. 132
Amino Acid
>AAI22740.1 Odorant-binding protein-like
Bos Taurus
MKILFLSLVLLVVCAAQETPAEIDPSKVVGEWRTIYAAADNKEKIVEGGPLRCYNRHIECINNCEQLSLS
FYIKFDGTCQFFSGVLQRQEGGVYFIEFEGKIYLQIIHVTDNILVFYYENDDGEKITKVTEGSAKGTSFT
PEEFQKYQQLNNERGIPNEN
SEQ ID NO. 133
Amino Acid
>XP_021045351.1 odorant-binding protein 1a-like, partial
Mus Pahari
MVKFLLLALAFGLAHAEFEGAWESVAIAADRVDKIERGGELRLYCRSLICENGCKEMKVTFYVLENGQCS
LITITGYLQEDGRTYKTQFQGDNHYELVKETPENLVFYSENVDRAGRTTKLLFVLGHESLTPEQKEVFAE
LAEEKGIPPENIRDVLVT
SEQ ID NO. 134
Amino Acid
>XP_004467463.1 odorant-binding protein 2b-like, partial
Dasypus novemcinctus
MPLALPQLTGTWYIKALVDTKEIPVEQRPDKVSPQTITALEGGNMAVTFTVMLQPTCLVLSGKKGQCHEM
NVLLEKTEEPGKYRAFNGTNLVQGEELPVKDHYAFIMEGQHRGRPFHMGKLIGRNLDVNFEALEEFKKFA
QSKGFLQENIFIPAQM
SEQ ID NO. 135
Amino Acid
>XP_021010322.1 odorant-binding protein la-like
Mus caroli
MAKFLLLALAFGLAHAALEGPKKTVAIAADRVDKIEESGELRLFCRRIVCEEECKKLIVTFYVLENGQCS
LTTITGYLQEDGKTYKTQYQGNNHFKLVKETPENVVFYSENVDRADWKTKLIFVLGNKPLTSEENERLVK
YAVSSHIPPENIQHVLGTDT
SEQ ID NO. 136
Amino Acid
>XP_005372051.1 odorant-binding protein 1b-like
Microtus ochrogaster
MVKFLLLTLAFGLAHAYTELEGAWFTTAIAADNVDTIEEEGPMRLYVRELTCSEACNEMDVTFYVNANGQ
CSETTVTGYRQEDGKYRTQFEGDNRFEPVYATSENIVFINKNVDRTGRTTNQIFVVGKGQPLTPEQYEKL
EEFAKQQNIPKENIRQVLDA
SEQ ID NO. 137
Amino Acid
>XP_021044251.1 odorant-binding protein 1a-like
Mus Pahari
MVKFLLLALAFGLAHAEFEGAWETVAIAADRVDKIEPSGELRLFCRSLDCEDGCKILKVTFYVLENGQCS
LTTVTGYLQEDGKTYKTQFQGDNHYELVKETPENLVFYSENVDRAGRTTKLIFVLGHKPLSSEQNERLVS
YAKSSHIPPENIRDVLGADT
SEQ ID NO. 138
Amino Acid
>KF022773.1 Odorant-binding protein, partial
Fukomys damarensis
STNLPSVNLPLQIDGNWRSMYLAADNVEKIEEGGELRNYVRQIECQDECRNISVRFYAKKNGVCQEFTVV
GVRDEASGDYFTEYLGENYFSIEYNTENIIIFHSTNVDEAGTTTNVILATGKSALLKVQELQKFARVVQD
YGIPKQNIRPVILTGRVITL
SEQ ID NO. 139
Amino Acid
>XP_004593691.1 PREDICTED: odorant-binding protein 2a
Ochotona princeps
MKALALTVALGLLAALQAQDPLALLLPEGQNITGTWYVKAVVGSKALPEGMRPKKLFPLTVTALDDGSLE
ATIVFEKHGQCFEKKFVMRQTEQPGEYIALDGKKRTCVEGLSTSDHYVFFCEKQRLGRVFRMAKLMGRSP
DPAPQATLEEFKELVQHKGF
SEQ ID NO. 140
Amino Acid
>XP_003515366.1 odorant-binding protein 1a-like, partial
Cricetulus griseus
MTSSYVYEQHIPGFYLLRSRQGKDSTCSMKIPSKLITQFYLLQKIKAGTTIAKILLLALAVCLAHALNEL
EGDWVSIAIAADNVEKIENQGTMRLYARQITCNEECDNLEITFYANLNGQCSETTVIGYKQEDGSYRTQY
EGDNVFKAVVITKDFLVFSS
SEQ ID NO. 141
Amino Acid
>XP_017899208.1 PREDICTED: odorant-binding protein-like
Capra hircus
MQANKMKVLFLTLVLGLVCSSQEIPAEPHHSQISGEWRTHYIASSNTDKTGENGPFNVYLRSIKFNDKGD
SLVFHFFVKNNGECTESSVSGRRIANNVYVAEYAGANQFHFILVSDDGLIVNTENVDDEGNRTRLIGLLG
KEDEVDDHDLERFLEEVRKL
SEQ ID NO. 142
Amino Acid
>XP_005346795.1 odorant-binding protein 2a-like [Microtus ochrogaster
MKRLLLTLILLGLVAVLKAQEFPSDDKEDYSGTWYPKAMIHNGSLPSHNIPSKFFPVKMTALEGGDLEAE
VIFWKNGQCHNVKILMKKTDEPGKFTSFDNKRFIYITALLVKDHYIMYCEGRLPGKLFGVGKLVGRNPEE
NPEAMEEFKKFVQRKGLKVE
SEQ ID NO. 143
Amino Acid
>XP 025118236.1 odorant-binding protein 2b-like
Bubalus bubalis
MKALLLPIALSLLAALRAQDPPSCPLEPQQIAGTWYVKAMVTDENLPKETRPRKVSPVTVTALGGGNLEL
MFTFLKEARCHEKRTRVQPTGEPGKYSSNGGKKQMHILELPVEGHYILYCEGQRQGKSVHVGKLIGRNPD
MNPEALEAFKKFVQRKGLSP
SEQ ID NO. 144
Amino Acid
>XP_021496742.1 odorant-binding protein 2a-like
Meriones unguiculatus
MKSLLLTVLLLGLVAVLKAQEDLPDDKEDFSGTWYTNAMVCDKDHTNGKKPKKVYLMTVTALEGGDLEIT
ITFQKNGQCHEKKIVIHKTDDPHKFTAFGGKKVIQIQATSQKDHYILYCEGKHKGKLHRKAKLLGRKPEK
SPEAMREFMEFVESKKLKTQ
SEQ ID NO. 145
Amino Acid
>XP_021496743.1 odorant-binding protein 2a-like
Meriones unguiculatus
MKSLLLTVLLLGLVAVLKAQEDLPDDKEDLSGTWYMKGMVHNGTLPKNKLPERVFPVTITALEEGNLEVK
IIKWKKGQCHEFKFKMEKTEEPNKYITFHGKRHVYIEKLNTKDHYIFYCEGHYKGKHFGMGKVMGRTSEE
SPEAMEEFKEFVKRKKIPQE
SEQ ID NO. 146
Amino Acid
>XP_015353183.1 PREDICTED: odorant-binding protein 2b
Marmota marmota marmot
MKSLFLTILLLDLLSALQAQDLLTFPSEELNITGTWYTKAFVVNMPLVPDWKGPGKVFPVTVTALEDGSW
EAKTTLLIQGRCLEKKVTLQKTEEPGRYSASTDHGKKFVYIEELPESDHCIFYCESQDPGKKFRMGKLMG
RSPEENLEALEEFRKFTQRK
SEQ ID NO. 147
Amino Acid
>XP_021117221.1 odorant-binding protein 2a-like
Heterocephalus glaber
MKTLLLTPVLLALVAALRAKDALSLQPEEPDITGTRYMKAIVINGNLTHGPRQAFPVTVMAWEGVNFETR
ITFMWRGGCYKDRLHLQKTTEPGKYTFWNHTHIHTEELAVKDHSACYAEHQLPLGETMHVGYLMGEDPGD
PSPGPAVSLWRS
SEQ ID NO. 148
Amino Acid
>EHA98383.1 Odorant-binding protein, partial
Heterocephalus glaber
MINGDWCSIYIAADNVEKIEERGELRAYFCHIECQDECRNLSGGDRIMRNKHCCVGLSFRLDGVCQEFTV
VGVKDEKSGVYITDYVGKNYFTVVESTEYITLFSNIIVDEKGTKMNVVLVAAKRDSLTEKEKQKFAQLAE
EKGIPTENIRNVIAT

1-14. (canceled)

15. A solubilized cannabinoid composition comprising:
a carrier protein having a β-barrel enclosed cannabinoid-binding site having an internal cavity, and an external loop scaffold structure bound to at least one cannabinoid to form a water-soluble protein-cannabinoid complex.

16. The composition of claim 15, wherein the carrier protein comprises a carrier protein having an amino acid sequence selected from the group of consisting of: SEQ ID NOs. 1-46, and 113-148, or a homolog having affinity towards at least one cannabinoid thereof.

17. (canceled)

18. The composition of claim 15, wherein the carrier protein is coupled with a secretion signal.

19. The composition of claim 18, wherein said secretion signal comprises a secretion signal having an amino acid sequence selected from the group consisting of: SEQ ID NO. 47, and SEQ ID NOs. 106-112.

20. The composition of claim 15, wherein the at least one cannabinoid comprises a cannabinoid selected from the group consisting of: cannabidiol (CBD), cannabidiolic acid (CBDA), Δ⁹-tetrahydrocannabinol (THC), tetrahydrocannabinolic acid (THCA), cannabigerol (CBG), and cannabigerolic acid CBGA).

21. The composition of claim 15, wherein said carrier protein having affinity towards at least one cannabinoid comprises an Olfactory Binding Protein (OBP)-carrier protein having a β-barrel enclosed cannabinoid-binding site having an internal cavity, and an external loop scaffold structure, or Lipocalin Cannabinoid (LC)-carrier protein having a β-barrel enclosed cannabinoid-binding site having an internal cavity, and an external loop scaffold structure.

22-46. (canceled)

47. A method of solubilizing a cannabinoid comprising the steps of:
generating a Lipocalin Carrier (LP)-carrier protein having affinity towards at least one cannabinoid; and
introducing said LC-carrier protein to said at least one cannabinoid, wherein said LC-carrier protein binds said at least one cannabinoid to form a water-soluble protein-cannabinoid complex.

48. The method of claim 47, wherein the LC-carrier protein comprises an LC-carrier protein having an amino acid sequence selected from the group of consisting of: SEQ ID NOs. 1-29, and 30-46, or a homolog having affinity towards at least one cannabinoid thereof.

49. (canceled)

50. The method of claim 47, wherein the LC-carrier protein is coupled with a secretion signal.

51-52. (canceled)

53. The method of claim 47, wherein the at least one cannabinoid comprises a cannabinoid selected from the group consisting of: cannabidiol (CBD), cannabidiolic acid (CBDA), Δ⁹-tetrahydrocannabinol (THC), tetrahydrocannabinolic acid (THCA), cannabigerol (CBG), and cannabigerolic acid (CBGA).

54. The method of claim 47, wherein said LC-carrier protein having affinity towards at least one cannabinoid comprises an LC-carrier protein having a β-barrel enclosed cannabinoid-binding site having an internal cavity, and an external loop scaffold structure.

55. The method of claim 47, wherein the LC-carrier comprises an engineered LC-carrier protein having a truncated LC-carrier protein forming a β-barrel enclosed cannabinoid-binding site having an internal cavity, and an external loop scaffold structure.

56. The method of claim 55, wherein said truncated LC-carrier protein comprises an truncated LC-carrier protein having an amino acid sequence selected from the group of consisting of: SEQ ID NOs. 30-46.

57-61. (canceled)

62. A method of solubilizing a cannabinoid comprising the steps of:
establishing a cell culture of genetically modified yeast, plant, or bacteria cells that express a nucleotide sequence, operably linked to a promoter, encoding a heterologous Lipocalin Carrier (LC)-carrier protein wherein said heterologous LC-carrier protein exhibits affinity towards one or more cannabinoids;
introducing one or more cannabinoids to the genetically modified yeast, plant, or bacteria cell culture; and
binding said LC-carrier protein with said one or more cannabinoids to form a water-soluble protein-cannabinoid complex;
wherein said LC-carrier protein includes a β-barrel enclosed cannabinoid-binding site having an internal cavity, and an external loop scaffold structure.

63-64. (canceled)

65. The method of claim 62, wherein said heterologous LC-carrier protein comprises a heterologous LC-carrier protein having an amino acid sequence selected from the group of consisting of: SEQ ID NOs. 1-29, and 30-46, or a homolog having affinity towards at least one cannabinoid thereof.

66-68. (canceled)

69. The method of claim 62, wherein the at least one cannabinoid comprises a cannabinoid selected from the group consisting of: cannabidiol (CBD), cannabidiolic acid (CBDA), Δ⁹-tetrahydrocannabinol (THC), tetrahydrocannabinolic acid (THCA), cannabigerol (CBG), and cannabigerolic acid (CBGA).

70. The method of claim 62, and further comprising the of step of genetically modifying the LC-carrier protein to form an engineered LC-carrier protein having enhanced affinity for at least one cannabinoid, such genetic modification comprising at least one of the following:
replacing one or more amino acid residues of the LC-carrier protein cannabinoid binding pocket with side chains orientated toward the binding cavity;
replacing one or more amino acid residues of the LC-carrier protein cannabinoid binding pocket having a hydrophilic side chain with amino acid residues having a hydrophobic side chain; and
replacing one or more small hydrophobic amino acid residues of the LC-carrier protein cannabinoid binding pocket with larger hydrophobic amino acid residues.

71-72. (canceled)

73. (canceled)

74. The method of claim 62, wherein the LC-carrier comprises an engineered LC-carrier protein further comprising a truncated LC-carrier protein forming a β-barrel enclosed cannabinoid-binding site having an internal cavity, and an external loop scaffold structure.

75. The method of claim 74, wherein said truncated LC-carrier protein comprises an truncated LC-carrier protein having an amino acid sequence selected from the group of consisting of: SEQ ID NOs. 30-46.

76-87. (canceled)

70. The method of claim 15, and further comprising the of step of genetically modifying the LC-carrier protein to form an engineered LC-carrier protein having enhanced affinity for at least one cannabinoid, such genetic modification comprising at least one of the following:
replacing one or more amino acid residues of the LC-carrier protein cannabinoid binding pocket with side chains orientated toward the binding cavity;
replacing one or more amino acid residues of the LC-carrier protein cannabinoid binding pocket having a hydrophilic side chain with amino acid residues having a hydrophobic side chain; and
replacing one or more small hydrophobic amino acid residues of the LC-carrier protein cannabinoid binding pocket with larger hydrophobic amino acid residues.


SEQUENCE LISTING (148 sequences)

SEQ ID NO: 1 - protein, 188 aa, Artificial Sequence
  Cluster63 Unique
MTSTEKKDMKAVKGLDLERYMGRWYEIASFPSRFQPKDGVDTRATYTLNPDGTVHVLNETWNGGKRGFIQGSAYKADPKSDEAKLKVKFFVPPFLPVIPVTGDYWVLYIDPEYQHAVIGQPSRSYLWILSRTAHMEEETYKQLVEKAVEEGYDVSKLHKTPQSDTPPESNTAPDDTKGVWWLKSIFGK

SEQ ID NO: 2 - protein, 353 aa, Arabidopsis thaliana
MILLSSSISLSRPVSSQSFSPPAATSTRRSHSSVTVKCCCSSRRLLKNPELKCSLENLFEIQALRKCFVSGFAAILLLSQAGQGIALDLSSGYQNICQLGSAAAVGENKLTLPSDGDSESMMMMMMRGMTAKNFDPVRYSGRWFEVASLKRGFAGQGQEDCHCTQGVYTFDMKESAIRVDTFCVHGSPDGYITGIRGKVQCVGAEDLEKSETDLEKQEMIKEKCFLRFPTIPFIPKLPYDVIATDYDNYALVSGAKDKGFVQVYSRTPNPGPEFIAKYKNYLAQFGYDPEKIKDTPQDCEVTDAELAAMMSMPGMEQTLTNQFPDLGLRKSVQFDPFTSVFETLKKLVPLYFK

SEQ ID NO: 3 - protein, 181 aa, Zea mays
MAMQVVRNLDLERYAGRWYEIACFPSRFQPKTGTNTRATYTLNPDGTVKVVNETWADGRRGHIEGTAWRADPASDEAKLKVRFYVPPFLPLIPVTGDYWVLHIDADYQYALVGQPSRNYLWILCRQPHMDESVYKELVERAKEEGYDVSKLRKTAHPDPPPESEQSPRDGGMWWVKSIFGK

SEQ ID NO: 4 - protein, 186 aa, Arabidopsis thaliana
MTEKKEMEVVKGLNVERYMGRWYEIASFPSRFQPKNGVDTRATYTLNPDGTIHVLNETWSNGKRGFIEGSAYKADPKSDEAKLKVKFYVPPFLPIIPVTGDYWVLYIDPDYQHALIGQPSRSYLWILSRTAQMEEETYKQLVEKAVEEGYDISKLHKTPQSDTPPESNTAPEDSKGVWWFKSLFGK

SEQ ID NO: 5 - protein, 179 aa, Oryza sativa Japonica Group
MKVVRNLDLERYMGRWYEIACFPSRFQPRDGTNTRATYTLAGDGAVKVLNETWTDGRRGHIEGTAYRADPVSDEAKLKVKFYVPPFLPIFPVVGDYWVLHVDDAYSYALVGQPSLNYLWILCRQPHMDEEVYGQLVERAKEEGYDVSKLKKTAHPDPPPETEQSAGDRGVWWIKSLFGR

SEQ ID NO: 6 - protein, 342 aa, Oryza sativa Japonica Group
MVLALLLGSSSSSLAAPHPACSSRRKCRPAGRNNFRCSLHDKVPLNAHGVLSTKLLSCLAASLVFISPPCQAIPAETFVQPKLCQVAVVAAIDKAAVPLKFDSPSDDGGTGLMMKGMTAKNFDPIRYSGRWFEVASLKRGFAGQGQEDCHCTQGVYSFDEKSRSIQVDTFCVHGGPDGYITGIRGRVQCLSEEDMASAETDLERQEMIKGKCFLRFPTLPFIPKEPYDVLATDYDNYAVVSGAKDTSFIQIYSRTPNPGPEFIEKYKSYAANFGYDPSKIKDTPQDCEVMSTDQLGLMMSMPGMTEALTNQFPDLKLSAPVAFNPFTSVFDTLKKLVELYFK

SEQ ID NO: 7 - protein, 188 aa, Brassica napus
MTSTEKKDMNAVKGLDLERYMGRWYEIASFPSRFQPKDGVDTRATYTLNPDGTVHVLNETWNGGKRGFIQGSAYKADPKSDEAKLKVKFFVPPFLPVIPVTGDYWVLYIDPQYQHAVIGQPSRSYLWILSRTAHMEEETYKQLVEKAVEEGYDVSKLHKTPQSDTPPESNTAPDDTKGVWWLKSIFGK

SEQ ID NO: 8 - protein, 235 aa, Physcomitrella patens
MASVGASSVWHCILLLAMVVLTGEGARAKRILHTEAPSPSQGVCSNPPTVSNVSLEAYSGVWYEIGSTALVKARIERDLICATARYSVIPDGDLAGSIRVRNEGYNIRTGEFAHAIGTATVVSPGRLEVKFFPGAPGGDYRIIYLSGKAEDKYNVAIVYSCDESVPGGSQSLFILSREPELDDEDDDDDDYDDDDETLSRLLNFVRDLGIVFEPNNEFILTPQDPITCGRNGYDD

SEQ ID NO: 9 - protein, 249 aa, Brassica napus
MMYVKVLMMVIAIWFVPMTYSNGAEAPAGDVAEAPGADAFNNDWYDARSTFYGDIHGGDTLKKKEEEKMTTQNKEMEVVKDLDLERYMGRWYEIASFPSIFQPKNGIDTRATYTLNPDGTVDVLNETWNSGKRVFIQGSAYKTDPKSDEAKFKVKFYVPPFLPIIPVTGDYWVLYIDPEYQHAVIGQPSRSYLWILSRTAHVEEETYKQLLEKAVEEGYDVSKLHKTPQSDTPPESNAAPNDTKDQMLK

SEQ ID NO: 10 - protein, 342 aa, Micractinium conductrix
MHVSTRQPCGAAPTAWPAQRPRSSPRRLACSAVLRDDARGVLQQAGLKLAAAAAAVLLAAPLHAGAASMPANAPLPALPPAPFDIEQSKQSKLLFDPMAYSGRWYEVASLKRGFAGEGQQDCHCTQGIYTPKEGGPEGAIKLEVDTFCVHGGPGGRLSGIQGSVSCADPLLLSYLPEFQTEMEMVEGFVAKCALRFDSLAFLPPEPYVVLRTDYTSYALVRGAKDRSFVQIYSRTPNPGAKFIAEQKAVLGQLGYPANDIVDTPQDCPEMAPQAMMAAMNRGMSSTPTMPASTPPALAMAGYDLGPAAVVLGEEAPAPVKGIAFDRLRNPLESLKNVFSLFN

SEQ ID NO: 11 - protein, 342 aa, Citrus unshiu
MVNVIHQTSPALLQCCPSPPFANSIYRGNPRKKVYKCSFDNPISNKMVTGHVTRHLLSGLAASIIFLSQTNQVVAADLPHFHNICQLASATDSMPTLPIELGSDERSGMLMMMRGMTAKDFDPVRYSGRWFEVASLKRGFAGQGQEDCHCTQGVYTFDKEKPAIQVDTFCVHGGPDGYITGIRGNVQCLPEEELEKNVTDLEKQEMIKGKCYLRFPTLPFIPKEPYDVIATDYDNFALVSGAKDKSFIQIYSRTPTPGPEFIEKYKSYLANFGYDPNKIKDTPQDCEVISNSQLAAMMSMSGMQQALTNQFPDLELKSPLALNPFTSVLDTLKKLLELYFKK

SEQ ID NO: 12 - protein, 340 aa, Zea mays
MVLLLLGCSPASSRPDCSPASRRRCSTAGQKMVRCSLNEETQLNKHGLVSKQLISCLAASLVFVSPPSQAIPAETFARPGLCQIATVAAIDSASVPLKFDNPSDDVSTGMMMRGMTAKNFDPVRYSGRWFEVASLKRGFAGQGQEDCHCTQGVYSFDEKARSIQVDTFCVHGGPDGYITGIRGRVQCLSEEDIASAETDLERQEMVRGKCFLRFPTLPFIPKEPYDVLATDYDNYAIVSGAKDTSFIQIYSRTPNPGPEFIDKYKSYVANFGYDPSKIKDTPQDCEYMSSDQIALMMSMPGMNEALTNQFPDLKLKAPVALNPFTSVFDTLKKLLELYFK

SEQ ID NO: 13 - protein, 327 aa, Macleaya cordata
MVLIQASPLSSPPLLRVIPANRTLACSLQQPASGTKVIAKHVLSGVAVSLIFLSQTNQVFAAEPSHYSNLCQLAAVTDKGVTLPLEEGSDGRKGQLMMMRGMSAKNFDPIRYSGRWFEVASLKRGFAGSGQEDCHCTQGVYTFDSEAPAIQVDTFCVHGGPDGYITGIRGKVQCLSEEDLEKNETDLEKRVMIREKCYLRFPTLPFIPKEPYDVIATDYDNFALVSGAKDTSFIQIYSRTPNPGPEFIEKYKSYLGNYGYDPSMIKDTPQDCEVMSNSQLAAMMSMSGMQQALTNQFPSLELKAPVEFNPFTSVFGTLKKLVELYFK

SEQ ID NO: 14 - protein, 331 aa, Helianthus annuus
MAYPQSAIATGKSLLLLAPSHSPPISRTNISFKCYSTQSPLSISTKDAAAAAKHVLAAGLAACFMLLSPSNQVLAIELSHNSLCQIASASNNVPTLEASNLMMMRGMTARNFDPVRYSGRWYEVASLKGGFAGQGQGDCHCTQGVYTIDMKTPAIQVDTFCVHGGPDGYITGIRGNVQCLSEEETEKTETDLERKEMIKEKCYLRFPTLPFIPKEPYDVLDTDYDNFALVSGAKDKSFIQIYSRTPNPGTEFIEKYKLVLADFGYDASKIKDTPQDCEVSDSRLAAMMSMNGMQQALTNQFPDLELKSAVEFNPFTSVFDTFKKLVQLYFK

SEQ ID NO: 15 - protein, 334 aa, Beta vulgaris subsp. vulgaris
MQVIKMSLPSPVLHRSSFSSSRGKPVNLVVRCSIDRPASENAIPKHIISGLVASCIFFSQANLVYGTDLPRHNSICQLADVSSNKVPFPLDENASDANDKVTMMMMRGMSAKNFDPVRYAGRWFEVASLKRGFAGQGQEDCHCTQGVYTFDMETPAIQVDTFCVHGGPDGYITGIRGKVQCLSEEDKELKETDLERQEMIKEKCYLRFPTLPFIPKEPYDVIATDYDHFALVSGAKDKSFIQIYSRTPNPGPEFIEKYKNYLADFGYDPNKTKDTPQDCQVMSNTQLASMMSQNGMQQVLNNQFPDLGLKASVEFNPFTSVLETLKKLVELYFK

SEQ ID NO: 16 - protein, 227 aa, Bathycoccus prasinos
MLQTRCCLRRKNDFASSSLLVALLAIAACASSFVTPALAGGLGRERRCPPVPTVSDVSIEAYASKPWYVQAQLPNRYQPVENLFCVRAVYTVTSPTTLDVFNFARKGSVEGEPSNEDMVLNAFIPDVDVKSKLKVGPKFVPRALYGDYWIVAYEEEEGWAIISGGQPTIFVSDGLCTTESGNQGLWLFTREKEVSEELVETMKKKANALGIDTSMLVTVQQTGCEYP

SEQ ID NO: 17 - protein, 179 aa, Gossypium arboreum
MEVVKNLDIQRYMGKWYEIASFPSFFQPKKGENTSAFYTLKEDGTVHVLNETFVNGKKDSIEGTAYKADPKSDEAKLKVKFYVPPFLPIIPVTGDYWVLYIDEDYQYVLVGGPTKKYLWILCRQKHMDEEIYNMLEQKAKDLGYDVSKLHKTPQSDSTPEGEHVPQEKGFWWIKSLFGK

SEQ ID NO: 18 - protein, 233 aa, Ostreococcus tauri
MTRRLRGHHAQRAVARLGAVALALALTRSHAFVLGVEASEECARVEPVDPFDLDAYVEAEWYVAAQKPTSYQPTRDLFCVRANYTVVDERTISIWNTANRDGVDGSPRNADGRFKLRGLIEDPNMPSKIAVGMRFLPRFLYGPYWVVATDVSPGDAEFDERGYSWAIISGGQPTISRGNGLCEPSGGLWLFVRDPEVSEEVVSKMKEKCESLGIDPDVLIPVTQEGCSFPTLP

SEQ ID NO: 19 - protein, 182 aa, Trifolium pratense
MGNNKEIEVVKGVDLERYMGRWYEIASFPSFFQPNNGENTRATYTLNSDGTVHVLNETWNKGKKNSIEGSAYKANPNSDEAKLKVKFYVPPFLPIIPVTGDYWILYLDEDYQYALIGGPTTKYLWILSRKTHLDDEIYNQLIEKAKEEGYDVTKLHKTPQTDPPPPEQEGPQPKGIWSLFGK

SEQ ID NO: 20 - protein, 156 aa, Trifolium pratense
MANKEMEVAKGVDLKRYMGRWYEIACFPSRFQPSDGCNTRATYTLKDDGTVNVLNETWSGGKRSYIEGTAYKADPNSDEAKLKVKFYVPPFLPIIPVTGDYWVLHLDDDYSYALIGQPSRNYLWSPLTIAQLGELSWERHHIWSLGWNPGDSTYSP

SEQ ID NO: 21 - protein, 178 aa, Brassica napus
MTTQKKEMEVVKDLDLERYMGRWYEIASFPSIFQPKNGVDTRATYTLNPDGTVHVLNETWNGGKRAFIQGSAYKTDPKSDEAKFKVKFYVPPFLPIIPVTGDYWVLYIDPEYQHAVIGQPSRSYLWILSRTAHVEEETYKQLLQKAVEEGYDGDTPPESNAAPDDTKGVWWFKSMFGK

SEQ ID NO: 22 - protein, 195 aa, Oryza sativa Japonica Group
MAAAAVEKKSGSEMTVVRGLDVARYMGRWYEIASLPNFFQPRDGRDTRATYALRPDGATVDVLNETWTSSGKRDYIKGTAYKADPASDEAKLKVKFYLPPFLPVIPVVGDYWVLYVDDDYQYALVGEPRRKDLWILCRQTSMDDEVYGRLLEKAKEEGYDVEKLRKTPQDDPPPESDAAPTDTKGTWWFKSLFGK

SEQ ID NO: 23 - protein, 184 aa, Parasponia andersonii
MAKKEMEVVKGLDLKRYMGKWYEIASFPSFFQPRNGVNTRATYTLNGDGTVKVLNETWSDDKRDYIEGTAYKADPNSDEAKLKVKFYVPPFLPIIPVVGDYWVLYIDDDYQVALIGQPSRKYLWILARQTHIDEEIYNQLVQRAKDEGYDVSKLNKTPQSDPPPEGDGPNDTKGIWWIKSLFGK

SEQ ID NO: 24 - protein, 185 aa, Cephalotus follicularis
MPKTVMKVVKDLDIPRYMGRWYEIASFPSRFQPKNGEDTRATYTLKEDGTINVLNETWTDGKRGYIEGTAYKADATSNEAKLKVKFYVPPFLPIIPVVGDYWVLFIDDDYQYALIGQPSRKYLWILSRKTHLDDEIYNELVEKAKGEGYDVSKLHKTIQHDPPPEGEDGPKDTKGIWWIKSILGK

SEQ ID NO: 25 - protein, 186 aa, Citrus sinensis
MASKKEMEVVRGLDIKRYMGRWYEIASFPSRNQPKNGADTRATYTLNEDGTVHVRNETWSDGKRGSIEGTAYKADPKSDEAKLKVKFYVPPFFPIIPVVGNYWVLYIDDNYQYALIGEPTRKYLWILCREPHMDEAIYNQLVEKATSEGYDVSKLHRTPQSDNPPEAEESPQDTKGIWWIKSIFGK

SEQ ID NO: 26 - protein, 344 aa, Panicum miliaceum
MVLVALGCSPASSLPARSLTSRRKCSTTRQRIVRCSLNEETPLNKHGVVSKQIISCVAASLVFISPPSQAIPAETSAQLGLCQIATVAAINSASVPLKFDSPSDEGSAGMMMMKGMTAKNFDPVRYSGRWFEVASLKRGFAGQGQEDCHCTQGVCSFDEKSRSIQVDTFCVHGGPDGYITGIRGREPYDVLATDYDNYAIVSGAKDTSFIQIYSRTPNPGPEFIKKYKSYVANFGYDPSKIKDTPQDCEYMSSDQLALMISMPGMNEALTNQFPDLKLKAPIALNPFTSQQNSSEPVTDGAQPLLQDLSGKATAGPPTTSEERAAYAMASRSATKRGWSFVGGG

SEQ ID NO: 27 - protein, 382 aa, Cynara cardunculus var. scolymus
MANKEMEVVKGVDLQRYMGRWYEIASFPSRFQPKDGINTRATYKLNEDGTINVLNETWSGGKRGYIEGTAYKADPKSDEAKLKVKFYVPPFLPIIPVTGDYWVLYLDDDYRYALIGQPSRRYLWILSRQNHLDEEIYNQLLEKAKEEGYDVSKLKKTTQTDPAPETDDAPADSKGDKAKAQEEQWQNTLEHKHILETCGLIKMEVAKGVDLERYMGRWYEIASIPSRDQPKNGTNTRATYTLNSDGTVHVLNETWSDGKRGFIEGTAYKADPKSDEAKLKVKFYVPPFLPIIPVTGDYWVLYLDDDYQYALIGQPSRNSLWILSRQNHLDEEIYEQLVQKAKEVGYDVSKLKKTTHADTPPETEDAPADNKGIWWLKSIFGK

SEQ ID NO: 28 - protein, 381 aa, Solanum lycopersicum
MAALSASAHVRIRTFFHSSFTNNKISNFSQQFKLENYTTITTITTSKRSISIPALAPKTTENSASQLQSTSDSVKDSENINLKGWAEFAKNVSGEWDGFGADFSKQGEPIELPESVVPGAYREWEVKVFDWQTQCPTLARDDDAFSFMYKFIRLLPTVGCEADAATRYSIDERNISDANVAAFAYQSTGCYVAAWSNNHDGNYNTAPYLSWELEHCLIDPGDKESRVRIVQVVRLQDSKLVLQNIKVFCEHWYGPFRNGDQLGGCAIQDSAFASTKALDPAEVIGVWEGKHAISSYNNAPEKVIQELVDGSTRKTVRDELDLVVLPRQLWCCLKGIAGGETCCEVGWLFDQGRAITSKCIFSDNGKLKEIAIACESAAPAQ

SEQ ID NO: 29 - protein, 346 aa, Brassica napus
MVSNIITSLSMTLVLPQSFTRPANTRCSVVRRINSRSHYSDRIICSLENPTESKEALRKHFVSGFAAILLLSQAGQGVALDLSSRYHNICQLGSASVEGNKPTLPLDDDPEAMMMMMMRGMTAKNFDPVRYSGRWFEVASLKRGFAGQGQEDCHCTQGVYTFDMKEPAIRVDTFCVHGSPDGYITGIRGKVQCVGAQDLEKTETDLEKQEMIKEKCYLRFPTIPFIPKLPYDVIATDYDNYALVSGAKDRSFVQVYSRTPNPGPEFIAKYKDYLAQFGYDPEKIKDTPQDCEVMSDGQLAAMMSMPGMEKTLTNQFPDLELRKSVQFDPFTSVFETLKKLVPLYFK

SEQ ID NO: 30 - protein, 176 aa, Micractinium conductrix
MAYSGRWYEVASLKRGFAGEGQQDCHCTQGIYTPKEGGPEGAIKLEVDTFCVHGGPGGRLSGIQGSVSCADPLLLSYLPEFQTEMEMVEGFVAKCALRFDSLAFLPPEPYVVLRTDYTSYALVRGAKDRSFVQIYSRTPNPGAKFIAEQKAVLGQLGYPANDIVDTPQDCPEMAPQ

SEQ ID NO: 31 - protein, 162 aa, Citrus unshiu
MVRYSGRWFEVASLKRGFAGQGQEDCHCTQGVYTFDKEKPAIQVDTFCVHGGPDGYITGIRGNVQCLPEEELEKNVTDLEKQEMIKGKCYLRFPTLPFIPKEPYDVIATDYDNFALVSGAKDKSFIQIYSRTPTPGPEFIEKYKSYLANFGYDPNKIKDTPQ

SEQ ID NO: 32 - protein, 182 aa, Ostreococcus tauri
MLDAYVEAEWYVAAQKPTSYQPTRDLFCVRANYTVVDERTISIWNTANRDGVDGSPRNADGRFKLRGLIEDPNMPSKIAVGMRFLPRFLYGPYWVVATDVSPGDAEFDERGYSWAIISGGQPTISRGNGLCEPSGGLWLFVRDPEVSEEVVSKMKEKCESLGIDPDVLIPVTQEGCSFPTLP

SEQ ID NO: 33 - protein, 162 aa, Macleaya cordata
MIRYSGRWFEVASLKRGFAGSGQEDCHCTQGVYTFDSEAPAIQVDTFCVHGGPDGYITGIRGKVQCLSEEDLEKNETDLEKRVMIREKCYLRFPTLPFIPKEPYDVIATDYDNFALVSGAKDTSFIQIYSRTPNPGPEFIEKYKSYLGNYGYDPSMIKDTPQ

SEQ ID NO: 34 - protein, 124 aa, Panicum miliaceum
MVRYSGRWFEVASLKRGFAGQGQEDCHCTQGVCSFDEKSRSIQVDTFCVHGGPDGYITGIRGREPYDVLATDYDNYAIVSGAKDTSFIQIYSRTPNPGPEFIKKYKSYVANFGYDPSKIKDTPQ

SEQ ID NO: 35 - protein, 170 aa, Solanum lycopersicum
MFAKNVSGEWDGFGADFSKQGEPIELPESVVPGAYREWEVKVFDWQTQCPTLARDDDAFSFMYKFIRLLPTVGCEADAATRYSIDERNISDANVAAFAYQSTGCYVAAWSNNHDGNYNTAPYLSWELEHCLIDPGDKESRVRIVQVVRLQDSKLVLQNIKVFCEHWYGPF

SEQ ID NO: 36 - protein, 154 aa, Cynara cardunculus var. scolymus
MVDLQRYMGRWYEIASFPSRFQPKDGINTRATYKLNEDGTINVLNETWSGGKRGYIEGTAYKADPKSDEAKLKVKFYVPPFLPIIPVTGDYWVLYLDDDYRYALIGQPSRRYLWILSRQNHLDEEIYNQLLEKAKEEGYDVSKLKKTTQTDPAP

SEQ ID NO: 37 - protein, 154 aa, Cynara cardunculus var. scolymus
MVDLERYMGRWYEIASIPSRDQPKNGTNTRATYTLNSDGTVHVLNETWSDGKRGFIEGTAYKADPKSDEAKLKVKFYVPPFLPIIPVTGDYWVLYLDDDYQYALIGQPSRNSLWILSRQNHLDEEIYEQLVQKAKEVGYDVSKLKKTTHADTPP

SEQ ID NO: 38 - protein, 162 aa, Beta vulgaris subsp. vulgaris
MVRYAGRWFEVASLKRGFAGQGQEDCHCTQGVYTFDMETPAIQVDTFCVHGGPDGYITGIRGKVQCLSEEDKELKETDLERQEMIKEKCYLRFPTLPFIPKEPYDVIATDYDHFALVSGAKDKSFIQIYSRTPNPGPEFIEKYKNYLADFGYDPNKTKDTPQ

SEQ ID NO: 39 - protein, 184 aa, Physcomitrella patens
MVSLEAYSGVWYEIGSTALVKARIERDLICATARYSVIPDGDLAGSIRVRNEGYNIRTGEFAHAIGTATVVSPGRLEVKFFPGAPGGDYRIIYLSGKAEDKYNVAIVYSCDESVPGGSQSLFILSREPELDDEDDDDDDYDDDDETLSRLLNFVRDLGIVFEPNNEFILTPQDPITCGRNGYDD

SEQ ID NO: 40 - protein, 162 aa, Oryza sativa Japonica Group
MIRYSGRWFEVASLKRGFAGQGQEDCHCTQGVYSFDEKSRSIQVDTFCVHGGPDGYITGIRGRVQCLSEEDMASAETDLERQEMIKGKCFLRFPTLPFIPKEPYDVLATDYDNYAVVSGAKDTSFIQIYSRTPNPGPEFIEKYKSYAANFGYDPSKIKDTPQ

SEQ ID NO: 41 - protein, 170 aa, Bathycoccus prasinos
MIEAYASKPWYVQAQLPNRYQPVENLFCVRAVYTVTSPTTLDVFNFARKGSVEGEPSNEDMVLNAFIPDVDVKSKLKVGPKFVPRALYGDYWIVAYEEEEGWAIISGGQPTIFVSDGLCTTESGNQGLWLFTREKEVSEELVETMKKKANALGIDTSMLVTVQQTGCEYP

SEQ ID NO: 42 - protein, 162 aa, Helianthus annuus
MVRYSGRWYEVASLKGGFAGQGQGDCHCTQGVYTIDMKTPAIQVDTFCVHGGPDGYITGIRGNVQCLSEEETEKTETDLERKEMIKEKCYLRFPTLPFIPKEPYDVLDTDYDNFALVSGAKDKSFIQIYSRTPNPGTEFIEKYKLVLADFGYDASKIKDTPQ

SEQ ID NO: 43 - protein, 162 aa, Arabidopsis thaliana
MVRYSGRWFEVASLKRGFAGQGQEDCHCTQGVYTFDMKESAIRVDTFCVHGSPDGYITGIRGKVQCVGAEDLEKSETDLEKQEMIKEKCFLRFPTIPFIPKLPYDVIATDYDNYALVSGAKDKGFVQVYSRTPNPGPEFIAKYKNYLAQFGYDPEKIKDTPQ

SEQ ID NO: 44 - protein, 162 aa, Zea mays
MVRYSGRWFEVASLKRGFAGQGQEDCHCTQGVYSFDEKARSIQVDTFCVHGGPDGYITGIRGRVQCLSEEDIASAETDLERQEMVRGKCFLRFPTLPFIPKEPYDVLATDYDNYAIVSGAKDTSFIQIYSRTPNPGPEFIDKYKSYVANFGYDPSKIKDTPQ

SEQ ID NO: 45 - protein, 154 aa, Brassica napus
MLDLERYMGRWYEIASFPSIFQPKNGIDTRATYTLNPDGTVDVLNETWNSGKRVFIQGSAYKTDPKSDEAKFKVKFYVPPFLPIIPVTGDYWVLYIDPEYQHAVIGQPSRSYLWILSRTAHVEEETYKQLLEKAVEEGYDVSKLHKTPQSDTPP

SEQ ID NO: 46 - protein, 162 aa, Brassica napus
MVRYSGRWFEVASLKRGFAGQGQEDCHCTQGVYTFDMKEPAIRVDTFCVHGSPDGYITGIRGKVQCVGAQDLEKTETDLEKQEMIKEKCYLRFPTIPFIPKLPYDVIATDYDNYALVSGAKDRSFVQVYSRTPNPGPEFIAKYKDYLAQFGYDPEKIKDTPQ

SEQ ID NO: 47 - protein, 85 aa, S. cerevisiae
MRFPSIFTAVLFAASSALAAPVNTTTEDETAQIPAEAVIGYSDLEGDFDVAVLPFSNSTNNGLLFINTTIASIAAKEEGVSLEKR

SEQ ID NO: 48 - protein, 492 aa, Arabidopsis thaliana
MDPYKYRPASSYNSPFFTTNSGAPVWNNNSSMTVGPRGLILLEDYHLVEKLANFDRERIPERVVHARGASAKGFFEVTHDISNLTCADFLRAPGVQTPVIVRFSTVIHARGSPETLRDPRGFAVKFYTREGNFDLVGNNFPVFFIRDGMKFPDIVHALKPNPKSHIQENWRILDFFSHHPESLNMFTFLFDDIGIPQDYRHMDGSGVNTYMLINKAGKAHYVKFHWKPTCGVKSLLEEDAIRLGGTNHSHATQDLYDSIAAGNYPEWKLFIQIIDPADEDKFDFDPLDVTKTWPEDILPLQPVGRMVLNKNIDNFFAENEQLAFCPAIIVPGIHYSDDKLLQTRVFSYADTQRHRLGPNYLQLPVNAPKCAHHNNHHEGFMNFMHRDEEVNYFPSRYDQVRHAEKYPTPPAVCSGKRERCIIEKENNFKEPGERYRTFTPERQERFIQRWIDALSDPRITHEIRSIWISYWSQADKSLGQKLASRLNVRPSI

SEQ ID NO: 49 - protein, 753 aa, Escherichia coli
MSQHNEKNPHQHQSPLHDSSEAKPGMDSLAPEDGSHRPAAEPTPPGAQPTAPGSLKAPDTRNEKLNSLEDVRKGSENYALTTNQGVRIADDQNSLRAGSRGPTLLEDFILREKITHFDHERIPERIVHARGSAAHGYFQPYKSLSDITKADFLSDPNKITPVFVRFSTVQGGAGSADTVRDIRGFATKFYTEEGIFDLVGNNTPIFFIQDAHKFPDFVHAVKPEPHWAIPQGQSAHDTFWDYVSLQPETLHNVMWAMSDRGIPRSYRTMEGFGIHTFRLINAEGKATFVRFHWKPLAGKASLVWDEAQKLTGRDPDFHRRELWEAIEAGDFPEYELGFQLIPEEDEFKFDFDLLDPTKLIPEELVPVQRVGKMVLNRNPDNFFAENEQAAFHPGHIVPGLDFTNDPLLQGRLFSYTDTQISRLGGPNFHEIPINRPTCPYHNFQRDGMHRMGIDTNPANYEPNSINDNWPRETPPGPKRGGFESYQERVEGNKVRERSPSFGEYYSHPRLFWLSQTPFEQRHIVDGFSFELSKVVRPYIRERVVDQLAHIDLTLAQAVAKNLGIELTDDQLNITPPPDVNGLKKDPSLSLYAIPDGDVKGRVVAILLNDEVRSADLLAILKALKAKGVHAKLLYSRMGEVTADDGTVLPIAATFAGAPSLTVDAVIVPCGNIADIADNGDANYYLMEAYKHLKPIALAGDARKFKATIKIADQGEEGIVEADSADGSFMDELLTLMAAHRVWSRIPKIDKIPA

SEQ ID NO: 50 - protein, 492 aa, Arabidopsis thaliana
MDPYRVRPSSAHDSPFFTTNSGAPVWNNNSSLTVGTRGPILLEDYHLLEKLANFDRERIPERVVHARGASAKGFFEVTHDITQLTSADFLRGPGVQTPVIVRFSTVIHERGSPETLRDPRGFAVKFYTREGNFDLVGNNFPVFFVRDGMKFPDMVHALKPNPKSHIQENWRILDFFSHHPESLHMFSFLFDDLGIPQDYRHMEGAGVNTYMLINKAGKAHYVKFHWKPTCGIKCLSDEEAIRVGGANHSHATKDLYDSIAAGNYPQWNLFVQVMDPAHEDKFDFDPLDVTKIWPEDILPLQPVGRLVLNKNIDNFFNENEQIAFCPALVVPGIHYSDDKLLQTRIFSYADSQRHRLGPNYLQLPVNAPKCAHHNNHHDGFMNFMHRDEEVNYFPSRLDPVRHAEKYPTTPIVCSGNREKCFIGKENNFKQPGERYRSWDSDRQERFVKRFVEALSEPRVTHEIRSIWISYWSQADKSLGQKLATRLNVRPNF

SEQ ID NO: 51 - protein, 492 aa, Arabidopsis thaliana
MDPYKYRPASSYNSPFFTTNSGAPVWNNNSSMTVGPRGPILLEDYHLVEKLANFDRERIPERVVHARGASAKGFFEVTHDISNLTCADFLRAPGVQTPVIVRFSTVIHERGSPETLRDPRGFAVKFYTREGNFDLVGNNFPVFFIRDGMKFPDMVHALKPNPKSHIQENWRILDFFSHHPESLNMFTFLFDDIGIPQDYRHMDGSGVNTYMLINKAGKAHYVKFHWKPTCGVKSLLEEDAIRVGGTNHSHATQDLYDSIAAGNYPEWKLFIQIIDPADEDKFDFDPLDVTKTWPEDILPLQPVGRMVLNKNIDNFFAENEQLAFCPAIIVPGIHYSDDKLLQTRVFSYADTQRHRLGPNYLQLPVNAPKCAHHNNHHEGFMNFMHRDEEVNYFPSRYDQVRHAEKYPTPPAVCSGKRERCIIEKENNFKEPGERYRTFTPERQERFIQRWIDALSDPRITHEIRSIWISYWSQADKSLGQKLASRLNVRPSI

SEQ ID NO: 52 - protein, 492 aa, Arabidopsis thaliana
MDPYKYRPSSAYNAPFYTTNGGAPVSNNISSLTIGERGPVLLEDYHLIEKVANFTRERIPERVVHARGISAKGFFEVTHDISNLTCADFLRAPGVQTPVIVRFSTVVHERASPETMRDIRGFAVKFYTREGNFDLVGNNTPVFFIRDGIQFPDVVHALKPNPKTNIQEYWRILDYMSHLPESLLTWCWMFDDVGIPQDYRHMEGFGVHTYTLIAKSGKVLFVKFHWKPTCGIKNLTDEEAKVVGGANHSHATKDLHDAIASGNYPEWKLFIQTMDPADEDKFDFDPLDVTKIWPEDILPLQPVGRLVLNRTIDNFFNETEQLAFNPGLVVPGIYYSDDKLLQCRIFAYGDTQRHRLGPNYLQLPVNAPKCAHHNNHHEGFMNFMHRDEEINYYPSKFDPVRCAEKVPTPTNSYTGIRTKCVIKKENNFKQAGDRYRSWAPDRQDRFVKRWVEILSEPRLTHEIRGIWISYWSQADRSLGQKLASRLNVRPSI

SEQ ID NO: 53 - protein, 28 aa, Cannabis
MNCSAFSFWFVCKIIFFFLSFHIQISIA

SEQ ID NO: 54 - protein, 28 aa, Cannabis
MKCSTFSFWFVCKIIFFFFSFNIQTSIA

SEQ ID NO: 55 - protein, 517 aa, Cannabis
NPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVDVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 56 - DNA, 1554 bp, Cannabis sativa
atgaatcctcgagaaaacttccttaaatgcttctcgcaatatattcccaataatgcaacaaatctaaaactcgtatacactcaaaacaacccattgtatatgtctgtcctaaattcgacaatacacaatcttagattcacctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagaatcttagtttggcggctgggtattgccctactgtttgcgcaggtggacactttggtggaggaggctatggaccattgatgagaaactatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttcagaatcagcaattccattccctcatcgagctggaatcttgtatgagttatggtacatatgtagttgggagaagcaagaagataacgaaaagcatctaaactggattagaaatatttataacttcatgactccttatgtgtccaaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataacttttttagaaacgaacaaagcatcccacctctaccacggcatcgtcattaa

SEQ ID NO: 57 - protein, 517 aa, Cannabis sativa
MNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 58 - DNA, 1074 bp, Cannabis
atgaagaagaacaaatcaactagtaataataagaacaacaacagtaataatatcatcaaaaacgacatcgtatcatcatcatcatcaacaacaacaacatcatcaacaactacagcaacatcatcatttcataatgagaaagttactgtcagtactgatcatattattaatcttgatgataagcagaaacgacaattatgtcgttgtcgtttagaaaaagaagaagaagaagaaggaagtggtggttgtggtgagacagtagtaatgatgctagggtcagtatctcctgctgctgctactgctgctgcagctgggggctcatcaagttgtgatgaagacatgttgggtggtcatgatcaactgttgttgttgtgttgttctgagaaaaaaacgacagaaatttcatcagtggtgaactttaataataataataataataataaggaaaatggtgacgaagtttcaggaccgtacgattatcatcatcataaagaagaggaagaagaagaagaagaagatgaagcatctgcatcagtagcagctgttgatgaagggatgttgttgtgctttgatgacataatagatagccacttgctaaatccaaatgaggttttgactttaagagaagatagccataatgaaggtggggcagctgatcagattgacaagactacttgtaataatactactattactactaatgatgattataacaataacttgatgatgttgagctgcaataataacggagattatgttattagtgatgatcatgatgatcagtactggatagacgacgtcgttggagttgacttttggagttgggagagttcgactactactgttattacccaagaacaagaacaagaacaagatcaagttcaagaacagaagaatatgtgggataatgagaaagagaaactgttgtctttgctatgggataatagtgataacagcagcagttgggagttacaagataaaagcaataataataataataataatgttcctaacaaatgtcaagagattacctctgataaagaaaatgctatggttgcatggcttctctcctga

SEQ ID NO: 59 - protein, 357 aa, Cannabis
MKKNKSTSNNKNNNSNNIIKNDIVSSSSSTTTTSSTTTATSSFHNEKVTVSTDHIINLDDKQKRQLCRCRLEKEEEEEGSGGCGETVVMMLGSVSPAAATAAAAGGSSSCDEDMLGGHDQLLLLCCSEKKTTEISSVVNFNNNNNNNKENGDEVSGPYDYHHHKEEEEEEEEDEASASVAAVDEGMLLCFDDIIDSHLLNPNEVLTLREDSHNEGGAADQIDKTTCNNTTITTNDDYNNNLMMLSCNNNGDYVISDDHDDQYWIDDVVGVDFWSWESSTTTVITQEQEQEQDQVQEQKNMWDNEKEKLLSLLWDNSDNSSSWELQDKSNNNNNNNVPNKCQEITSDKENAMVAWLLS

SEQ ID NO: 60 - protein, 462 aa, Humulus lupulus
MGRAPCCEKVGLKKGRWTSEEDEILTKYIQSNGEGCWRSLPKNAGLLRCGKSCRLRWINYLRADLKRGNISSEEEDIIIKLHSTLGNRWSLIASHLPGRTDNEIKNYWNSHLSRKIHTFRRCNNTTTHHHHLPNLVTVTKVNLPIPKRKGGRTSRLAMKKNKSSTSNQNSSVIKNDVGSSSSTTTTSVHQRTTTTTPTMDDQQKRQLSRCRLEEKEDQDGASTGTVVMMLGQAAAVGSSCDEDMLGHDQLSFLCCSEEKTTENSMTNLKENGDHEVSGPYDYDHRYEKETSVDEGMLLCFNDIIDSNLLNPNEVLTLSEESLNLGGALMDTTTSTTTNNNNYSLSYNNNGDCVISDDHDQYWLDDVVGVDFWSWESSTTVTQEQEQEQEQEQEQEQEQEQEQEHHHQQDQKKNTWDNEKEKMLALLWDSDNSNWELQDNNNYHKCQEITSDKENAMVAWLLS

SEQ ID NO: 61 - protein, 371 aa, Arabidopsis thaliana
MGRAPCCEKVGIKRGRWTAEEDQILSNYIQSNGEGSWRSLPKNAGLKRCGKSCRLRWINYLRSDLKRGNITPEEEELVVKLHSTLGNRWSLIAGHLPGRTDNEIKNYWNSHLSRKLHNFIRKPSISQDVSAVIMTNASSAPPPPQAKRRLGRTSRSAMKPKIHRTKTRKTKKTSAPPEPNADVAGADKEALMVESSGAEAELGRPCDYYGDDCNKNLMSINGDNGVLTFDDDIIDLLLDESDPGHLYTNTTCGGDGELHNIRDSEGARGFSDTWNQGNLDCLLQSCPSVESFLNYDHQVNDASTDEFIDWDCVWQEGSDNNLWHEKENPDSMVSWLLDGDDEATIGNSNCENFGEPLDHDDESALVAWLLS

SEQ ID NO: 62 - protein, 243 aa, Arabidopsis thaliana
MNISRTEFANCKTLINHKEEVEEVEKKMEIEIRRGPWTVEEDMKLVSYISLHGEGRWNSLSRSAGLNRTGKSCRLRWLNYLRPDIRRGDISLQEQFIILELHSRWGNRWSKIAQHLPGRTDNEIKNYWRTRVQKHAKLLKCDVNSKQFKDTIKHLWMPRLIERIAATQSVQFTSNHYSPENSSVATATSSTSSSEAVRSSFYGGDQVEFGTLDHMTNGGYWFNGGDTFETLCSFDELNKWLIQ

SEQ ID NO: 63 - DNA, 1515 bp, Mus musculus
atgaacttgttttctgctttgtctttggatactttggttttgttggctattattttggttttgttgtacagatacggtactagaactcatggtttgtttaagaagcaaggtattccaggtccaaagccattgccatttttgggtactgttttgaactactacactggtatttggaagtttgatatggaatgttacgaaaagtacggtaagacttggggtttgtttgatggtcaaactccattgttggttattactgatccagaaactattaagaacgttttggttaaggattgtttgtctgtttttactaacagaagagaatttggtccagttggtattatgtctaaggctatttctatttctaaggatgaagaatggaagagatacagagctttgttgtctccaacttttacttctggtagattgaaggaaatgtttccagttattgaacaatacggtgatattttggttaagtacttgagacaagaagctgaaaagggtatgccagttgctatgaaggatgttttgggtgcttactctatggatgttattacttctacttcttttggtgttaacgttgattctttgaacaacccagaagatccatttgttgaagaagctaagaagtttttgagagttgatttttttgatccattgttgttttctgttgttttgtttccattgttgactccagtttacgaaatgttgaacatttgtatgtttccaaacgattctattgaattttttaagaagtttgttgatagaatgcaagaatctagattggattctaaccaaaagcatagagttgattttttgcaattgatgatgaactctcataacaactctaaggataaggattctcataaggctttttctaacatggaaattactgttcaatctattatttttatttctgctggttacgaaactacttcttctactttgtcttttactttgtactgtttggctactcatccagatattcaaaagaagttgcaagctgaaattgataaggctttgccaaacaaggctactccaacttgtgatactgttatggaaatggaatacttggatatggttttgaacgaaactttgagattgtacccaattgttactagattggaaagagtttgtaagaaggatgttgaattgaacggtgtttacattccaaagggttctatggttatgattccatcttacgctttgcatcatgatccacaacattggccagatccagaagaatttcaaccagaaagattttctaaggaaaacaagggttctattgatccatacgtttacttgccatttggtattggtccaagaaactgtattggtatgagatttgctttgatgaacatgaagttggctgttactaaggttttgcaaaacttttcttttcaaccatgtcaagaaactcaaattccattgaagttgtctagacaaggtattttgcaaccagaaaagccaattgttttgaaggttgttccaagagatgctgttattactggtgcttaa

SEQ ID NO: 64 - protein, 504 aa, Mus musculus
MNLFSALSLDTLVLLAIILVLLYRYGTRTHGLFKKQGIPGPKPLPFLGTVLNYYTGIWKFDMECYEKYGKTWGLFDGQTPLLVITDPETIKNVLVKDCLSVFTNRREFGPVGIMSKAISISKDEEWKRYRALLSPTFTSGRLKEMFPVIEQYGDILVKYLRQEAEKGMPVAMKDVLGAYSMDVITSTSFGVNVDSLNNPEDPFVEEAKKFLRVDFFDPLLFSVVLFPLLTPVYEMLNICMFPNDSIEFFKKFVDRMQESRLDSNQKHRVDFLQLMMNSHNNSKDKDSHKAFSNMEITVQSIIFISAGYETTSSTLSFTLYCLATHPDIQKKLQAEIDKALPNKATPTCDTVMEMEYLDMVLNETLRLYPIVTRLERVCKKDVELNGVYIPKGSMVMIPSYALHHDPQHWPDPEEFQPERFSKENKGSIDPYVYLPFGIGPRNCIGMRFALMNMKLAVTKVLQNFSFQPCQETQIPLKLSRQGILQPEKPIVLKVVPRDAVITGA

SEQ ID NO: 65 - DNA, 2037 bp, Mus musculus
atgggtgattctcatgaagatacttctgctactgttccagaagctgttgctgaagaagtttctttgttttctactactgatattgttttgttttctttgattgttggtgttttgacttactggtttatttttaagaagaagaaggaagaaattccagaattttctaagattcaaactactgctccaccagttaaggaatcttcttttgttgaaaagatgaagaagactggtagaaacattattgttttttacggttctcaaactggtactgctgaagaatttgctaacagattgtctaaggatgctcatagatacggtatgagaggtatgtctgctgatccagaagaatacgatttggctgatttgtcttctttgccagaaattgataagtctttggttgttttttgtatggctacttacggtgaaggtgatccaactgataacgctcaagatttttacgattggttgcaagaaactgatgttgatttgactggtgttaagtttgctgtttttggtttgggtaacaagacttacgaacattttaacgctatgggtaagtacgttgatcaaagattggaacaattgggtgctcaaagaatttttgaattgggtttgggtgatgatgatggtaacttggaagaagattttattacttggagagaacaattttggccagctgtttgtgaattttttggtgttgaagctactggtgaagaatcttctattagacaatacgaattggttgttcatgaagatatggatactgctaaggtttacactggtgaaatgggtagattgaagtcttacgaaaaccaaaagccaccatttgatgctaagaacccatttttggctgctgttactactaacagaaagttgaaccaaggtactgaaagacatttgatgcatttggaattggatatttctgattctaagattagatacgaatctggtgatcatgttgctgtttacccagctaacgattctactttggttaaccaaattggtgaaattttgggtgctgatttggatgttattatgtctttgaacaacttggatgaagaatctaacaagaagcatccatttccatgtccaactacttacagaactgctttgacttactacttggatattactaacccaccaagaactaacgttttgtacgaattggctcaatacgcttctgaaccatctgaacaagaacatttgcataagatggcttcttcttctggtgaaggtaaggaattgtacttgtcttgggttgttgaagctagaagacatattttggctattttgcaagattacccatctttgagaccaccaattgatcatttgtgtgaattgttgccaagattgcaagctagatactactctattgcttcttcttctaaggttcatccaaactctgttcatatttgtgctgttgctgttgaatacgaagctaagtctggtagagttaacaagggtgttgctacttcttggttgagaactaaggaaccagctggtgaaaacggtagaagagctttggttccaatgtttgttagaaagtctcaatttagattgccatttaagccaactactccagttattatggttggtccaggtactggtgttgctccatttatgggttttattcaagaaagagcttggttgagagaacaaggtaaggaagttggtgaaactttgttgtactacggttgtagaagatctgatgaagattacttgtacagagaagaattggctagatttcataaggatggtgctttgactcaattgaacgttgctttttctagagaacaagctcataaggtttacgttcaacatttgttgaagagagataaggaacatttgtggaagttgattcatgaaggtggtgctcatatttacgtttgtggtgatgctagaaacatggctaaggatgttcaaaacactttttacgatattgttgctgaatttggtccaatggaacatactcaagctgttgattacgttaagaagttgatgactaagggtagatactctttggatgtttggtcttaa

SEQ ID NO: 66 - protein, 678 aa, Mus musculus
MGDSHEDTSATVPEAVAEEVSLFSTTDIVLFSLIVGVLTYWFIFKKKKEEIPEFSKIQTTAPPVKESSFVEKMKKTGRNIIVFYGSQTGTAEEFANRLSKDAHRYGMRGMSADPEEYDLADLSSLPEIDKSLVVFCMATYGEGDPTDNAQDFYDWLQETDVDLTGVKFAVFGLGNKTYEHFNAMGKYVDQRLEQLGAQRIFELGLGDDDGNLEEDFITWREQFWPAVCEFFGVEATGEESSIRQYELVVHEDMDTAKVYTGEMGRLKSYENQKPPFDAKNPFLAAVTTNRKLNQGTERHLMHLELDISDSKIRYESGDHVAVYPANDSTLVNQIGEILGADLDVIMSLNNLDEESNKKHPFPCPTTYRTALTYYLDITNPPRTNVLYELAQYASEPSEQEHLHKMASSSGEGKELYLSWVVEARRHILAILQDYPSLRPPIDHLCELLPRLQARYYSIASSSKVHPNSVHICAVAVEYEAKSGRVNKGVATSWLRTKEPAGENGRRALVPMFVRKSQFRLPFKPTTPVIMVGPGTGVAPFMGFIQERAWLREQGKEVGETLLYYGCRRSDEDYLYREELARFHKDGALTQLNVAFSREQAHKVYVQHLLKRDKEHLWKLIHEGGAHIYVCGDARNMAKDVQNTFYDIVAEFGPMEHTQAVDYVKKLMTKGRYSLDVWS

SEQ ID NO: 67 - DNA, 1509 bp, Human
atggctttgattcctgatttggctatggaaactagattgttgttggctgtttcattggttttgttgtatttgtatggaactcattcacatggattgtttaaaaaattgggaattcctggacctactcctttgccttttttgggaaatattttgtcatatcataaaggattttgcatgtttgatatggaatgccataaaaaatatggaaaagtttggggattttatgatggacaacaacctgttttggctattactgatcctgatatgattaaaactgttttggttaaagaatgctattcagtttttactaatagaagaccttttggacctgttggatttatgaaatcagctatttcaattgctgaagatgaagaatggaaaagattgagatcattgttgtcacctacttttacttcaggaaaattgaaagaaatggttcctattattgctcaatatggagatgttttggttagaaatttgagaagagaagctgaaactggaaaacctgttactttgaaagatgtttttggagcttattcaatggatgttattacttcaacttcatttggagttaatattgattcattgaataatcctcaagatccttttgttgaaaatactaaaaaattgttgagatttgattttttggatcctttttttttgtcaattactgtttttccttttttgattcctattttggaagttttgaatatttgcgtttttcctagagaagttactaattttttgagaaaatcagttaaaagaatgaaagaatcaagattggaagatactcaaaaacatagagttgattttttgcaattgatgattgattcacaaaattcaaaagaaactgaatcacataaagctttgtcagatttggaattggttgctcaatcaattatttttatttttgctggatgcgaaactacttcatcagttttgtcatttattatgtatgaattggctactcatcctgatgttcaacaaaaattgcaagaagaaattgatgctgttttgcctaataaagctcctcctacttatgatactgttttgcaaatggaatatttggatatggttgttaatgaaactttgagattgtttcctattgctatgagattggaaagagtttgcaaaaaagatgttgaaattaatggaatgtttattcctaaaggagttgttgttatgattccttcatatgctttgcatagagatcctaaatattggactgaacctgaaaaatttttgcctgaaagattttcaaaaaaaaataaagataatattgatccttatatttatactccttttggatcaggacctagaaattgcattggaatgagatttgctttgatgaatatgaaattggctttgattagagttttgcaaaatttttcatttaaaccttgcaaagaaactcaaattcctttgaaattgtcattgggaggattgttgcaacctgaaaaacctgttgttttgaaagttgaatcaagagatggaactgtttcaggagct

SEQ ID NO: 68 - protein, 503 aa, Human
MALIPDLAMETRLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGCETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 69 - DNA, 2040 bp, Human
atgattaatatgggagattcacatgttgatacttcatcaactgtttcagaagctgttgctgaagaagtttcattgttttcaatgactgatatgattttgttttcattgattgttggattgttgacttattggtttttgtttagaaaaaaaaaagaagaagttcctgaatttactaaaattcaaactttgacttcatcagttagagaatcatcatttgttgaaaaaatgaaaaaaactggaagaaatattattgttttttatggatcacaaactggaactgctgaagaatttgctaatagattgtcaaaagatgctcatagatatggaatgagaggaatgtcagctgatcctgaagaatatgatttggctgatttgtcatcattgcctgaaattgataatgctttggttgttttttgcatggctacttatggagaaggagatcctactgataatgctcaagatttttatgattggttgcaagaaactgatgttgatttgtcaggagttaaatttgctgtttttggattgggaaataaaacttatgaacattttaatgctatgggaaaatatgttgataaaagattggaacaattgggagctcaaagaatttttgaattgggattgggagatgatgatggaaatttggaagaagattttattacttggagagaacaattttggttggctgtttgcgaacattttggagttgaagctactggagaagaatcatcaattagacaatatgaattggttgttcatactgatattgatgctgctaaagtttatatgggagaaatgggaagattgaaatcatatgaaaatcaaaaacctccttttgatgctaaaaatccttttttggctgctgttactactaatagaaaattgaatcaaggaactgaaagacatttgatgcatttggaattggatatttcagattcaaaaattagatatgaatcaggagatcatgttgctgtttatcctgctaatgattcagctttggttaatcaattgggaaaaattttgggagctgatttggatgttgttatgtcattgaataatttggatgaagaatcaaataaaaaacatccttttccttgccctacttcatatagaactgctttgacttattatttggatattactaatcctcctagaactaatgttttgtatgaattggctcaatatgcttcagaaccttcagaacaagaattgttgagaaaaatggcttcatcatcaggagaaggaaaagaattgtatttgtcatgggttgttgaagctagaagacatattttggctattttgcaagattgcccttcattgagacctcctattgatcatttgtgcgaattgttgcctagattgcaagctagatattattcaattgcttcatcatcaaaagttcatcctaattcagttcatatttgcgctgttgttgttgaatatgaaactaaagctggaagaattaataaaggagttgctactaattggttgagagctaaagaacctgttggagaaaatggaggaagagctttggttcctatgtttgttagaaaatcacaatttagattgccttttaaagctactactcctgttattatggttggacctggaactggagttgctccttttattggatttattcaagaaagagcttggttgagacaacaaggaaaagaagttggagaaactttgttgtattatggatgcagaagatcagatgaagattatttgtatagagaagaattggctcaatttcatagagatggagctttgactcaattgaatgttgctttttcaagagaacaatcacataaagtttatgttcaacatttgttgaaacaagatagagaacatttgtggaaattgattgaaggaggagctcatatttatgtttgcggagatgctagaaatatggctagagatgttcaaaatactttttatgatattgttgctgaattgggagctatggaacatgctcaagctgttgattatattaaaaaattgatgactaaaggaagatattcattggatgtttggtca

SEQ ID NO: 70 - protein, 680 aa, Human
MINMGDSHVDTSSTVSEAVAEEVSLFSMTDMILFSLIVGLLTYWFLFRKKKEEVPEFTKIQTLTSSVRESSFVEKMKKTGRNIIVFYGSQTGTAEEFANRLSKDAHRYGMRGMSADPEEYDLADLSSLPEIDNALVVFCMATYGEGDPTDNAQDFYDWLQETDVDLSGVKFAVFGLGNKTYEHFNAMGKYVDKRLEQLGAQRIFELGLGDDDGNLEEDFITWREQFWLAVCEHFGVEATGEESSIRQYELVVHTDIDAAKVYMGEMGRLKSYENQKPPFDAKNPFLAAVTTNRKLNQGTERHLMHLELDISDSKIRYESGDHVAVYPANDSALVNQLGKILGADLDVVMSLNNLDEESNKKHPFPCPTSYRTALTYYLDITNPPRTNVLYELAQYASEPSEQELLRKMASSSGEGKELYLSWVVEARRHILAILQDCPSLRPPIDHLCELLPRLQARYYSIASSSKVHPNSVHICAVVVEYETKAGRINKGVATNWLRAKEPVGENGGRALVPMFVRKSQFRLPFKATTPVIMVGPGTGVAPFIGFIQERAWLRQQGKEVGETLLYYGCRRSDEDYLYREELAQFHRDGALTQLNVAFSREQSHKVYVQHLLKQDREHLWKLIEGGAHIYVCGDARNMARDVQNTFYDIVAELGAMEHAQAVDYIKKLMTKGRYSLDVWS

SEQ ID NO: 71 - DNA, 1554 bp, Cannabis sativa
atgaatcctcgagaaaacttccttaaatgcttctcgcaatatattcccaataatgcaacaaatctaaaactcgtatacactcaaaacaacccattgtatatgtctgtcctaaattcgacaatacacaatcttagattcacctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagaatcttagtttggcggctgggtattgccctactgtttgcgcaggtggacactttggtggaggaggctatggaccattgatgagaaactatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttcagaatcagcaattccattccctcatcgagctggaatcttgtatgagttatggtacatatgtagttgggagaagcaagaagataacgaaaagcatctaaactggattagaaatatttataacttcatgactccttatgtgtccaaaaattcaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataacttttttagaaacgaacaaagcatcccacctcaaccacggcatcgtcattaa

SEQ ID NO: 72 - protein, 517 aa, Cannabis sativa
MNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNSRLAYLNYRDLDIGINDPKNPNNYTQARIWGEKYFGKNFDRLVKVKTLVDPNNFFRNEQSIPPQPRHRH

SEQ ID NO: 73 - DNA, 1374 bp, Stevia rebaudiana
atggaaaataaaactgaaactactgttagaagaagaagaagaattattttgtttcctgttccttttcaaggacatattaatcctattttgcaattggctaatgttttgtattcaaaaggattttcaattactatttttcatactaattttaataaacctaaaacttcaaattatcctcattttacttttagatttattttggataatgatcctcaagatgaaagaatttcaaatttgcctactcatggacctttggctggaatgagaattcctattattaatgaacatggagctgatgaattgagaagagaattggaattgttgatgttggcttcagaagaagatgaagaagtttcatgcttgattactgatgctttgtggtattttgctcaatcagttgctgattcattgaatttgagaagattggttttgatgacttcatcattgtttaattttcatgctcatgtttcattgcctcaatttgatgaattgggatatttggatcctgatgataaaactagattggaagaacaagcttcaggatttcctatgttgaaagttaaagatattaaatcagcttattcaaattggcaaattttgaaagaaattttgggaaaaatgattaaacaaactagagcttcatcaggagttatttggaattcatttaaagaattggaagaatcagaattggaaactgttattagagaaattcctgctccttcatttttgattcctttgcctaaacatttgactgcttcatcatcatcattgttggatcatgatagaactgtttttcaatggttggatcaacaacctccttcatcagttttgtatgtttcatttggatcaacttcagaagttgatgaaaaagattttttggaaattgctagaggattggttgattcaaaacaatcatttttgtgggttgttagacctggatttgttaaaggatcaacttgggttgaacctttgcctgatggatttttgggagaaagaggaagaattgttaaatgggttcctcaacaagaagttttggctcatggagctattggagctttttggactcattcaggatggaattcaactttggaatcagtttgcgaaggagttcctatgattttttcagattttggattggatcaacctttgaatgctagatatatgtcagatgttttgaaagttggagtttatttggaaaatggatgggaaagaggagaaattgctaatgctattagaagagttatggttgatgaagaaggagaatatattagacaaaatgctagagttttgaaacaaaaagctgatgtttcattgatgaaaggaggatcatcatatgaatcattggaatcattggtttcatatatttcatcattg

SEQ ID NO: 74 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTRASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 75 - protein, 485 aa, Nicotiana tabacum
MGSIGAELTKPHAVCIPYPAQGHINPMLKLAKILHHKGFHITFVNTEFNHRRLLKSRGPDSLKGLSSFRFETIPDGLPPCEADATQDIPSLCESTTNTCLAPFRDLLAKLNDTNTSNVPPVSCIVSDGVMSFTLAAAQELGVPEVLFWTTSACGFLGYMHYCKVIEKGYAPLKDASDLTNGYLETTLDFIPGMKDVRLRDLPSFLRTTNPDEFMIKFVLQETERARKASAIILNTFETLEAEVLESLRNLLPPVYPIGPLHFLVKHVDDENLKGLRSSLWKEEPECIQWLDTKEPNSVVYVNFGSITVMTPNQLIEFAWGLANSQQTFLWIIRPDIVSGDASILPPEFVEETKNRGMLASWCSQEEVLSHPAIVGFLTHSGWNSTLESISSGVPMICWPFFAEQQTNCWFSVTKWDVGMEIDSDVKRDEVESLVRELMVGGKGKKMKKKAMEWKELAEASAKEHSGSSYVNIEKLVNDILLSSKH

SEQ ID NO: 76 - DNA, 1458 bp, Nicotiana tabacum
atgggttccattggtgctgaattaacaaagccacatgcagtttgcataccatatcccgcccaaggccatattaaccccatgttaaagctagccaaaatccttcatcacaaaggctttcacatcacttttgtcaatactgaatttaaccaccgacgtctccttaaatctcgtggccctgattctctcaagggtctttcttctttccgttttgagaccattcctgatggacttccgccatgtgaggcagatgccacacaagatataccttctttgtgtgaatctacaaccaatacttgcttggctccttttagggatcttcttgcgaaactcaatgatactaacacatctaacgtgccacccgtttcgtgcatcgtctcggatggtgtcatgagcttcaccttagccgctgcacaagaattgggagtccctgaagttctgttttggaccactagtgcttgtggtttcttaggttacatgcattactgcaaggttattgaaaaaggatatgctccacttaaagatgcgagtgacttgacaaatggatacctagagacaacattggattttataccaggcatgaaagacgtacgtttaagggatcttccaagtttcttgagaactacaaatccagatgaattcatgatcaaatttgtcctccaagaaacagagagagcaagaaaggcttctgcaattatcctcaacacatttgaaacactagaggctgaagttcttgaatcgctccgaaatcttcttcctccagtctaccccatagggcccttgcattttctagtgaaacatgttgatgatgagaatttgaagggacttagatccagcctttggaaagaggaaccagagtgtatacaatggcttgataccaaagaaccaaattctgttgtttatgttaactttggaagcattactgttatgactcctaatcagcttattgagtttgcttggggacttgcaaacagccagcaaacattcttatggatcataagacctgatattgtttcaggtgatgcatcgattcttccacccgaattcgtggaagaaacgaagaacagaggtatgcttgctagttggtgttcacaagaagaagtacttagtcaccctgcaatagtaggattcttgactcacagtggatggaattcgacactcgaaagtataagcagtggggtgcctatgatttgctggccatttttcgctgaacagcaaacaaattgttggttttccgtcactaaatgggatgttggaatggagattgacagtgatgtgaagagagatgaagtggaaagccttgtaagggaattgatggttgggggaaaaggcaaaaagatgaagaaaaaggcaatggaatggaaggaattggctgaagcatctgctaaagaacattcagggtcatcttatgtgaacattgaaaagttggtcaatgatattcttctttcatccaaacattaa

SEQ ID NO: 77 - protein, 485 aa, Nicotiana tabacum
MGSIGAEFTKPHAVCIPYPAQGHINPMLKLAKILHHKGFHITFVNTEFNHRRLLKSRGPDSLKGLSSFRFETIPDGLPPCDADATQDIPSLCESTTNTCLGPFRDLLAKLNDTNTSNVPPVSCIISDGVMSFTLAAAQELGVPEVLFWTTSACGFLGYMHYYKVIEKGYAPLKDASDLTNGYLETTLDFIPCMKDVRLRDLPSFLRTTNPDEFMIKFVLQETERARKASAIILNTYETLEAEVLESLRNLLPPVYPIGPLHFLVKHVDDENLKGLRSSLWKEEPECIQWLDTKEPNSVVYVNFGSITVMTPNQLIEFAWGLANSQQSFLWIIRPDIVSGDASILPPEFVEETKKRGMLASWCSQEEVLSHPAIGGFLTHSGWNSTLESISSGVPMICWPFFAEQQTNCWFSVTKWDVGMEIDCDVKRDEVESLVRELMVGGKGKKMKKKAMEWKELAEASAKEHSGSSYVNIEKVVNDILLSSKH

SEQ ID NO: 78 - DNA, 1458 bp, Nicotiana tabacum
atgggttccattggtgctgaatttacaaagccacatgcagtttgcataccatatcccgcccaaggccatattaaccccatgttaaagctagccaaaatccttcatcacaaaggctttcacatcacttttgtcaatactgaatttaaccacagacgtctgcttaaatctcgtggccctgattctctcaagggtctttcttctttccgttttgagacaattcctgatggacttccgccatgtgatgcagatgccacacaagatataccttctttgtgtgaatctacaaccaatacttgcttgggtccttttagggatcttcttgcgaaactcaatgatactaacacatctaacgtgccacccgtttcgtgcatcatctcagatggtgtcatgagcttcaccttagccgctgcacaagaattgggagtccctgaagttctgttttggaccactagtgcttgtggtttcttaggttacatgcattattacaaggttattgaaaaaggatacgctccacttaaagatgcgagtgacttgacaaatggatacctagagacaacattggattttataccatgcatgaaagacgtacgtttaagggatcttccaagtttcttgagaactacaaatccagatgaattcatgatcaaatttgtcctccaagaaacagagagagcaagaaaggcttctgcaattatcctcaacacatatgaaacactagaggctgaagttcttgaatcgctccgaaatcttcttcctccagtctaccccattgggcccttgcattttctagtgaaacatgttgatgatgagaatttgaagggacttagatccagcctttggaaagaggaaccagagtgtatacaatggcttgataccaaagaaccaaattctgttgtttatgttaactttggaagcattactgttatgactcctaatcaacttattgaatttgcttggggacttgcaaacagccaacaatcattcttatggatcataagacctgatattgtttcaggtgatgcatcgattcttccccccgaattcgtggaagaaacgaagaagagaggtatgcttgctagttggtgttcacaagaagaagtacttagtcaccctgcaataggaggattcttgactcacagtggatggaattcgacactcgaaagtataagcagtggggtgcctatgatttgctggccatttttcgctgaacagcaaacaaattgttggttttccgtcactaaatgggatgttggaatggagattgactgtgatgtgaagagggatgaagtggaaagccttgtaagggaattgatggttgggggaaaaggcaaaaagatgaagaaaaaggcaatggaatggaaggaattggctgaagcatctgctaaagaacattcagggtcatcttatgtgaacattgagaaggtggtcaatgatattcttctttcgtccaaacattaa

SEQ ID NO: 79 - protein, 496 aa, Nicotiana tabacum
MATQVHKLHFILFPLMAPGHMIPMIDIAKLLANRGVITTIITTPVNANRFSSTITRAIKSGLRIQILTLKFPSVEVGLPEGCENIDMLPSLDLASKFFAAISMLKQQVENLLEGINPSPSCVISDMGFPWTTQIAQNFNIPRIVFHGTCCFSLLCSYKILSSNILENITSDSEYFVVPDLPDRVELTKAQVSGSTKNTTSVSSSVLKEVTEQIRLAEESSYGVIVNSFEELEQVYEKEYRKARGKKVWCVGPVSLCNKEIEDLVTRGNKTAIDNQDCLKWLDNFETESVVYASLGSLSRLTLLQMVELGLGLEESNRPFVWVLGGGDKLNDLEKWILENGFEQRIKERGVLIRGWAPQVLILSHPAIGGVLTHCGWNSTLEGISAGLPMVTWPLFAEQFCNEKLVVQVLKIGVSLGVKVPVKWGDEENVGVLVKKDDVKKALDKLMDEGEEGQVRRTKAKELGELAKKAFGEGGSSYVNLTSLIEDIIEQQNHKEK

SEQ ID NO: 80 - DNA, 1491 bp, Nicotiana tabacum
atggcaactcaagtgcacaaacttcatttcatactattccctttaatggctccaggccacatgattcctatgatagacatagctaaacttctagcaaatcgcggtgtcattaccactatcatcaccactccagtaaacgccaatcgtttcagttcaacaattactcgtgccataaaatccggtctaagaatccaaattcttacactcaaatttccaagtgtagaagtaggattaccagaaggttgcgaaaatattgacatgcttccttctcttgacttggcttcaaagttttttgctgcaattagtatgctgaaacaacaagttgaaaatctcttagaaggaataaatccaagtccaagttgtgttatttcagatatgggatttccttggactactcaaattgcacaaaattttaatatcccaagaattgtttttcatggtacttgttgtttctcacttttatgttcctataaaatactttcctccaacattcttgaaaatataacctcagattcagagtattttgttgttcctgatttacccgatagagttgaactaacgaaagctcaggtttcaggatcgacgaaaaatactacttctgttagttcttctgtattgaaagaagttactgagcaaatcagattagccgaggaatcatcatatggtgtaattgttaatagttttgaggagttggagcaagtgtatgagaaagaatataggaaagctagagggaaaaaagtttggtgtgttggtcctgtttctttgtgtaataaggaaattgaagatttggttacaaggggtaataaaactgcaattgataatcaagattgcttgaaatggttagataattttgaaacagaatctgtggtttatgcaagtcttggaagtttatctcgtttgacattattgcaaatggtggaacttggtcttggtttagaagagtcaaataggccttttgtatgggtattaggaggaggtgataaattaaatgatttagagaaatggattcttgagaatggatttgagcaaagaattaaagaaagaggagttttgattagaggatgggctcctcaagtgcttatactttcacaccctgcaattggtggagtattgactcattgcggatggaattctacattggaaggtatttcagcaggattaccaatggtaacatggccactatttgctgagcaattttgcaatgagaagttagtagtccaagtgctaaaaattggagtgagcctaggtgtgaaggtgcctgtcaaatggggagatgaggaaaatgttggagttttggtaaaaaaggatgatgttaagaaagcattagacaaactaatggatgaaggagaagaaggacaagtaagaagaacaaaagcaaaagagttaggagaattggctaaaaaggcatttggagaaggtggttcttcttatgttaacttaacatctctgattgaagacatcattgagcaacaaaatcacaaggaaaaatag

SEQ ID NO: 81 - protein, 479 aa, Nicotiana tabacum
MKTAELVFIPAPGMGHLVPTVEVAKQLVDRHEQLSITVLIMTIPLETNIPSYTKSLSSDYSSRITLLPLSQPETSVTMSSFNAINFFEYISSYKGRVKDAVSETSFSSSNSVKLAGFVIDMFCTAMIDVANEFGIPSYVFYTSSAAMLGLQLHFQSLSIECSPKVHNYVEPESEVLISTYMNPVPVKCLPGIILVNDESSTMFVNHARRFRETKGIMVNTFTELESHALKALSDDEKIPPIYPVGPILNLENGNEDHNQEYDAIMKWLDEKPNSSVVFLCFGSKGSFEEDQVKEIANALESSGYHFLWSLRRPPPKDKLQFPSEFENPEEVLPEGFFQRTKGRGKVIGWAPQLAILSHPSVGGFVSHCGWNSTLESVRSGVPIATWPLYAEQQSNAFQLVKDLGMAVEIKMDYREDFNTRNPPLVKAEEIEDGIRKLMDSENKIRAKVTEMKDKSRAALLEGGSSYVALGHFVETVMKN

SEQ ID NO: 82 - DNA, 1440 bp, Nicotiana tabacum
atgaagacagcagagttagtattcattcctgctcctgggatgggtcaccttgtaccaactgtggaggtggcaaagcaactagtcgacagacacgagcagctttcgatcacagttctaatcatgacaattcctttggaaacaaatattccatcatatactaaatcactgtcctcagactacagttctcgtataacgctgcttccactctctcaacctgagacctctgttactatgagcagttttaatgccatcaatttttttgagtacatctccagctacaagggtcgtgtcaaagatgctgttagtgaaacctcctttagttcgtcaaattctgtgaaacttgcaggatttgtaatagacatgttctgcactgcgatgattgatgtagcgaacgagtttggaatcccaagttatgtgttctacacttctagtgcagctatgcttggactacaactgcattttcaaagtcttagcattgaatgcagtccgaaagttcataactacgttgaacctgaatcagaagttctgatctcaacttacatgaatccggttccagtcaaatgtttgcccggaattatactagtaaatgatgaaagtagcaccatgtttgtcaatcatgcacgaagattcagggagacgaaaggaattatggtgaacacgttcactgagcttgaatcacacgctttgaaagccctttccgatgatgaaaaaatcccaccaatctacccagttggacctatacttaaccttgaaaatgggaatgaagatcacaatcaagaatatgatgcgattatgaagtggcttgacgagaagcctaattcatcagtggtgttcttatgctttggaagcaaggggtctttcgaagaagatcaggtgaaggaaatagcaaatgctctagagagcagtggctaccacttcttgtggtcgctaaggcgaccgccaccaaaagacaagctacaattcccaagcgaattcgagaatccagaggaagtcttaccagagggattctttcaaaggactaaaggaagaggaaaggtgataggatgggcaccccagttggctattttgtctcatccttcagtaggaggattcgtgtcgcattgtgggtggaattcaactctggagagcgttcgaagtggagtgccgatagcaacatggccattgtatgcagagcaacagagcaatgcatttcaactggtgaaggatttgggtatggcagtagagattaagatggattacagggaagattttaatacgagaaatccaccactggttaaagctgaggagatagaagatggaattaggaagctgatggattcagagaataaaatcagggctaaggtgacggagatgaaggacaaaagtagagcagcactgctggagggcggatcatcatatgtagctcttgggcattttgttgagactgtcatgaaaaactag

SEQ ID NO: 83 - protein, 478 aa, Nicotiana tabacum
MKTTELVFIPAPGMGHLVPTVEVAKQLVDRDEQLSITVLIMTLPLETNIPSYTKSLSSDYSSRITLLQLSQPETSVSMSSFNAINFFEYISSYKDRVKDAVNETFSSSSSVKLKGFVIDMFCTAMIDVANEFGIPSYVFYTSNAAMLGLQLHFQSLSIEYSPKVHNYLDPESEVAISTYINPIPVKCLPGIILDNDKSGTMFVNHARRFRETKGIMVNTFAELESHALKALSDDEKIPPIYPVGPILNLGDGNEDHNQEYDMIMKWLDEQPHSSVVFLCFGSKGSFEEDQVKEIANALERSGNRFLWSLRRPPPKDTLQFPSEFENPEEVLPVGFFQRTKGRGKVIGWAPQLAILSHPAVGGFVSHCGWNSTLESVRSGVPIATWPLYAEQQSNAFQLVKDLGMAVEIKMDYREDFNKTNPPLVKAEEIEDGIRKLMDSENKIRAKVMEMKDKSRAALLEGGSSYVALGHFVETVMKN

SEQ ID NO: 84 - DNA, 1437 bp, Nicotiana tabacum
atgaagacaacagagttagtattcattcctgctcctggcatgggtcaccttgtacccactgtggaggtggcaaagcaactagtcgacagagacgaacagctttcaatcacagttctcatcatgacgcttcctttggaaacaaatattccatcatatactaaatcactgtcctcagactacagttctcgtataacgctgcttcaactttctcaacctgagacctctgttagtatgagcagttttaatgccatcaatttttttgagtacatctccagctacaaggatcgtgtcaaagatgctgttaatgaaacctttagttcgtcaagttctgtgaaactcaaaggatttgtaatagacatgttctgcactgcgatgattgatgtggcgaacgagtttggaatcccaagttatgtcttctacacttctaatgcagctatgcttggactccaactccattttcaaagtcttagtattgaatacagtccgaaagttcataattacctagaccctgaatcagaagtagcgatctcaacttacattaatccgattccagtcaaatgtttgcccgggattatactagacaatgataaaagtggcaccatgttcgtcaatcatgcacgaagattcagggagacgaaaggaattatggtgaacacattcgctgagcttgaatcacacgctttgaaagccctttccgatgatgagaaaatcccaccaatctacccagttgggcctatacttaaccttggagatgggaatgaagatcacaatcaagaatatgatatgattatgaagtggctcgacgagcagcctcattcatcagtggtgttcctatgctttggaagcaagggatctttcgaagaagatcaagtgaaggaaatagcaaatgctctagagagaagtggtaaccggttcttgtggtcgctaagacgaccgccaccaaaagacacgctacaattcccaagcgaattcgagaatccagaggaagtcttgccggtgggattctttcaaaggactaaaggaagaggaaaggtgataggatgggcaccccagttggctattttgtctcatcctgcagtaggaggattcgtgtcgcattgtgggtggaattcaactttggagagtgttcgtagtggagtaccgatagcaacatggccattgtatgcagagcaacagagcaatgcatttcaactggtgaaggatttggggatggcagtggagattaagatggattacagggaagattttaataagacaaatccaccactggttaaagctgaggagatagaagatggaattaggaagctgatggattcagagaataaaatcagggctaaggtgatggagatgaaggacaaaagtagagcagcgttattagaaggcggatcatcatatgtagctctcgggcattttgttgagactgtcatgaaaaactaa

SEQ ID NO: 85 - protein, 482 aa, Nicotiana tabacum
MKETKKIELVFIPSPGIGHLVSTVEMAKLLIAREEQLSITVLIIQWPNDKKLDSYIQSVANFSSRLKFIRLPQDDSIMQLLKSNIFTTFIASHKPAVRDAVADILKSESNNTLAGIVIDLFCTSMIDVANEFELPTYVFYTSGAATLGLHYHIQNLRDEFNKDITKYKDEPEEKLSIATYLNPFPAKCLPSVALDKEGGSTMFLDLAKRFRETKGIMINTFLELESYALNSLSRDKNLPPIYPVGPVLNLNNVEGDNLGSSDQNTMKWLDDQPASSVVFLCFGSGGSFEKHQVKEIAYALESSGCRFLWSLRRPPTEDARFPSNYENLEEILPEGFLERTKGIGKVIGWAPQLAILSHKSTGGFVSHCGWNSTLESTYFGVPIATWPMYAEQQANAFQLVKDLRMGVEIKMDYRKDMKVMGKEVIVKAEEIEKAIREIMDSESEIRVKVKEMKEKSRAAQMEGGSSYTSIGGFIQIIMENSQ

SEQ ID NO: 86 - DNA, 1449 bp, Nicotiana tabacum
atgaaagaaaccaagaaaatagagttagtcttcattccttcaccaggaattggccatttagtatccacagttgaaatggcaaagcttcttatagctagagaagagcagctatctatcacagtcctcatcatccaatggcctaacgacaagaagctcgattcttatatccaatcagtcgccaatttcagctcgcgtttgaaattcattcgactccctcaggatgattccattatgcagctactcaaaagcaacattttcaccacgtttattgccagtcataagcctgcagttagagatgctgttgctgatattctcaagtcagaatcaaataatacgctagcaggtattgttatcgacttgttctgcacctcaatgatagacgtggccaatgagttcgagctaccaacctatgttttctacacgtctggtgcagcaacccttggtcttcattatcatatacagaatctcagggatgaatttaacaaagatattaccaagtacaaagacgaacctgaagaaaaactctctatagcaacatatctcaatccatttccagcaaaatgtttgccgtctgtagccttagacaaagaaggtggttcaacaatgtttcttgatctcgcaaaaaggtttcgagaaaccaaaggtattatgataaacacatttctagagctcgaatcctatgcattaaactcgctctcacgagacaagaatcttccacctatataccctgtcggaccagtattgaaccttaacaatgttgaaggtgacaacttaggttcatctgaccagaatactatgaaatggttagatgatcagcccgcttcatctgtagtgttcctttgttttggtagtggtggaagctttgaaaaacatcaagttaaggaaatagcctatgctctggagagcagtgggtgtcggtttttgtggtcgttaaggcgaccaccaaccgaagatgcaagatttccaagcaactatgaaaatcttgaagaaattttgccagaaggattcttggaaagaacaaaagggattggaaaagtgataggatgggcacctcagttggcgattttgtcacataaatcgacggggggatttgtgtcgcactgtggatggaattcgactttggaaagtacatattttggagtgccaatagcaacctggccaatgtacgcggagcaacaagcgaatgcatttcaattggttaaggatttgagaatgggagttgagattaagatggattataggaaggatatgaaagtgatgggcaaagaagttatagtgaaagctgaggagattgagaaagcaataagagaaattatggattccgagagtgaaattcgggtgaaggtgaaagagatgaaggagaagagcagagcagcacaaatggaaggtggctcttcttacacttctattggaggtttcatccaaattatcatggagaattctcaataa

SEQ ID NO: 87 - protein, 470 aa, Nicotiana tabacum
MVQPHVLLVTFPAQGHINPCLQFAKRLIRMGIEVTFATSVFAHRRMAKTTTSTLSKGLNFAAFSDGYDDGFKADEHDSQHYMSEIKSRGSKTLKDIILKSSDEGRPVTSLVYSLLLPWAAKVAREFHIPCALLWIQPATVLDIYYYYFNGYEDAIKGSTNDPNWCIQLPRLPLLKSQDLPSFLLSSSNEEKYSFALPTFKEQLDTLDVEENPKVLVNTFDALEPKELKAIEKYNLIGIGPLIPSTFLDGKDPLDSSFGGDLFQKSNDYIEWLNSKANSSVVYISFGSLLNLSKNQKEEIAKGLIEIKKPFLWVIRDQENGKGDEKEEKLSCMMELEKQGKIVPWCSQLEVLTHPSIGCFVSHCGWNSTLESLSSGVSVVAFPHWTDQGTNAKLIEDVWKTGVRLKKNEDGVVESEEIKRCIEMVMDGGEKGEEMRRNAQKWKELAREAVKEGGSSEMNLKAFVQEVGKGC

SEQ ID NO: 88 - DNA, 1413 bp, Nicotiana tabacum
atggtgcaaccccatgtcctcttggtgacttttccagcacaaggccatattaatccatgtctccaatttgccaagaggctaattagaatgggcattgaggtaacttttgccacgagcgttttcgcccatcgtcgtatggcaaaaactacgacttccactctatccaagggcttaaattttgcggcattctctgatgggtacgacgatggtttcaaggccgatgagcatgattctcaacattacatgtcggagataaaaagtcgcggttctaaaaccctaaaagatatcattttgaagagctcagacgagggacgtcctgtgacatccctcgtctattctcttttgcttccatgggctgcaaaggtagcgcgtgaatttcacataccgtgcgcgttactatggattcaaccagcaactgtgctagacatatattattattacttcaatggctatgaggatgccataaaaggtagcaccaatgatccaaattggtgtattcaattgcctaggcttccactactaaaaagccaagatcttccttcttttttactttcttctagtaatgaagaaaaatatagctttgctctaccaacatttaaagagcaacttgacacattagatgttgaagaaaatcctaaagtacttgtgaacacatttgatgcattagagccaaaggaactcaaagctattgaaaagtacaatttaattgggattggaccattgattccttcaacatttttggacggaaaagaccctttggattcttcctttggtggtgatctttttcaaaagtctaatgactatattgaatggttgaactcaaaggctaactcatctgtggtttatatctcatttgggagtctcttgaatttgtcaaaaaatcaaaaggaggagattgcaaaagggttgatagagattaaaaagccattcttgtgggtaataagagatcaagaaaatggtaagggagatgaaaaagaagagaaattaagttgtatgatggagttggaaaagcaagggaaaatagtaccatggtgttcacaacttgaagtcttaacacatccatctataggatgtttcgtgtcacattgtggatggaattcgactctggaaagtttatcgtcaggcgtgtcagtagtggcatttcctcattggacggatcaagggacaaatgctaaactaattgaagatgtttggaagacaggtgtaaggttgaaaaagaatgaagatggtgtggttgagagtgaagagataaaaaggtgcatagaaatggtaatggatggtggagagaaaggagaagaaatgagaagaaatgctcaaaaatggaaagaattggcaagggaagctgtaaaagaaggcggatcttcggaaatgaatctaaaagcttttgttcaagaagttggcaaaggttgctga

SEQ ID NO: 89 - protein, 545 aa, Cannabis
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVDVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 90 - DNA, 1437 bp, Nicotiana tabacum
atgaaaacaacagaacttgtcttcatacccgcccccggtatgggtcaccttgtacccacagtcgaagtcgccaaacaactagttgatagagacgaacagttgtctattaccgtcttgataatgacgttacccctggagactaatatcccaagttacaccaagagtttgtcctctgactattcatcccgtatcacgttgttacaactaagtcaacctgagacgagtgtctcaatgagtagttttaacgccataaacttcttcgaatacattagttcctataaggatcgtgttaaagatgccgtaaacgagacattctcctcttcatcctccgtcaaacttaaaggatttgtaatcgacatgttttgcacggcaatgatagacgtggccaacgagttcggtattccatcttatgtattctacacgtccaacgctgccatgctaggcctacaacttcacttccaatccttgtccatcgaatattcacctaaggttcataattatttagaccctgaatctgaggtagctatatcaacgtacattaacccaataccagtaaaatgcttacccggtataattcttgacaatgataagagtggcactatgttcgtaaaccatgccaggagattccgtgaaacaaagggtataatggtaaatacttttgcagaattagaaagtcacgccctaaaggcacttagtgacgatgagaaaattcctccaatctatcccgtcggacccattctaaacttgggtgatggtaatgaggatcataaccaagagtacgacatgataatgaaatggctggatgaacaaccacacagttcagtggttttcctgtgcttcggttccaaaggttcatttgaagaagaccaggttaaagagatagcaaatgctttagagagatcaggcaataggttcctgtggagtttaagacgtccccctcccaaggatactcttcaattcccttccgaatttgaaaaccccgaggaagtgctacctgtaggattttttcaaagaaccaaaggcagaggaaaagtcatcggatgggcaccacagcttgcaattctatctcaccctgccgtcggtggattcgtttcccactgcggctggaatagtactttggaatcagttagatcaggtgtacccatagcaacatggcctctttatgcagagcagcagtccaatgcatttcaattggtcaaggatctaggtatggccgtcgaaattaaaatggattaccgtgaggactttaacaagactaatcctccattggtaaaggcagaggaaatagaagacggcattaggaagttgatggactccgagaataagattagggcaaaggtgatggaaatgaaagataagtccagagctgcattactggaaggaggatcctcctatgttgcactgggtcacttcgtggagaccgtaatgaagaactaa

SEQ ID NO: 91 - protein, 478 aa, Nicotiana tabacum; identical to SEQ ID NO: 83

SEQ ID NO: 92 - DNA, 1413 bp, Nicotiana tabacum
atggttcaaccacacgtcttactggttacttttccagcacaaggccatatcaacccttgcctacaattcgccaaaagactaataaggatgggcatcgaagtaacttttgccacgagtgtattcgcacataggcgtatggctaaaactacgacatcaactttgtccaaaggactaaacttcgccgccttcagtgatggctatgacgatggattcaaagccgacgaacatgacagtcaacactacatgagtgaaataaagtcccgtggatctaaaacacttaaggatattatacttaaatcctccgatgagggaagacccgttacctctttagtttattcactgttactgccctgggctgcaaaagtcgccagagagtttcatattccttgcgctttattgtggatccaaccagctacggtattagacatctactattactacttcaatggatacgaggatgcaataaagggatcaacaaacgaccccaactggtgtattcaactgcctagacttcctctattaaaaagtcaggacttacctagttttttactgtcatccagtaacgaagaaaaatattcattcgctttacccaccttcaaagagcagcttgacactttggatgttgaagagaaccccaaggttttggtcaatacttttgacgctttggagccaaaagagctaaaggctattgaaaaatataaccttatcggcataggacctttaatcccctctactttcttagatggcaaagaccctctagattcaagtttcggaggtgatttgtttcaaaagagtaacgattatatcgagtggctaaatagtaaagccaactccagtgtggtctacatttctttcggaagtcttctgaatttatcaaaaaaccaaaaggaagagatcgcaaaaggactgatagagataaaaaaacctttcttatgggtgatcagagaccaggaaaacggtaaaggcgatgagaaggaggaaaaactgtcctgtatgatggagctagagaaacaaggaaaaatcgttccctggtgttcacagttagaagtgttaacccatccatccataggttgcttcgtatcacattgtggttggaatagtacacttgaaagtctttcatcaggcgtctctgtcgtcgcattcccccactggacggaccagggcacaaacgccaaactgatcgaagatgtatggaagacgggcgtcaggctaaaaaaaaatgaggatggcgtggtagagagtgaagagataaagcgttgcatagaaatggtcatggatggcggtgaaaagggagaggaaatgaggcgtaacgcacaaaagtggaaggaactagcccgtgaagcagtgaaagaaggaggttctagtgagatgaatttaaaagctttcgtgcaggaagttggaaaaggctgctga

SEQ ID NO: 93 - protein, 470 aa, Nicotiana tabacum; identical to SEQ ID NO: 87

SEQ ID NO: 94 - DNA, 1449 bp, Nicotiana tabacum
atgaaagagactaaaaaaattgagttagtttttatccccagtcctggtataggacacttagtctcaactgtggagatggccaaactgttgatagcccgtgaagagcaactttctattactgtcctgattatacaatggcctaatgataaaaagctagacagttatatccagtccgtcgcaaactttagttctagactgaagtttatacgtctgccccaagatgactcaatcatgcaacttttgaaatcaaacattttcacgacattcatcgcctctcacaagccagctgtaagagacgccgttgctgacatactaaagagtgaaagtaataacacattggcaggcattgtaatcgatcttttctgcacatccatgatcgatgtagccaatgagtttgagctgcctacttatgtgttttacactagtggcgcagccacgttgggtctgcactaccatattcaaaatctgcgtgatgagtttaataaagacattaccaaatataaggatgagccagaagaaaaattaagtatagccacgtaccttaacccattccctgctaagtgtctaccctccgtggcattggataaggaaggaggatcaacgatgttcctagacttagctaagaggttcagggagaccaaaggcataatgattaacacttttcttgagctggaatcatacgctctaaactcattgtctagagataaaaacttgccccctatataccctgtaggccctgttttgaacttgaacaacgttgagggtgataacttgggctctagtgatcaaaataccatgaaatggctggacgaccagccagcttcttccgttgtgttcctatgttttggctcaggaggaagtttcgaaaaacaccaagtcaaagaaatagcttatgccttagaatcttccggatgcaggttcttgtggagtttgcgtagaccccccacggaagatgctaggttcccttctaattacgaaaacttagaggaaattttaccagagggatttctggaaagaacgaaaggcattggtaaggtcattggatgggccccacagttagcaatcttgtctcacaagtccacaggaggattcgtgtctcattgcggatggaactctacccttgaaagtacctatttcggcgttcctattgctacttggccaatgtatgctgaacaacaggccaacgcttttcaacttgttaaagatttgaggatgggtgttgagatcaaaatggattataggaaggatatgaaggtaatgggcaaggaggttatcgttaaggcagaagaaattgaaaaggccataagggaaatcatggactcagaatcagaaatcagggtcaaggtcaaagagatgaaggagaaaagtcgtgcagcccaaatggaaggaggatcatcatatacctctatcggcggcttcattcaaataatcatggagaactcacagtaa

SEQ ID NO: 95 - protein, 482 aa, Nicotiana tabacum; identical to SEQ ID NO: 85

SEQ ID NO: 96 - DNA, 1491 bp, Nicotiana tabacum
atggctactcaggtgcataaattgcatttcattctgttcccactgatggctcccggtcacatgatccctatgatagacatcgcaaaactattggctaaccgtggcgtgataactaccataataactacgcccgttaacgccaatcgtttttcctctacgatcactagggccattaaatcaggcctaagaatccagattttaaccttaaaattcccatcagttgaggtaggcctgcctgaaggatgtgaaaacatcgacatgttgccatctttggacttagcctctaaattctttgctgctatttctatgcttaaacaacaagtggagaacttgctagagggtattaaccctagtccctcatgcgttatttctgacatgggcttcccatggacgacacagatcgctcaaaatttcaatattcctcgtatcgtatttcatggcacgtgttgcttttctcttctttgttcttacaaaatcctgtcatccaatatcttagagaacattactagtgactcagagtattttgtcgtgccagatctgccagaccgtgtcgagctaactaaggcccaagtctctggatctacaaagaatactacatcagtaagtagttcagtactgaaggaggttacagagcagatcaggcttgcagaggaatcatcctacggtgtgatagttaattccttcgaagaactggaacaggtgtatgaaaaagagtacagaaaagccaggggcaaaaaggtctggtgcgtgggtcctgtctctttgtgcaacaaggagattgaagatcttgttactagaggaaacaaaaccgctatagacaatcaggattgtcttaagtggttagacaacttcgagactgaatccgtcgtctatgcaagtttaggctcactaagtaggcttacgttactgcaaatggttgagctgggattgggactggaggagagtaataggccatttgtatgggttctgggaggaggagacaaactaaatgatcttgagaaatggatattggagaatggctttgaacagcgtataaaggagagaggtgtcctgatacgtggctgggcacctcaagtattgattttaagtcaccccgcaattggaggagttttaacgcattgtggatggaactctacattagagggcatttcagccggactacccatggtcacctggccactatttgccgaacagttctgtaacgaaaaattagtagtgcaggttcttaaaatcggtgtctcacttggagtgaaggtccctgttaagtggggtgacgaagagaacgtaggtgtcttagtgaaaaaggatgacgttaaaaaagcactggataagctaatggatgagggtgaggagggccaggttaggaggaccaaagccaaagagcttggtgagttagctaaaaaagcctttggagagggcggatcatcctacgtgaacctaacgtccctaattgaagatataatcgagcagcagaaccataaggagaagtag

SEQ ID NO: 97 - protein, 496 aa, Nicotiana tabacum; identical to SEQ ID NO: 79

SEQ ID NO: 98 - DNA, 1458 bp, Nicotiana tabacum
atgggctctatcggtgcagaactaaccaagccacacgccgtatgcattccctatcccgcccagggacacataaatcctatgctgaagttagctaagatactgcatcacaagggcttccatataaccttcgtaaatacggaatttaatcacaggcgtctgctgaagtccagaggtcctgactccctgaaaggtctttcaagtttcaggttcgagacgatacctgacggactgcccccatgcgaagctgacgctacacaggacattccttcactgtgtgaatccacgactaatacatgtctagctccttttagagacctacttgctaagctaaatgatacgaatacttctaacgtccctcccgtaagttgtattgtcagtgacggagtgatgtcatttacccttgcagctgcacaggaactgggtgtcccagaggttttattttggactacatctgcttgtggattcttaggttacatgcactattgcaaagtcattgaaaaaggatatgctccattaaaagacgcatcagacctgacgaatggctatcttgagacaaccttggacttcatccccggcatgaaggacgtcaggctgagagacttaccttcctttcttaggaccaccaatccagacgaatttatgattaagtttgtactacaggaaactgagcgtgctcgtaaggccagtgccataatacttaatacctttgaaaccttagaggcagaggtattagaatcattaaggaaccttctaccccccgtctatccaatcggccccttgcatttccttgtcaaacacgtagacgatgagaacctaaaaggtctacgttcctcactttggaaggaggaacctgaatgtattcaatggttagacaccaaagaacctaactctgtcgtgtacgtgaatttcggatccattactgtgatgactcccaatcaattaatagagttcgcttggggactggcaaactctcaacagaccttcctttggatcataaggcctgacatcgtaagtggtgatgcttccatattacctcccgagtttgttgaggagactaagaacagaggcatgcttgcctcctggtgctctcaggaggaggtactatcccatcccgcaatagtgggatttttgacgcactctggttggaactcaactttagaatcaatttctagtggcgtccccatgatctgttggcctttctttgctgagcagcaaacgaactgctggttttcagtgacgaagtgggacgttggaatggaaattgattcagatgtgaagagagatgaagtagagagtttagtaagagagttaatggtgggtggtaaaggcaagaagatgaagaagaaggcaatggagtggaaggaactggccgaggcttcagcaaaagaacactctggctcctcttacgtcaatatcgagaagttggttaacgatatattactatctagtaagcactaa

SEQ ID NO: 99 - protein, 485 aa, Nicotiana tabacum; identical to SEQ ID NO: 75

SEQ ID NO: 100 - DNA, 1377 bp, Stevia rebaudiana
atggagaacaaaaccgagacaaccgttaggcgtagacgtaggataatattgtttcccgtgccctttcaaggccatataaacccaatcctgcagctagccaacgtattgtactcaaagggcttcagtataacgatcttccacaccaactttaataagccaaaaacgtctaattatccacacttcacatttagatttatacttgataacgacccacaggatgaaagaatatcaaacttgcccacgcacggcccactagccggaatgagaataccaataatcaatgagcatggcgccgacgagttgcgtagagagctggaattgttgatgctagccagtgaggaagacgaagaggtgtcctgcttaataacggatgcactttggtattttgctcaatctgtggccgactcccttaacctgaggcgtcttgtccttatgacctccagtctattcaactttcatgcccatgtctcattgccccaatttgatgagcttggctatttggatcctgatgacaaaactaggctggaggaacaggcttccggttttcccatgctaaaggttaaggacatcaaatccgcctactcaaactggcagatccttaaggaaattcttggcaaaatgatcaaacagacgagggcatccagtggcgtcatctggaactcctttaaggaacttgaagaatcagaacttgaaacagtaatcagagaaatacctgccccaagtttcttgatccctctacctaagcaccttacggcttctagttcttctttgttggaccacgatcgtactgtctttcaatggttagatcagcaacccccctcatcagtgctatatgtgtcattcggtagtacatcagaagtggacgaaaaggatttccttgagatagcccgtggattggtggactctaaacagtcctttttatgggttgtgagacctggatttgtaaagggatccacgtgggtcgaacccttgcccgatggtttcctgggtgaaagaggaaggatagtgaagtgggtccctcagcaagaggtactggcccatggtgctataggtgctttctggacccactccggctggaatagtacactagaatccgtttgcgagggtgtccctatgattttttctgattttggtttagatcaacccctgaatgctaggtacatgtcagacgtccttaaagtcggcgtctacctagaaaatggctgggagaggggtgagatagcaaacgctatcagacgtgttatggtagacgaagagggagagtacataaggcaaaacgccagggtcctgaaacaaaaagccgatgtgtccttgatgaagggcggctcttcatacgaaagtctagaaagtcttgtttcttatatttcctcactataa

SEQ ID NO: 101 - protein, 458 aa, Stevia rebaudiana; identical to SEQ ID NO: 74

SEQ ID NO: 102 - DNA, 1434 bp, Lycium barbarum
atgggtcaattgcatttttttttgtttccaatgatggctcaaggtcatatgattccaactttggatatggctaagttgattgcttctagaggtgttaaggctactattattactactccattgaacgaatctgttttttctaaggctattcaaagaaacaagcaattgggtattgaaattgaaattgaaattagattgattaagtttccagctttggaaaacgatttgccagaagattgtgaaagattggatttgattccaactgaagctcatttgccaaacttttttaaggctgctgctatgatgcaagaaccattggaacaattgattcaagaatgtagaccagattgtttggtttctgatatgtttttgccatggactactgatactgctgctaagtttaacattccaagaattgtttttcatggtactaactactttgctttgtgtgttggtgattctatgagaagaaacaagccatttaagaacgtttcttctgattctgaaacttttgttgttccaaacttgccacatgaaattaagttgactagaactcaagtttctccatttgaacaatctgatgaagaatctgttatgtctagagttttgaaggaagttagagaatctgatttgaagtcttacggtgttatttttaactctttttacgaattggaaccagattacgttgaacattacactaaggttatgggtagaaagtcttgggctattggtccattgtctttgtgtaacagagatgttgaagataaggctgaaagaggtaagaagtcttctattgataagcatgaatgtttggaatggttggattctaagaagccatcttctattgtttacgtttgttttggttctgttgctaactttactgttactcaaatgagagaattggctttgggtttggaagcttctggtttggattttatttgggctgttagagctgataacgaagattggttgccagaaggttttgaagaaagaactaaggaaaagggtttgattattagaggttgggctccacaagttttgattttggatcatgaatctgttggtgcttttgttactcattgtggttggaactctactttggaaggtatttctgctggtgttccaatggttacttggccagtttttgctgaacaattttttaacgaaaagttggttactcaagttatgagaactggtgctggtgttggttctgttcaatggaagagatctgcttctgaaggtgttgaaaaggaagctattgctaaggctattaagagagttatggtttctgaagaagctgaaggttttagaaacagagctagagcttacaaggaaatggctagacaagctattgaagaaggtggttcttcttacactggtttgactactttgttggaagatatttcttcttacgaatctttgtcttctgattaa

SEQ ID NO: 103 - protein, 477 aa, Lycium barbarum
MGQLHFFLFPMMAQGHMIPTLDMAKLIASRGVKATIITTPLNESVFSKAIQRNKQLGIEIEIEIRLIKFPALENDLPEDCERLDLIPTEAHLPNFFKAAAMMQEPLEQLIQECRPDCLVSDMFLPWTTDTAAKFNIPRIVFHGTNYFALCVGDSMRRNKPFKNVSSDSETFVVPNLPHEIKLTRTQVSPFEQSDEESVMSRVLKEVRESDLKSYGVIFNSFYELEPDYVEHYTKVMGRKSWAIGPLSLCNRDVEDKAERGKKSSIDKHECLEWLDSKKPSSIVYVCFGSVANFTVTQMRELALGLEASGLDFIWAVRADNEDWLPEGFEERTKEKGLIIRGWAPQVLILDHESVGAFVTHCGWNSTLEGISAGVPMVTWPVFAEQFFNEKLVTQVMRTGAGVGSVQWKRSASEGVEKEAIAKAIKRVMVSEEAEGFRNRARAYKEMARQAIEEGGSSYTGLTTLLEDISSYESLSSD

SEQ ID NO: 104 - DNA, 1377 bp, Stevia rebaudiana
atggaaaataaaaccgaaaccaccgtccgccgtcgtcgccgtatcattctgttcccggtcccgttccagggccacatcaacccgattctgcaactggcgaacgtgctgtattcgaaaggtttcagcatcaccatcttccatacgaacttcaacaagccgaagaccagcaattacccgcactttacgttccgttttattctggataacgacccgcaggatgaacgcatctctaatctgccgacccacggcccgctggcgggtatgcgtattccgattatcaacgaacacggcgcagatgaactgcgtcgcgaactggaactgctgatgctggccagcgaagaagatgaagaagtttcttgcctgatcaccgacgcactgtggtattttgcccagtctgttgcagatagtctgaacctgcgtcgcctggtcctgatgaccagcagcctgttcaattttcatgcccacgttagtctgccgcagttcgatgaactgggttatctggacccggatgacaaaacccgcctggaagaacaggcgagcggctttccgatgctgaaagtcaaggatattaagtcagcgtactcgaactggcagattctgaaagaaatcctgggtaaaatgattaagcaaaccaaagcaagttccggcgtcatctggaatagtttcaaagaactggaagaatccgaactggaaacggtgattcgtgaaatcccggctccgagttttctgattccgctgccgaagcatctgaccgcgagcagcagcagcctgctggatcacgaccgcacggtgtttcagtggctggatcagcaaccgccgagttccgtgctgtatgttagcttcggtagtacctcggaagtggatgaaaaggactttctggaaatcgctcgtggcctggttgatagcaaacaatctttcctgtgggtggttcgcccgggttttgtgaagggctctacgtgggttgaaccgctgccggacggcttcctgggtgaacgtggccgcattgtcaaatgggtgccgcagcaagaagtgctggcgcatggcgcgattggcgcgttttggacccactccggttggaactcaacgctggaatcggtttgtgaaggtgtcccgatgattttctcagattttggcctggaccagccgctgaatgcacgttatatgtcggatgttctgaaagtcggtgtgtacctggaaaacggttgggaacgcggcgaaattgcgaatgccatccgtcgcgttatggtcgatgaagaaggcgaatacattcgtcagaatgctcgcgtcctgaaacaaaaggcggacgtgagcctgatgaaaggcggttcatcgtatgaaagtctggaatccctggtttcatacatcagctctctgtaa

SEQ ID NO: 105 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 106 - protein, 88 aa, S. cerevisiae
MRQVWFSWIVGLFLCFFNVSSAAPVNTTTEDETAQIPAEAVIGYSDLEGDFDVAVLPFSNSTNNGLLFINTTIASIAAKEEGVSLEKR

SEQ ID NO: 107 - protein, 22 aa, S. cerevisiae
MRQVWFSWIVGLFLCFFNVSSA

SEQ ID NO: 108 - protein, 22 aa, E. Coli
MEFFKKTALAALVMGFSGAALA

SEQ ID NO: 109 - protein, 36 aa, E. Coli
MKLSRRSFMKANAVAAAAAAAGLSVPGVARAVVGQQ

SEQ ID NO: 110 - protein, 25 aa, Arabidopsis thalinia
MSSSFLSSTAFFLLLCLGFCHVSSS

SEQ ID NO: 111 - protein, 23 aa, barley (Hordeum vulgare)
MGKKSHICCFSLLLLLFAGLASG

SEQ ID NO: 112 - protein, 28 aa, rice
MKNTSSLCLLLLVVLCSLTCNSGQAAQV

SEQ ID NO: 113 - protein, 160 aa, Mus musculus
MMVKFLLLALVFGLAHVHAHDHPELQGQWKTTAIMADNIDKIETSGPLELFVREITCDEGCQKMKVTFYVKQNGQCSLTTVTGYKQEDGKTFKNQYEGENNYKLLKATSENLVFYDENVDRASRKTKLLYILGKGEALTHEQKERLTELATQKGIPAGNL

SEQ ID NO: 114 - protein, 160 aa, Rattus norvegicus
MKSRLLTVLLLGLMAVLKAQEAPPDDQEDFSGKWYTKATVCDRNHTDGKRPMKVFPMTVTALEGGDLEVRITFRGKGHCHLRRITMHKTDEPGKYTTFKGKKTFYTKEIPVKDHYIFYIKGQRHGKSYLKGKLVGRDSKDNPEAMEEFKKFVKSKGFREE

SEQ ID NO: 115 - protein, 160 aa, Mus musculus
MAKFLLLALAFGLAHAAMEGPWKTVAIAADRVDKIERGGELRIYCRSLTCEKECKEMKVTFYVLENGQCSLTTITGYLQEDGKTCKTQYQGDNHYELVKETPENLVFYSENVDRADRKTKLIFVLGNKPLTSEENERLVKYAVSSHIPPENIRHVLGTDT

SEQ ID NO: 116 - protein, 160 aa, Cricetulus griseus
MEKFLLLALAVSLAHALSELEGDWVSTAIDADNVAKIANQGTLRLYFHKMTCLEGYDKLEITFYVNLSGQCSKTTVVVYKQEDGNYRTQYEGDTIFKPMIITKEILVFTNENVDRDSLETHLIFVAGKGDHLTHEQYGRLEEHAKEQKIPSESIRKLLVS

SEQ ID NO: 117 - protein, 138 aa, Peromyscus maniculatus bairdii
MVKFLLLALALGVSCAHHNNPEITPSEVDGNWRTLYIGADNVEKVLKGGPLRAYFQHMECSDECQTLTITFKVKVEGECQTHTVVGRKEKDGLYMTDYSGKNYFRVIEKADGIIIFHNVNVDNSGKETNVILVAAVLS

SEQ ID NO: 118 - protein, 138 aa, Echinops telfairi
MQTLVLTMLSLIGTLQAQEPLSFAMEEATITGTWYIKAMVSNKDRDVRERTLSRSPLIVTALDHGDLEISITFLKNGQCREKKILMENTGEPGKFSAFGSKKQITFLELPGKDHIIVFCEGERNGKSLRKAKLLGEQL

SEQ ID NO: 119 - protein, 139 aa, Equus przewalskii
MVLSSSVSWVQDQLGHLDYGAVSRAKAAEKLKRSRMFPNVSNIFCSNEDTKYQFSLCLSADGGKRHVYILDLPVKDHHIFYCEGQLGGKAIRMAKLVGINPDMSLEALEEFKKFTERKGLPQDIIIMPVQTESCIPESD

SEQ ID NO: 120 - protein, 116 aa, Chrysochloris asiatica
MQYTSNNEILSFGFYFKYDGECLPRYEYTKRQTGNYFTGIGPLNNTFKPVYVTEDVMIGLYINVSVQGVTSYIMQLLAKENSVSQEVFDMYMDYTRQVGIPEENLIDIIKRERTGI

SEQ ID NO: 121 - protein, 134 aa, Mus caroli
MVKFLLLELAFGLAHAQMYGPWKTIAIAADNVDKMEISGELRLYFHQITCEKECKKMNVTFYVDENGQCSLTTITGYLQDDGKTYRSQFQGDNHYATVRTTPENIVFYSENVDRAGRKTKLVYVVGKNGSGSLK

SEQ ID NO: 122 - protein, 160 aa, Fukomys damarensis
MRILLLALAVGFACADSQINPARINGEWRSIAEAADNVEKIQEGGPLRAYLRSLNCFQGCRKLSVNFYVKLNEDWREFSVLSEKRPSDGVYTAVYSGQNFFNISSPDDGITVFSSTNVDENGRRTRLLLLGARKDSLTQAEESKFRQLAVENGIPEENIV

SEQ ID NO: 123 - protein, 160 aa, Urocitellus parryii
MGESGRGQGDSCLDLLQITGTWYPKAFVVNMPSVPDWKGPRKVFPVTVTALEDGSWEAKTTLLIKGRCLEKKVTLQKTEEPGRYSASTDHGKKLVYIEELPESHHCIFYCESQGPGKKFRMGKLMGRSPEENLEALEEFRKFTQRKGLLAETIFTPEQTD

SEQ ID NO: 124 - protein, 160 aa, Bubalus bubalis
MKVLLLSAVLGMLYAGHGEAQLLLKPFSGKWKTHYIAASNKDKITEGGPFHVYVRHVEFHANNTVDIDFYVKSDGECVKKQVTGVKQKFFVYQVEYAGQNEGRILHLSRDAIIVSIHNVDEEGKETVFVAIISMEPAISEMWSIDVHQDSVHCIPYRLLY

SEQ ID NO: 125 - protein, 160 aa, Ursus arctos horribilis
MKILLLSLVLAVVCDAQLPLIHQLTQLPGQWETMYLAASNPDKISDNGPFKGYMRRIEVDMARRQISFHFYAKINGQCTEKSVVGGIGTNNAITVDYEGTNDFQIIDMTPNSIIGYDVNVDEEGNTTDIVLLFGRGAQADEKAVEKFKQFTRQRNIPEEN

SEQ ID NO: 126 - protein, 160 aa, Enhydra lutris kenyoni
MKVLLLSLVLVAVCDAQLSLRNALIQLPGQWKTIHLAANNAEKLSENSPFRAYVRHVDVDMTRRKIFFNFFIKVNGECIEKSVMGTVGLYNVIHVDYEGTNNFQVVRITPNIMLAYDINVDEEGRTTDLVILAGRTHEVDEESIEKFKELVRQRNIPEEN

SEQ ID NO: 127 - protein, 160 aa, Peromyscus maniculatus bairdii
MKNLLIFLLLGLVAVLKAQEVPSDDQEELSGTWHIKALVCDKNHTEREGPKKVFPMTVTALEGGDLEVEITFWKKGQCHKKKIVMHKTDEPGKYTAFKGKKVIYIQELSVKDHYIFYCEGQHHGKSRRMGKLVGRNPEENPEALEEFKKFAQGKGLRQEN

SEQ ID NO: 128 - protein, 160 aa, Ceratotherium simum simum
MKILLLTLVLGLVCAAQEPQSETNFSLVSGEWKTLYVASSNIEKISENGPFRAFVRRLDFDSEGDTIAFTFLVKVNGQCTIIHSVATKIEGNVYISDYAGINGFKILDLSENAIIGYILNVDEEGLVTKIIALLGKGNDINEEDIEKFKELTRQRGIPEE

SEQ ID NO: 129 - protein, 160 aa, Chrysochloris asiatica
MKTLLVTLVLGIICAAQDSLLQDPCTQVTGPWRTTYTASDNKEAIEENHPMRVYFRYMQCMSLGLAIRVDFYSKENDQCILQHQLGLKTSENFYTTNYAGMVDFTILYYSDRFMVMYGINTNNGKTSKVIGAITQNDDISDAEYQIFLSLTKAKEIPEDS

SEQ ID NO: 130 - protein, 160 aa, Bos Taurus
MKALLLSLVLGLLAASQGDVIDASQFTGRWLTHFIAAENIDKITEGAPFHIFMRYIEFDEENGTIHFHFYIKKNGECIEKYVSGLKEENFYAVDYSGHNEFQVISGDKNTLITHNLNVDEDGRETEMVGLFGLSDVVDPNRIEEFKNVVREKGIPEENIR

SEQ ID NO: 131 - protein, 160 aa, Bubalus bubalis
MKVLLLSAVLGLLYAGHGEAQLLLKPFSGKWKTHYIAASNKDKITEGGPFHVYVRHVEFHANNTVDINFYVKSDGECVKKQVTGVKQKFFVYQVEYAGQNEVRILHLSPDTIIVSIHNVDEEGKETVFVAIIGKRDRISNLDNWKFKKETEDRGIPEENI

SEQ ID NO: 132 - protein, 160 aa, Bos Taurus
MKILFLSLVLLVVCAAQETPAEIDPSKVVGEWRTIYAAADNKEKIVEGGPLRCYNRHIECINNCEQLSLSFYIKFDGTCQFFSGVLQRQEGGVYFIEFEGKIYLQIIHVTDNILVFYYENDDGEKITKVTEGSAKGTSFTPEEFQKYQQLNNERGIPNEN

SEQ ID NO: 133 - protein, 158 aa, Mus Pahari
MVKFLLLALAFGLAHAEFEGAWESVAIAADRVDKIERGGELRLYCRSLTCENGCKEMKVTFYVLENGQCSLTTITGYLQEDGRTYKTQFQGDNHYELVKETPENLVFYSENVDRAGRTTKLLFVLGHESLTPEQKEVFAELAEEKGIPPENIRDVLVT

SEQ ID NO: 134 - protein, 156 aa, Dasypus novemcinctus
MPLALPQLTGTWYIKALVDTKEIPVEQRPDKVSPQTITALEGGNMAVTFTVMLQPTCLVLSGKKGQCHEMNVLLEKTEEPGKYRAFNGTNLVQGEELPVKDHYAFIMEGQHRGRPFHMGKLIGRNLDVNFEALEEFKKFAQSKGFLQENIFIPAQM

SEQ ID NO: 135 - protein, 160 aa, Mus caroli
MAKFLLLALAFGLAHAALEGPKKTVAIAADRVDKIEESGELRLFCRRIVCEEECKKLIVTFYVLENGQCSLTTITGYLQEDGKTYKTQYQGNNHFKLVKETPENVVFYSENVDRADWKTKLIFVLGNKPLTSEENERLVKYAVSSHIPPENIQHVLGTDT

SEQ ID NO: 136 - protein, 160 aa, Microtus ochrogaster
MVKFLLLTLAFGLAHAYTELEGAWFTTAIAADNVDTIEEEGPMRLYVRELTCSEACNEMDVTFYVNANGQCSETTVTGYRQEDGKYRTQFEGDNRFEPVYATSENIVFTNKNVDRTGRTTNQIFVVGKGQPLTPEQYEKLEEFAKQQNIPKENIRQVLDA

SEQ ID NO: 137 - protein, 160 aa, Mus Pahari
MVKFLLLALAFGLAHAEFEGAWETVAIAADRVDKIEPSGELRLFCRSLDCEDGCKILKVTFYVLENGQCSLTTVTGYLQEDGKTYKTQFQGDNHYELVKETPENLVFYSENVDRAGRTTKLIFVLGHKPLSSEQNERLVSYAKSSHIPPENIRDVLGADT

SEQ ID NO: 138 - protein, 160 aa, Fukomys damarensis
STNLPSVNLPLQIDGNWRSMYLAADNVEKIEEGGELRNYVRQIECQDECRNISVRFYAKKNGVCQEFTVVGVRDEASGDYFTEYLGENYFSIEYNTENIIIFHSTNVDEAGTTTNVILATGKSALLKVQELQKFARVVQDYGIPKQNIRPVILTGRVTTL

SEQ ID NO: 139 - protein, 160 aa, Ochotona princeps
MKALALTVALGLLAALQAQDPLALLLPEGQNITGTWYVKAVVGSKALPEGMRPKKLFPLTVTALDDGSLEATIVFEKHGQCFEKKFVMRQTEQPGEYIALDGKKRTCVEGLSTSDHYVFFCEKQRLGRVFRMAKLMGRSPDPAPQATLEEFKELVQHKGF

SEQ ID NO: 140 - protein, 160 aa, Cricetulus griseus
MTSSYVYEQHIPGFYLLRSRQGKDSTCSMKIPSKLITQFYLLQKIKAGTTIAKILLLALAVCLAHALNELEGDWVSIAIAADNVEKIENQGTMRLYARQITCNEECDNLEITFYANLNGQCSETTVIGYKQEDGSYRTQYEGDNVFKAVVITKDFLVFSS

SEQ ID NO: 141 - protein, 160 aa, Capra hircus
MQANKMKVLFLTLVLGLVCSSQEIPAEPHHSQISGEWRTHYIASSNTDKTGENGPFNVYLRSIKFNDKGDSLVFHFFVKNNGECTESSVSGRRIANNVYVAEYAGANQFHFILVSDDGLIVNTENVDDEGNRTRLIGLLGKEDEVDDHDLERFLEEVRKL

SEQ ID NO: 142 - protein, 160 aa, Microtus ochrogaster
MKRLLLTLILLGLVAVLKAQEFPSDDKEDYSGTWYPKAMIHNGSLPSHNIPSKFFPVKMTALEGGDLEAEVIFWKNGQCHNVKILMKKTDEPGKFTSFDNKRFIYITALLVKDHYIMYCEGRLPGKLFGVGKLVGRNPEENPEAMEEFKKFVQRKGLKVE

SEQ ID NO: 143 - protein, 160 aa, Bubalus bubalis
MKALLLPIALSLLAALRAQDPPSCPLEPQQIAGTWYVKAMVTDENLPKETRPRKVSPVTVTALGGGNLELMFTFLKEARCHEKRTRVQPTGEPGKYSSNGGKKQMHILELPVEGHYILYCEGQRQGKSVHVGKLIGRNPDMNPEALEAFKKFVQRKGLSP

SEQ ID NO: 144 - protein, 160 aa, Meriones unguiculatus
MKSLLLTVLLLGLVAVLKAQEDLPDDKEDFSGTWYTNAMVCDKDHTNGKKPKKVYLMTVTALEGGDLEITITFQKNGQCHEKKIVIHKTDDPHKFTAFGGKKVIQIQATSQKDHYILYCEGKHKGKLHRKAKLLGRKPEKSPEAMREFMEFVESKKLKTQ

SEQ ID NO: 145 - protein, 160 aa, Meriones unguiculatus
MKSLLLTVLLLGLVAVLKAQEDLPDDKEDLSGTWYMKGMVHNGTLPKNKLPERVFPVTITALEEGNLEVKIIKWKKGQCHEFKFKMEKTEEPNKYITFHGKRHVYIEKLNTKDHYIFYCEGHYKGKHFGMGKVMGRTSEESPEAMEEFKEFVKRKKIPQE

SEQ ID NO: 146 - protein, 160 aa, Marmota marmota marmot
MKSLFLTILLLDLLSALQAQDLLTFPSEELNITGTWYTKAFVVNMPLVPDWKGPGKVFPVTVTALEDGSWEAKTTLLIQGRCLEKKVTLQKTEEPGRYSASTDHGKKFVYIEELPESDHCIFYCESQDPGKKFRMGKLMGRSPEENLEALEEFRKFTQRK

SEQ ID NO: 147 - protein, 152 aa, Heterocephalus glaber
MKTLLLTPVLLALVAALRAKDALSLQPEEPDITGTRYMKAIVTNGNLTHGPRQAFPVTVMAWEGVNFETRITFMWRGGCYKDRLHLQKTTEPGKYTFWNHTHIHTEELAVKDHSACYAEHQLPLGETMHVGYLMGEDPGDPSPGPAVSLWRS

SEQ ID NO: 148 - protein, 155 aa, Heterocephalus glaber
MINGDWCSIYIAADNVEKIEERGELRAYFCHIECQDECRNLSGGDRIMRNKHCCVGLSFRLDGVCQEFTVVGVKDEKSGVYITDYVGKNYFTVVESTEYITLFSNIIVDEKGTKMNVVLVAAKRDSLTEKEKQKFAQLAEEKGIPTENIRNVIAT